SEQUENCE LISTING (67 sequences)

SEQ ID NO: 1 - protein, 147 aa, Homo sapiens
MVMGLGVLLLVFVLGLGLTPPTLAQDNSRYTHFLTQHYDAKPQGRDDRYCESIMRRRGLTSPCKDINTFIHGNKRSIKAICENKNGNPHRENLRISKSSFQVTTCKLHGGSPWPPCQYRATAGFRNVVVACENGLPVHLDQSIFRRP

SEQ ID NO: 2 - DNA, 20 bp, Artificial Sequence
  Description of Artificial Sequence Synthetic primer
agcgaatggaagcccttaca

SEQ ID NO: 3 - DNA, 20 bp, Artificial Sequence
  Description of Artificial Sequence Synthetic primer
ctcatcgaagtggacaggca

SEQ ID NO: 4 - DNA, 23 bp, Artificial Sequence
  Description of Artificial Sequence Synthetic primer
agggtggaacttcaggattcaag

SEQ ID NO: 5 - DNA, 21 bp, Artificial Sequence
  Description of Artificial Sequence Synthetic primer
gaagttatccgcgggaagttc

SEQ ID NO: 6 - DNA, 28 bp, Artificial Sequence
  Description of Artificial Sequence Synthetic probe
ggcgagaattctaccactgaaccaccaa

SEQ ID NO: 7 - RNA, 33 nt, Artificial Sequence
  Description of Artificial Sequence Synthetic oligonucleotide
auuggugguucagugguagaauucucgccugcc

SEQ ID NO: 8 - DNA, 21 bp, Artificial Sequence
  Description of Artificial Sequence Synthetic primer
tggagtcaggcgcagatccac

SEQ ID NO: 9 - DNA, 22 bp, Artificial Sequence
  Description of Artificial Sequence Synthetic primer
aggcaaactctgaggaccggca

SEQ ID NO: 10 - DNA, 20 bp, Artificial Sequence
  Description of Artificial Sequence Synthetic primer
cgaggagcaggacgagaatc

SEQ ID NO: 11 - DNA, 20 bp, Artificial Sequence
  Description of Artificial Sequence Synthetic primer
ggtatcctccgacccaccac

SEQ ID NO: 12 - DNA, 20 bp, Artificial Sequence
  Description of Artificial Sequence Synthetic primer
ggcgaggatggagttcgaca

SEQ ID NO: 13 - DNA, 21 bp, Artificial Sequence
  Description of Artificial Sequence Synthetic primer
aaaccagaccactcctgaaca

SEQ ID NO: 14 - DNA, 24 bp, Artificial Sequence
  Description of Artificial Sequence Synthetic primer
gcgtaccctgacaccaatctcctc

SEQ ID NO: 15 - DNA, 20 bp, Artificial Sequence
  Description of Artificial Sequence Synthetic primer
tcccgactacttcactcctg

SEQ ID NO: 16 - DNA, 20 bp, Artificial Sequence
  Description of Artificial Sequence Synthetic primer
cgcgacctcagatcagacgt

SEQ ID NO: 17 - DNA, 20 bp, Artificial Sequence
  Description of Artificial Sequence Synthetic primer
gcttgtttctcccgattgcg

SEQ ID NO: 18 - DNA, 20 bp, Artificial Sequence
  Description of Artificial Sequence Synthetic primer
gcgcacttttctcaagtggt

SEQ ID NO: 19 - DNA, 20 bp, Artificial Sequence
  Description of Artificial Sequence Synthetic primer
ccctggctgagcactacctt

SEQ ID NO: 20 - DNA, 20 bp, Artificial Sequence
  Description of Artificial Sequence Synthetic primer
tgggatgcctttgtggaact

SEQ ID NO: 21 - DNA, 21 bp, Artificial Sequence
  Description of Artificial Sequence Synthetic primer
ggctgggacacttttgtggat

SEQ ID NO: 22 - DNA, 20 bp, Artificial Sequence
  Description of Artificial Sequence Synthetic primer
ccctcccccatcctaatcag

SEQ ID NO: 23 - DNA, 20 bp, Artificial Sequence
  Description of Artificial Sequence Synthetic primer
aatggcatctggacaaggac

SEQ ID NO: 24 - DNA, 21 bp, Artificial Sequence
  Description of Artificial Sequence Synthetic primer
tggagctgcagaggatgattg

SEQ ID NO: 25 - DNA, 22 bp, Artificial Sequence
  Description of Artificial Sequence Synthetic primer
gaagacgagctgcagacagatg

SEQ ID NO: 26 - DNA, 19 bp, Artificial Sequence
  Description of Artificial Sequence Synthetic primer
ttggagctctgcggtcctt

SEQ ID NO: 27 - DNA, 20 bp, Artificial Sequence
  Description of Artificial Sequence Synthetic primer
ggagtgcaccggacataact

SEQ ID NO: 28 - DNA, 18 bp, Artificial Sequence
  Description of Artificial Sequence Synthetic primer
gcggcggagacaagaaga

SEQ ID NO: 29 - DNA, 22 bp, Artificial Sequence
  Description of Artificial Sequence Synthetic primer
gacggccaggtcatcactattg

SEQ ID NO: 30 - DNA, 21 bp, Artificial Sequence
  Description of Artificial Sequence Synthetic primer
cgccatgagcgcatcgcaatc

SEQ ID NO: 31 - DNA, 21 bp, Artificial Sequence
  Description of Artificial Sequence Synthetic primer
tgctccacagtgccagcgttc

SEQ ID NO: 32 - DNA, 21 bp, Artificial Sequence
  Description of Artificial Sequence Synthetic primer
gaagaagtcgttcgcattggc

SEQ ID NO: 33 - DNA, 19 bp, Artificial Sequence
  Description of Artificial Sequence Synthetic primer
ccagccagggcagagatcc

SEQ ID NO: 34 - DNA, 20 bp, Artificial Sequence
  Description of Artificial Sequence Synthetic primer
tgacagggctgatggtctgg

SEQ ID NO: 35 - DNA, 26 bp, Artificial Sequence
  Description of Artificial Sequence Synthetic primer
tcttcttctcttcatctcatttttga

SEQ ID NO: 36 - DNA, 24 bp, Artificial Sequence
  Description of Artificial Sequence Synthetic primer
acctcctcttcgcacttctgctcc

SEQ ID NO: 37 - DNA, 21 bp, Artificial Sequence
  Description of Artificial Sequence Synthetic primer
caagagaacacaacgagcgac

SEQ ID NO: 38 - DNA, 20 bp, Artificial Sequence
  Description of Artificial Sequence Synthetic primer
gctcttccctgttcactcgc

SEQ ID NO: 39 - DNA, 20 bp, Artificial Sequence
  Description of Artificial Sequence Synthetic primer
acacatccacaaggaccacg

SEQ ID NO: 40 - DNA, 20 bp, Artificial Sequence
  Description of Artificial Sequence Synthetic primer
tgaaacacgtgagggcacaa

SEQ ID NO: 41 - DNA, 18 bp, Artificial Sequence
  Description of Artificial Sequence Synthetic primer
ctgcatgcttggcttgga

SEQ ID NO: 42 - DNA, 22 bp, Artificial Sequence
  Description of Artificial Sequence Synthetic primer
acagccaggagaaatcaaacag

SEQ ID NO: 43 - DNA, 17 bp, Artificial Sequence
  Description of Artificial Sequence Synthetic primer
gcgctcctggcctttcc

SEQ ID NO: 44 - DNA, 24 bp, Artificial Sequence
  Description of Artificial Sequence Synthetic primer
agtaacaatggaaagcatgccaat

SEQ ID NO: 45 - DNA, 20 bp, Artificial Sequence
  Description of Artificial Sequence Synthetic primer
gttcctgctggtggaggtaa

SEQ ID NO: 46 - DNA, 17 bp, Artificial Sequence
  Description of Artificial Sequence Synthetic primer
agctgccacccggaaga

SEQ ID NO: 47 - DNA, 25 bp, Artificial Sequence
  Description of Artificial Sequence Synthetic primer
aatctggctctattcttccttggtt

SEQ ID NO: 48 - DNA, 19 bp, Artificial Sequence
  Description of Artificial Sequence Synthetic primer
cagcggaggtggtgtgaat

SEQ ID NO: 49 - DNA, 20 bp, Artificial Sequence
  Description of Artificial Sequence Synthetic primer
ttgagcacactcgtccttca

SEQ ID NO: 50 - DNA, 26 bp, Artificial Sequence
  Description of Artificial Sequence Synthetic primer
agtcccatgaagagattgtacatgac

SEQ ID NO: 51 - DNA, 20 bp, Artificial Sequence
  Description of Artificial Sequence Synthetic primer
aggaaggctggaaaagagcc

SEQ ID NO: 52 - DNA, 22 bp, Artificial Sequence
  Description of Artificial Sequence Synthetic primer
gtcactgtcttgtacccttgtg

SEQ ID NO: 53 - DNA, 25 bp, Artificial Sequence
  Description of Artificial Sequence Synthetic primer
tgcaaccgacgattcttctactcaa

SEQ ID NO: 54 - DNA, 20 bp, Artificial Sequence
  Description of Artificial Sequence Synthetic primer
agagatcagcgcctgagaag

SEQ ID NO: 55 - DNA, 22 bp, Artificial Sequence
  Description of Artificial Sequence Synthetic primer
accacaaccacactctggagga

SEQ ID NO: 56 - DNA, 20 bp, Artificial Sequence
  Description of Artificial Sequence Synthetic primer
cggcttgcaccattcagcta

SEQ ID NO: 57 - DNA, 20 bp, Artificial Sequence
  Description of Artificial Sequence Synthetic primer
aatccccacctgatgtgtgt

SEQ ID NO: 58 - DNA, 25 bp, Artificial Sequence
  Description of Artificial Sequence Synthetic primer
agctcctgtgctgcgaagtggaaac

SEQ ID NO: 59 - DNA, 20 bp, Artificial Sequence
  Description of Artificial Sequence Synthetic primer
agcgagcatcccccaaagtt

SEQ ID NO: 60 - DNA, 21 bp, Artificial Sequence
  Description of Artificial Sequence Synthetic primer
cggcgtttggagtggtagaaa

SEQ ID NO: 61 - DNA, 29 bp, Artificial Sequence
  Description of Artificial Sequence Synthetic primer
caagcagtgatgtatctgataaacaagga

SEQ ID NO: 62 - DNA, 20 bp, Artificial Sequence
  Description of Artificial Sequence Synthetic primer
gggctctttgggctctaaac

SEQ ID NO: 63 - DNA, 22 bp, Artificial Sequence
  Description of Artificial Sequence Synthetic primer
tcggtttctggtctggatgcct

SEQ ID NO: 64 - DNA, 25 bp, Artificial Sequence
  Description of Artificial Sequence Synthetic primer
tgcagaagtgagtatcacagccatc

SEQ ID NO: 65 - DNA, 20 bp, Artificial Sequence
  Description of Artificial Sequence Synthetic primer
gctggtctccaggtaacgaa

SEQ ID NO: 66 - DNA, 25 bp, Artificial Sequence
  Description of Artificial Sequence Synthetic primer
agtgttcaatgaaatcgtgcggggt

SEQ ID NO: 67 - DNA, 20 bp, Artificial Sequence
  Description of Artificial Sequence Synthetic primer
gggcacgaaggctcatcatt

(19) United States
(12) Patent Application Publication
(10) Pub. No.: US 2019/0000885 A1
(43) Pub. Date: Jan. 3, 2019

(54) TREATMENT WITH ANGIOGENIN TO ENHANCE HEMATOPOIETIC RECONSTITUTION

(71) Applicants: THE GENERAL HOSPITAL CORPORATION, Boston, MA (US); TUFTS MEDICAL CENTER, INC., Boston, MA (US); TRUSTEES OF TUFTS COLLEGE, Medford, MA (US); PRESIDENT AND FELLOWS OF HARVARD COLLEGE, Cambridge, MA (US)

(72) Inventors: David SCADDEN, Boston, MA (US); Peter KHARCHENKO, Brookline, MA (US); Lev SILBERSTEIN, Brookline, MA (US); Guo-fu HU, Wellesley, MA (US); Kevin GONCALVES, Southport, CT (US)

(73) Assignees: THE GENERAL HOSPITAL CORPORATION, Boston, MA (US); TUFTS MEDICAL CENTER, INC., Boston, MA (US); TRUSTEES OF TUFTS COLLEGE, Medford, MA (US); PRESIDENT AND FELLOWS OF HARVARD COLLEGE, Cambridge, MA (US)

(21) Appl. No.: 15/779,935

(22) PCT Filed: Nov. 29, 2016
(86) PCT No.: PCT/US16/63941

§ 371 Date: May 30, 2018

Related U.S. Application Data
(60) Provisional application No. 62/260,838, filed on Nov. 30, 2015.
(60) Provisional application No. 62/315,281, filed on Mar. 30, 2016.

(51) Int. Cl. A61K 35/28; C12N 5/0789; A61K 38/18; A61N 5/10

(52) U.S. Cl. CPC A61K 35/28; C12N 5/0647; A61K 38/1891; A61N 2005/1098; C12N 2501/10; C12N 2501/73; A61N 5/10

(57) ABSTRACT

Aspects of the technology disclosed herein generally (and in part) relates to use of Angiogenin (ANG) for increasing hematopoietic reconstitution of in vivo hematopoietic cells and transplanted hematopoietic cells. Provided herein are methods and compositions useful in treatment of diseases characterized by decreased levels of hematopoietic cells, decreased levels of hematopoietic reconstitution, blood cell deficiency and prevention and treatment of radiation injury. One aspect relates to angiogenin treated hematopoietic cell compositions and methods of their use in stem cell transplantation. Treatment of hematopoietic cells with angiogenin enhances quiescence and reduces proliferative capacity of primitive hematopoietic stem cells while increasing proliferation of myeloid restricted progenitor cells. Another aspect relates to use of ANG in prophylactic and therapeutic treatment methods for radiation injury.

### CROSS-REFERENCE TO RELATED APPLICATION

This application claims benefit under 35 U.S.C. § 119(e) of U.S. Provisional Application Nos. 62/260,838, filed Nov. 30, 2015 and 62/315,281, filed Mar. 30, 2016, the contents of which are incorporated herein by reference in their entireties.

### GOVERNMENT SUPPORT

This invention was made with government support under Grant No. R01DK050234, R01DK050234, R01DK050234, R01DK050234, R01DK050234, R01HL097794, R01CA105241, R01NS065237 and F31HL128127 awarded by the National Institutes of Health (NIH). The government has certain rights in the invention.

### TECHNICAL FIELD

The technology described herein relates to use of Angiogenin in methods and compositions for enhancing hematopoietic reconstitution, and for prevention and treatment of radiation injury.

### SEQUENCE LISTING

The instant application contains a Sequence Listing which has been submitted electronically in ASCII format and is hereby incorporated by reference in its entirety. Said ASCII copy, created on Nov. 11, 2016, is named 030258-086192-PCT_SL.txt and is 16,675 bytes in size.

### BACKGROUND

Hematopoietic stem cells possess the ability of both “multi-potency” and “self-renewal”. Multi-potency is the ability to differentiate into all functional blood cells and self-renewal is the ability to give rise to HSCs itself without differentiation. Since mature blood cells are predominantly short lived, HSCs continuously provide more differentiated progenitors while maintaining the HSCs pool size throughout life by precisely balancing self-renewal and differentiation.

Hematopoietic stem cell transplantation (HSCT) or bone marrow transplantation is a procedure to restore impaired bone marrow and its function and therefore the immune system of patients who have suffered a decrease in hematopoietic cells or mature blood cells due to a disease, radiation or chemotherapy. Low transplantation efficiency can result in poor survival outcome for patients undergoing HSCT. For e.g., the number of hematopoietic stem and progenitor cells (HSPCs) in umbilical cord blood (CB) is often low and post-transplantation patient survival can be improved by doubling the number of CB units (Smith and Wagner, 2009). One potential strategy therefore for improved recovery can be to expand the numbers of HSPCs prior to administration (Boitano et al., 2010; Delaney et al., 2010; Fares et al., 2014; Frisch et al., 2009; Himburg et al., 2010; Hoggatt et al., 2009; North et al., 2007). This approach however results in loss of stem cell properties of “multi-potency” and “self-renewal” which are critical for successful post-transplant reconstitution. Active cycling results in faster exhaustion due to differentiation into progressively more mature marrow cells and loss of proliferative, renewal, and reconstitution potential of the HSPCs to be transplanted (Nakamura-IsiZulu, A. et al., (2014). Development 141, 4656-4666., Passage, E. et al., (2005). J. Exp. Med. 202, 1599-1611.)

In order to improve post-transplant hematopoietic reconstitution, efforts have been made to modulate the growth control properties of hematopoietic stem cells. Cell cycle and epigenetic regulators as well as pathways involved in growth control, including cyclin dependent kinases and inhibitors, Rb, PI3K, and p53, have been demonstrated as cell-intrinsic regulators of HSPC proliferation (Ito and Suda, 2014; Nakamura-Ishizu et al., 2014). A variety of secreted and cell-surface factors which are produced by bone marrow (BM), including angiopoetin-1, thrombopoietin, SCF, and CXCL12 (Ito and Suda, 2014; Mendelson and Frenette, 2014; Morrison and Scadden, 2014), has been shown to extrinsically regulate HSPC. Cytokines SCF and TPO can both support survival and proliferation of purified mouse HSCs assayed in serum-free culture at the single cell level (Seita J, et al. Proc Natl Acad Sci USA. 2007; 104(7):2349-2354). Functional effects of many cytokines including IL-3, IL-6, IL-11, Flt-3 ligand in combinations with either SCF and/or TPO have been reported. Although exposing HSCs to these cytokines resulted in survival and proliferation of cells, in most studies, these cells immediately lose long-term reconstitution potential as assessed in transplantation assays. The Flt-3 receptor is not expressed on HSCs (Adolfsson J, et al. 2001; 15(4):659-669). Similarly, the IL-11 receptor knockout mice showed normal hematopoiesis, questioning an essential functional role for this receptor-ligand system on HSC function. It has now become clear that many cytokines have redundant functions at the level of either receptor binding or intracellular signal transduction.

In vivo culture studies have revealed inhibitory effect of TGF-β on HSC proliferation without inducing apoptosis. Moreover, neutralization of TGF-β has been shown to facilitate rapid proliferation of HSPC in vivo by releasing them from quiescence (Hatzfeld J, et al. J Exp Med. 1991; 174(4):925-929), U.S. Pat. No. 6,841,542 B2). US 2010/0034778 A1 reports the use of a modulator of the retinoic acid receptor RXR to enable stem cell expansion in vivo. Pleiotrophin is a growth factor shown to enhance HSC self-renewal and/or expansion in vivo (US 2011/0293574A1). CXCR4 antagonists have been shown to increase the rate of hematopoietic stem or progenitor cellular multiplication, self-renewal, expansion and proliferation (US 20020156034A1). Modulators of PI 3-kinase activity can be used to expand populations of renewable stem cells (US 2005/0054103 A1). Tie2/angiopoeitin-1 signaling regulates HSC quiescence in the bone marrow niche (Arai F, et al. Cell. 2004; 118(2):149-161).

The success of HSCT depends upon rapid reconstitution of mature blood cells to avoid infections and bleeding complications and long-term reconstitution of mature blood cells from durable restored source stem cells. (Doulatov et al., 2012; Smith and Wagner, 2009). Cell preparations intended for transplant are desired to comprise HSPCs who have their “multi-potency” and “self-renewal” capacities preserved and have retained an ability to achieve short-term recovery as well as improved long-term, multilineage hematopoietic reconstitution upon in vivo administration. Committed progenitors are responsible for the initial hematopoietic recovery, whereas the long-term repopulating HSCs (LT-HSCs) are responsible for establishing life-long multilineage hematopoiesis.

In contrast to high turnover of lineage-restricted progenitors, most of the HSCs reside in the “quiescent” G0 phase of the cell-cycle (Rossi D J, et al. Cell Cycle. 2007; 6(19):2371-2376., Nakamura-Ishizu, A et al., (2014). Development 141, 4656-4666). Quiescence contributes to HSC longevity and function, perhaps by minimizing stresses due to cellular respiration and genome replication (Eliasson, P., and J.-I. Jönsson. 2010. J. Cell. Physiol. 222:17-22.). Disruption of HSC quiescence leads to defects in HSC self-renewal and often results in HSC exhaustion (Orford, K. W., and D. T. Scadden. 2008. Nat. Rev. Genet. 9:115-128.). Therefore it follows that a proper balance of pools of HSPCs with quiescence and proliferative properties can result in successful transplantation outcomes. However, a non-cell autonomous regulator of hematopoiesis with cell-context specific effects for e.g., a modulator, which simultaneously preserves HSC stemness by quiescence while enabling progenitor expansion, has not been identified till date. Such a modulator can enhance post-transplant reconstitution of the cells to be administered by promoting quiescence and self-renewal of primitive HSPC including LT-HSCs, and proliferative expansion of myeloid-restricted progenitors. As such there is an unmet need of methods of producing the hematopoietic stem cell composition which is characterized by preserved stemness of the HSC such that the compositions enable short-term recovery and enhanced long-term multilineage post-transplantation reconstitution and therefore successful outcome.

Enhanced hematopoietic reconstitution is also required after IR-induced hematopoietic failure, which is a primary cause of death after exposure to a moderate or high dose of total body irradiation (TBD. Within a few hours or days after exposure to a significant dose of TBI, a series of characteristic clinical complications termed the acute radiation syndrome (ARS) appear. The hematopoietic syndrome occurs at TBI doses in the range of 2-7.5 Gy in humans (3-10 Gy in rodents) and is caused by severe depletion of blood elements due to BM suppression; the gastrointestinal syndrome occurs after doses >5.5 Gy of TBI; and the neurovascular syndrome occurs following large doses of TBI (>20 Gy), indicating that the hematopoietic system is the most radiosensitive tissue of the body. In addition, exposure to a moderate- or high-dose TBI also induces residual (or long-term) BM injury manifested by a decrease in HSC reserves and fitness and impairment in HSC self-renewal. Currently, there are no FDA-approved drugs to treat severely irradiated individuals (Singh et al., 2015). A number of hematopoietic growth factors have been shown in various animal models to mitigate hematopoietic syndrome of acute radiation syndrome, however only pleiotrophin has been reported to improve survival when administered 24 hours post-irradiation (Himburg et al., 2014). Moreover, current standard-of-care approaches, including granulocyte colony-stimulating factor (G-CSF) and its derivatives, target a limited progenitor cell pool and requires repeated doses to combat radiation-induced neutropenia (Singh et al., 2015). Therefore, there is an unmet need for a prophylactic and therapeutic to improve hematopoietic reconstitution and survival of subject post-exposure to radiation.

### SUMMARY

The technology described herein is based in part on the discovery that in vivo or ex vivo, exposure of hematopoietic stem cells and/or progenitor cells to Angiogenin (ANG), results in enhanced hematopoietic reconstitution, including repopulation of cells of all blood lineage and their functions, as well as enhanced self-replication of the HSCs to repopulate and maintain the stem cell pool, for example, after in vivo administration of the treated cells.

Described herein are uses, methods and compositions comprising of Angiogenin as a regulator of hematopoietic reconstitution. In one aspect, the technology described herein relates to hematopoietic cell compositions comprising, hematopoietic stem cells and/or progenitor cells contacted with, or cultured in presence of Angiogenin or an agonist thereof, where the cells are ex vivo or in vitro. The compositions are characterized by at least one of: increased quiescence of primitive hematopoietic stem cells, and increased proliferation of myeloid restricted progenitors. The technology disclosed herein also relates to methods to enhance the short term and long term hematopoietic reconstitution upon in vivo administration of the said compositions.

Another aspect of the technology herein relates to use of ANG protein or an agonist thereof to treat subjects that suffer from a disease characterized by at least one of: decreased levels of hematopoietic stem cells and/or progenitor cells, decreased levels of hematopoietic reconstitution, blood cell deficiency or have been exposed to, or likely to be exposed to ionization radiation. Accordingly, provided herein are methods and pharmaceutical compositions comprising ANG or a functional fragment thereof, or an agonist thereof, for at least one of: increasing in vivo levels of hematopoietic stem and/or progenitor cells, increasing in vivo levels of hematopoietic reconstitution, increasing in vivo levels of blood cells, or treatment of one or more disorders disclosed herein. In some embodiments, provided herein are methods and pharmaceutical compositions comprising ANG or a functional fragment thereof, or an agonist thereof, for preventing, or treating radiation induced hematopoietic injury, e.g., as a result of radio- or chemotherapy as a treatment for a disease or a result of accidental exposure to radiation, wherein the pharmaceutical composition is administered in an therapeutically effective amount.

Thus in one aspect, described herein is a method of increasing hematopoietic reconstitution in a human subject, the method comprising: (i) contacting a population of hematopoietic cells ex vivo, with an effective amount of an Angiogenin (ANG) protein or an ANG agonist; (ii) administering cells from step (i) to a subject, wherein the subject is in need of hematopoietic reconstitution. In some embodiments, the subject is in need of hematopoietic reconstitution.

In some embodiments, a population of hematopoietic cells is obtained from any of; bone marrow, peripheral blood, cord blood, amniotic fluid, placental blood, embryonic stem cells (ESCs), or induced pluripotent stem cells (iPSCs). In some embodiments, a population of hematopoietic cells is human. In some embodiments, a population of hematopoietic cells comprises at least one or more of long-term hematopoietic stem cells (LT-HSCs), short-term hematopoietic stem cells (ST-HSCs), multipotent progenitors (MPPs), common myeloid progenitors (CMPs), common lymphoid progenitors (CLPs), granulocyte-macrophage progenitors (GMPs) and megakaryocyte-erythroid progenitors (MEPs). In some embodiments, the population of hematopoietic cells is autologous or allogeneic to the subject.

In one aspect, the methods described herein further comprises culturing the population of hematopoietic cells in presence of ANG protein or ANG agonist for a pre-determined time, prior to step (ii). In some embodiments, the population of hematopoietic cells are cultured in presence of ANG protein or ANG agonist for a pre-determined time of at least 2 hrs. In another embodiment, the population of hematopoietic cells are cultured in presence of ANG protein or ANG agonist for a pre-determined time of about 2 days or more. In another embodiment, the population of hematopoietic cells are cultured in presence of ANG protein or ANG agonist for a pre-determined time of at least 7 days. In some embodiments, the population of hematopoietic cells are cryopreserved prior to, or after, the contacting with ANG protein or ANG agonist. In some embodiments, the subject is susceptible to, or has decreased levels of hematopoietic stem cells and hematopoietic progenitor cells as compared to a healthy subject. In some embodiments, the subject has undergone, or will undergo abone marrow or stem cell transplantation, or has undergone, or will undergo chemotherapy or radiation therapy. In some embodiments, the subject has a disease or disorder selected from the group consisting of leukemia, lymphoma, myeloma, solid tumor, a blood disorder (e.g., myelodysplasia), immune disorders and anemia.

In some embodiments of the technology described herein, the ANG protein is human ANG protein of at least 85% amino acid sequence identity to SEQ ID NO: 1 or a functional fragment thereof with a biological activity of at least 80% of human ANG protein to increase hematopoietic reconstitution in a human subject. In some embodiments, the ANG protein is a human recombinant ANG polypeptide. In some embodiments, the human ANG protein of at least 85% amino acid sequence identity to SEQ ID NO: 1 comprises a mutation K33A. In some embodiments, the functional fragment comprises an amino acid sequence of at least 80% of human ANG of SEQ ID NO: 1. In some embodiments, the functional fragment of human ANG protein comprises at least 80% sequence identity to amino acids 1-147 of SEQ ID NO 1. In other embodiments, the functional fragment of human ANG protein comprises at least 90% sequence identity to amino acids 1-147 of SEQ ID NO 1. In other embodiments, the functional fragment of human ANG protein comprises at least 95% sequence identity to amino acids 1-147 of SEQ ID NO 1. In other embodiments, the functional fragment of human ANG comprises at least 98% sequence identity to amino acids 1-147 of SEQ ID NO 1.

In some embodiments of the foregoing aspects the hematopoietic reconstitution is multi-lineage hematopoietic reconstitution. In some embodiments, the hematopoietic reconstitution is long-term multi-lineage hematopoietic reconstitution. In some embodiments, the hematopoietic reconstitution comprises reconstitution of short-term hematopoietic stem cells (ST-HSC) and/or long-term (LT-HSC) hematopoietic stem cells.

In another aspect, described herein are methods for expanding a population of hematopoietic cells in a biological sample, the method comprising contacting the hematopoietic cells with an Angiogenin (ANG) protein or an ANG agonist, wherein the population comprises primitive hematopoietic stem cells and myeloid restricted progenitors, and wherein the contacting is for a sufficient amount of time to allow for primitive hematopoietic stem cells quiescence and myeloid restricted progenitor proliferation.

In some embodiments, the primitive hematopoietic stem cells are selected from the group, LT-HSC, ST-HSC, MPP or a combination thereof. In some embodiments, the myeloid restricted progenitor are selected from the group, CMP, GMP, MEP or a combination thereof.

In some embodiments, the biological sample is selected from the group of: cord blood, bone marrow, peripheral blood, amniotic fluid, or placental blood.

In some embodiments, the method for expanding a population of hematopoietic cells in a biological sample further comprises collecting the population of expanded hematopoietic cells.

In another aspect, described herein is a population of primitive hematopoietic stem cells produced by the methods disclosed herein.

In another aspect, described herein is a population of myeloid restricted progenitors produced by the methods disclosed herein.

In another aspect, described herein is a cryopreserved population of hematopoietic cells comprising primitive hematopoietic stem cells and/or myeloid restricted progenitors in the presence of an angiogenin protein or ANG agonist.

In another aspect, disclosed herein is a blood bank comprising the said population of hematopoietic cells.

In another aspect, disclosed herein is a method of administering a population of hematopoietic cells to a subject, comprising administering an effective amount of the population of hematopoietic cells to the subject, wherein the population of hematopoietic cells have been contacted ex vivo or in vitro with an Angiogenin (ANG) protein or ANG agonist, wherein the population of hematopoietic stem cells comprises at least one or both of primitive hematopoietic stem cells and myeloid restricted progenitors, and wherein the Angiogenin protein increases primitive hematopoietic stem cells quiescence and increases myeloid restricted progenitor proliferation.

In another aspect, disclosed herein is a method of increasing reconstitution potential of transplanted hematopoietic stem cells and hematopoietic progenitor cells in a subject, the method comprising the step of administering Angiogenin (ANG) protein or an ANG agonist to the subject, prior to, during or after transplantation of hematopoietic stem cells and hematopoietic progenitor cells, wherein the subject is a candidate for bone marrow or stem cell transplant.

In another aspect, disclosed herein are uses of Angiogenin (ANG) protein to increase hematopoietic reconstitution potential of a population of hematopoietic cells in a human subject in need thereof. In some embodiments, the population of hematopoietic cells are obtained from bone marrow, peripheral blood, cord blood, amniotic fluid, placental blood, embryonic stem cells (ESCs), or induced pluripotent stem cells (iPSCs). In some embodiments, the population of hematopoietic cells are human. In some embodiments, the population of hematopoietic cells comprises at least one or more of long-term hematopoietic stem cells (LT-HSCs), short-term hematopoietic stem cells (ST-HSCs), multipotent progenitors (MPPs), common myeloid progenitors (CMPs), common lymphoid progenitors (CLPs), granulocyte-macrophage progenitors (GMPs) and megakaryocyte-erythroid progenitors (MEPs). In some embodiments of the foregoing aspects, the population of hematopoietic cells are autologous or allogeneic to the subject.

In some embodiments, the population of hematopoietic cells is cultured in presence of Angiogenin protein or ANG agonist. In some embodiments, of the use of Angiogenin, the population of hematopoietic cells are cultured in presence of Angiogenin protein or ANG agonist for at least 2 hrs. In some embodiments, the population of hematopoietic cells are cultured in presence of Angiogenin protein or ANG agonist for about 2 days or more. In some embodiments, the population of hematopoietic cells are cultured in presence of Angiogenin protein or ANG agonist for at least 7 days. In some embodiments, the population of hematopoietic cells are cryopreserved prior to, or after, the contacting with ANG protein or ANG agonist. In some embodiments, the population of hematopoietic cells are cryopreserved in the presence of ANG protein or ANG agonist.

In some embodiments, the subject is susceptible to, or has decreased levels of hematopoietic stem cells and hematopoietic progenitor cells as compared to a healthy subject. In some embodiments, the subject has undergone, or will undergo bone marrow or stem cell transplantation, or has undergone, or will undergo chemotherapy or radiation therapy. In some embodiments, the subject has a disease or disorder selected from the group consisting of leukemia, lymphoma, myeloma, solid tumor, a blood disorder, myelodysplasia, immune disorders or anemia. In some embodiments, the anemia is sickle cell anemia, thalassemia or aplastic anemia.

In some embodiments, of the foregoing aspect, the ANG protein is human ANG protein of at least 85% amino acid sequence identity to SEQ ID NO: 1 or a functional fragment thereof with a biological activity of at least 80% of human ANG protein to increase hematopoietic reconstitution in a human subject. In some embodiments, the ANG protein is a human recombinant ANG polypeptide. In some embodiments, the functional fragment comprises at least amino acids 1-147 of SEQ ID NO: 1. In some embodiments, the human ANG protein of at least 85% amino acid sequence identity to SEQ ID NO: 1 comprises a mutation K33A. In some embodiments, the functional fragment comprises an amino acid sequence of at least 80% of human ANG of SEQ ID NO: 1. In some embodiments, the functional fragment of human ANG protein comprises at least 80% sequence identity to amino acids 1-147 of SEQ ID NO: 1. In some embodiments, the functional fragment of human ANG protein comprises at least 90% sequence identity to amino acids 1-147 of SEQ ID NO: 1. In some embodiments, the functional fragment of human ANG protein comprises at least 95% sequence identity to amino acids 1-147 of SEQ ID NO: 1. In some embodiments, the functional fragment of human ANG comprises at least 98% sequence identity to amino acids 1-147 of SEQ ID NO: 1.

In some embodiments, the hematopoietic reconstitution is multi-lineage hematopoietic reconstitution. In some embodiments, the hematopoietic reconstitution is long-term multi-lineage hematopoietic reconstitution. In some embodiments, the hematopoietic reconstitution comprises reconstitution of short-term hematopoietic stem cells (ST-HSC) and/or long-term (LT-HSC) hematopoietic stem cells.

In one aspect, described herein is a method of prevention or treatment of radiation injury by exposure to ionizing radiation in a subject, the method comprising administering an effective amount of an Angiogenin (ANG) protein or Angiogenin agonist to the subject. In some embodiments, the subject has been exposed to, will be exposed to or is at a risk of exposure to ionizing radiation. In some embodiments, the subject is a mammal. In some embodiments, the subject will undergo, or has undergone, radiation therapy for the treatment of a disease or disorder. In some embodiments, the subject will undergo, or has undergone radiation therapy as part of an ablative regimen for hematopoietic stem cell or bone marrow transplant or chemotherapy. In some embodiments, the subject will undergo, or has under gone total body radiation. In some embodiments, the subject will undergo, or has been exposed to a radiation accident or chemotherapy.

In some embodiments, the hematopoietic stem and progenitor cells are selected from the group consisting of Long-term hematopoietic stem cells (LT-HSCs), Short-term hematopoietic stem cells (ST-HSCs), Multipotent progenitor cells (MPPs), Common myeloid progenitor (CMPs), CLPs, Granulocyte-macrophage progenitor (GMPs) and Megakaryocyte-erythroid progenitor (MEPs).

In some embodiments, the ANG protein or ANG agonist is administered to the subject prior to, during or after exposure, or a combination thereof, to an ionizing radiation. In some embodiments, the ANG protein or ANG agonist is administered for between 12 hours and 3 days prior to exposure to ionizing radiation. In some embodiments, the exposure to ionizing radiation occurs within about 24 hours after the last administration of ANG protein or ANG agonist. In some embodiments, the ANG protein or ANG agonist is administered immediately after the exposure to ionizing radiation. In some embodiments, the ANG protein or ANG agonist is administered about 24 hours after exposure to ionizing radiation.

In some embodiments, the ANG protein or ANG agonist is administered for at least 3 days or more.

In some embodiments, administration of the effective amount of ANG protein or ANG agonist results in increased hematopoietic reconstitution after exposure to ionizing radiation as compared to in absence of administration. In some embodiments, the administration of the effective amount of ANG protein or ANG agonist increases primitive hematopoietic stem cells quiescence and increases myeloid restricted progenitor proliferation as compared to in absence of administration.

In some embodiments, the ANG protein is human ANG protein of at least 85% amino acid sequence identity to SEQ ID NO: 1 or a functional fragment thereof with a biological activity of at least 80% of human ANG protein to increase hematopoietic reconstitution in a human subject. In some embodiments, the ANG protein is a human recombinant ANG polypeptide. In some embodiments, the functional fragment comprises at least amino acids 1-147 of SEQ ID NO: 1. In some embodiments, the human ANG protein of at least 85% amino acid sequence identity to SEQ ID NO: 1 comprises a mutation K33A. In some embodiments, the functional fragment comprises an amino acid sequence of at least 80% of human ANG of SEQ ID NO: 1. In some embodiments, the functional fragment of human ANG protein comprises at least 80% sequence identity to amino acids 1-147 of SEQ ID NO: 1. In some embodiments, the functional fragment of human ANG protein comprises at least 90% sequence identity to amino acids 1-147 of SEQ ID NO: 1. In some embodiments, the functional fragment of human ANG protein comprises at least 95% sequence identity to amino acids 1-147 of SEQ ID NO: 1. In some embodiments, the functional fragment of human ANG comprises at least 98% sequence identity to amino acids 1-147 of SEQ ID NO: 1.

In another aspect, disclosed herein is a method, of increasing the dose of an ionizing radiation treatment, comprising administering to the subject an effective amount of an Angiogenin (ANG) protein or Angiogenin agonist before, after or during the ionizing radiation, wherein the dose of the ionizing radiation treatment is higher as compared to the dose in absence of Angiogenin (ANG) protein or Angiogenin agonist administration.

In another aspect, disclosed herein is a composition comprising a population of hematopoietic cells generated by the methods of the foregoing aspects and a pharmaceutically acceptable carrier.

In one aspect, disclosed herein is a pharmaceutical composition comprising a population of hematopoietic cells and an effective amount of ANG protein or ANG agonist, wherein the population of hematopoietic cell comprises at least one or both of primitive hematopoietic stem cells and myeloid restricted progenitor cells, and wherein the effective amount ANG protein or ANG agonist increases quiescence of primitive hematopoietic cells and proliferation of myeloid restricted cells.

In some embodiments, the primitive hematopoietic cells are selected from the group, long-term hematopoietic stem cells (LT-HSCs), short-term hematopoietic stem cells (ST-HSCs), multipotent progenitors (MPPs) or a combination thereof. In some embodiments, the myeloid-restricted progenitor cells are selected from the group, common myeloid progenitors (CMPs), granulocyte-macrophage progenitors (GMPs), megakaryocyte-erythroid progenitors (MEPs) and combination thereof.

In another aspect, disclosed herein is a pharmaceutical composition comprising an effective amount of ANG protein or ANG agonist for use in promoting hematopoietic reconstitution, wherein the effective amount is capable of increasing primitive hematopoietic cell quiescence and proliferation of myeloid restricted cells.

In another aspect, disclosed herein is a pharmaceutical composition comprising an effective amount of ANG protein or ANG agonist for use in treatment of a disease or disorder characterized by decreased levels of hematopoietic stem cells and hematopoietic progenitor cells.

In some embodiments, the disease or disorder is selected from the group consisting of leukemia, lymphoma, myeloma, solid tumor, a blood disorder, myelodysplasia, immune disorders or anemia. In some embodiments, the anemia is sickle cell anemia, thalassemia or aplastic anemia.

In another aspect, provided herein are stem cell collection bags, stem cell separation and stem cell washing buffers supplemented with an effective amount of ANG protein or ANG agonist, wherein the effective amount is capable of increasing primitive hematopoietic cell quiescence and proliferation of myeloid progenitor cells. In some embodiments, the stem cell collection bags are further supplemented with nutrients and cytokines. In some embodiments, the cytokines are selected from the group consisting of granulocyte colony stimulating factor, granulocyte macrophage colony stimulating factor and erythropoietin.

In another aspect, disclosed herein is a method of treating a subject suffering with a disease or disorder characterized by decreased in vivo levels of hematopoietic stem cells and progenitor cells or decreased in vivo hematopoietic reconstitution, the method comprising, administering an effective amount of ANG protein or ANG agonist to the subject, wherein the effective amount increases hematopoietic stem cell quiescence and proliferation of myeloid restricted progenitor cells, thereby increasing the in vivo levels of hematopoietic stem cells and progenitor cells or hematopoietic reconstitution.

### BRIEF DESCRIPTION OF THE DRAWINGS

FIGS. 1A-1B show proximity based single cell analysis of the bone marrow niche. FIG. 1A shows the experimental schema. DiI-labeled adult bone marrow LKS CD34-Flk2-LT-HSCs were intravenously injected into irradiated col2.3GFP pups (P2). Forty-eight hours later, fresh sections of the femori were obtained, individual proximal and distal OLCs were identified and harvested for single cell RNA-Seq analysis. Selected differentially expressed genes were validated in vivo. FIG. 1B shows micropipette aspiration of proximal OLC. Shown are overlaid single color (GFP and DiI) images before and after retrieval of proximal OLC (panel i) The proximal GFP+ OLC (green was identified based on proximity to the DiI-labeled HSPC (red). Panel (ii) shows the results following in-situ enzymatic dissociation, the HSPC was dislodged from its original location, other hematopoietic cells became loose and OLCs partially detached from the endosteal surface. Panel (iii) shows a proximal OLC aspirated into a micropipette.

FIGS. 2A-2B show statistical analysis. FIG. 2A shows Bayesian approach to estimate the posterior distribution of expression levels in individual proximal and distal OLCs (colored lines). The joint posteriors (black lines) describe the overall estimation of likely expression levels in each group and are used to estimate the posterior of the expression-fold difference (middle plot). The shaded area under the fold-difference posterior shows 95% confidence region. Expression of Vcam-1 gene is shown as an example. FIG. 2B shows results of gene set enrichment analysis (GSEA) of differentially expressed genes between proximal and distal OLCs. GSEA plots referring to expression of gene sets “Surface proteins” and “Immune response” in proximal OLCs (p<0.0005) are shown.

FIGS. 3A-3D show Proximal and distal OLCs are transcriptionally distinct. FIG. 3A shows classification of individual OLCs based on the top 200 differentially expressed genes. Each row represents a gene, with the most likely gene expression levels indicated by color (blue—high, white—low absent. FIG. 3B shows an unbiased genome-wide classification of proximal and distal OLCs. The receiver-operator curve is shown for the Support Vector Machine classification where all successive pairs of cells (one proximal and one distal were classified based on the training data provided by other cells (P<0.005. FIG. 3C and FIG. 3D show expression analysis of known niche-derived HSPC regulators and OLC maturation genes. The violin plots show the posterior distribution of the expression fold-difference (y-axis, log 2 scale for each gene, with the shaded area marking the 95% confidence region). The horizontal solid red lines show the most likely fold-change value.

FIGS. 4A-4F show conditional deletion of Ang from niche cell subsets leads to the loss of quiescence in LT HSCs and CLPs. FIG. 4A shows comparison of Ang expression in proximal and distal OLCs. FIG. 4B shows LT-HSC number per femur and FIG. 4C shows LT-HSC cell cycle status following conditional deletion of Ang from distinct niche cell subsets, as per the color-coded legend (n=4-10). Non-shaded graphs: control animals, shaded graphs: Ang-deleted animals. FIG. 4D shows CLP number per femur and FIG. 4E shows CLP cell cycle status following conditional deletion of Ang from distinct niche cell subsets (n=4-10). FIG. 4F shows long-term reconstitution following competitive (1:1) transplantation of bone marrow from control animals (solid lines) and animals with conditional deletion of Ang (broken lines) into WT congenic recipients (n=8). *P<0.05, **P<0.01, ***P<0.001.

FIGS. 5A-5D show immunophenotypic analysis. FIG. 5A shows FACS gating strategy used for quantification of primitive hematopoietic subsets. FIG. 5B shows the number (per femur) of STBHSC (i), MPP (ii) and common myeloid progenitors (CMP) following conditional deletion Ang from niche cell subsets, as indicated by the color scheme on the right (n=8). FIG. 5C shows FACS gating strategy used for cell cycle studies in primitive hematopoietic cells using Ki67/DAPI staining. FIG. 5D shows cell cycle status of STBHSC (i), MPP (ii) and CMP (iii) following conditional deletion Ang from niche cell subsets, as indicated by the color scheme on the right (n=8).

FIGS. 6A-6G show in vivo analysis of Interleukin 18 function in HSPC regulation. FIG. 6A shows comparison of IL18 expression in proximal and distal OLC. FIG. 6B shows BrdU incorporation by HSPC in IL18KO mice (n=5). FIG. 6C shows IL18 receptor expression in HSPC. Representative histograms are shown (n=3). A comparable cell population from IL18R KO mouse was used as a negative control (shaded histogram. FIG. 6D shows flow cytometric assessment of multi-lineage response to 5-FU in IL18KO mice. The statistical significance was assessed by ANOVA. Boxplots illustrating log ratios of cell numbers between 5-FU-treated and vehicle-treated animals in WT and IL18 groups are shown (n=7). FIG. 6E shows enumeration of apoptotic LKS cells and lin-negative cells in WT animals pre-treated with rIL18 prior to 5-FU exposure (n=5). FIG. 6F shows Myeloid and lymphoid reconstitution in IL18KO mice following transplantation of (WT) LKS cells (n=7). FIG. 6G shows multi-lineage donor chimerism following transplantation of LKS cells from IL18R1KO or WT animals into WT hosts (n=8) per group. *P<0.05, **P<0.01.

FIGS. 7A-7H. show effect of IL18. FIG. 7A shows peripheral blood analysis of IL18KO mice (n=12). FIG. 7B, FIG. 7C show quantification of primitive and mature cells in IL18KO mice (n=6). FIG. 7D shows experimental schema and cumulative donor chimerism following noncompetitive transplantation of WT BM marrow cells into WT or IL18KO hosts (n=5-7). FIGS. 7E-7G show estimation of in vivo growth kinetics and localization following transplantation of fluorescently labeled LKS cells into WT or IL18KO host by intra-vital microscopy (n=6). FIG. 7H show survival of WT and IL18KO animals following limiting dose bone marrow transplantation. *P<0.05, **P<0.01, ns—not significant.

FIGS. 8A-8C show the effect of IL18. FIG. 8A shows quantification, and FIG. 8B shows representative FACS plots from cell cycle studies in newborn IL18KO mice (n=6). FIG. 8C shows flow cytometric assessment of primitive hematopoietic subsets in P1 pups following in-utero exposure to busulphan (n=6).*P<0.05, **P<0.01.

FIG. 9 shows expression of human IL18 receptor in primitive hematopoietic cells. Representative histograms of cord blood and bone marrow analysis are shown (shaded histogram—isotype control, n=3).

FIGS. 10A-10F show Embigin regulates HSPC localization and homing. FIG. 10A shows comparison of Embigin expression in proximal and distal OLC. FIG. 10B shows enumeration of myeloid (kit+linSca1−) progenitor cell frequency and FIG. 10C shows enumeration of CFC number in peripheral blood following treatment with anti-Embigin or isotype control antibody (n=5). FIGS. 10D and 10E show quantification of HSPC homing to calvarial bone marrow 24 hours after transplantation using intravital microscopy. FIG. 10D show animals which were either injected with anti-Embigin or isotype control antibody prior to transplantation of LKS cells, or FIG. 10E show animals transplanted with anti-Embigin or isotype control-treated LKS cells (cumulative of two independent experiments, 2 animals per condition in each experiment. Each dot on the calvarial map represents location of an individual cell and each color—an individual mouse (n=4). Representative images and quantification of cell number are shown below. FIG. 10F shows proliferation of transplanted LKS cells in animals pre-treated with anti-Embigin (n=4) between 24 and 48 hours post-transplantation. *P<0.05, **P<0.01, ***P<0.001

FIGS. 11A-11E show Embigin regulates HSPC quiescence. FIG. 11A shows the number of primitive hematopoietic cells and FIG. 11B shows colony-forming cells 24 hours after treatment with anti-Embigin or isotype control antibody (n=5). FIG. 11C shows BrdU incorporation and FIG. 11D shows cell cycle analysis of primitive hematopoietic cells following treatment with anti-Embigin or isotype control antibody (n=5 mice). FIG. 11E shows competitive (1:1) transplant of the bone marrow from animals treated with anti-Embigin or isotype control antibody (n=10).

FIGS. 12A-12I show Ang deficiency results in loss of HSPC quiescence and defective transplantation FIG. 12A shows quantification of primitive hematopoietic cells (n=12) and FIG. 12B shows cell cycle status (n=8) in Ang−/− mice. FIG. 12C shows quantification of stem and progenitor in Ang−/− mice on day 7 post-exposure to 150 mg/kg 5-FU (n=8). FIG. 12D shows survival of Ang−/− mice following weekly 5-FU (150 mg/kg) exposure (n=10). Arrows indicate day of injection. FIG. 12E shows experimental schema of serial transplant using WT or Ang−/− hosts. FIG. 12F shows multi-lineage donor cell chimerism, FIG. 12G shows HSPC number and FIG. 12H shows HSPC cell cycle status after competitive primary transplantation of LT-HSCs into lethally-irradiated WT or Ang−/− recipients (n=8). FIG. 12I shows chimerism after secondary transplantation of sorted LT-HSCs from primary recipients into WT or Ang−/− secondary recipients (n=8). See also FIGS. 13A-13O and Tables 1-2.

FIGS. 13A-13O show ANG deficiency results in loss of HSPC quiescence and defective transplantation potential in young and aged mice (and is related to FIGS. 12A-12I). FIG. 13A shows representative gating schema of stem and progenitor cells. FIG. 13B shows BrdU incorporation in Ang−/− HSPC (n=5). FIG. 13C shows frequency of apoptotic HSPCs, lymphoid-restricted progenitors, and myeloid-restricted progenitors in WT or Ang−/− mice (n=10). FIG. 13D shows quantification of primitive hematopoietic cells (n=12) and FIG. 13E shows cell cycle status (n=12) in Ang−/− mice using SLAM/CD48 staining. FIG. 13F shows quantification of HSPC, lymphoid- and myeloid-restricted progenitors (n=5) and FIG. 13G shows cell cycle status (n=5) in 22-month old WT or Ang−/− mice (n=5). FIG. 13H shows colony formation of BM isolated from 22-month old WT or Ang−/− mice (n=5). FIG. 13I shows serial re-plating of BM from 22-month old WT or Ang−/− mice (n=5). Colonies were harvested on day 7 and re-plated in equal numbers. Colonies were then scored again on day 14. FIG. 13J shows experimental schema for transplantation of BM from aged WT and Ang−/− mice. FIG. 13K shows competitive transplant (1:1) of whole BM from 22-month old WT or Ang−/− donors (n=5). FIG. 13L shows experimental schema for non-competitive whole BM primary and secondary transplants into 8-week old WT or Ang−/− mice. FIG. 13M shows multi-lineage donor cell chimerism following non-competitive primary transplant of WT BM into WT or Ang−/− recipients (n=7-8). FIG. 13N shows homing analysis following transplantation of CFSE-labeled WT CD45.1 lineage-negative cells into WT or Ang−/− recipients 16-hours post-transplant (n=5). FIG. 13O shows survival of animals following secondary transplantation of BM from primary recipients into respective WT or Ang−/− secondary recipients (n=10).

FIGS. 14A-14C show dichotomous effect of ANG in LKS and myeloid-restricted progenitor cell cycling. FIG. 14A shows cell cycle status of LKS cells and myeloid-restricted progenitors (n=8) and FIG. 14B shows cell cycle status of MPP1-4 cells (n=6) from WT and Ang−/− mice. FIG. 14C is a heat map of results of qRT-PCR analysis of self-renewal transcripts from sorted LKS cells or myeloid-restricted progenitors treated with mouse ANG protein (0-600 ng/ml, n=6). See also FIGS. 15A-15K.

FIGS. 15A-15K show effect of ANG on quiescence is cell-context specific (and is related to FIGS. 14A-14C. FIG. 15A shows BrdU incorporation in WT or Ang−/− LKS cells and myeloid-restricted progenitors (n=5). FIG. 15B shows lymphoid-restricted progenitor cell number (n=6), FIG. 15C shows cell cycle status (n=6), and FIG. 15D shows BrdU incorporation (n=5) in WT and Ang−/− mice. FIG. 15E shows myeloid-restricted progenitor cell number (n=9), FIG. 15F shows cell cycle status (n=6 mice), and FIG. 15G shows BrdU incorporation (n=5) in WT and Ang−/− mice. Heat maps of qRT-PCR analysis of self-renewal transcripts from sorted WT or Ang−/− LKS cells and myeloid-restricted progenitors is shown in FIG. 15H, that of uncultured or cultured WT LT-HSCs in the presence of mouse ANG protein (0-600 ng/ml) for 2 h in PBS is shown in FIG. 15I, that of uncultured or cultured WT LT-HSCs in the presence of mouse ANG protein (0-600 ng/ml) for 2 h, 48 h or 7 days in S-clone media is shown in FIG. 15J, and that of WT or Ang−/− LT-HSCs cultured in the presence or absence of 300 ng/ml ANG is shown in FIG. 15K (n=6).

FIGS. 16A-16C show ANG-mediated regulation of protein synthesis is cell context-specific. FIG. 16A show in vivo OP-Puro incorporation in WT or Ang−/− LKS cells and myeloid-restricted progenitors. Cells were sorted 1 h after OP-Puro administration. Bar graphs are relative values to WT LKS (n=5). FIG. 16B show in vivo OP-Puro incorporation following 2 h ANG treatment of LKS cells and myeloid-restricted progenitors. Bar graphs are relative values to untreated LKS (n=6). FIG. 16C show qRT-PCR analysis of rRNA species following 2 h ANG treatment of LKS cells and myeloid-restricted progenitors, using various primer sets (n=3). See also FIGS. 17A-19D.

FIGS. 17A-17H show ANG-mediated regulation of protein synthesis is correlated with cell context-specific RNA processing (and is related to FIGS. 16A-16C and FIGS. 18A-18E). FIG. 17A shows OP-Puro incorporation in WT or Ang−/− stem, progenitor, and mature cell subsets 1 h after in vivo administration. Bar graphs are relative values to WT LKS (n=5). FIG. 17B-17C show BM cellularity (FIG. 17B) and LT-HSC frequency (FIG. 17C) lh after in vivo OP-Puro administration (n=5). FIG. 17D shows qRT-PCR analysis of rRNA species in WT or Ang−/− LT-HSCs, myeloid-restricted progenitors, or whole BM (n=3). FIG. 17E shows small RNA production in WT Lin+ cells treated with or without 300 ng/ml ANG protein for 2 h, using 15 μg RNA for electrophoresis (n=3). FIG. 17F shows small RNA production in WT or Ang−/− LKS cells (n=3). FIG. 17G shows small RNA production in WT LKS cells and myeloid-restricted progenitors treated with or without sodium arsenite (500 μM) and/or ANG protein (300 ng/ml) for 2 h (n=3). FIG. 17H shows colony formation of whole BM transfected with inactive (d)5′-P or active 5′-P tiRNA (n=3).

FIGS. 18A-18E show ANG-mediated regulation of protein synthesis is correlated with cell context-specific tiRNA production. FIG. 18A shows small RNA production (n=3) and FIG. 18B shows Northern blot analysis of tiRNA-Gly-GCC (n=3) following 2 h treatment of LKS cells and myeloid-restricted progenitors with ANG. Bar graphs are relative values to untreated LKS. FIG. 18C shows OP-Puro incorporation (n=5), and FIG. 18D shows heat maps of qRT-PCR analysis of self-renewal, pro-survival, and pro-apoptotic transcripts (n=5) in LKS cells and myeloid-restricted progenitors transfected with inactive (d)5′-P tiRNA or active 5′-P tiRNA. FIG. 18E shows post-transplant reconstitution of LKS cells transfected with inactive (d)5′-P tiRNA or active 5′-P tiRNA (n=7). See also FIGS. 17A-19D.

FIGS. 19A-19D show ANG is associated with RNH1 in the nucleus of HSPC and in the cytoplasm of myeloid-restricted progenitors and is related to FIGS. 16A-16C and FIGS. 18A-18E. FIG. 19A shows ANG and PABP localization in LKS cells and myeloid-restricted progenitors by immunofluorescence (n=5). FIG. 19B shows RNH1 and PABP localization in LKS cells and myeloid-restricted progenitors by immunofluorescence (n=5). FIG. 19C shows ANG and RNH1 localization in LKS cells and myeloid-restricted progenitors by immunofluorescence (n=5). FIG. 19D shows ANG/RNH1 FRET (n=10 cells from 3 mice). Scale bar: 1 μm. Increased sensitivity of Ang−/− mice to γ-irradiation, Related to FIGS. 20A-20K.

FIGS. 20A-20K shows survival of irradiated mice. FIG. 20A shows Kaplan-Meier survival curves of WT or Ang−/− mice subjected to 7.5 Gy (left), 7.75 Gy (middle), or 8.0 Gy (right) radiation (n=12). FIG. 20B shows blood leukocyte recovery on day 7 in WT or Ang−/− mice treated with 8.0 Gy (n=10). FIGS. 20C-20K show BM cellularity (FIG. 20C), HSPC number (FIG. 20D), HSPC cycling (FIG. 20E), lymphoid-restricted progenitor number (FIG. 20F), lymphoid-restricted progenitor cycling (FIG. 20G), myeloid-restricted progenitor number (FIG. 20H), myeloid-restricted progenitor cell cycling (FIG. 20I), apoptotic activity (FIG. 20J), and colony formation (FIG. 20K) of WT or Ang−/− mice treated with 4.0 Gy TBI (n=6). Animals were sacrificed and analyzed on day 7 post-irradiation.

FIGS. 21A-21L show ANG enhances radioprotection and radioresistance. FIG. 21A shows survival of WT or Ang−/− mice treated with ANG daily for three successive days 24 h pre-TBI (n=10). FIG. 21B shows survival of WT or Ang−/− mice treated with ANG daily for three successive days 24 h post-TBI (n=10). FIG. 21C-21G show H&E and BM cellularity of femurs (FIG. 21C), LKS and myeloid-restricted progenitor cell number (FIG. 21D), cell cycling (FIG. 21E), apoptotic activity (FIG. 21F), and post-transplant reconstitution (FIG. 21G) of WT mice treated with ANG daily for three successive days 24 h post-TBI (n=6). Scale bar=100 μm. FIG. 21H shows survival of WT mice treated with ANG daily for three successive days 24 h prior or post-12 Gy. FIG. 21I shows H&E and BM cellularity of femurs of WT mice treated with ANG daily for three successive days 24 h post-12.0 Gy TBI (n=6). Scale bar=100 μm. FIG. 21J shows LD50 of mice treated with ANG daily for three successive days beginning 24 h post-TBI (n=8). FIG. 21K is a heat map showing results from qRT-PCR analysis of self-renewal, pro-survival, pro-apoptotic, and rRNA transcripts (n=6), and FIG. 21L shows tiRNA production (n=3) in LKS or myeloid-restricted progenitors sorted from irradiated mice (4.0 Gy) and treated with 300 ng/ml ANG. See also FIGS. 19A-21L and Tables 7-9.

FIGS. 22A-22S show ANG enhances radioprotection and radioresistance (and is related to FIGS. 21A-21L). FIGS. 22A-22J show BM cellularity (FIG. 22A), HSPC number (FIG. 22B), HSPC cycling (FIG. 22C), lymphoid-restricted progenitor number (FIG. 22D), lymphoid-restricted progenitor cycling (FIG. 22E), myeloid-restricted progenitor number (FIG. 22F), myeloid-restricted progenitor cell cycling (FIG. 22G), apoptotic cell percentage (FIG. 22H), colony formation (FIG. 22I), and post-transplant reconstitution (FIG. 22J) of WT mice pre-treated with ANG daily for three successive days 24 h before 4.0 Gy TBI (n=6). Animals were sacrificed and analyzed on day 7 post-irradiation. FIG. 22K is a Kaplan-Meier survival curve of WT mice treated with ANG immediately following 8.0 Gy TBI (n=10).

FIGS. 22L-22S show HSPC number (FIG. 22L), HSPC cycling (FIG. 22M), lymphoid-restricted progenitor number (FIG. 22N), lymphoid-restricted progenitor cycling (FIG. 22O), myeloid-restricted progenitor number (FIG. 22P), myeloid-restricted progenitor cell cycling (FIG. 22Q), apoptotic cell percentage (FIG. 22R), and colony formation (FIG. 22S) of WT mice treated with ANG daily for three successive days beginning 24 h after 4.0 Gy TBI (n=6). Animals were sacrificed and analyzed on day 7 post-irradiation.

FIGS. 23A-23E. show ANG enhances post-transplant reconstitution. FIG. 23A shows cell density on day 7 from sorted WT or Ang−/− LT-HSCs (1875 cells/ml) cultured in the presence of various doses of ANG (n=6). FIG. 23B shows tiRNA levels following 7 day culture with 0 or 300 ng/ml ANG. After culture, cells were harvested and again treated with 0 or 300 ng/ml ANG (indicated by + or −) for 2 h prior to analysis by electrophoresis (n=3). FIG. 23C shows post-transplant reconstitution of LT-HSCs after 2 h ex vivo treatment with ANG (n=8-9). FIG. 23D shows secondary transplant without further ex vivo ANG treatment (n=7-8). FIG. 23E shows post-transplant reconstitution of WT or Ang−/− LT-HSCs which were cultured in the presence or absence of 300 ng/ml ANG for 2 h and competitively transplanted in WT hosts (n=7). See also FIGS. 22A-22S.

FIGS. 24A-24H show ANG enhances post-transplant reconstitution (and is related to FIGS. 23A-23E and FIG. 25A-25D). FIG. 24A shows post-transplant reconstitution of human CD34+ CB cells following 2 h ex vivo treatment with 300 ng/ml ANG (n=7). Cells were grown in culture for 7 days (2,500 cells/ml). At day 7, cells were harvested, washed with PBS, and replated in S-clone media without addition of ANG. FIG. 24B shows cell density and FIG. 24C is a heat map showing results of self-renewal transcripts (n=6). FIG. 24D shows BM homing 16 h post-transplant with CFSE-labeled Lin− cells that were cultured in the presence or absence of 300 ng/ml ANG for 2 h (n=5). FIG. 24E shows qRT-PCR analysis of self-renewal transcripts in human CD34+ CB cells following 7-day culture with human WT ANG protein and variants (n=6). FIG. 24F shows colony formation of human CD34+ CB cells plated in the presence or absence of 300 ng/ml human ANG (n=6). FIGS. 24G-24H show human CD19 (FIG. 24G) and human CD33 (FIG. 24H) frequencies in BM of NSG mice transplanted with human CD34+ CB cells treated with or without human ANG protein (300 ng/ml) for 2 hours. BM was harvested 16 weeks post-transplant.

FIGS. 25A-25D show ANG enhances post-transplant reconstitution of human CD34+ CB cells. FIG. 25A shows cell density on day 7 from human CD34+ CB cells (2,500 cells/ml) cultured in the presence of various doses of ANG or ANG variants: K40Q (enzymatic variant), R70A (receptor-binding variant), or R33A (nuclear localization variant) at 300 ng/ml (n=6). FIG. 25B is a heat map show results of qRT-PCR analysis of self-renewal transcripts in human CD34+ CB cells following 2 h culture with human ANG protein (n=6). (FIG. 25C) Human CD45 cells in BM of NSG mice transplanted with human CD34+ CB cells treated with or without human ANG (300 ng/ml) for 2 h. BM was harvested 16 weeks post-transplant (n=9-10). (FIG. 25D) LT-HSC frequencies (black line) and 95% confidence intervals (shaded boxes) for each transplant condition from FIG. 7C (p=8.28×10-5). See also FIGS. 24A-24H.

### DETAILED DESCRIPTION

Hematopoietic stem cells (HSCs) give rise to all other blood cells within the mammalian blood system, through the process of hematopoiesis. HSCs can carry out this function as they possess the unique ability of both “multi-potency” and “self-renewal”. Multi-potency is the ability to differentiate into all functional blood cells. Self-renewal is the ability to give rise to new HSC cells without differentiation. Since mature blood cells are predominantly short lived, HSCs continuously provide more differentiated progenitors while maintaining the HSCs pool size properly throughout life by precisely balancing self-renewal and differentiation. These properties together define the “stemness” of HSCs and are harnessed in the medical process of hematopoietic stem cells transplant which involves administration of HSCs in patients whose bone marrow or immune system is damaged or defective, in order to reestablish hematopoietic function.

In one aspect, the technology described herein generally relates to methods and use of protein Angiogenin (ANG) to improve hematopoietic reconstitution of hematopoietic cells in a subject, wherein the hematopoietic cells can be resident in vivo cells of the subject or are cells transplanted into the subject. In another aspect, the technology described herein generally relates to use of Angiogenin as a prophylactic and/or therapeutic agent, for example in methods to increase levels of hematopoietic cells, for hematopoietic constitution and/or treat blood cell deficiency associated with a disease or disorder as disclosed herein, or in a method to treat a radiation injury due to past, or predicted future exposure to radiation and promote survival of irradiation-exposed subject.

### Definitions

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

As used herein, the term “ex vivo” refers to a process in which cells are removed from a living organism and are treated outside the organism (e.g., in a test tube). The ex vivo conditions can involve providing the cells with nutrients (e.g. Cytokines). Methods of ex vivo culturing stem cells of different tissue origins are well known in the art of cell culturing to this effect, see for example the text book: Culture of Animal cells—A manual of basic Technique” by Freshney, Wiley-Liss, N.Y. (1994), Third edition, the teachings of which are hereby incorporated by reference. Concomitant with treating the cells with conditions which allow for ex vivo the stem cells to proliferate, the cells are short-term treated or long-term treated with Angiogenin.

As used herein, the term “stem cell” refers to an undifferentiated cell which has the capacity to develop to any cell lineage present in the organism from which they are derived, given the right growth conditions, by the process of differentiation and can undergo self-renewal to produce daughter stem cell having the parental undifferentiated state and properties. Typically to self-renew, the stem cell can undergo an asymmetric cell division with one daughter cell maintaining the parental stem state and the other daughter expressing some distinct other specific function and phenotype (e.g., a progenitor cell). Alternatively, the stem cell can divide symmetrically into two daughter stem cells. Thus self-renewal maintains the number of stem cells in a population while other cells in the population give rise to differentiated progeny only. The stem cell therefore is capable of proliferation and giving rise to progenitor cells having the capacity to generate a large number of mother cells which in turn can give rise to differentiated or differentiable daughter cells. The daughter cells can further undergo proliferation to produce progeny that then can differentiate into one or more mature cell types. The capability of differentiation into a specialized cell type is defined as “potency”. The more the cell types a cell can differentiate into, the more the potency. Stem cell can therefore be totipotent, pluripotent, and multipotent.

The term “Totipotent cells” as used herein, refers to cells that can grow and differentiate into any cell in the body, and thus can grow into an entire organism. They have the ability to give rise to all the cell types of the body plus all of the cell types that make up the extraembryonic tissues such as the placenta. These cells are not capable of self-renewal. In mammals, only the zygote and early embryonic cells are totipotent.

The term “Pluripotent cells” as used herein, refers to are stem cells, with the potential to make nearly any differentiated cell in the body for e.g. Cells derived from any of the three germ layers namely endoderm, mesoderm and ectoderm. They cannot however give rise to an entire organism like the totipotent cells.

The term “Multipotent cells” as used herein, refers to cells that can develop into more than one cell type, but are more limited than pluripotent cells; adult stem cells and cord blood stem cells are considered multipotent. “Multipotent stem cells” are cells that self-renew as well as differentiate to regenerate adult tissues. They are able to give rise to a subset of cell lineages, but all within a particular tissue, organ or physiological system. For example, hematopoietic stem cells (HSC) can produce progeny that include HSC (by self-renewal), blood cell restricted oligopotent progenitors, and all cell types and elements (e.g., platelets) that are normal components of the blood. The term “stem cells” as used herein, refers to multipotent stem cells of mammalian origin capable of self-renewal and to generate differentiated progeny. The term “Oligopotent cells” as used herein, refers to cells that can differentiate into only a few cell types e.g., lymphoid or myeloid progenitor cells.

The term “progenitor” or “precursor” cells are used interchangeably herein and refers to cells that have cellular phenotype that is more primitive (i.e. in earlier step along the developmental pathway) relative to the cell type it can give rise upon differentiation. They can also have high proliferative potential and can give rise to multiple distinct differentiated cell types or to a single differentiated cell type depending on the developmental pathway and on the environment in which the cells develop and differentiate.

The term “hematopoietic cells” as used herein broadly refers to cells pertaining to or affecting the formation of blood cells or “hematopoiesis”. As used herein, the term “hematopoietic cells”, encompasses “hematopoietic stem cells”, “primitive hematopoietic stem cells”, “hematopoietic progenitor cells” and “lineage restricted progenitor cells”.

The term “hematopoietic stem cells” or “HSCs” as used herein, refers to hematopoietic cells that are pluripotent stem cells or multipotent stem cells or lymphoid or myeloid (derived from bone marrow) cells that can differentiate into a hematopoietic progenitor cell (HPC) of a lymphoid, erythroid or myeloid cell lineage or proliferate as a stem cell population without initiation of further differentiation. HSCs can obtained e.g., from bone marrow, peripheral blood, umbilical cord blood, amniotic fluid, or placental blood or embryonic stem cells. HSCs are capable of self-renewal and differentiating into or starting a pathway to becoming a mature blood cell e.g. Erythrocytes (red blood cells), platelets, granulocytes (such as neutrophils, basophils and eosinophils), macrophages, B-lymphocytes, T-lymphocytes, and Natural killer cells through the process of hematopoiesis. The term “hematopoietic stem cells” or “HSCs” as used herein encompasses “primitive hematopoietic stem cells” i.e., long-term hematopoietic stem cells (LT-HSCs), short-term hematopoietic stem cells (ST-HSCs) and multipotent progenitor cells (MPP).

The term “long-term hematopoietic stem cells” or LT-HSCs as used herein, refers to hematopoietic stem cell with long-term (typically more than three months) hematopoietic reconstitution potential. The LT-HSCs can have unlimited self-renewal lasting throughout adulthood, contribute to long-term multilineage reconstitution after transplant and can maintain reconstitution potential after serial transplantation into another subject. The LT-HSCs can be less actively dividing and/or quiescent relative to other HSCs. The LT-HSCs can be distinguished based on their surface markers known in the art, for example LT-HSCs can be CD34−, CD38−, SCA-1+, Thy1.1+/lo, C-kit+, lin−, CD135−, Slamf1/CD150+ (Lin−) and exhibit absence of Flk-2 (Proc Natl Acad Sci USA. 2001).

The term “short-term hematopoietic stem cells” or ST-HSCs as used herein, refers to hematopoietic stem cell with hematopoietic reconstitution potential not exceeding three months and/or that is not multi-lineage. The ST-HSCs can be more actively dividing, more proliferating and less quiescent and have limited self-renewal capability relative to the LT-HSCs. ST-HSCs can be distinguished based on their surface markers known in the art, for example, ST-HSCs can be CD34+, CD38+, SCA-1+, Thy1.1+/lo, C-kit+, lin−, CD135−, Slamf1/CD150+, Mac-1 (CD11b)lo and exhibit presence of Flk-2+(Proc Natl Acad Sci USA. 2001). Loss of Thy-1.1 expression with full expression of Flk-2 characterizes the next differentiation step to the multipotent progenitor (MPP).

The term “hematopoietic progenitor cells” or “HPCs” as used herein, refers to hematopoietic cells that have differentiated to a developmental stage that, when the cells are further exposed to an appropriate cytokine or a group of cytokines, they will differentiate further along the hematopoietic cell lineage by the process of hematopoiesis. In contrast to primitive hematopoietic stem cells, hematopoietic progenitor cells are only capable of limited self-renewal. “Hematopoietic progenitor cells” as used herein can also include “precursor cells” that are derived from differentiation of hematopoietic progenitor cells and are the immediate precursors of mature differentiated hematopoietic cells. “Hematopoietic progenitor cells”, as used herein can also include, but are not limited to, multipotent progenitors (MPPs), Common lymphoid progenitors (CMPs), Common myeloid progenitors (CMPs), Common Myelolymphoid Progenitors (CMLPs), common myeloid-erythroid progenitor (CMEPs), granulocyte-macrophage progenitor (GMPs), megakaryocyte-erythroid progenitors (MEPs), granulocyte-macrophage colony-forming cell (GM-CFC), megakaryocyte colony-forming cell (Mk-CFC), burst-forming unit erythroid (BFU-E), B cell colony-forming cell (B-CFC) and T cell colony-forming cell (T-CFC). “Precursor cells” can include, but are not limited to, colony-forming unit-erythroid (CFU-E), granulocyte colony forming cell (G-CFC), colony-forming cell-basophil (CFC-Bas), colony forming cell-eosinophil (CFC-Eo) and macrophage colony forming cell (M-CFC) cells. “Hematopoietic progenitor cells” as used herein also includes “lineage restricted progenitor cells”.

The phrase “lineage restricted progenitor cells” as used herein, refers to cells having a defined lineage and that divide to produce cells having the same lineage. In other words, a lineage restricted progenitor cell has committed to a certain lineage and hence is not a pluripotent cell that can produce different cell types. Rather, a lineage restricted progenitor cell divides to produce cells of the same lineage as the lineage restricted progenitor cell. Lineage restricted progenitor cells are identifiable by certain markers, such as, expression of one or more marker proteins that are known in the art to be characteristic of a progenitor cell for their cell lineage. In addition, progenitor cells are typically mitotic, and thus incorporate BrdU into their DNA and/or express one or more markers, e.g. proteins that are typically expressed in mitotic cells, e.g. Ki67, PCNA, Anillin, AuroraB, and Survivin. An example of lineage-restricted progenitor cell is “myeloid restricted progenitor cells”, i.e., a myeloid progenitor cell, refers generally to a class of hematopoietic cells that differentiate into cells of a myeloid lineage (monocytes, granulocytes and megakaryocytes), and which lack the potential to differentiate into lymphoid lineages, which class includes CMP, GMP, MEP and MKP cells. Other non-limiting examples of lineage restricted progenitor cells include lymphoid restricted progenitor cells, erythroid restricted progenitor cells.

The term “Hematopoiesis” as used herein, refers to the highly orchestrated process of blood cell development and homeostasis. Prenatally, hematopoiesis occurs in the yolk sack, then liver, and eventually the bone marrow. In normal adults it occurs in bone marrow and lymphatic tissues. All blood cells develop from pluripotent stem cells. Pluripotent cells differentiate into hematopoietic stem cells that are committed to three, two or one hematopoietic differentiation pathway.

The terms “hematopoietic stem and/or progenitor cells” or “HSPCs” as used herein, refer to a population of cells comprising of hematopoietic stem cells and/or hematopoietic progenitor cells. In various embodiments of the aspects disclosed herein, it is contemplated that “HSC” and HSPCs can be used interchangeably.

As used herein, the term “population of hematopoietic cells” refers to cell population comprising at least one or combination of long-term hematopoietic stem cells (LT-HSCs), short-term hematopoietic stem cells (ST-HSCs), multipotent progenitors (MPPs), common myeloid progenitors (CMPs), common lymphoid progenitors (CLPs), granulocyte-macrophage progenitors (GMPs) and megakaryocyte-erythroid progenitors (MEPs). In some embodiments, population of hematopoietic cells comprises primitive hematopoietic stem cells, myeloid restricted progenitor cells or combination thereof. The cells may be contained in or obtained from e.g., bone marrow, peripheral blood, cord blood, amniotic fluid, or placental blood. The cells can be isolated using cell surface markers known in the art. The markers and methods of isolation are known to those skilled in the art.

The term “autologous” as used herein, refers to cells having originated from the same subject (e.g., recipient subject in whom the cells are to be transplanted). Thus, autologous cells are harvested from a subject and then returned to the same subject.

The term “allogeneic” as used herein, refers to cells originated from genetically non-identical subject from the same species as that of the recipient subject (i.e., subject in whom the cells are being administered or transplanted).

As used herein, the term “differentiation” refers to relatively generalized or specialized changes during development. Cell differentiation of various lineages is a well-documented process and requires no further description herein. As used herein, the term “differentiation of hematopoietic stem cells and/or hematopoietic progenitors” refers to both the change of hematopoietic stem cells into hematopoietic progenitors and the change of hematopoietic progenitors into unipotent hematopoietic progenitors and/or cells having characteristic functions, namely mature cells including erythrocytes, leukocytes and megakaryocytes. Differentiation of hematopoietic stem cells into a variety of blood cell types involves sequential activation or silencing of several sets of genes. Hematopoietic stem cells choose either a lymphoid or myeloid lineage pathway at an early stage of differentiation.

As used herein, the terms “hematopoietic reconstitution” or “hematopoietic repopulation” relates to the recovery of and/or repopulation of pool of HSCs by self-renewal, pool of HPC by differentiation of HSCs, and repopulation of all hematopoietic cell lineages for example; erythroid, myeloid and lymphoid lineages by differentiation of HSPCs and hematopoiesis within the bone marrow. Hematopoietic reconstitution in general therefore results in restoration of the normal functions of the bone marrow and immune system. Hematopoietic reconstitution comprises HSCs gaining access to the bone marrow (BM) in a process termed homing, take up residence in the BM, undergo self-renewing cell divisions to produce a larger pool of HSCs, and their differentiation into more committed progenitors, resulting in multilineage hematopoiesis. In various embodiments of the technology described herein, reconstitution of a given cell type can refer e.g., to its absolute count in the peripheral blood reaching a number of cells accepted by those of skill in the art as within the normal range for the subject. The reconstitution as referred herein can occur e.g., in a subject following a myeloablative regimen (for example chemotherapy or radiation therapy) and/or following in vivo administration of a population of hematopoietic cells (for example bone marrow transplantation). Reconstitution efficiency can depend upon several factors, including but not limited to the underlying disease and disease status, patient's age, preparative regimen (myeloablative vs nonmyeloablative), the intensity of prior therapy such as chemotherapy or radiation therapy, and the stem cell source, transplant type (autologous vs allogeneic), major histocompatibility complex (HLA) disparity resulting in graft-versus-host disease (GVHD); and infection. Non-limiting examples of methods to measure successful hematopoietic reconstitution can include measurement of complete blood count, differential blood counts, platelet counts, bone marrow biopsy tests, chest-x-rays, known to those skilled in the art.

As used herein, the term “long-term hematopoietic reconstitution” refers to reconstitution for more than three months. In some embodiments, “long-term hematopoietic reconstitution” can be for a lifetime of the subject. The primitive HSCs contributing to long-term hematopoietic reconstitution can be, for example, LT-HSCs.

As used herein, the term “multi-lineage hematopoietic reconstitution” refers to the ability of hematopoietic cells to repopulate cells of all hematopoietic lineages for example; erythroid, myeloid and lymphoid lineages.

As used herein, the term “short-term hematopoietic reconstitution” refers to reconstitution for a period of less than three months. The primitive HSCs contributing to short-term hematopoietic reconstitution can be for example, ST-HSCs.

The phrase “expanding a population of hematopoietic cells” is used herein to describe a process of cell proliferation substantially devoid of cell differentiation. Cells that undergo expansion hence maintain their cell renewal properties and are oftentimes referred to herein as renewable cells, e.g., renewable stem cells.

As used herein, the term “culturing the population of hematopoietic cells” refers to maintaining the hematopoietic cells under in vitro culture conditions that can e.g., facilitate expansion by proliferation, maintain potency of stem cells and at least preserve the viability of said hematopoietic cells. The viability can be determined by an assay for cell viability routinely used by those of skill in the art, e.g., a presidium iodide assay, by an in vitro culture assay in medium containing exogenously provided cytokines. With regard, maintaining the potency of “said hematopoietic cells”, the term means preservation of hematopoietic cells (e.g., LT-HSCs) into the same cell state as the cells used to initiate the culture, substantially devoid of cell differentiation e.g., an immunophenotype characteristic of human LT-HSC, for example, CD34−, CD38−, SCA-1+, Thy1.1+/lo, C-kit+, lin−, CD135−, Slamf1/CD150+ (Lin−), Flk-2−. The culture conditions can maintain potency of the cells by preserving them into a quiescence cell state or allowing cell proliferation devoid of cell differentiation for example proliferation of myeloid progenitors in the present disclosure.

As used herein the term “comprising” or “comprises” is used in reference to compositions, methods, and respective component(s) thereof, that are useful to an embodiment, yet open to the inclusion of unspecified elements, whether useful or not.

As used herein the term “consisting essentially of” refers to those elements required for a given embodiment. The term permits the presence of elements that do not materially affect the basic and novel or functional characteristic(s) of that embodiment of the disclosure.

As used herein the term “consisting of” refers to compositions, methods, and respective components thereof as described herein, which are exclusive of any element not recited in that description of the embodiment.

The terms “disease”, “disorder”, or “condition” are used interchangeably herein, refer to any alternation in state of the body or of some of the organs, interrupting or disturbing the performance of the functions and/or causing symptoms such as discomfort, dysfunction, distress, or even death to the person afflicted or those in contact with a person. A disease or disorder can also be related to a distemper, ailing, ailment, malady, disorder, sickness, illness, complaint, or affectation.

The term “in need thereof” when used in the context of a therapeutic or prophylactic treatment, means having a disease, being diagnosed with a disease, or being in need of preventing a disease, e.g., for one at risk of developing the disease. Thus, a subject in need thereof can be a subject in need of treating or preventing a disease.

The term “effective amount” as used herein, refers to an amount sufficient to affect a beneficial or desired clinical result upon treatment. Specifically, the term “effective amount” means an amount of an agent e.g., Angiogenin or an agonist thereof, sufficient to measurably at least one of; i. maintain primitive HSCs (e.g., LT-HSCs) in undifferentiated state and/or quiescent state, ii. allow self-renewal and expansion of hematopoietic stem and/or progenitor cells, or iii) enhance short-term and/or long-term in vivo hematopoietic reconstitution by at least 3 fold, at least 2.5 fold, at least 2 fold, at least 1.5 fold upon treatment of hematopoietic cells, ex vivo or in vivo with effective amount relative to absence of treatment. The enhanced hematopoietic reconstitution can result in a measurable effect in terms of reconstitution of hematopoietic cells and functions thereof in a treated subject against for e.g., cancer of blood and bone marrow and/or hemaglobinopathy and/or thalassemia. The effective amounts may vary, as recognized by those skilled in the art, depending on the number of hematopoietic cells to be treated, the duration of treatment, source of hematopoietic cells, the specific underlying disease to be treated by transplantation of treated hematopoietic cells, intensity of prior therapy such as chemotherapy or radiotherapy. In some embodiments, “effective amount” refers to amount of ANG or agonist thereof capable of reducing or eliminating the toxicity associated with radiation in healthy hematopoietic stem and/or progenitor cells in the subject. In some embodiments, effective amount is the amount required to temporarily (e.g., for a few hours or days) inhibit the proliferation of primitive hematopoietic stem cells (i.e., to induce a quiescent state in hematopoietic stem cells) in a subject. In some embodiments, the effective amount is the amount required to temporarily (e.g., for a few hours or days) increase proliferation of myeloid restricted progenitor cells in a subject

An effective amount can therefore result in a clinical outcome of at least one selected from; increasing hematopoietic reconstitution, normalizing the numbers of HSCs and HPCs and other blood cell types and their functions and cause treatment, reverse, alleviate, ameliorate, inhibit, slow down or stop the progression or severity of the disease resulting in or due to improper functioning of the bone marrow and the immune system or their symptoms. Effects that can be measured are absolute counts for individual blood cell types (white blood cells, red blood cells and platelets) in the peripheral blood reaching a number of cells accepted by those of skill in the art as within the normal range for the subject. Methods of conducting a complete blood count are known to those skilled in the art.

As used herein, the terms “treat,” “treatment,” “treating,” or “amelioration” refer to therapeutic treatments, wherein the object is to reverse, alleviate, ameliorate, inhibit, slow down or stop the progression or severity of a disorder or syndrome, (e.g., radiation injury, bone marrow failure, blood cancer, blood cell deficiencies and other blood disorders) characterized by or making a patient susceptible to decrease in levels of HSCs, HPCs and/or blood cells. The term “treating” includes reducing or alleviating at least one adverse effect or symptom of a syndrome. Treatment is generally “effective” if one or more symptoms or clinical markers are reduced. In the case of low blood cell counts or low HSCs, “effective treatment” refers to a treatment that normalizes the cell counts of the blood cells (e.g., cells of lymphoid and myeloid lineage) and maintains them within the normal range for at least one week. Alternatively, or in addition, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality. For example, treatment is considered effective if the condition is stabilized. The term “treatment” of a disease also includes providing relief from the symptoms or side-effects of the disease (including palliative treatment).

As used herein, a “subject”, “patient”, “individual” and like terms are used interchangeably and refers to a vertebrate, preferably a mammal, e.g., a primate, e.g., a human. Mammals include, without limitation, humans, primates, rodents, wild or domesticated animals, including feral animals, farm animals, sport animals, and pets. Primates include, for example, chimpanzees, cynomologous monkeys, spider monkeys, and macaques, e.g., Rhesus. Rodents include, for example, mice, rats, woodchucks, ferrets, rabbits and hamsters. Domestic and game animals include, for example, cows, horses, pigs, deer, bison, buffalo, feline species, e.g., domestic cat, and canine species, e.g., dog, fox, wolf, avian species, e.g., chicken, emu, ostrich, and fish, e.g., trout, catfish and salmon. The terms, “individual,” “patient” and “subject” are used interchangeably herein. A subject can be male or female.

Mammals other than humans can be advantageously used as subjects that represent animal models of conditions or disorders associated with stem cell transplantation or disorders associated with impaired bone marrow or immune system function. Such models are known in the art and are described in e.g., Biol Blood Marrow Transplant. 2008 January; Biol Blood Marrow Transplant. 1999.

A subject can be one who has been previously diagnosed with or identified as suffering from or under medical supervision for a disorder causing damaged bone marrow or immune system function such as leukemias, lymphomas, myeloma, aplastic anemia, sickle cell anemia, thalassemia, immune deficiency disorders, and some solid tumor cancers. A subject can be one who is diagnosed with or suffering from a blood cell deficiency (e.g., neutropenia). A subject can be one who is diagnosed and currently being treated for, or seeking treatment, monitoring, adjustment or modification of an existing therapeutic treatment, or is at a risk of developing such a disorder. A subject can be one who has undergone chemotherapy or radiation therapy. A subject can also be a person or individual has been exposed to, is being exposed to and/or to likely to be exposed to radiation or a radiation injury. (e.g. disaster response team members).

As used herein, the term “administering,” refers to the placement of an agent (e.g., ANG or agonist thereof) or a cell preparation (e.g., Hematopoietic cells which have been contacted, or are in contact with ANG or agonist thereof) as disclosed herein into a subject by a method or route that results in at least partial delivery of the agent at a desired site. Pharmaceutical compositions comprising the agent or cell preparation disclosed herein can be administered by any appropriate route which results in an effective treatment in the subject, e.g., intracerebroventricular (“icy”) administration, intranasal administration, intracranial administration, intracelial administration, intracerebellar administration, or intrathecal administration. In one aspect, the term “administering,” refers to the placement of preparation of hematopoietic cells treated with ANG or agonist thereof, as disclosed herein into a subject by a method or route that results in at least partial delivery of the cells at a desired site. Typically the hematopoietic cells are administered via intravenous route through a catheter much like blood transfusion. If the hematopoietic cells are cryopreserved, they are thawed prior to administration. In another aspect, “administering” refers to delivering angiogenin or an agonist thereof to a subject in need thereof (e.g., a subject who has been, is being or is likely to be exposed to radiation). Administration can be continuous or intermittent. In various aspects, a preparation or an agent can be administered therapeutically; that is, administered to treat an existing disease or condition. In further various aspects, a preparation can be administered prophylactically; that is, administered for prevention of a disease or condition (e.g., radiation injury).

The term “contacting” as used herein, refers to bringing a disclosed agent (e.g. ANG or an agonist thereof) and a cell, a target receptor, or other biological entity together in such a manner that the compound can affect the activity of the target (e.g., enzyme, cell, etc.), either directly; i.e., by interacting with the target itself, or indirectly; i.e., by interacting with another molecule, co-factor, factor, or protein on which the activity of the target is dependent.

As used herein, the terms “protein”, “peptide” and “polypeptide” are used interchangeably to designate a series of amino acid residues connected to each other by peptide bonds between the alpha-amino and carboxy groups of adjacent residues. The terms “protein”, “peptide” and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein”, “peptide” and “polypeptide” are used interchangeably herein when referring to a gene product and fragments thereof.

The term “agonist” is used in the broadest sense and includes any molecule that mimics or stimulates a biological activity of a native polypeptide disclosed herein. Agonists include, but are not limited to small molecules, proteins, nucleic acids, carbohydrates, lipids or any other molecules which bind or interact with biologically active molecules. For example, agonists can alter the activity of gene transcription by interacting with RNA polymerase directly or through a transcription factor or signal transduction pathway. Agonists can mimic the action of a “native” or “natural” compound (e.g., ANG protein). Agonists may be homologous to these natural compounds in respect to conformation, charge or other characteristics. Thus, agonists may be recognized by, e.g., nuclear receptors. This recognition may result in physiologic and/or biochemical changes within the cell, such that the cell reacts to the presence of the agonist in the same manner as if the natural compound was present.

The term “ANG agonist” as defined herein can be a compound that enhances or stimulates the normal biological activity of ANG by increasing transcription or translation of ANG-encoding nucleic acid, and/or by inhibiting or blocking activity of a molecule that inhibits ANG expression or ANG activity, and/or by enhancing normal ANG biological activity (including, but not limited to enhancing the stability of ANG or enhancing binding of ANG to a receptor and/or directly binding to and activating a potential ANG receptor (e.g., Plexin-B2 or PlXNB2). The “biological activity” can be defined herein as including at least one of the activity selected from e.g., enhancing the hematopoietic reconstitution potential of the hematopoietic cells, maintaining primitive HSCs quiescence or enabling progenitor proliferation, upon contact with a population of hematopoietic cells or source containing a population of hematopoietic cells. The activity of the agonist can be for example, at least 80%, at least 85%, at least 90%, at least 95%, at least 97% or at least 99% of the biological activity of human ANG of SEQ ID NO:1.

ANG agonists can also include ANG analogs and ANG derivatives. By “ANG analog” it is meant a peptide whose sequence is derived from that of ANG including insertions, substitutions, extensions, and/or deletions, having at least some amino acid identity to ANG or region of an ANG peptide. Analogs may have at least 50 or 55% amino acid sequence identity with a native ANG (e.g., human ANG, SEQ ID NO: 1) or at least 70%, 80%, 90%, or 95% amino acid sequence identity with a native ANG. In one embodiment, such analogs may comprise conservative or non-conservative amino acid substitutions (including non-natural amino acids and L and D forms). ANG agonist analogs are analogs as herein described and function as an ANG agonist.

An “ANG derivative” is defined as a molecule having the amino acid sequence of a wild-type ANG (e.g., human ANG, SEQ ID NO: 1) or analog thereof, but additionally having a chemical modification of one or more of its amino acid side groups, .alpha.-carbon atoms, terminal amino group, or terminal carboxylic acid group for example by ubiquitination, labeling, pegylation (derivatization with polyethylene glycol) or addition of other molecules. A chemical modification includes, but is not limited to, adding chemical moieties, creating new bonds, and removing chemical moieties. Such modifications can improve the molecule's solubility, absorption, biological half-life, etc. The modifications can alternatively decrease the toxicity of the molecule, or eliminate or attenuate an undesirable side effect of the molecule, etc. Moieties capable of mediating such effects are disclosed in Remington's Pharmaceutical Sciences, 18th edition, A. R. Gennaro, Ed., MackPubl., Easton, Pa. (1990). Furthermore, one or more side groups, or terminal groups, may be protected by protective groups known to the ordinarily-skilled synthetic chemist. The term “functional” when used in conjunction with “derivative” or “variant” refers to a polypeptide which possesses a therapeutically or physiologically relevant biological activity that is substantially similar to a biological activity of the entity or molecule of which it is a derivative or variant. By “substantially similar” in this context is meant that at least 50% of the relevant or desired biological activity of a corresponding wild-type peptide is retained. In some embodiments, the derivatives retains at least 60%, at least 70%, at least 80%, at least 90%, at least 95%, or more, including 100% or even more (i.e., the derivative or variant has improved activity relative to wild-type) of the ANG.

As used herein the term “ionizing radiation” refers to radiation of sufficient energy that, when absorbed by cells and tissues, typically induces formation of reactive oxygen species and DNA damage. Ionizing radiation can include X-rays, gamma rays, and particle bombardment (e.g., neutron beam, electron beam, protons, mesons, and others), and is used for purposes including, but not limited to, medical testing and treatment, scientific purposes, industrial testing manufacturing and sterilization, and weapons and weapons development. Radiation is generally measured in units of absorbed dose, such as therador gray (Gy), orin units of dose equivalence, such as rem or sievert (Sv).

By “at risk of exposure to ionization radiation” is meant a subject scheduled for (such as by scheduled radiotherapy sessions) exposure to ionizing radiation (IR) in the future, or a subject at risk of being exposed to IR inadvertently in the future. Inadvertent exposure includes accidental or unplanned environmental or occupational exposure (e.g., terrorist attack with a radiological weapon or exposure to a radiological weapon on the battlefield or exposure of a member of a disaster response team).

As used herein, the term “radiation injury” refers to any type of hematopoietic damage or toxicity caused by exposure to ionizing radiation. Non-limiting examples include decreased levels of hemtopoietic stem and progenitor cells, thombocytopenia, leucopenia, anemia, neutropenia, blood-cell deficiency, bone marrow malfunction, disruption of hematopoiesis and the like. Radiation injury for example causes hematopoietic syndrome which comprises decrease in levels of hematopoietic stem and progenitor cells leading to severe shortage of white blood cells, followed by a shortage of platelets and then red blood cells. The shortage of white blood cells can lead to severe infections. The shortage of platelets may cause uncontrolled bleeding. The shortage of red blood cells (anemia) causes fatigue, weakness, paleness, and difficulty breathing during physical exertion. Radiation injury leads to increases risk of cancer e.g., blood cancer.

As used herein, the term “healthy subject” refers to an individual who is known not to suffer from decreased levels of hematopoietic stem and progenitor cells or decreased levels of one or more types of blood cells or any disease or disorder characterized by the same, for example blood disorder, such knowledge being derived from clinical data on the individual including, but not limited to, a blood cell count. The healthy individual is also preferably asymptomatic with respect to the early symptoms associated with one or more diseases disclosed herein.

The terms “increased”, “increase”, “increasing” or “enhance” are all used herein to generally mean an increase by a statically significant amount; for the avoidance of doubt, the terms “increased”, “increase”, or “enhance”, mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. The increase can be, for example, at least 10%, at least 20%, at least 30%, at least 40% or more, and is preferably to a level accepted as within the range of normal for an individual without a given disease.

The terms, “decrease”, “reduce”, “reduction”, “lower” or “lowering,” or “inhibit” are all used herein generally to mean a decrease by a statistically significant amount. For example, “decrease”, “reduce”, “reduction”, or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

The term “statistically significant” or “significantly” refers to statistical significance and generally means a difference of two standard deviations (2SD) or more.

Unless otherwise stated, the present invention was performed using standard procedures, as described, for example in Sambrook et al., Molecular Cloning: A Laboratory Manual (3 ed.), Cold Spring Harbor Laboratory Press, Cold Spring Harbor, N.Y., USA (2001); Davis et al., Basic Methods in Molecular Biology, Elsevier Science Publishing, Inc., New York, USA (1995); Current Protocols in Protein Science (CPPS) (John E. Coligan, et. al., ed., John Wiley and Sons, Inc.), Current Protocols in Cell Biology (CPCB) (Juan S. Bonifacino et. al. ed., John Wiley and Sons, Inc.), and Culture of Animal Cells: A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, 1st edition, 1998) which are all incorporated by reference herein in their entireties.

Other than in the operating examples, or where otherwise indicated, all numbers expressing quantities of ingredients or reaction conditions used herein should be understood as modified in all instances by the term “about.” The term “about” when used in connection with percentages means ±1% of the value being referred to. For example, about 100 means from 99 to 101.

Although methods and materials similar or equivalent to those described herein can be used in the practice or testing of this disclosure, suitable methods and materials are described below. The abbreviation, “e.g.,” is derived from the Latin exempli gratia, and is used herein to indicate a non-limiting example. Thus, the abbreviation “e.g.,” is synonymous with the term “for example.”

As used in this specification and appended claims, the singular forms “a,” “an”, and “the” include plural references unless the context clearly dictates otherwise. Thus for example, reference to “the method” included one or more methods, and/or steps of the type described herein and/or which will become apparent to those persons skilled in the art upon reading this disclosure and so forth.

In this application and the claims, the use of the singular includes the plural unless specifically stated otherwise. In addition, use of “or” means “and/or” unless stated otherwise. Moreover, the use of the term “including”, as well as other forms, such as “includes” and “included”, is not limiting. Also, terms such as “element” or “component” encompass both elements and components comprising one unit and elements and components that comprise more than one unit unless specifically stated otherwise.

The technology described herein is based in part on the discovery that in vivo or ex vivo, exposure of hematopoietic stem cells and/or progenitor cells to Angiogenin (ANG), results in enhanced hematopoietic reconstitution, including repopulation of cells of all blood lineage and their functions, as well as enhanced self-replication of the HSCs to repopulate and maintain the stem cell pool, after in vivo administration of the treated cells.

Described herein are uses, methods and compositions comprising of Angiogenin as a regulator of hematopoietic reconstitution. In one aspect, the technology described herein relates to hematopoietic cell compositions comprising, hematopoietic stem and/or progenitor cells contacted with, or cultured in presence of Angiogenin or an agonist thereof ex vivo or in vitro, wherein the compositions are characterized by reduced proliferation and maintenance of primitive hematopoietic stem cells in quiescent state and enhancing their self-renewal while increased proliferation and therefore expansion of myeloid restricted progenitors without differentiation. The technology disclosed herein also relates to methods to enhance the short term and long term hematopoietic reconstitution upon in vivo administration of the said compositions.

Another aspect of the technology herein relates to use of ANG protein or an agonist thereof to treat subjects that suffer from disease characterized by decreased levels of hematopoietic stem and/or progenitor cells, decreased levels of hematopoietic reconstitution, blood cell deficiency or have been exposed to or likely to be exposed to ionization radiation. Accordingly, provided herein are methods and pharmaceutical compositions comprising ANG or a functional fragment thereof, or an agonist thereof for increasing in vivo levels of hematopoietic stem and/or progenitor cells, increasing in vivo levels of hematopoietic reconstitution, increasing in vivo levels of blood cells, treatment of one or more disorders disclosed herein and preventing or treating radiation induced hematopoietic injury, e.g., as a result of radio- or chemotherapy as a treatment for a disease or a result of accidental exposure to radiation, wherein the pharmaceutical composition is administered in an therapeutically effective amount.

### Hematopoietic Cells

The hematopoietic cells of the various methods and compositions disclosed herein encompass hematopoietic stem cells and hematopoietic progenitor cells.

Hematopoietic stem cell is a multipotent immature hematopoietic cell that can differentiate into a progenitor cell and therefore can develop into all types of blood cells, including white blood cells, red blood cells, and platelets and can self-renew. Classic studies in mice describe two populations of HSCs; LT-HSCs and ST-HSCs. A long-term stem cell typically includes the long-term contribution to multi-lineage reconstitution after transplantation, which is for more than at least three months. The LT-HSCs can be less actively dividing and/or quiescent relative to other HSCs. A short-term stem cell is typically anything that confers hematopoietic restoration for shorter than three months and/or is not multi-lineage. The ST-HSCs can be more actively dividing, more proliferating and less quiescent and have limited self-renewal capability relative to LT-HSCs.

Hematopoietic progenitor cells are a class of hematopoietic stem cells that have limited self-renewal capacity but remain multipotent and thus can differentiate into all mature cell types found in the blood. These are called multipotent progenitor (MPP) or also can be called LMPP (lymphoid-primed multipotent progenitor) or CMLP cells (common myelolymphoid progenitor cells). LT-HSCs, ST-HSCs and MPP can also be called primitive hematopoietic stem cells. Hematopoietic progenitor cells, as used herein can include, but are not limited to, multipotent progenitors (MPPs) and lineage restricted progenitor cells (e.g. myeloid restricted progenitor and lymphoid restricted progenitor cells). Non-limiting examples of lineage restricted progenitor cells include Common lymphoid progenitors (CMPs), Common myeloid progenitors (CMPs), Common Myelolymphoid Progenitors (CMLPs), common myeloid-erythroid progenitor (CMEPs), granulocyte-macrophage progenitor (GMPs), megakaryocyte-erythroid progenitors (MEPs), granulocyte-macrophage colony-forming cell (GM-CFC), megakaryocyte colony-forming cell (Mk-CFC), burst-forming unit erythroid (BFU-E), B cell colony-forming cell (B-CFC) and T cell colony-forming cell (T-CFC). “Precursor cells” include, but are not limited to, colony-forming unit-erythroid (CFU-E), granulocyte colony forming cell (G-CFC), colony-forming cell-basophil (CFC-Bas), colony forming cell-eosinophil (CFC-Eo) and macrophage colony forming cell (M-CFC) cells. Due to lack of long-term self-renewal capacity, hematopoietic progenitor cells cannot sustain long-term reconstitution, and are important for recovery in the period immediately following a hematopoietic stem cell transplant in an individual. Hematopoietic progenitor cells are therefore useful for transplantation and therefore for use in methods and compositions herein can be obtained from a variety of sources including, for example, bone marrow, peripheral blood, and umbilical cord blood.

HSPCs mostly live in the bone marrow (the spongy center of certain bones), where they divide to make new blood cells. Once blood cells mature, they leave the bone marrow and enter the bloodstream. A small number of stem cells also get into the bloodstream. These are called peripheral blood stem cells. In some embodiments, hematopoietic cells encompassed for use in the methods and compositions disclosed herein include one or more of the cell types described above. In some embodiments, the hematopoietic cells of the methods and compositions disclosed herein, can be a heterogeneous population of one or more of these cell types. In some embodiments, the hematopoietic cells encompassed for use in the methods and compositions disclosed herein comprises of a population of hematopoietic cells enriched in one or more cells types described above (for example, enriched in LT-HSCs or myeloid restricted progenitor cells). As used herein, “enriched” means to selectively concentrate or to increase the amount of one or more materials by elimination of the unwanted materials or selection and separation of desirable materials from a mixture (i.e. separate cells with specific cell markers from a heterogeneous cell population in which not all cells in the population express the marker). The population of hematopoietic cells can have for example, at least about 50% cells, at least about 60% cells, at least about 75% cells, at least about 85% cells or at least about 95% cells of a selected phenotype. In some embodiments, the selected cells will comprise a single myeloid restricted progenitor, e.g. CMP. In other embodiment, the selected cells will comprise two or more myeloid restricted progenitors, e.g., CMP and GMP; CMP and MEP; CMP, MEP and MKP; CMP, GMP and MEP; and the like. In some embodiments, the selected cells can comprise single primitive hematopoietic stem cells, e.g., LT-HSC. In other embodiments, the selected cells can comprise two or more primitive hematopoietic stem cells, e.g., LT-HSC and ST-HSC; LT-HSC and MPP; ST-HSC and MPP; LT-HSC, ST-HSC and MPP. In some embodiments, the hematopoietic cells used in the methods and compositions described herein comprise LT-HSCs or myeloid restricted progenitors or a combination thereof.

The hematopoietic cells of the various aspects of the technology described herein, can be a heterogeneous population of one or more these cell types or can be a population which is enriched for a one or more of the cell types described herein. The different types of hematopoietic stem and progenitor cells can be distinguished and isolated and enriched from any of their sources for example, bone marrow, peripheral blood, cord blood, prior to transplantation and for use in the present disclosure by using surface markers specific for the known stem/progenitor cell type, which are known in the art. Numerous methods for human hematopoietic stem cell enrichment/isolation are known in the art and generally include obtaining bone marrow, newborn cord blood, fetal liver or adult human peripheral blood which contains hematopoietic cells. Once obtained, hematopoietic stem cell component may be enriched by performing various separation techniques such as density gradient separation, immunoaffinity purification using positive and/or negative selection by panning, FACS, or magnetic bead separation. FACS-based cell sorting allows the recognition, quantification and purification of a small population of HSC and/or lineage committed progenitor cells and/or fully matured hematopoietic cells in a heterogeneous population of cells. Previous studies have also demonstrated that primitive hematopoietic stem cells, characterized as high proliferative potential colony-forming cells (HPP-CFC, in vivo) may be isolated by selecting a fraction of density gradient-enriched, lineage-depleted marrow cells, further selecting a cell population based on a single step fluorescence-activated cell sorter (FACS) fractionation for cells that bind low levels of the DNA binding dye, Hoechst 33342 (Hoechstlo) and low levels of the mitochondrial binding dye, Rhodamine 123 (Rholo; Wolf et al., 1993). The methods for stem cell isolation and enrichment can comprise selection of the required population based on identity of known markers on their surface for example by using commercially available magnetic beads coupled surface marker specific monoclonal antibodies for e.g., anti-CD34 beads (Dynal, Lake success, NY) and/or using techniques such as flow cytometry. The heterogeneous population of cells or enriched hematopoietic cells can be expanded in vitro prior to transplantation using the methods and compositions disclosed herein. In other aspects, they can be frozen in liquid nitrogen and stored for long periods of time, such that they can be thawed and used later.

The phenotypic markers which can characterize HSC are reported in the literature. Murine HSC are defined as KSL cells, which are c-Kit+, Sca-1+, and negative for lineage markers of mature blood cell types. The addition of the Flk-2/Flt3 receptor tyrosine kinase to the KSL markers enhances separation of ST-HSC (Flk-2+) from LT-HSC (Flk-2−). There is no human homolog for murine Sca-1. Instead, human HSC are typically identified on the basis of CD34 expression. Interestingly, more primitive HSC in mice have low or absent expression of CD34. The DNA-binding dye Hoechst 33342 can be used to identify low staining “side populations” (SP) of HSPC. Hoechst staining is often combined with KSL markers to further enrich HSC numbers, so called SPKLS cells. The purity of HSC in sorted SP, KSL or CD34+ HSPC can be increased by using the signaling lymphocyte activation molecule (SLAM) family proteins CD150, CD244, and CD48. The presence of CD150 distinguishes HSC from HPC; multipotent progenitors are CD150−CD244+CD48− and more committed progenitors are CD150−CD244+CD48+, though there is even variability among CD150+ HSC in their ability to provide balanced repopulation of irradiated bone marrow in mice.

In humans, for example, CD34 is an adhesion molecule that is expressed on HSC and progenitor cells. It plays a central role in HSC and progenitor cell recognition. CD90 is another important cell surface marker expressed on early stage hematopoietic cells. On the other hand, the absence of CD38 is normally associated with an earlier stage of hematopoiesis. CD10 and CD7 are important markers for early lymphoid lineage development. CD123, an interleukin-3 receptor, and CD135 (which is also called Flt3) have been shown to be important for myeloid lineage development. CD110, a thrombopoietin receptor, is important for platelet development. The CD34+ fraction of human bone marrow contains lineage-committed progenitors as well as long-term multi-lineage HSC, many laboratories have sought additional markers to further enrich the CD34+ population for long-term HSC. CD90/Thy1, Tie, CD117/c-kit, and CD133/AC133 have been found as positive markers to enrich long-term-HSC whereas several negative markers including CD38 have been reported. Human HSC from cord blood with a marker set of Lin− CD34+CD38− CD45RA CD90/Thy1+ Rhodamin123Low CD49f+ with long-term multilineage engraftment capabilities in NOD/SCID/IL2 receptor common-γ chain null mice have been reported (Notta et al., 2011). Non-limiting examples of characteristic marker combinations for humans include; CD34+CD38−CD90+CD45RA−CD49f+ (HSC), CD34+CD38−CD90−CD45RA−CD49f− (MPP). CD34+CD10+CD7+ (CLP), CD34+CD38+CD123medCD135+CD45RA− (CMP), CD34+CD38+CD123medCD135+CD45RA+ (GMP), CD34+CD38+CD123−CD135−CD45RA−CD110+ (MEP). An accurate detection, enumeration and isolation of subpopulations bearing these surface marker compositions can be achieved using flow cytometry. The enumeration of these cells within the blood post-transplantation can be indicator of successful hematopoietic reconstitution. The markers used for different hematopoietic stem and progenitor cell types in the methods and compositions herein are disclosed in the examples.

Following such enrichment steps, the cell population is typically characterized both phenotypically and functionally. In vitro assays generally measure HPC rather than primitive HSC, while long-term in vivo assays are a measure of LT-HSC. Colony-forming cell (CFC) assays determine the capacity of cells to form lineage-restricted colonies in a semi-solid, usually methylcellulose-based, media, but do not identify HSC, rather only HPC. The colony forming cell (CFC) assay, also referred to as the methylcellulose assay, is an in vitro assay used in the study of hematopoietic stem cells. The assay is based on the ability of hematopoietic progenitors to proliferate and differentiate into colonies in a semi-solid media in response to an agent for example Angiogenin. The colonies formed can be enumerated and characterized according to their unique morphology. While proliferation, and expansion can be measured by increase in cell number, loss of quiescence can be assayed by increase in actively dividing cells. A loss of quiescence can result in; (i) increase in cell numbers of the same type of HSC by self-renewal as assayed by proliferation assays or FACS analysis, (ii) active cell division and proliferation as assayed for example by incorporation of BrDU into newly synthesizing DNA and/or (iii) differentiation of HSC into lineage committed cells, which can be assayed by increase in the numbers of lineage committed cells by FACs analysis. In other words, increase in quiescence can be assayed for example, by decrease or no change in numbers of lineage committed cells, absence of active cell division and proliferation by proliferation assays or FACS analysis. One exemplary way for differentiating LT-HSC from ST-HSC and progenitors is their ability to engraft in vivo into irradiated hosts and maintain multilineage hematopoiesis indefinitely and through serial transplantation into new hosts for example the NOD/SCID mouse model.

### Sources of Hematopoietic Cells

Blood products—HSCs can be obtained from blood products. A blood product includes a product obtained from the body or an organ of the body containing cells of hematopoietic origin. Examples of such sources include but are not limited to unfractionated bone marrow, peripheral blood mononuclear cells, umbilical cord blood, umbilical cord tissue, peripheral blood (e.g., G-CSF mobilize peripheral blood), liver, thymus, lymph and spleen. In some embodiments, the aforementioned blood products can be directly used in the methods and compositions disclosed herein. In some embodiments, the aforementioned crude or unfractionated blood products can be enriched for cells having hematopoietic stem cell characteristics in a number of ways, for example, the mature differentiated cells can be selected against based on the surface markers that they express, as described above. Exemplary method includes fractionation of the blood product by selecting CD34+ cells. CD34+ cells include a sub-population of cells capable of self-renewal and multi-potentiality. Such selection can be done for example by using commercially available magnetic anti-CD34 beads. Unfractionated blood products can be obtained directly from a donor or retrieved from a cryopreservative storage. In some embodiments, the population of HSCs comprise of CD34+ cells.

Bone marrow—Bone marrow can be obtained or harvested by anesthetizing the stem cell donor, puncturing bone with a needle and harvesting bone marrow cells with a syringe. Most sites used for bone marrow harvesting are located in the hip bones and the sternum. The bone marrow aspirate can contain, LT-HSC, stromal cells, stromal stem cells, hematopoietic progenitor cells, mature and maturing white and red blood cells and their progenitors. Once obtained the bone marrow aspirate can be treated as a whole using the methods described herein, or hematopoietic cells can be isolated prior to use in the methods by using surface specific markers for the HSCs and progenitor cells known to those skilled in the art, also described in previous sections. Alternatively the harvested bone marrow or cells isolated from bone marrow can be cryopreserved for later use in the current disclosure.

Peripheral blood—Hematopoietic cells can be contained in or obtained from peripheral, circulating blood. Prior to harvesting, stem cells can be mobilized from marrow into the blood stream by injecting the donor with compounds including cytokines. Such mobilization can be accomplished by using for example, one or more of granulocyte colony-stimulating factor (G-CSF), stem cell factor (SCF), thrombopoietin (Tpo), and a chemotherapeutic agent (i.e., cyclophosphamide). Typically, the donor is injected a few days prior to the harvest. To collect the cells, an intravenous tube is inserted into the donor's vein and donor's blood is passed through a filtering system that pulls out CD34+ white blood cells and returns the red blood cells to the donor. The methods of collection are well known to those skilled in the art. Once collected, the cells can be used as a whole or can be further fractionated into specific cell types and/or cryopreserved for later use in the methods and compositions described herein.

Umbilical cord and/or placental blood—Hematopoietic cells can be obtained from umbilical cord and/or placental blood, i.e. the blood that remains in the placenta and in the attached umbilical cord after childbirth (Nakahata & Ogawa, J. Clin. Invest. 1982; Prindull et al., 6Acta. Paediatr. Scand. 1978; Tchernia et al., J. Lab. Clin. Med. 1981). Several methods of cord blood collections are known in the art. The blood remaining in the delivered placenta is safely and easily collected and stored. The predominant collection procedure currently practiced involves a relatively simple venipuncture, followed by gravity drainage into a standard sterile anti-coagulant-filled blood bag, using a closed system, similar to the one utilized on whole blood collection. After aliquots have been removed for routine testing, the units can be cryopreserved and stored in liquid nitrogen See, e.g., U.S. Pat. No. 7,160,714; U.S. Pat. No. 5,114,672; U.S. Pat. No. 5,004,681; U.S. patent application Ser. No. 10/076,180, Pub. No. 20030032179. Stem and progenitor cells in cord blood appear to have a greater proliferative capacity in culture than those in adult bone marrow (Salahuddin et al., Blood (1981); Cappellini et al., Brit. J. Haematol. (1984)). Umbilical cord blood stem cells have been used to reconstitute hematopoiesis in children with malignant and nonmalignant diseases after treatment with myeloablative doses of chemo-radiotherapy. Sirchia & Rebulla, Haematologica (1999). See also Laughlin Bone Marrow Transplant. (2001); U.S. Pat. No. 6,852,534. The placenta and umbilical cord tissues are also a source of hematopoietic stem and progenitor cells (Robin, C. et al. Cell Stem Cell. 2009.). CN104711226A; U.S. Pat. No. 7,045,148; U.S. Pat. No. 8,673,547B2.

Alternatively, fetal blood can be taken from the fetal circulation at the placental root with the use of a needle guided by ultrasound (Daffos et al., Am. J. Obstet. Gynecol. (1985); Daffos et al., Am. J. Obstet. Gynecol. (1983)), by placentocentesis (Valenti, Am. J. Obstet. Gynecol. (1973); Cao et al., J. Med. Genet. (1982)), by fetoscopy (Rodeck, in Prenatal Diagnosis, Rodeck & Nicolaides, eds., Royal College of Obstetricians & Gynaecologists, London, 1984)). Indeed, the chorionic villus and amniotic fluid, in addition to cord blood and placenta, are sources of pluripotent fetal stem cells (see WO 2003 042405) that may be useful in the methods and compositions herein.

Various kits and collection devices are known for the collection, processing, and storage of cord blood. See, e.g., U.S. Pat. No. 7,147,626; U.S. Pat. No. 7,131,958. Collections should be made under sterile conditions, and the blood may be treated with an anticoagulant. Such an anticoagulants include citrate-phosphate-dextrose, acid citrate-dextrose, Alsever's solution (Alsever & Ainslie, 41 N. Y. St. J. Med. 126-35 (1941), DeGowin's Solution (DeGowin et al., 114 J.A.M.A. 850-55 (1940)), Edglugate-Mg (Smith et al., 38 J. Thorac. Cardiovasc. Surg. 573-85 (1959)), Rous-Turner Solution (Rous & Turner 23 J. Exp. Med. 219-37 (1916)), other glucose mixtures, heparin, or ethyl biscoumacetate. See Hurn Storage of Blood 26-160 (Acad. Press, N Y, 1968).

Various procedures are known in the art and can be used to enrich collected cord blood for hematopoietic cells. These include but are not limited to equilibrium density centrifugation, velocity sedimentation at unit gravity, immune rosetting and immune adherence, counterflow centrifugal elutriation, T lymphocyte depletion, and fluorescence-activated cell sorting, alone or in combination. See, e.g., U.S. Pat. No. 5,004,681. Typically, collected blood is prepared for cryogenic storage by addition of cryoprotective agents such as DMSO (Lovelock & Bishop, 183 Nature 1394-95 (1959); Ashwood-Smith 190 Nature 1204-05 (1961)), glycerol, polyvinylpyrrolidine (Rinfret 85 Ann. N.Y. Acad. Sci. 576 94 (1960)), polyethylene glycol (Sloviter & Ravdin 196 Nature 899-900 (1962)), albumin, dextran, sucrose, ethylene glycol, i-erythritol, D-ribitol, D-mannitol (Rowe, 3(1) Cryobiology 12-18 (1966)), D-sorbitol, inositol, D-lactose, choline chloride (Bender et al., 15 J. Appl. Physiol. 520 24 (1960)), amino acids (Phan & Bender, 20 Exp. Cell Res. 651-54 (1960)), methanol, acetamide, glycerol monoacetate (Lovelock, 56 Biochem. J. 265-70 (1954)), and inorganic salts (Phan & Bender, 104 Proc. Soc. Exp. Biol. Med. (1960)). Addition of plasma (e.g., to a concentration of 20-25%) may augment the protective effect of DMSO.

Collected blood should be cooled at a controlled rate for cryogenic storage. Different cryoprotective agents and different cell types have different optimal cooling rates. See e.g., Rapatz, 5(1) Cryobiology 18-25 (1968), Rowe & Rinfret, 20 Blood 636-37 (1962); Rowe, 3(1) Cryobiology 12-18 (1966); Lewis et al., 7(1) Transfusion 17-32 (1967); Mazur 168 Science 939 49 (1970). Considerations and procedures for the manipulation, cryopreservation, and long-term storage of HSC sources are known in the art. See e.g., U.S. Pat. No. 4,199,022; U.S. Pat. No. 3,753,357; U.S. Pat. No. 4,559,298; U.S. Pat. No. 5,004,681. There are also various devices with associated protocols for the storage of blood. U.S. Pat. No. 6,226,997; U.S. Pat. No. 7,179,643. Accordingly, in some embodiments the HSPC populations used in the methods and composition disclosed herein are obtained or enriched from or are contained in biological source such as bone marrow, peripheral blood, cord blood, amniotic fluid, or placental blood or tissues such as the placenta.

Considerations in the thawing and reconstitution of hematopoietic cells sources are also known in the art. U.S. Pat. No. 7,179,643; U.S. Pat. No. 5,004,681. The HSC source blood may also be treated to prevent clumping (see Spitzer, 45 Cancer 3075-85 (1980); Stiff et al., 20 Cryobiology 17-24 (1983), and to remove toxic cryoprotective agents (U.S. Pat. No. 5,004,681). Further, there are various approaches to determining an engrafting cell dose of HSC transplant units. See U.S. Pat. No. 6,852,534; Kuchler Biochem. Methods in Cell Culture & Virology 18-19 (Dowden, Hutchinson & Ross, Strodsburg, P A, 1964); 10 Methods in Medical Research 39-47 (Eisen, et al., eds., Year Book Med. Pub., Inc., Chicago, Ill., 1964). Thus, not being limited to any particular collection, treatment, or storage protocols, an embodiment of the various aspects disclosed herein provides for the addition of ANG or an agonist thereof to the source of HSPCs. This may be done at collection time, or at the time of preparation for storage, or upon thawing and before infusion. For example, stem cells isolated from a subject, e.g., with or without prior treatment of the subject with ANG, may be incubated in the presence of ANG to maintain HSC quiescence, prevent differentiation, progenitor proliferation and/or expand the number of HSCs. Treated and/or expanded HSCs may be subsequently reintroduced into the subject from which they were obtained (autologous transplantation) or may be introduced into another subject (allogeneic transplantation).

A subject from whom a source of hematopoietic cells can be derived can include anyone who is a candidate for autologous stem cell or bone marrow transplantation during the course of treatment for malignant disease or as a component of gene therapy. Other possible candidates are subjects who donate stem cells or bone marrow to patients for allogeneic transplantation for malignant disease or gene therapy. Subjects may have undergone irradiation therapy, for example, as a treatment for malignancy of cell type other than hematopoietic. Subjects may be suffering from anemia, e.g., sickle cell anemia, thalessemia, aplastic anemia, or other deficiency of HSC derivatives.

### Angiogenin (ANG)

Angiogenin, a 14.1-kD protein, is a potent inducer of neovascularization in vivo. ANG, also known as ribonuclease 5 (RNase5), is a member of the secreted vertebrate specific ribonuclease superfamily, with a 33% sequence homology to the pancreatic ribonuclease A. Angiogenin has angiogenic (Fett et al., 1985), neurogenic (Subramanian and Feng, 2007), neuroprotective (Subramanian et al., 2008), and immune-regulatory functions (Hooper et al., 2003). RNase activity of ANG is important for its angiogenic activity. Endogenous ANG is required for cell proliferation induced by other angiogenic proteins such as vascular endothelial growth factor (VEGF; 192240). Like VEGF, ANG is induced by hypoxia to elicit angiogenesis and is expressed in motor neurons (Lambrechts et al., 2003). The role of Angiogenin as a regulator of hematopoiesis is not known.

Accordingly, as used herein the term “Angiogenin”, “ANG” or “Angiogenin protein” generally refers to an Angiogenin polypeptide that is similar or identical in sequence to a wild-type ANG. In some embodiments, the term “Angiogenin” refers to a Angiogenin polypeptide having an amino acid sequence that is at least 80%, at least 85%, at least 90%, at least 95%, at least 97%, at least 99%, or 100%, identical to that of a wild-type ANG and that retains the ability, at a minimum, to maintain quiescence of primitive HSC (e.g., of LT-HSC) and/or promote proliferation of progenitor cells (e.g., of myeloid restricted progenitor cells) and/or enhance hematopoietic reconstitution in vivo. Accordingly in some embodiments, “ANG” can be full length ANG. In some embodiments, “ANG” can be a functional fragment of a full length ANG, a species homologue and/or functional fragments thereof, an ortholog of ANG and/or functional fragments thereof. The ANG polypeptide can be a mammalian ANG protein. The ANG polypeptide can also be a functional isoform of the full length ANG or functional fragment thereof.

In some embodiments, “ANG” is a wild-type ANG of human origin, having the following amino acid sequence (SEQ ID NO:1), or a functional fragment thereof.

(SEQ ID NO: 1)

| | |
|---|---|
| 1 | MVMGLGVLLL VFVLGLGLTP PTLAQDNSRY THFLTQHYDA |
| | KPQGRDDRYC ESIMRRRGLT |
| 61 | SPCKDINTFI HGNKRSIKAI CENKNGNPHR ENLRISKSSF |
| | QVTTCKLHGG SPWPPCQYRA |
| 121 | TAGFRNVVVA CENGLPVHLD QSIFRRP |

(See GenBank Accession No. AAA51678.1, which is incorporated herein by reference in its entirety).

A “functional fragment” refers to fragment of the full length ANG (e.g. corresponding to SEQ ID NO:1) of at least 10, at least 20, at least 30, at least 40, at least 50, at least 60, at least 70, at least 80, at least 90, at least 100, at least 110, at least 120, at least 130, at least 140 consecutive amino acids of full length wild-type ANG, that has at least about 70%, 80%, 90%, 100% or more than 100% of the function of wild-type ANG (e.g., of SEQ ID NO:1) at reconstituting hematopoietic cells in vivo or in vitro. The functional activity can be tested by one of ordinary skill in the art by the assays described in the examples.

The polypeptide and coding nucleic acid sequences of ANG and of other members of the family of human origin and those of a number of animals are publically available, e.g., from the NCBI website and are contemplated for use in the methods and compositions herein. Examples include, but are not limited to, Mouse (GenBank Accession No. AAA91366.1), Rat (GenBank Accession No. AAR28758.1), Bovine (GenBank Accession No. AAG47631.1).

In some embodiments, the ANG polypeptide can be a mammalian homolog of human ANG or a functional fragment thereof. In some embodiments, the ANG polypeptide has an amino acid sequence at least 85%, at least 90%, at least 95%, at least 97% or at least 99% identical to the amino acid sequence of SEQ ID NO:1 and maintains quiescence of primitive HSCs (e.g., LT-HSCs, ST-HSCs, MPP) and promotes proliferation of myeloid restricted progenitors (e.g., CMP, GMP, MEP). In some embodiments, the ANG polypeptide has an amino acid sequence that has at least 85%, at least 90%, at least 95%, at least 97% or at least 99% amino acid sequence homology to amino acid sequence of SEQ ID NO: 1 and maintains quiescence of primitive HSCs and promotes proliferation of myeloid restricted progenitors. In some embodiments, ANG is a functional fragment of SEQ ID NO:1 of at least 10, at least 20, at least 30, at least 40, at least 50, at least 60, at least 70, at least 80, at least 90, at least 100, at least 110, at least 120, at least 130, at least 140 consecutive amino acids of SEQ ID NO:1, that has at least about 50%, 60%, 70%, 80%, 90%, 100% or more than 100% of the function of wild type ANG (e.g., human ANG of SEQ ID NO:1) at reconstituting hematopoietic cells in vivo or in vitro. The functional activity can be tested by one of ordinary skill in the art by the assays described in the examples.

Percent (%) amino acid sequence identity for a given polypeptide sequence relative to a reference sequence is defined as the percentage of identical amino acid residues identified after aligning the two sequences and introducing gaps if necessary, to achieve the maximum percent sequence identity, and not considering any conservative substitutions as part of the sequence identity. Percent (%) amino acid sequence homology for a given polypeptide sequence relative to a reference sequence is defined as the percentage of identical or strongly similar amino acid residues identified after aligning the two sequences and introducing gaps if necessary, to achieve the maximum percent homology. Non identities of amino acid sequences include conservative substitutions, deletions or additions that do not affect the biological activity of ANG. Strongly similar amino acids can include, for example, conservative substitutions known in the art. Percent identity and/or homology can be calculated using alignment methods known in the art, for instance alignment of the sequences can be conducted using publicly available software software such as BLAST, Align, ClustalW2. Those skilled in the art can determine the appropriate parameters for alignment, but the default parameters for BLAST are specifically contemplated.

In one embodiment, “ANG polypeptide” useful in the methods and compositions described herein consists of, consists essentially of, or comprises an amino acid sequence, or is a fragment thereof derived from SEQ ID NO:1, provided that the polypeptide retains at least one biological activity of full length ANG of SEQ ID NO: 1, the biological activity being selected from at a minimum, to maintain quiescence of primitive HSC (e.g., of LT-HSC) and/or promote proliferation of myeloid restricted progenitor cells and/or enhance hematopoietic reconstitution in vivo.

The polypeptides described herein can comprise conservative amino acid substitutions at one or more amino acid residues, e.g., at essential or non-essential amino acid residues but will retain a therapeutically or physiologically relevant activity of an inhibitory peptide as that term is described herein. A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, in a conservative substitution variant, a nonessential amino acid residue in the polypeptide is preferably replaced with another amino acid residue from the same side chain family.

In some embodiments, ANG can be a variant of wild type ANG. The term “variant” as used herein refers to a polypeptide or nucleic acid that is “substantially similar” to a wild-type ANG. A molecule is said to be “substantially similar” to another molecule if both molecules have substantially similar structures (i.e., they are at least 50% similar in amino acid sequence as determined by BLASTp alignment set at default parameters) and are substantially similar in at least one therapeutically or physiologically relevant biological activity. A variant differs from the naturally occurring polypeptide or nucleic acid by one or more amino acid or nucleic acid deletions, additions, substitutions or side-chain modifications, yet retains one or more therapeutically relevant, specific functions or desired biological activities of the naturally occurring molecule (e.g., maintains primitive HSCs in a quiescent state, enhances hematopoietic reconstitution in vivo).

Amino acid substitutions include alterations in which an amino acid is replaced with a different naturally-occurring or a non-conventional amino acid residue. Some substitutions can be classified as “conservative,” in which case an amino acid residue contained in a polypeptide is replaced with another naturally occurring amino acid of similar character either in relation to polarity, side chain functionality or size. Substitutions encompassed by variants as described herein can also be “non-conservative,” in which an amino acid residue which is present in a peptide is substituted with an amino acid having different properties (e.g., substituting a charged or hydrophobic amino acid with an uncharged or hydrophilic amino acid), or alternatively, in which a naturally-occurring amino acid is substituted with a non-conventional amino acid. Also encompassed within the term “variant,” when used with reference to a polynucleotide or polypeptide, are variations in primary, secondary, or tertiary structure, as compared to a reference polynucleotide or polypeptide, respectively (e.g., as compared to a wild-type polynucleotide or polypeptide). Polynucleotide changes can result in amino acid substitutions, additions, deletions, fusions and truncations in the polypeptide encoded by the reference sequence. Variants can also include insertions, deletions or substitutions of amino acids in the peptide sequence. To be therapeutically useful, such variants will retain a therapeutically or physiologically relevant activity as that term is used herein.

The ANG polypeptide can be recombinant, purified, isolated, naturally occurring or synthetically produced. The term “recombinant” when used in reference to a nucleic acid, protein, cell or a vector indicates that the nucleic acid, protein, vector or cell containing them have been modified by introduction of a heterologous nucleic acid or protein or the alteration of a native nucleic acid or a protein, or that the cell is derived from a cell so modified. The term “heterologous” (meaning ‘derived from a different organism’) refers to the fact that often the transferred protein was initially derived from a different cell type or a different species from the recipient. Typically the protein itself is not transferred, but instead the genetic material coding for the protein (often the complementary DNA or cDNA) is added to the recipient cell. Methods of generating and isolating recombinant polypeptides are known to those skilled in the art and can be performed using routine techniques in the field of recombinant genetics and protein expression. For standard recombinant methods, see Sambrook et al, Molecular Cloning: A Laboratory Manual, Cold Spring Harbor Laboratory Press, NY (1989); Deutscher, Methods in Enzymology 182:83-9 (1990); Scopes, Protein Purification: Principles and Practice, Springer-Verlag, NY (1982).

In some embodiments, ANG can be an agonist of wild-type ANG, an analog or a derivative thereof. In some embodiments, the agonist of wild-type ANG, an analog or a derivative thereof, retains at least one biological activity of full length ANG of SEQ ID NO: 1, the biological activity being selected from at a minimum, to maintain quiescence of primitive HSC (e.g., of LT-HSC) and/or promote proliferation of myeloid restricted progenitor cells and/or enhance hematopoietic reconstitution in vivo. In some embodiments, the agonist of wild-type ANG, an analog or a derivative thereof, retains at least 50%, at least 60%, at least 70%, at least 80%, at least 90%, at least 100% or more than 100% of the biological activity of full length ANG of SEQ ID NO:1.

### Biological Activity of Angiogenin

The minimum, central biological activity and/or biological effect of the ANG protein as described herein at is least one of, i. maintaining primitive HSCs quiescence and ii. increasing progenitor proliferation, upon contact with a population of hematopoietic cells or source containing a population of hematopoietic cells. In some embodiments, the ANG protein restricts proliferation of primitive hematopoietic stem cells and/or lymphoid-biased progenitors e.g., MPP4s. In some embodiments, the ANG protein increases proliferation of myeloid restricted progenitors for example CMP, GMP, MEP, myeloid biased progenitors e.g., MPP3. In some embodiments, ANG protein maintains LT-HSCs in a quiescent state. In some embodiments, ANG protein increases primitive HSCs quiescence. In some embodiments, ANG protein restricts proliferation and/or differentiation of the LT-HSCs. In some embodiments the ANG protein enables in vitro and in vivo expansion of a population of hematopoietic stem and/or progenitor cells. In some embodiments ANG protein enhances the reconstitution potential of the transplanted hematopoietic cells population. In some embodiments ANG protein results in enhanced hematopoietic reconstitution upon in vivo administration of a population of hematopoietic cells contacted with or cultured ex vivo in presence of ANG. In some embodiments, ANG is a regulator of HSPC stemness. In some embodiments, ANG results in enhanced hematopoietic reconstitution in vivo (e.g. Long-term and/or short-term reconstitution). In some embodiments, ANG maintains the self-renewal capacity of hematopoietic cells. In some embodiments, ANG results in multi-lineage hematopoietic reconstitution of the treated hematopoietic cells population. In some embodiments, ANG results in short-term reconstitution of the treated hematopoietic cells upon their administration in vivo. In some embodiments, the ANG results in long-term reconstitution of the treated hematopoietic cells upon their administration in vivo. Methods for determining hematopoietic reconstitution are known in the art and disclosed above. In some embodiments, ANG protein is a regulator of HSPCs. In some embodiments, the ANG is a regulator of hematopoiesis. In some embodiments, the ANG polypeptide retains at least 50%, at least 60%, at least 70%, at least 80%, at least 90%, at least 95%, at least 99% or even 100% or greater of wild-type ANG (e.g. human ANG of SEQ ID NO:1).

In some embodiments, the ANG polypeptide or fragment thereof used in the methods and compositions disclosed herein retains the ribonucleolytic activity. The ribonucleolytic activity of the ANG used for example can be at least 80%, 85%, 90%, 95%, 99%, 100% of that of the native full length polypeptide of SEQ ID NO:1. In some embodiments, the ANG polypeptide or fragment thereof used in the present disclosure retains receptor binding activity. The receptor binding activity of the ANG used for example can be at least 80%, 85%, 90%, 95%, 99%, 100% of that of the native full length polypeptide of SEQ ID NO:1.

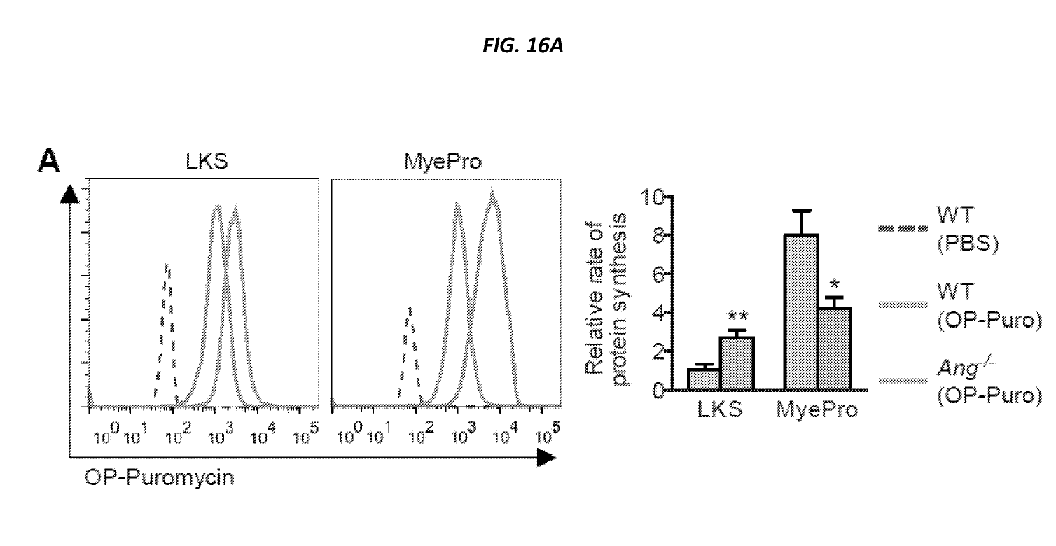

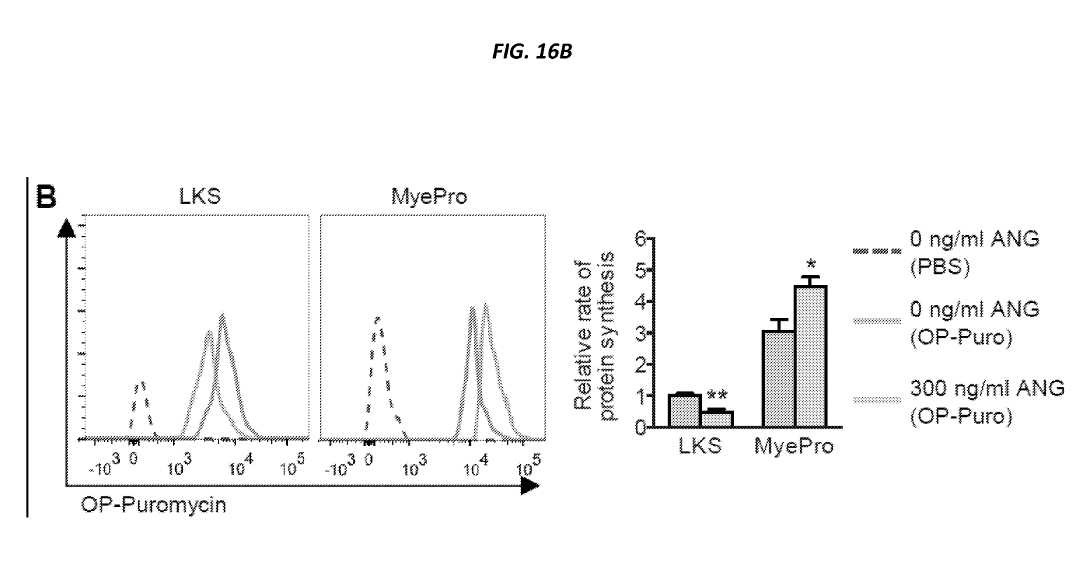

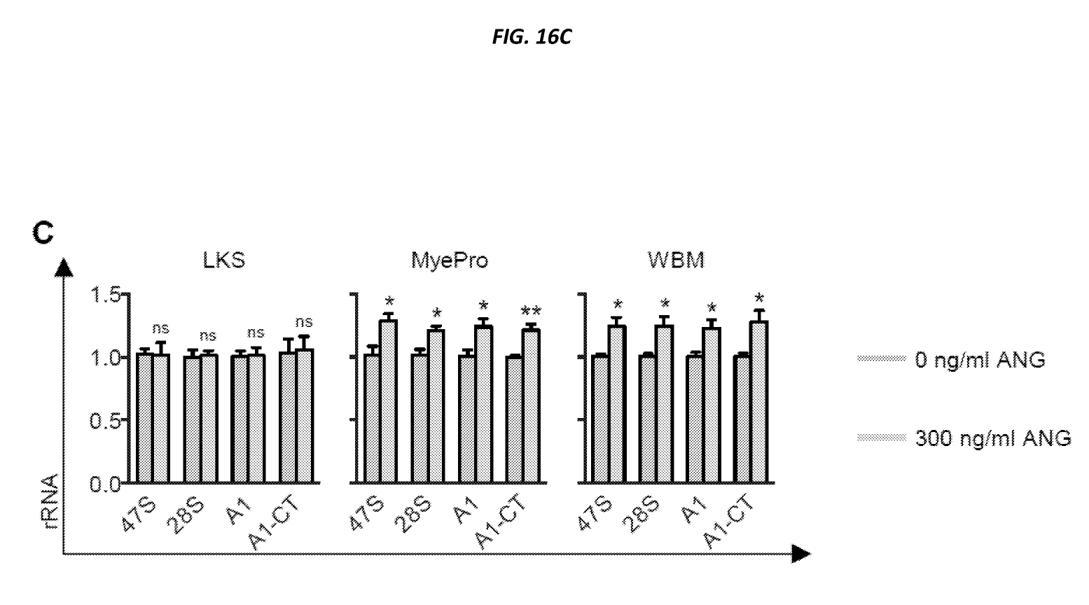

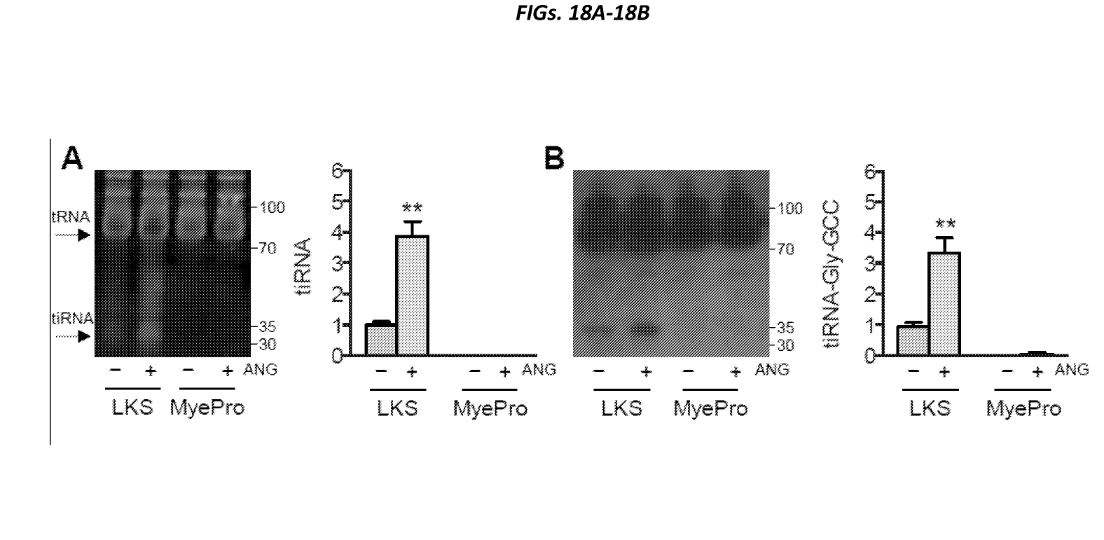

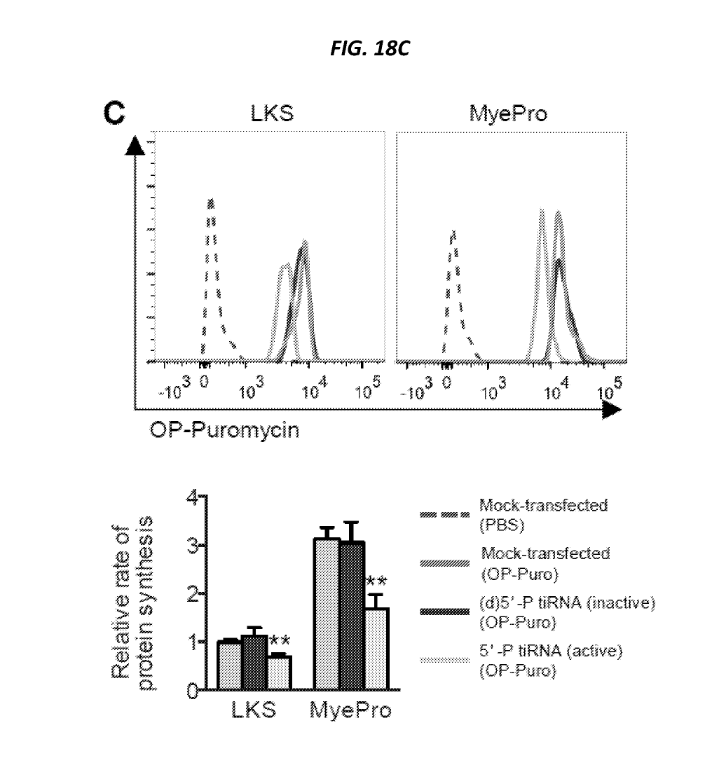

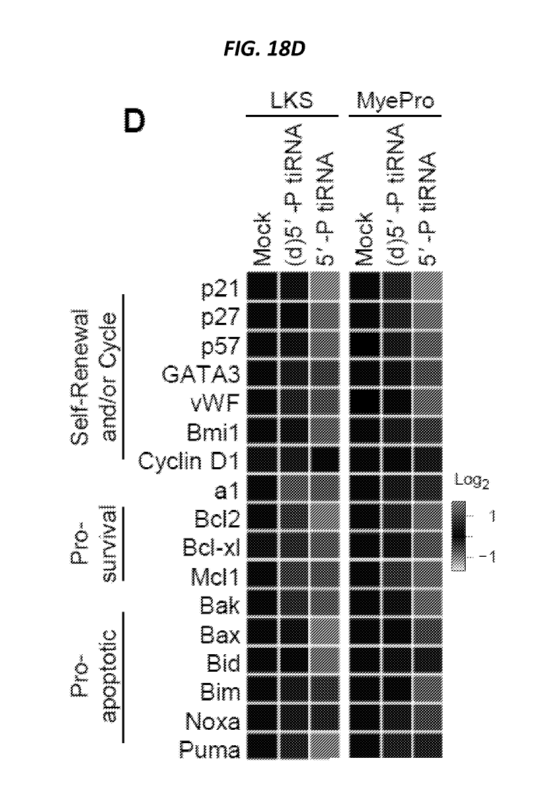

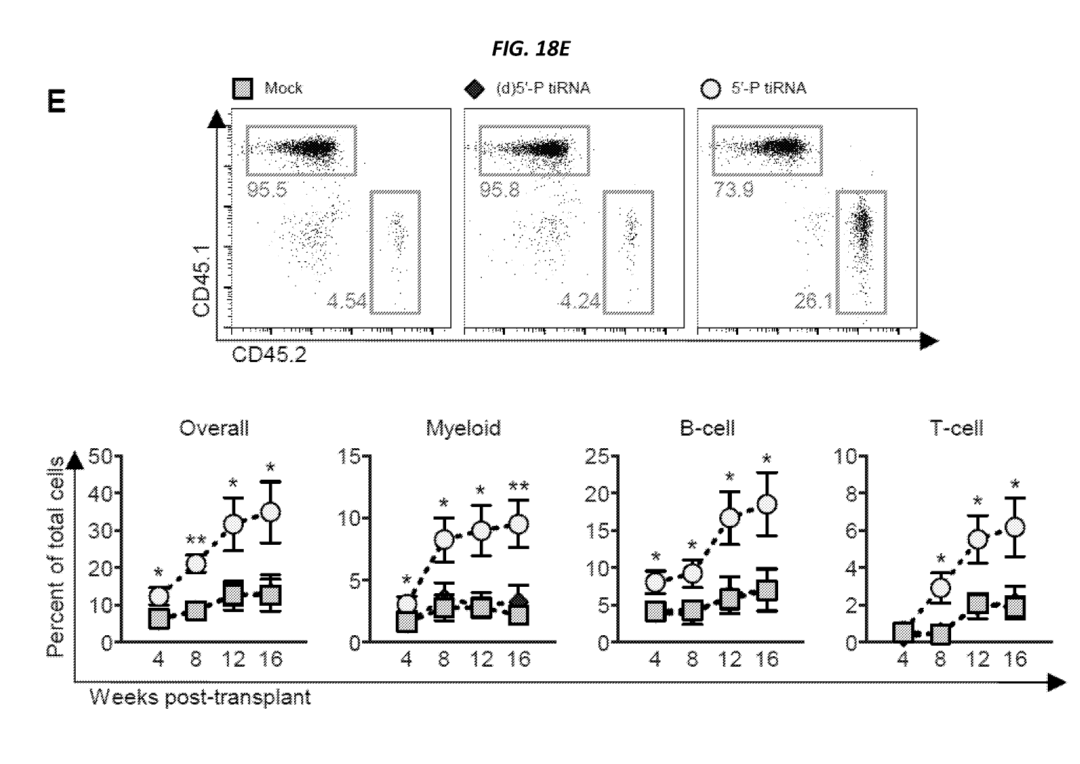

Without wishing to be bound by theory, ANG has been reported in other cell types to regulate global protein synthesis. A higher rate of protein synthesis was observed Ang−/− LKS cells, while Ang−/− myeloid-restricted progenitors demonstrated reduced protein synthesis (FIG. 16A). Accordingly, in some embodiments, ex vivo contact with ANG results in decrease in protein synthesis in hematopoietic cells and increase in protein synthesis of myeloid restricted progenitor cells. While not wishing to be bound by theory, ANG has been shown to reprogram protein synthesis as a stress response to promote survival under adverse conditions. This function of ANG is mediated by tiRNA, a noncoding small RNA that specifically permits translation of anti-apoptosis genes while global protein translation is suppressed so that stressed cells have adequate time and energy to repair damage, collectively promoting cell survival (Emara et al., 2010; Fu et al., 2009; Ivanov et al., 2011; Yamasaki et al., 2009). Addition of ANG led to markedly elevated tiRNA levels in LKS cells (FIG. 18A). Accordingly, in some embodiments, the methods disclosed herein comprise increasing the tiRNA levels in the HSCs for example LT-HSC and/or decreasing tiRNA levels in myeloid restricted progenitor cells. In some embodiments, the increasing of tiRNA levels in LT-HSCs and decreasing of tiRNA levels in myeloid restricted progenitors comprises of contact of the said population of hematopoietic cells with an effective amount of ANG.

### Exposing Hematopoietic Cells or Source Containing Hematopoietic Cells to ANG Ex Vivo

The technology described herein is based in part on the discovery that in vivo or ex vivo, exposure of hematopoietic cells or a population of hematopoietic cells to ANG, results in enhanced hematopoietic reconstitution including repopulation of cells of all blood lineage and their functions as well as enhanced self-replication of the HSCs to repopulate and maintain the stem cell pool, after in vivo administration of the treated cells. Accordingly, in one aspect, the technology herein relates to a population of hematopoietic cells, that has been contacted with, exposed to or treated with ANG ex vivo, which can be transplanted into a patient in need of improved hematopoietic reconstitution. While not wishing to be bound by theory, the exposure to ANG results in restricted proliferation, increase of quiescence and self-renewal capacities of the primitive HSCs, while preserving their viability and differentiation state. Accordingly, one aspect of the technology herein relates to a population of primitive HSCs generated after ex vivo exposure to ANG. In some embodiments, the technology described herein relates to method of generating the said population of quiescent primitive HSC. Furthermore, ex vivo exposure to ANG results in promotion of proliferation and expansion of progenitor cells, (e.g., myeloid restricted progenitor). Accordingly, one aspect of the technology herein relates to a population of progenitor cells (e.g., myeloid restricted progenitor cells) with enhanced proliferative capacity after exposure to ANG ex vivo. In some embodiments, provided herein are methods of generating said population of proliferative progenitor cells. A further embodiment of the technology herein provides a method for expanding a population of hematopoietic cells comprising primitive hematopoietic stem cells and/or myeloid restricted progenitors, preferably myeloid restricted progenitors ex vivo upon contacting with an effective amount of ANG for a sufficient time such that the contacting results in quiescence of primitive HSCs and proliferation of myeloid restricted progenitors.

A population of hematopoietic cells obtained after ex vivo exposure with ANG can be administered to the subject in need of stem cell transplantation and/or improved hematopoietic reconstitution. In one aspect provided herein is a method of administering to a subject a population of hematopoietic cells s that has been treated/exposed ex vivo to ANG. In some embodiments, a population of hematopoietic cells obtained upon treatment with ANG can be cryopreserved, such that they can later be thawed and used, e.g., for administration to a patient. In general, the cells are stored in a typical freezing medium, e.g., 10% DMSO, 50% fetal calf serum (FCS), and 40% cell culture medium. The exposed cell population or a source containing exposed cell population e.g., blood product can be deposited into a blood bank. Accordingly, in one aspect provided herein is a blood bank comprising a population of hematopoietic cells obtained upon ex vivo exposure to ANG. Another aspect of the technology described herein provides for a kit comprising a container suitable for hematopoietic cells source sample storage in which the container is preloaded with an effective amount of ANG. An additional embodiment provides a kit comprising a container suitable for hematopoietic cells source sample storage and a vial containing a suitable amount of ANG.

In some embodiments, a population of hematopoietic cells is cultured in presence of ANG or agonist thereof. Methods of culturing hematopoietic cells in vitro are well known in the art. The cells can be cultured for example, in Phosphate buffered saline, or a commercially available media such as StemSpan SFEM (Stem Cell Technologies). The media can be further supplemented with other known modulators of HSPCs. Non-limiting examples of other modulators include one or more of interleukin-3 (IL-3), interleukin-6 (IL-6), interleukin-11 (IL-11), interleukin-12 (IL-12), stem cell factor (SCF), fms-like tyrosine kinase-3 (fit-3), transforming growth factor-β (TGF-B), an early acting hematopoietic factor, described, for example in WO 91/05795, and thrombopoietin (Tpo). The effective dosage of ANG used in in vitro culture can be described as a dosage necessary to maintain primitive HSCs in an undifferentiated state and/or quiescent state, and/or a dosage necessary to enhance proliferation and expansion of myeloid restricted progenitor cells and/or a dosage necessary to enhance the post-transplant reconstitution of the treated cell upon in vivo administration. The effective amounts may vary, as recognized by those skilled in the art, depending on the number of hematopoietic cells to be treated, the duration of treatment, source of hematopoietic cells, the specific underlying disease to be treated by transplantation, intensity of prior therapy such as chemotherapy or radiotherapy.

The effective duration of ex vivo contact with ANG can be determined by those of skill in the art. For example, the population of hematopoietic cells can be maintained in contact with ANG for a period of about 2 hours, about 4 hours, about 6 hours, about 24 hours, about 2 days or longer, at least 7 days. In one embodiment, cells can be treated for at least 2 hours prior to changing to medium without ANG.

In some embodiments, the cells can be maintained in culture in absence of ANG before addition of ANG, and then transplanted in vivo. In some embodiments, the cells can be cultured in presence of ANG and then can be maintained in absence of ANG prior to transplantation in vivo. In some embodiments, the cells may be administered in vivo along with ANG. In some embodiments, in addition to ANG, the cells can be cultured in combination with one or more regulators disclosed in the present disclosure for example, Embigin, IL8. The effective concentration and duration of treatment can vary for each of the regulators and can be easily determined by one skilled in the art. The cells can be treated simultaneously with these factors or on different times.

The contacting with ANG in methods disclosed herein can be done at initial collection of the source and/or the cells, during processing, at storage, upon thawing, prior to in vivo administration, or during in vivo administration. Methods to determine cellular proliferation and/or increase of quiescence and/or expansion are known in the art. Briefly the cell number of a desired cell population can be enumerated using a hemocytometer before and after the treatment with ANG. Cellular expansion and proliferation is indicated by an increase in cell number. Quiescence of primitive HSCs can be indicated by decreased or no change in cell numbers of lineage restricted progenitor cells.

### Stem Cell Transplants

Stem cell transplants are used to restore the stem cell reservoir when the bone marrow has been destroyed by disease, chemotherapy (chemo), or radiation. Depending on the source of the stem cells, this procedure may be called a bone marrow transplant, a peripheral blood stem cell transplant, or a cord blood transplant. They can all be called hematopoietic stem cell transplants (HSCT). Hematopoietic stem cells (HSC) and progenitors are commonly used to replace the hematopoietic system in patients with hematopoietic malignancies, or patients undergoing high dose chemotherapy. Hematopoietic reconstitution after transplantation encompasses the recovery of optimal numbers of hematopoietic stem cells and hematopoietic cells of both the myeloid and lymphoid lineages and their functions, thereby restoring a functional bone marrow. In one aspect, the methods and compositions described herein result in enhanced hematopoietic reconstitution in vivo. In some embodiments the reconstitution potential obtained using the methods and compositions described herein is multi-lineage. Multi-lineage reconstitution or repopulation or differentiation can be defined as an ability to differentiate in multiple mature blood cell types. Exemplary method for assessment of HSCs multi-potentiality and/or multi-lineage reconstitution, includes detection of human CD45+ cells, represented by at least myeloid and lymphoid lineages in blood or/and in bone marrow. Commonly used set of lineage markers in combination with human pan-leukocyte CD45 can include myeloid lineage: CD33 or CD13, B-cell lymphoid: CD19, T-cell lymphoid: CD4+CD8 or CD3, erythroid: GlyA (CD235a). In some embodiments the post-transplantation hematopoietic reconstitution can be short-term recovery or sustained long-term reconstitution. In human patients, sustained and/or long-term reconstitution can be assessed by persistence of human-derived lymphoid and myeloid cells in the blood and/or HSCs and their mature progeny in bone marrow at least 12-20 weeks after primary transplant. In some embodiments, the long-term hematopoietic reconstitution can be for example, at least 12 weeks (or 3 months), at least 13 weeks, at least 14 weeks, at least 15 weeks, at least 16 weeks (or 4 months), at least 17 weeks, at least 18 weeks, at least 18 weeks, at least 20 weeks (or 5 months), at least 6 months, at least 1 year or more. In some embodiments, the methods and compositions disclosed herein can result in result in sustained hematopoietic reconstitution after a single transplant. In some embodiments, the hematopoietic reconstitution is short-term i.e. for a period not exceeding three months. The short-term reconstitution can be for example, less than 3 months (12 weeks), less than 11 weeks, less than 10 weeks, less than 9 weeks, less than 8 weeks (or 2 months), less than 7 weeks, less than 6 weeks, less than 5 weeks, 4 weeks or less. In some embodiments, the methods and compositions disclosed herein can enhance the self-renewal capacity of the HSC population after transplantation in vivo. Self-renewal can be defined as an ability of human-derived cells to multilineage repopulation and/or engraftment in bone marrow in serial transplantation (at least after secondary).

### Methods to Assess Hematopoietic Reconstitution

Methods to determine successful transplant and/or hematopoietic reconstitution are known in the art. The long term repopulating ability of candidate hematopoietic stem cells can be evaluated, e.g., in an in vivo sheep model or an in vivo NOD-SCID mouse model for human HSC. The NOD/SCID mouse is an immunodeficient recipient, which allows the introduction of human, NHP or mouse cells and the determination of stem cell functionality through engraftment, proliferation and differentiation into at least two distinct lineages (typically myeloid and lymphoid). This in vivo reconstitution assay is typically known as the Competitive Repopulating Unit (CRU) or SCID Repopulating Cell (SRC) assay. In humans, for example, successful hematopoietic reconstitution can be determined, by measurement of absolute counts for individual blood cell types (white blood cells, red blood cells and platelets) in the peripheral blood, reaching a number of cells accepted by those of skill in the art as within the normal range for the subject. Methods of conducting a complete blood count, differential leukocyte count i.e. including counts of each type of white blood cell, for e.g., neutrophils, eosinophils, basophils, monocytes, and lymphocytes, and platelet counts are known to those skilled in the art. Briefly, post-transplantation, the blood can be collected at regular intervals in a tube containing an anti-coagulant like the EDTA, the cells can be counted using an automated blood count analyzer or manually using a hemocytometer. Neutrophils are a type of white blood cell that are a marker of engraftment; the absolute neutrophil count (ANC) must be at least 500 for three days in a row to say that engraftment has occurred. This can occur as soon as 10 days after transplant, although 15 to 20 days is common for patients who are given bone marrow or peripheral blood cells. Umbilical cord blood recipients usually require between 21 and 35 days for neutrophil engraftment. Platelet counts are also used to determine when engraftment has occurred. The platelet count must be between 20,000 and 50,000 (without a recent platelet transfusion). This usually occurs at the same time or soon after neutrophil engraftment, but can take as long as eight weeks and even longer in some instances for people who are given umbilical cord blood.

Alternatively analysis of chimerism status can be monitored for example following allogeneic transplantation. Analysis of chimerism involves discrimination between donor- and recipient-derived hematopoiesis based on molecular methods for example using cytogenetics, isoenzyme analysis, blood group phenotyping, sex chromosome differentiation using fluorescence in situ hybridization, or using PCR-based methods relying on the amplification of highly polymorphic repetitive DNA sequences such as short tandem repeats (STR), variable number of tandem repeat (VNTR) sequences. The methods for whole blood chimerism analysis are known to those skilled in the art. Exemplary method involves, obtaining blood samples at routine points post-transplant, or when there is a suspicion of disease relapse. DNA is extracted from EDTA blood sample for example, using a magnetic purification method (Qiagen EZ1). Forensic kits, comprising, PCR reactions using three STR markers are commercially available (Promega PwerPlex16 Monoplex System). The differentially sized PCR products can be detected and analyzed on a capillary system genetic analyser (Applied Biosystems 3130xl). Lineage specific chimerism analysis can be done by separating the leukocyte lineages by cell separation using AutoMACS immune magnetic separation technology. Positive chimerism analysis performed on patients who underwent transplant to ameliorate a malignant disease can indicate signal of appearance of malignant cells or give a measure of efficiency of transplantation.

In some embodiments enhanced hematopoietic reconstitution; treats, reverse, alleviate, ameliorate, inhibit, slow down or stop the progression or severity of the disease resulting in improper functioning of the bone marrow and the immune system or their symptoms. The efficacy of a given therapeutic regimen involving methods and compositions described herein may be monitored, for example by convention FACS assays for phenotypes of cells in the blood circulation of the subject under treatment. Such analysis is useful to monitor changes in the numbers of cells of various lineages, e.g., myeloid lineage or lymphoid lineage.

Patient Selection and Treatment

While the methods and compositions described herein can be used to enhance hematopoietic reconstitution of in vivo hematopoietic cells or transplanted hematopoietic cells, in some embodiments, they can be described to be of use in one or more of the following situations; (1) Replace diseased, nonfunctioning bone marrow with healthy functioning bone marrow (for example in conditions such as leukemia, aplastic anemia, and sickle cell anemia), (2) Regenerate a new immune system that will fight existing or residual disorder for example leukemia or other cancers not killed by the chemotherapy or radiation, (3) Replace the bone marrow and restore its normal function after high doses of chemotherapy and/or radiation are given to treat a malignancy (for diseases such as lymphoma and neuroblastoma). This process can be called rescue or hematopoietic reconstitution. (4) Replace bone marrow with genetically healthy functioning bone marrow to prevent further damage from a genetic disease process (for example Hurler's syndrome and adrenoleukodystrophy).

A subject having or susceptible to decreased levels of HSCs and/or HPCs and/or blood cell deficiency can benefit from the methods and compositions disclosed herein. Decreased levels of HSCs and/or HPCs and/or blood cell deficiency can be caused due to a number of conditions, for example due to hematological diseases also called as blood disorders and hematological malignancies. In one aspect, the technology herein relates to methods and compositions useful in the treatment and prevention of blood disorders and/or to ameliorate symptoms and disorders related to decreased levels of HSCs and/or HPCs and/or blood cell deficiency, for example hematological disorders. In some embodiments, the subject is suffering or is at a risk of suffering from one or more disorders described herein.

Hemoglobinopathies and thalassemia can both be characterized as “blood disorders”. Blood disorders include disorders that can be treated, prevented, or otherwise ameliorated by the administration of compositions disclosed herein. A blood disorder is any disorder of the blood and blood-forming organs. The term blood disorder includes nutritional anemias (e.g., iron deficiency anemia, sideropenic dysphasia, Plummer-Vinson syndrome, vitamin B12 deficiency anemia, vitamin B12 deficiency anemia due to intrinsic factor, pernicious anemia, folate deficiency anemia, and other nutritional anemias), myelodysplastic syndrome, bone marrow failure or anemia resulting from chemotherapy, radiation or other agents or therapies, hemolytic anemias (e.g., anemia due to enzyme disorders, anemia due to phosphate dehydrogenase (G6PD) deficiency, favism, anemia due to disorders of glutathione metabolism, anemia due to disorders of glycolytic enzymes, anemias due to disorders of nucleotide metabolism and anemias due to unspecified enzyme disorder), thalassemia, α-thalassemia, β-thalassemia, δβ-thalassemia, thalassemia trait, hereditary persistence of fetal hemoglobin (HPFP), and other thalassemias, sickle cell disorders (sickle cell anemia with crisis, sickle cell anemia without crisis, double heterozygous sickling disorders, sickle cell trait and other sickle cell disorders), hereditary hemolytic anemias (hereditary spherocytosis, hereditary elliptocytosis, other hemoglobinopathies and other specified hereditary hemolytic anemias, such as stomatocyclosis), acquired hemolytic anemia (e.g., drug-induced autoimmune hemolytic anemia, other autoimmune hemolytic anemias, such as warm autoimmune hemolytic anemia, drug-induced non-autoimmune hemolytic anemia, hemolytic-uremic syndrome, and other non-autoimmune hemolytic anemias, such as microangiopathic hemolytic anemia); aplastic anemias (e.g., acquired pure red cell aplasia (erythoblastopenia), other aplastic anemias, such as constitutional aplastic anemia and fanconi anemia, acute posthemorrhagic anemic, and anemias in chronic diseases), coagulation defects (e.g., disseminated intravascular coagulation (difibrination syndrome)), hereditary factor VIII deficiency (hemophilia A), hereditary factor IX deficiency (Christmas disease), and other coagulation defects such as Von Willebrand's disease, hereditary factor Xi deficiency (hemophilia C), purpura (e.g., qualitative platelet defects and Glanzmann's disease), neutropenia, agranulocytosis, functional disorders of polymorphonuclear neutrophils, other disorders of white blood cells (e.g., eosinophilia, leukocytosis, lymophocytosis, lymphopenia, monocytosis, and plasmacyclosis), diseases of the spleen, methemoglobinemia, other diseases of blood and blood forming organs (e.g., familial erythrocytosis, secondary polycythemia, essential thrombocytosis and basophilia), thrombocytopenia, infectious anemia, hypoproliferative or hypoplastic anemias, hemoglobin C, D and E disease, hemoglobin lepore disease, and HbH and HbS diseases, anemias due to blood loss, radiation therapy or chemotherapy, or thrombocytopenias and neutropenias due to radiation therapy or chemotherapy, sideroblastic anemias, myelophthisic anemias, antibody-mediated anemias, and certain diseases involving lymphoreticular tissue and reticulohistiocytic system (e.g., Langerhans' cell hystiocytosis, eosinophilic granuloma, Hand-Schuller-Christian disease, hemophagocytic lymphohistiocytosis, and infection-associated hemophagocytic syndrome).

In some embodiments, the blood deficiencies are acquired or genetic deficiencies. Genetic blood disorders are well known by persons of ordinary skill in the art, and include, without limitation, Thalassemias, Sickle cell disease, hereditary spherocytosis, G6PD Deficiency hemolytic anemia, Kostman's syndrome, Swachman-Diamond Syndrome, Cyclic neutropenia, Hereditary neutropenia, Dyskeratosis Congenita, Hereditary thrombocytopenia syndromes, Wiskott-Aldrich Syndrome, May-Hegglin anomaly, Thrombocytopenia with Absent Radii Syndrome, Fanconi's anemia and other hereditary blood disorders.

In some embodiments, the compositions and methods as disclosed herein can be used for the treatment of neutropenia. Neutrophenia is a disorder of low white blood cell count in a subject, and is characterized by one or more of the following: an absolute neutrophil count (ANC) of less than 1500/μL. People suffering or diagnosed with neutrophia may result in hospitalization for treatment of fever, neutropenic sepsis, and can cause potentially fatal infection. Neutropenia is very common in subjects undergone or currently undergoing chemotherapy, transplants, radiation therapy and the like.

In some embodiments, the methods and composition disclosed herein can be used for the treatment of low platelet count, for example, but not limited to, a low platelet count occurring in thrombocytopenia and/or platelet dysfunction. There is currently no or inadequate drug therapy, and the only current treatment is a platelet transfusion. In some embodiments, the methods and compositions disclosed herein can be used for the treatment of low platelet count which occurs as a consequence of other disorders, for example, but not limited to, AIDS (acquired immunodeficiency syndrome); ITP (immune thrombocytopenic purpura); DIC (disseminated intravascular coagulation); TTP (thrombotic thrombocytopenic purpura) and the like.

In some embodiments, the methods and compositions as disclosed herein can be used for the treatment of cytopenias. Significant cytopenias are associated with radiation therapies and also occur after or during chemotherapy and chemo-radiation.

In some embodiments, the methods and compositions disclosed herein can be used to treat a subject suffering from malignancy for example, hematological malignancy. Examples of malignancies that can benefit from the technology detailed herein include but are not limited to, lymphoma (Hodgkin's disease, Burkitt's lymphoma, Anaplastic large cell lymphoma, Splenic marginal zone lymphoma, Hepatosplenic T-cell lymphoma, Angioimmunoblastic T-cell lymphoma), myeloma (Plasmacytoma, Waldenstrom macroglobulinemia, Multiple myeloma), Leukemia (Aggressive NK-leukemia, T-cell large granular lymphocyte leukemia, Acute lymphocytic leukemia, Chronic lymphocytic leukemia, Acute myelogenous leukemia, Chronic myelogenous leukemia, Chronic idiopathic myelofibrosis, Chronic myelogenous leukemia, T-cell prolymphocytic leukemia, B-cell prolymphocytic leukemia, Chronic neutrophilic leukemia, Hairy cell leukemia). In some embodiments, the methods and compositions disclosed herein can be used to treat subjects suffering from solid tumors. Non-limiting examples of solid tumors can include solid tumors of childhood (Peripheral Neuroblastoma, Ewing's Sarcoma and the Ewing Family of Tumors, Rhabdomyosarcoma, Wilms Tumor, Osteosarcoma, Retinoblastoma), Lung cancer, any histology Colon cancer, Rectal cancer, Pancreas cancer, Stomach cancer, Esophageal cancer, Gall bladder cancer, Cancer of the bile duct, Renal cell cancer, Cervical cancer, Uterine cancer, Cancer of the fallopian tubes, Epithelial ovarian cancer, Breast cancer, Prostate cancer, Nasopharyngeal cancer, Paranasal sinus cancer, Neuroendocrine tumors, Soft tissue sarcomas, Thyroid tumors, Tumors of the thymus, Tumors of unknown primary origin, Malignant melanoma, Glioma.

Radiation therapy and chemotherapy are usually considered treatment options for patients suffering from cancer, which may result in ablation of bone marrow. Additionally chemotherapy or radiation therapy may be given prior to a stem cell transplant as part of the myeloablative conditioning regimen, in order to eradicate the patient's disease and suppress immune reaction prior to HSC transplant. Accordingly, in some embodiments, the methods and compositions described herein can be used to treat a subject who has undergone or will undergo bone marrow transplantation, or has undergone, or will undergo chemotherapy or radiation therapy.

In some embodiments, the methods and compositions can be used for accelerating the recovery of, or preventing the development of a blood cell deficiency or a blood disorder in a subject, where the subject has been exposed to any one of the following: radiation therapy, chemotherapy, and radiation as a pretreatment to ablate the immune system prior to transplantation. In some embodiments, the methods and compositions can also be used to treat a subject who is or will be treated with non-myeloablative transplantation, usually with allogeneic transplantation.

In some embodiments, the methods and compositions disclosed herein are used to treat a subject suffering from immune disorder. Non-limiting examples of immunodeficiencies include Ataxia telangiectasia, DiGeorge syndrome, Severe combined immunodeficiency (SCID). Wiskott-Aldrich syndrome, Kostmann syndrome, Shwachman-Diamond syndrome, Griscelli syndrome, type II, NF-Kappa-B Essential Modulator.

In some embodiments the subject can be a candidate for autologous transplantation, i.e. the stem cell population is obtained from the patient himself. The stem cells or the source containing stem cells can be collected prior to chemotherapy and/or radiation therapy. In some embodiments, the subject can be a candidate for allogeneic transplantation, i.e. the stem cells to be transplanted are obtained from another healthy person (the donor). The donor can be related or complete stranger to the patient undergoing transplantation. Accordingly in some embodiments the population of hematopoietic cells used in the methods and compositions herein can be is autologous or allogeneic to the subject.

In some embodiments, the subject is a human subject. In some embodiments, the methods are applicable to treatment of any condition wherein increasing the hematopoietic reconstitution i.e. self-replication and differentiation of in vivo hematopoietic cells or transplanted hematopoietic cells, would be effective to result in an improved therapeutic outcome for the subject under treatment. The technology herein provides a method of increasing the hematopoietic reconstitution of an in vivo population of hematopoietic cells in a human subject, for e.g. by administration of effective amount of ANG to the subject. In some embodiments, provided herein is a method of increasing the hematopoietic reconstitution of hematopoietic cells to be transplanted in a subject e.g., upon contacting the population with an effective amount of protein ANG prior to in vivo administration. In some embodiments, the subject can be administered ANG before, during or after transplantation of a population of hematopoietic cells which may or may not be contacted with, or cultured in presence of ANG ex vivo.

### Methods and Use of Angiogenin for Treatment of Radiation Injury

Another aspect of the technology described herein relates to use of ANG protein or an agonist thereof to treat subjects that have been exposed to or likely to be exposed to ionization radiation. Accordingly, one aspect of the technology herein relates to a pharmaceutical composition comprising ANG or a functional fragment thereof, or an agonist thereof for preventing radiation induced hematopoietic injury, e.g., as a result of radio- or chemotherapy as a treatment for a disease or a result of accidental exposure to radiation, wherein the pharmaceutical composition is administered in an therapeutically effective amount. In one aspect, provided herein is a method of treating a subject who has been exposed to ionizing radiation or is at risk of being exposed to ionizing radiation, the method comprising administering to the subject a therapeutically effective amount of ANG.

In some embodiments, a composition comprising ANG or an agonist thereof can be used in methods for treatment of thrombocytopenia (deficiency in platelets), or neutropenia (deficiency in neurtrophils), anemia and the like, for example, where these disorders are a result of any, or a combination of: exposure to radiation (e.g., accidental radiation exposure), radiation therapy, chemotherapy, and radiation as a pretreatment to ablate the immune system prior to a transplantation.

In some embodiments, a composition comprising ANG or an agonist thereof can be used in methods for accelerating the recovery of, or preventing the development of a blood cell deficiency or a blood disorder in a subject, where the subject has been exposed to any one of the following: radiation (e.g., accidental radiation exposure), radiation therapy, chemotherapy, and radiation as a pretreatment to ablate the immune system prior to a transplantation. Accordingly, in some embodiments, a composition comprising of ANG or an agonist thereof can be used in treating “first responders” or rescue personnel to assist a disaster recovery operation at a radiation accident, e.g., military and rescue personnel who attend to a the location of radiation accident, or are likely to be exposed to radiation at a site of a radiation accident or leakage.

In some embodiments, a composition comprising of ANG or an agonist thereof can be used in methods for treating a blood cell deficiency as a complication or side effect of where the subject has been exposed to any one of the following: radiation (e.g., accidental radiation exposure), radiation therapy, chemotherapy, and radiation as a pretreatment to ablate the immune system prior to a transplantation. In some embodiments, the blood cell deficiency is a complication or side effect of AIDS (acquired immunodeficiency syndrome); ITP (immune thrombocytopenic purpura); DIC (disseminated intravascular coagulation); TTP (thrombotic thrombocytopenic purpura) and the like.

In some embodiments, a composition comprising ANG or an agonist thereof can be administered to a subject prior to, during or after exposure to radiaition or a combination thereof. In some embodiments, treatment of a subject with a composition comprising of ANG or an agonist thereof can be according to the methods as disclosed herein can be therapeutic treatment, e.g., a method of treatment of a blood disorder in a subject, for example, a subject with neutropenia or low platelet count. In some embodiments, therapeutic treatment involves administration of a composition comprising ANG or an agonist thereof according to the methods as disclosed herein to a patient suffering from one or more symptoms of or having been diagnosed as being afflicted with a blood disease or disorder. Relief and even partial relief from one or more of a symptom or a blood disorder may correspond to an increased life span or, simply, an increased quality of life. Further, treatments that alleviate a pathological symptom can allow for other treatments to be administered.

In some embodiments, a composition comprising ANG or an agonist thereof can be administered to the subject after exposure to ionizing radiation. In some embodiments, a composition comprising an effective amount of ANG or an agonist thereof can be administered immediately, about 2 hrs, about 4 hrs, about 6 hrs, about 10 hrs, about 12 hrs, about 16 hrs, about 20 hrs, at least about 24 hrs after exposure to ionizing radiation. The time interval and duration for administration can be determined by those skilled in the art and among other factors can depend on the age of the subject, gender of the subject, strength of the ionizing radiation exposed, severity of the disease symptoms etc. In some embodiments, for example, a composition comprising ANG or an agonist thereof can be administered every 2 hrs, every 4 hrs, every 6 hrs, every 10 hrs, at least every 24 hrs for a period of at least 1 day, at least 2 days, at least 3 days after starting the treatment post exposure to radiation. In some embodiments, the treatment is started 24 preferable 24 hrs after irradiation.

In alternative embodiments, a composition comprising ANG or an agonist thereof can be administered according to the methods as disclosed herein and can be a prophylactic treatment, for example, to prevent low platelet count of a subject with cancer who has undergone or will undergo a cancer treatment, such as for example chemotherapy, radiotherapy and the like. In some embodiments, a prophylactic treatment comprises administration of a composition comprising of ANG or an agonist thereof according to the methods described herein to a subject who has been recommended to have, or has undergone a cancer treatment, where it is desirable to prevent the loss or decrease of white blood cells in the subject as a side-effect of the cancer treatment. Administration of a composition comprising of ANG or an agonist thereof can begin at the start or after, or during (e.g., concurrent with) administration of a cancer therapy (e.g., chemotherapy, radiation therapy) etc., and can continue, if necessary, after cancer treatment, and if necessary for life. In some embodiments, prophylactic treatment is also useful where a subject is likely to be exposed to radiation, for example, subjects who are in or located near an area of a radiation disaster accident, or subjects who are working in a recovery effort in an area that has had a radiation disaster or working in or near a radiation exposure.

In some embodiments, administration of the compositions comprising ANG or an agonist thereof can be prior to or during the exposure to ionizing radiation. The time and interval of administering a composition comprising an effective amount of ANG or an agonist thereof can be determined by those skilled in the art and can depend for example on factor such as age, gender of the subject to be treated, the strength of the ionizing radiation that is expected to effect the subject. For example, a composition comprising ANG or an agonist thereof can be administered at for example before 3 days, before 2 days, before 24 hrs (1 day), before 12 hrs, before 10 hrs, before 8 hrs, before 6 hrs, before 4 hrs, before 2 hrs, or immediately before exposure to ionizing radiation. In some embodiments, treatment can be carried out for at least 3 consecutive days, at least 2 consecutive days, at least 1 day prior to exposure to ionizing radiation. Exemplary schedule for treatment can be administering a composition comprising an effective amount of ANG or an agonist thereof for 3 consecutive days, at an interval of 24 hrs, until 24 hrs before the exposure to radiation.

In some embodiments, the administration of compositions disclosed herein can enhance the hematopoietic reconstitution, colony formation, cell survival, bone marrow cellularity, restrict proliferation of primitive HSCs and/or enhance proliferation of myeloid restricted progenitor cells after exposure to radiation. In some embodiments, in vivo administration of ANG or an agonist thereof can increase hematopoietic reconstitution of cell administered during HSCT. The hematopoietic reconstitution of the transplanted hematopoietic cell compositions is enhanced with or without myeloablative radiation regimen as part of the treatment. In some embodiments, the subject undergoing HSCT transplant can be treated with ANG prior to, during or after transplantation or a combination thereof.

In some embodiments, a composition comprising ANG or an agonist thereof can be used in methods for treating a subject who will or has undergone total body radiation (TBI). TBI doses used as a preparative regimen for HSCT typically ranges from 10 to higher than 12 Gy, which destroys the bone marrow function of the subject. The total dose of radiation may be spread over multiple sessions between intervals of time between each session. Accordingly, a therapeutic administration of ANG can be done as a single dose or multiple doses for example, administered each time prior to multiple cycles of chemotherapy or radiation therapy. The non-myeloablative regimen uses low doses of chemotherapy and radiation, for example, typically about 2 Gy, which do not destroy the subject's bone marrow. In some embodiments, the compositions comprising ANG or an agonist there and methods comprising in vivo administration of the said composition can be used to enhance hematopoietic reconstitution post myeloablative regimen, non myeloablative regimen or in absence to radiation treatment prior to HSCT. In other aspect, the subject to be treated with composition and methods disclosed herein can be, will be or has been subject to single or multiple dose of for example, 2 Gy, 4 Gy, 6 Gy, 8 Gy, 10 Gy, 12 Gy, lethal dose of irradiation. The LD50 dose is defined as a measure of a lethal dose of radiation required to kill half the members or a tested population after specified test duration. A lower LD50 is indicative of increased toxicity. In some embodiments, the treatment with compositions and methods disclosed herein can increase the LD50 for a specific dose of radiation. Accordingly, in some embodiments, the methods disclosed herein can be used to administer higher doses of ionizing radiation treatment than that would be feasible without treatment with ANG.

### Methods of Ex Vivo Expansion and Stem Cell Administration

The technology described herein relates in part on the discovery that ANG induces quiescence of primitive hematopoietic stem cells while increasing proliferation of myeloid progenitor cells. Accordingly, additional applications of the technology proposed herein include the possibility for ex-vivo expansion of stem and progenitor cells. In one aspect, the technology disclosed herein is related to expansion of hematopoietic cells ex vivo, the method comprising contacting a starting hematopoietic cell population with ANG or agonist thereof for a time sufficient to allow for primitive hematopoietic stem cell quiescence and proliferation of myeloid restricted progenitor cells, to form an expanded hematopoietic cell population. In its contemplated that the number of hematopoietic cells in the expanded population has increased than in the starter hematopoietic cell population. The phrase “cell expansion” is used herein to describe a process of cell proliferation substantially devoid of cell differentiation. Cells that undergo expansion hence maintain their cell renewal properties. Expansion is done for from about 1 day to about 30 days, from about 5 days to about 15 days, from about 7 days to about 10 days or until the indicated fold expansion. Such Hematopoietic cell expansion results in an increase of hematopoietic cells compared to the number of hematopoietic cells in the initial population. In certain aspects, the expansion results in an increase of LT-HSCs compared to the number of LT-HSCs in the initial population. In certain aspects, the expansion results in an increase of myeloid restricted progenitor cells compared to that in the initial population. In certain aspects, the expansion results in an increase of LT-HSCs and myeloid restricted progenitor cells compared to the number of LT-HSCs and myeloid restricted progenitor in the initial population. Preferably, there is an increase of 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 or more fold. In certain aspects, there is an increase of about 1.5 to 5 fold. In some aspects, there is an increase of about 1, 1.25, 1.5, 1.75, 2.0, 2.25, 2.5, 2.75, 3.0, 3.5, 4.0, 4.5, or 5.0 fold. Ex-vivo expansion of hematopoietic cells can be advantageously utilized in hematopoietic cells transplantation or implantation. Hence, according to another aspect of the technology described herein, there is provided a method of hematopoietic cells transplantation or implantation or administration into a recipient.

### Compositions

Additionally, the methods described herein can be utilized to produce transplantable or pharmaceutical hematopoietic cell preparations, such that according to yet another aspect of the technology herein there is provided a composition comprising a population of hematopoietic cells, ex vivo cultured in presence of, or contacted with an effective amount of ANG or agonist thereof. It will be appreciated in the context of the present disclosure, that a hematopoietic cell population can be provided along with the culture medium containing ANG or agonist thereof, isolated from the culture medium, and combined with a pharmaceutically acceptable carrier. In another aspect, provided herein is a composition comprising a population of hematopoietic cells and an effective amount of ANG or agonist thereof, wherein the effective amount increases quiescence of primitive hematopoietic stem cells and proliferation of myeloid restricted progenitor cells. In one aspect of the technology described herein, provided herein is a composition comprising an effective amount of ANG or agonist thereof.

The compositions provided herein can be prepared in a variety of ways depending on the intended use of the compositions. For example, a composition useful in practicing the technology herein may be a liquid comprising an agent disclosed herein, e.g., ANG or an agonist thereof, a population of hematopoietic derived using the methods described herein, or a population of hematopoietic cells in combination with ANG or agonist thereof, in solution, in suspension, or both (solution/suspension). The term “solution/suspension” refers to a liquid composition where a first portion of the active agent is present in solution and a second portion of the active agent is present in particulate form, in suspension in a liquid matrix. A liquid composition also includes a gel. The liquid composition may be aqueous or in the form of an ointment, salve, cream, or the like. An aqueous suspension or solution/suspension useful for practicing the methods disclosed herein may contain one or more polymers as suspending agents. Useful polymers include water-soluble polymers such as cellulosic polymers and water-insoluble polymers such as cross-linked carboxyl-containing polymers. An aqueous suspension or solution/suspension of the present disclosure can be viscous or muco-adhesive, or both viscous and muco-adhesive.

### Pharmaceutical Compositions

In some embodiments, the compositions herein are pharmaceutical compositions and comprise a pharmaceutically acceptable carrier. It is contemplated, that the compositions herein can be formulated as therapeutic compositions for increasing the hematopoietic reconstitution or treatment of one or more disorders disclosed herein or treatment and/or prevention of radiation injury in a subject. The technology herein provides pharmaceutical compositions comprising e.g., ANG or an agonist thereof, a population of hematopoietic cells derived by the methods herein, or a population of hematopoietic cells in combination with ANG or agonist thereof, or combinations thereof, and a pharmaceutically acceptable carrier. The term “pharmaceutically acceptable” means approved by a regulatory agency of the Federal or a state government or listed in the U.S. Pharmacopeia or other generally recognized pharmacopeia for use in animals, and more particularly, in humans. The phrase “pharmaceutically acceptable carrier” as used herein means a pharmaceutically acceptable material, composition or vehicle, such as a liquid or solid filler, diluent, excipient, solvent, media, encapsulating material, manufacturing aid (e.g., lubricant, talc magnesium, calcium or zinc stearate, or steric acid), or solvent encapsulating material, involved in maintaining the stability, solubility, or activity of, active agents in the compositions. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) excipients, such as cocoa butter and suppository waxes; (8) oils, such as peanut oil, cottonseed oil, safflower oil, sesame oil, olive oil, corn oil and soybean oil; (9) glycols, such as propylene glycol; (10) polyols, such as glycerin, sorbitol, mannitol and polyethylene glycol (PEG); (11) esters, such as ethyl oleate and ethyl laurate; (12) agar; (13) buffering agents, such as magnesium hydroxide and aluminum hydroxide; (14) alginic acid; (15) pyrogen-free water; (16) isotonic saline; (17) Ringer's solution; (19) pH buffered solutions; (20) polyesters, polycarbonates and/or polyanhydrides; (21) bulking agents, such as polypeptides and amino acids (22) serum components, such as serum albumin, HDL and LDL; (23) C2-C12 alcohols, such as ethanol; and (24) other non-toxic compatible substances employed in pharmaceutical formulations. Release agents, coating agents, preservatives, and antioxidants can also be present in the formulation. The terms such as “excipient”, “carrier”, “pharmaceutically acceptable carrier” or the like are used interchangeably herein. Examples of suitable pharmaceutical carriers are described in “Remington's Pharmaceutical Sciences” by E. W. Martin, and still others are familiar to skilled artisans.

These compositions can take the form of solutions, suspensions, emulsion, tablets, pills, capsules, powders, sustained-release formulations and the like, including those adapted for the following: (1) parenteral administration, for example, by subcutaneous, intramuscular, intravenous or epidural injection as, for example, a sterile solution or suspension, or sustained-release formulation; (2) topical application, for example, as a cream, ointment, or a controlled-release patch or spray applied to the skin; (3) intravaginally or intrarectally, for example, as a pessary, cream or foam; (4) ocularly; (5) transdermally; (6) transmucosally; or (7) nasally. The pharmaceutical compositions of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The pharmaceutical compositions can be administered in various ways, depending on the preference for local or systemic treatment, and on the area to be treated. Administration may be done topically (including opthalmically, vaginally, rectally, intranasally), orally, by inhalation, or parenterally, for example by intravenous drip or intraperitoneal, subcutaneous, subdural, intramuscular or intravenous injection, or via an implantable delivery device. Formulations for topical administration may include, but are not limited to, lotions, ointments, gels, creams, suppositories, drops, liquids, sprays and powders Conventional pharmaceutical carriers, aqueous, powder or oily bases, thickeners and the like may be necessary or desirable. Compositions for oral administration include powders or granules, suspensions or solutions in water or nonaqueous media, sachets, capsules or tablets. Thickeners, diluents, flavorings, dispersing aids, emulsifiers or binders may be desirable. Formulations for parenteral administration may include, but are not limited to, sterile solutions, which may also contain buffers, diluents and other suitable additives. Formulations for implantable delivery devices may similarly include, but are not limited to, sterile solutions, which may also contain buffers, diluents and other suitable additives.

In some embodiments, a therapeutic composition for reconstituting hematopoiesis, treatment of one or more disorders disclosed herein or radiation injury in a subject comprises a composition as described above in a pharmaceutically acceptable medium suitable for administration to a recipient subject. Pharmaceutically acceptable mediums suitable for administration to a subject are known in the art. In some embodiments, compositions comprising hematopoietic cells disclosed herein can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene, glycol, liquid polyethylene glycol, and the like) and suitable mixtures thereof.

Sterile injectable solutions can be prepared by incorporating the compositions disclosed herein in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “Remington's Pharmaceutical Science”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, may be added. Prevention of the action of microorganisms can be ensured by various antibacterial and antifungal agents, for example, parabens, chlorobutanol, phenol, sorbic acid, and the like. The compositions can be isotonic, i.e., they can have the same osmotic pressure as blood and lacrimal fluid. The desired isotonicity of the compositions herein may be accomplished using sodium chloride, or other pharmaceutically acceptable agents such as dextrose, boric acid, sodium tartrate, propylene glycol or other inorganic or organic solutes. Sodium chloride is preferred particularly for buffers containing sodium ions.

Parenteral dosage forms of the compositions can also be administered to a subject by various routes, including, but not limited to subcutaneous, intravenous (including bolus injection), intramuscular, and intraarterial. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to solutions ready for injection, dry products ready to be dissolved or suspended in a pharmaceutically acceptable vehicle for injection, suspensions ready for injection, controlled-release parenteral dosage forms, and emulsions. Suitable vehicles that can be used to provide parenteral dosage forms of the disclosure are well known to those skilled in the art. Examples include, without limitation: sterile water; water for injection USP; saline solution; glucose solution; aqueous vehicles such as but not limited to sodium chloride injection, Ringer's injection, dextrose Injection, dextrose and sodium chloride injection, and lactated Ringer's injection; water-miscible vehicles such as, but not limited to ethyl alcohol, polyethylene glycol, and propylene glycol; and non-aqueous vehicles such as, but not limited to corn oil, cottonseed oil, peanut oil, sesame oil, ethyl oleate, isopropyl myristate, and benzyl benzoate.

Compositions provided herein can be packaged in a pressurized aerosol container together with suitable propellants, for example, hydrocarbon propellants like propane, butane, or isobutane with conventional adjuvants. Compositions can also be administered in a non-pressurized form such as in a nebulizer or atomizer. Compositions can also be administered directly to the airways in the form of a dry powder, for example, by use of an inhaler. Suitable powder compositions include, by way of illustration, powdered preparations of an agent (e.g., ANG or agonist thereof) thoroughly intermixed with lactose, or other inert powders acceptable for intrabronchial administration. The powder compositions can be administered via an aerosol dispenser or encased in a breakable capsule which can be inserted by the subject into a device that punctures the capsule and blows the powder out in a steady stream suitable for inhalation. The compositions can include propellants, surfactants, and co-solvents and can be filled into conventional aerosol containers that are closed by a suitable metering valve.

Aerosols for the delivery to the respiratory tract are known in the art. See for example, Adjei, A. and Garren, J. Pharm. Res., 1: 565-569 (1990); Zanen, P. and Lamm, J.-W. J. Int. J. Pharm., 114: 111-115 (1995); Gonda, I. “Aerosols for delivery of therapeutic and diagnostic agents to the respiratory tract,” in Critical Reviews in Therapeutic Drug Carrier Systems, 6:273-313 (1990); Anderson et al., Am. Rev. Respir. Dis., 140: 1317-1324 (1989)) and have potential for the systemic delivery of peptides and proteins as well (Patton and Platz, Advanced Drug Delivery Reviews, 8:179-196 (1992)); Timsina et. al., Int. J. Pharm., 101: 1-13 (1995); and Tansey, I. P., Spray Technol. Market, 4:26-29 (1994); French, D. L., Edwards, D. A. and Niven, R. W., Aerosol Sci., 27: 769-783 (1996); Visser, J., Powder Technology 58: 1-10 (1989)); Rudt, S, and R. H. Muller, J. Controlled Release, 22: 263-272 (1992); Tabata, Y, and Y. Ikada, Biomed. Mater. Res., 22: 837-858 (1988); Wall, D. A., Drug Delivery, 2: 10 1-20 1995); Patton, J. and Platz, R., Adv. Drug Del. Rev., 8: 179-196 (1992); Bryon, P., Adv. Drug. Del. Rev., 5: 107-132 (1990); Patton, J. S., et al., Controlled Release, 28: 15 79-85 (1994); Damms, B. and Bains, W., Nature Biotechnology (1996); Niven, R. W., et al., Pharm. Res., 12(9); 1343-1349 (1995); and Kobayashi, S., et al., Pharm. Res., 13(1): 80-83 (1996), contents of all of which are herein incorporated by reference in their entirety.

The formulations of the compositions disclosed herein further encompass anhydrous pharmaceutical compositions and dosage forms comprising the disclosed compounds as active ingredients, since water can facilitate the degradation of some compounds. For example, the addition of water (e.g., 5%) is widely accepted in the pharmaceutical arts as a means of simulating long-term storage in order to determine characteristics such as shelf life or the stability of formulations over time. See, e.g., Jens T. Carstensen, Drug Stability: Principles & Practice, 379-80 (2nd ed., Marcel Dekker, NY, N.Y.: 1995). Anhydrous pharmaceutical compositions and dosage forms of the disclosure can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected. Anhydrous compositions are preferably packaged using materials known to prevent exposure to water such that they can be included in suitable formulary kits. Examples of suitable packaging include, but are not limited to hermetically sealed foils, plastics, unit dose containers (e.g., vials) with or without desiccants, blister packs, and strip packs.

In some embodiments of the aspects described herein, the compositions can be administered to a subject by controlled- or delayed-release means. Ideally, the use of an optimally designed controlled-release preparation in medical treatment is characterized by a minimum of drug substance being employed to cure or control the condition in a minimum amount of time. Advantages of controlled-release formulations include: 1) extended activity of the active agent; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. (Kim, Chemg-ju, Controlled Release Dosage Form Design, 2 (Technomic Publishing, Lancaster, Pa.: 2000)). Controlled-release formulations can be used to control a compound of formula (I)'s onset of action, duration of action, plasma levels within the therapeutic window, and peak blood levels. In particular, controlled- or extended-release dosage forms or formulations can be used to ensure that the maximum effectiveness of a compound of formula (I) is achieved while minimizing potential adverse effects and safety concerns, which can occur both from under-dosing a drug (i.e., going below the minimum therapeutic levels) as well as exceeding the toxicity level for the drug.

A variety of known controlled- or extended-release dosage forms, formulations, and devices can be adapted for use with the compositions described herein. Examples include, but are not limited to those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1, each of which is incorporated herein by reference in their entireties. These dosage forms can be used to provide slow or controlled-release of one or more active ingredients using, for example, hydroxypropylmethyl cellulose, other polymer matrices, gels, permeable membranes, osmotic systems (such as OROS). (Alza Corporation, Mountain View, Calif. USA)), multilayer coatings, microparticles, liposomes, or microspheres or a combination thereof to provide the desired release profile in varying proportions. Additionally, ion exchange materials can be used to prepare immobilized, adsorbed salt forms of the disclosed compounds and thus effect controlled delivery of the drug. Examples of specific anion exchangers include, but are not limited to Duolite. A568 and Duolite. AP143 (Rohm&Haas, Spring House, Pa. USA).

In some embodiments, compositions described herein can be administered to a subject by sustained release or in pulses. Pulse therapy is not a form of discontinuous administration of the same amount of a composition over time, but comprises administration of the same dose of the composition at a reduced frequency or administration of reduced doses. Sustained release or pulse administrations are particularly preferred when the disorder occurs continuously in the subject, for example where the subject has continuous or chronic symptoms of a viral infection. Each pulse dose can be reduced and the total amount of a ANG protein or ANG agonist can be administered over the course of treatment to the patient is minimized.

The interval between pulses, when necessary, can be determined by one of ordinary skill in the art. Often, the interval between pulses can be calculated by administering another dose of the composition when the composition or the active component of the composition is no longer detectable in the subject prior to delivery of the next pulse. Intervals can also be calculated from the in vivo half-life of the composition. Intervals can be calculated as greater than the in vivo half-life, or 2, 3, 4, 5 and even 10 times greater the composition half-life. Various methods and apparatus for pulsing compositions by infusion or other forms of delivery to the patient are disclosed in U.S. Pat. Nos. 4,747,825; 4,723,958; 4,948,592; 4,965,251 and 5,403,590.

Provided herein are compositions that are useful for at least one of increasing hematopoietic reconstitution, treatment of one or more disorders disclosed herein or treatment or prevention of radiation injury. In one embodiment, the composition is a pharmaceutical composition. The composition can comprise a therapeutically or prophylactically effective amount of an agent disclosed herein (e.g., ANG or agonist thereof, a population of hematopoietic cells prepared by the methods disclosed herein, a population of hematopoietic cells in contact with ANG or an agonist thereof or combinations thereof). The composition can optionally include a carrier, such as a pharmaceutically acceptable carrier. Pharmaceutically acceptable carriers are determined in part by the particular composition being administered, as well as by the particular method used to administer the composition. Accordingly, there is a wide variety of suitable formulations of pharmaceutical compositions disclosed herein. Formulations suitable for parenteral administration, such as, for example, by intraarticular (in the joints), intravenous, intramuscular, intradermal, intraperitoneal, and subcutaneous routes, and carriers include aqueous isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, preservatives, liposomes, microspheres and emulsions.

The compositions described herein include, but are not limited to therapeutic compositions useful for practicing the therapeutic methods described herein. Therapeutic compositions contain a physiologically tolerable carrier together with an active agent as described herein, dissolved or dispersed therein as an active ingredient. In one embodiment, the therapeutic composition is not immunogenic (e.g., allergenic) when administered to a mammal or human patient for therapeutic purposes. As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like. A pharmaceutically acceptable carrier will not promote the raising of an immune response to an agent with which it is admixed, unless so desired. The preparation of a pharmacological composition that contains active ingredients dissolved or dispersed therein is well understood in the art and need not be limited based on formulation. Typically such compositions are prepared as injectable either as liquid solutions or suspensions, however, solid forms suitable for solution, or suspensions, in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Suitable excipients include, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient. The therapeutic compositions described herein can include pharmaceutically acceptable salts of the components therein.

Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent used in the methods described herein that will be effective in the treatment of a particular disorder or condition will depend on the nature of the disorder or condition, and can be determined by standard clinical techniques.

While any suitable carrier known to those of ordinary skill in the art can be employed in the pharmaceutical compositions provided herein, the type of carrier will vary depending on the mode of administration. Compositions can be formulated for any appropriate manner of administration, including for example, topical, oral, nasal, intravenous, intracranial, intraperitoneal, subcutaneous or intramuscular administration. For parenteral administration, such as subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., polylactate polyglycolate) can also be employed as carriers for the pharmaceutical compositions of this invention. Suitable biodegradable microspheres are disclosed, for example, in U.S. Pat. Nos. 4,897,268 and 5,075,109. Such compositions can also comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, chelating agents such as EDTA or glutathione, adjuvants (e.g., aluminum hydroxide) and/or preservatives. Alternatively, compositions as described herein can be formulated as a lyophilizate. Compounds can also be encapsulated within liposomes using well known technology. The compositions described herein can be administered as part of a sustained release formulation (i.e., a formulation such as a capsule or sponge that effects a slow release of compound following administration). Such formulations can generally be prepared using well known technology and administered by, for example, oral, rectal or subcutaneous implantation, or by implantation at the desired target site. Sustained-release formulations can contain a polypeptide, polynucleotide dispersed in a carrier matrix and/or contained within a reservoir surrounded by a rate controlling membrane. Carriers for use within such formulations are biocompatible, and can also be biodegradable; preferably the formulation provides a relatively constant level of active component release. The amount of active compound contained within a sustained release formulation depends upon the site of implantation, the rate and expected duration of release and the nature of the condition to be treated or prevented.

### Dosage and Administration

The methods disclosed herein comprises administrations of agents to increase the hematopoietic reconstitution, treatment of disease or disorder characterized by decreased levels of hematopoietic stem and/or progenitor cells or blood cell deficiency or for prevention and treatment of radiation injury. The agents of the methods disclosed herein comprise of ANG or agonist thereof, hematopoietic cells derived upon ex vivo contact with or culturing with ANG or agonist thereof, or hematopoietic cells in combination with ANG or agonist thereof.

Agents of the technology disclosed herein can be administered to a subject in need thereof, by any appropriate route which results in an effective treatment in the subject. As used herein, the terms “administering,” and “introducing” are used interchangeably and refer to the placement of an agent into a subject by a method or route which results in at least partial localization of such agents at a desired site, such that a desired effect(s) is produced.

In some embodiments, the agents described herein is administered to a subject by any mode of administration that delivers the agent systemically or to a desired surface or target, and can include, but is not limited to injection, infusion, instillation, and inhalation administration. To the extent that polypeptide agents can be protected from inactivation in the gut, oral administration forms are also contemplated. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, intratumoral, and intrasternal injection and infusion. In some embodiments, the agents for use in the methods described herein are administered by intravenous infusion or injection.

The phrases “parenteral administration” and “administered parenterally” as used herein, refer to modes of administration other than enteral and topical administration, usually by injection. The phrases “systemic administration,” “administered systemically”, “peripheral administration” and “administered peripherally” as used herein refer to the administration of an agent (e.g., ANG) or a composition disclosed herein other than directly into a target site, tissue, or organ, such as a tumor site, such that it enters the subject's circulatory system and, thus, is subject to metabolism and other like processes.

For the clinical use of the methods described herein, administration of the agents can include formulation into pharmaceutical compositions or pharmaceutical formulations for parenteral administration, e.g., intravenous; mucosal, e.g., intranasal; ocular, or other mode of administration. In some embodiments, the agents described herein can be administered along with any pharmaceutically acceptable carrier compound, material, or composition which results in an effective treatment in the subject. Thus, a pharmaceutical formulation for use in the methods described herein can contain an agent as described herein in combination with one or more pharmaceutically acceptable ingredients e.g., pharmaceutically acceptable carrier or solution.

Dosing is dependent on responsiveness of the condition for treatment, but will normally be one or more doses per day, with course of treatment lasting from several days to several months or until a required effect is achieved. Persons ordinarily skilled in the art can easily determine optimum dosages, dosing methodologies and repetition rates. Slow release administration regimes may be advantageous in some applications. Hematopoietic cells or a mixture comprising such cell types may be administered to a subject according to methods known in the art. Such compositions may be administered by any conventional route, including injection or by gradual infusion over time. The administration may, depending on the composition being administered, for example, be, pulmonary, intravenous, intraperitoneal, intramuscular, intracavity, subcutaneous, or transdermal. The hematopoietic cells are administered in “effective amounts”, or the amounts that either alone or together with further doses produce the desired therapeutic response. Administered cells may be autologous (“self”) or heterologous/non-autologous (“non-self,” e.g., allogeneic, syngeneic or xenogeneic). In some embodiments, administration of the cells can occur within a short period of time following contact with or culture in presence of ANG or agonist thereof (e.g., 1, 2, 5, 10, 24, 48 hours, 1 week or 2 weeks contact with or culture in presence of ANG or agonist thereof) and according to the requirements of each desired treatment regimen. For example, where radiation or chemotherapy is conducted prior to administration, treatment, and transplantation of compositions comprising hematopoietic cells should optimally be provided within about one month of the cessation of therapy. However, transplantation at later points after treatment has ceased may be done with derivable clinical outcomes.

The quantity of cells to be administered will vary for the subject being treated. The precise determination of what would be considered an effective dose may be based on factors individual to each patient, including their size, age, sex, weight, and condition of the particular patient. As few as 100-1000 cells may be administered for certain desired applications among selected patients. Therefore, dosages can be readily ascertained by those skilled in the art from this disclosure and the knowledge in the art. The skilled artisan can readily determine the amount of cells and optional additives, vehicles, and/or carrier in compositions and to be administered in methods of the invention. Skilled artisans will recognize that any and all of the standard methods and modalities for bone marrow transplantation, blood transfusion and therapeutic use of blood components currently in clinical practice and clinical development are suitable for using the compositions and practicing the methods of the invention. The compositions disclosed herein can be administered by injection into a target site of a subject, preferably via a delivery device, such as a tube, e.g., catheter. In a preferred embodiment, the tube additionally contains a needle, e.g., a syringe, through which the compositions can be introduced into the subject at a desired location. Specific, non-limiting examples of administering cells to subjects may also include administration by subcutaneous injection, intramuscular injection, intravenous injection, intraarterial intramuscular, intracardiac injection, infusion, intradermal injection, intrathecal injection, epidural injection, intraperitoneal injection, or intracerebral injection. If administration is intravenous, an injectable liquid suspension of the compositions can be prepared and administered by a continuous drip or as a bolus.

Pharmaceutical compositions described herein can be administered in a manner compatible with the dosage formulation, and in a therapeutically effective amount, for example intravenously, intraperitoneally, intramuscularly, subcutaneously, and intradermally. It may also be administered by any of the other numerous techniques known to those of skill in the art, see for example the latest edition of Remington's Pharmaceutical Science, the entire teachings of which are incorporated herein by reference. For example, for injections, the pharmaceutical composition disclosed herein may be formulated in adequate solutions including but not limited to physiologically compatible buffers such as Hank's solution, Ringer's solution, or a physiological saline buffer. The solutions may contain formulatory agents such as suspending, stabilizing, and/or dispersing agents. Alternatively, the pharmaceutical composition of the present disclosure may be in powder form for combination with a suitable vehicle, e.g., sterile pyrogen free water, before use. Further, the compositions herein may be administered per se or may be applied as an appropriate formulation together with pharmaceutically acceptable carriers, diluents, or excipients that are well known in the art. In addition, other pharmaceutical delivery systems such as liposomes and emulsions that are well known in the art, and a sustained-release system, such as semi-permeable matrices of solid polymers containing a therapeutic agent, may be employed. Various sustained-release materials have been established and are well-known to one skilled in the art. Further, the compositions and agents disclosed herein can be administered alone or together with another therapy conventionally used for the treatment of a disease/condition associated with decreased levels of hematopoietic stem and/or progenitor cells, blood cell deficiency, or hematopoietic reconstitution, or in which expansion and/or differentiation of HSCs is desirable.

As used herein, the term “treatment” includes prophylaxis and therapy. Prophylaxis or treatment can be accomplished by a single direct injection at a single time point or multiple time points. Administration can also be nearly simultaneous to multiple sites. Patients or subjects include mammals, such as human, bovine, equine, canine, feline, porcine, and ovine animals as well as other veterinary subjects. Preferably, the patients or subjects are human. In one aspect, provided herein are methods for treating a disease or disorder characterized by decreased levels of hematopoietic stem and/or progenitor cells, hematopoietic reconstitution or blood cell deficiency in a subject. In some embodiments, the subject can be a mammal. In some embodiments, the mammal can be a human, although the approach is effective with respect to all mammals. The method comprises administering to the subject an effective amount of an agent disclosed herein. The dosage range for the ANG or agonist thereof depends upon the potency, and includes amounts large enough to produce the desired effect, e.g., treatment of radiation injury. The dosage should not be so large as to cause unacceptable adverse side effects. Generally, the dosage will vary with the age, condition, and sex of the patient. The dosage can be determined by one of skill in the art and can also be adjusted by the individual physician in the event of any complication. Typically, the dosage ranges from 0.001 mg/kg body weight to 5 g/kg body weight. In some embodiments, the dosage range is from 0.001 mg/kg body weight to 1 g/kg body weight, from 0.001 mg/kg body weight to 0.5 g/kg body weight, from 0.001 mg/kg body weight to 0.1 g/kg body weight, from 0.001 mg/kg body weight to 50 mg/kg body weight, from 0.001 mg/kg body weight to 25 mg/kg body weight, from 0.001 mg/kg body weight to 10 mg/kg body weight, from 0.001 mg/kg body weight to 5 mg/kg body weight, from 0.001 mg/kg body weight to 1 mg/kg body weight, from 0.001 mg/kg body weight to 0.1 mg/kg body weight, from 0.001 mg/kg body weight to 0.005 mg/kg body weight. Alternatively, in some embodiments the dosage range is from 0.1 g/kg body weight to 5 g/kg body weight, from 0.5 g/kg body weight to 5 g/kg body weight, from 1 g/kg body weight to 5 g/kg body weight, from 1.5 g/kg body weight to 5 g/kg body weight, from 2 g/kg body weight to 5 g/kg body weight, from 2.5 g/kg body weight to 5 g/kg body weight, from 3 g/kg body weight to 5 g/kg body weight, from 3.5 g/kg body weight to 5 g/kg body weight, from 4 g/kg body weight to 5 g/kg body weight, from 4.5 g/kg body weight to 5 g/kg body weight, from 4.8 g/kg body weight to 5 g/kg body weight. In one embodiment, the dose range is from 5 μg/kg body weight to 30 μg/kg body weight. Alternatively, the dose range will be titrated to maintain serum levels between 5 μg/mL and 30 μg/milk

Administration of the doses recited above can be repeated for a limited period of time. In some embodiments, the doses are given once a day, or multiple times a day, for example but not limited to three times a day. In another embodiment, the doses recited above are administered daily for several weeks or months. The duration of treatment depends upon the subject's clinical progress and responsiveness to therapy. Continuous, relatively low maintenance doses are contemplated after an initial higher therapeutic dose. In some embodiments, the ANG/or agonist thereof can be administered prior to, during or after the subject has undergone another treatment such as chemotherapy, radiation therapy or stem cell transplantation. A therapeutically effective amount is an amount of an agent that is sufficient to produce a statistically significant, measurable change in at least one symptom of a disorder or disease disclosed herein. Such effective amounts can be gauged in clinical trials as well as animal studies for a given agent. It is contemplated herein that the compositions can be delivered intravenously (by bolus or continuous infusion), orally, by inhalation, intranasally, intraperitoneally, intramuscularly, subcutaneously, intracavity, and can be delivered by peristaltic means, if desired, or by other means known by those skilled in the art. The agents or compositions comprising the said agents can be administered systemically, if so desired.

In one embodiment, the compositions can be administered to a subject for an extended period of time. Sustained contact with an ANG or ANG agonist composition can be achieved by, for example, repeated administration of ANG or ANG agonist composition over a period of time, such as one week, several weeks, one month or longer. In some embodiments, a pharmaceutically acceptable formulation used to administer the active agent provides sustained delivery, such as “slow release” of the agent to a subject. For example, the formulation can deliver the agent or composition for at least one, two, three, or four weeks after the pharmaceutically acceptable formulation is administered to the subject. In some embodiments, a subject to be treated in accordance with the methods described herein is treated with the active composition for at least 30 days (either by repeated administration or by use of a sustained delivery system, or both). Preferred approaches for sustained delivery include use of a polymeric capsule, a minimum to deliver the formulation, a biodegradable implant, or implanted transgenic autologous cells (as described in e.g., U.S. Pat. No. 6,214,622). Implantable infusion pump systems (such as e.g., Infused™; see such as Zierski, J. et al, 1988; Kanoff, R. B., 1994) and osmotic pumps (sold by Alza Corporation™) are available in the art. Another mode of administration is via an implantable, externally programmable infusion pump. Suitable infusion pump systems and reservoir systems are also described in e.g., U.S. Pat. No. 5,368,562 by Blomquist and U.S. Pat. No. 4,731,058 by Doan, developed by Pharmacia Deltec™ Inc.

Therapeutic compositions containing at least one agent can be conventionally administered in a unit dose. The term “unit dose” when used in reference to a therapeutic composition refers to physically discrete units suitable as unitary dosage for the subject, each unit containing a predetermined quantity of active material calculated to produce the desired therapeutic effect in association with the required physiologically acceptable diluent, i.e., carrier, or vehicle. The compositions are administered in a manner compatible with the dosage formulation, and in a therapeutically effective amount. The quantity to be administered and timing depends on the subject to be treated, capacity of the subject's system to utilize the active ingredient, and degree of therapeutic effect desired. An agent can be targeted by means of a targeting moiety, such as e.g., an antibody or targeted liposome technology. In some embodiments, an agent can be targeted to a tissue by using bispecific antibodies, for example produced by chemical linkage of an anti-ligand antibody (Ab) and an Ab directed toward a specific target. The addition of an antibody to an agent permits the agent to accumulate additively at the desired target site (e.g., tumor site). Antibody-based or non-antibody-based targeting moieties can be employed to deliver a ligand or the inhibitor to a target site. Preferably, a natural binding agent for an unregulated or disease associated antigen is used for this purpose. Precise amounts of active ingredient required to be administered depend on the judgment of the practitioner and are particular to each individual. However, suitable dosage ranges for systemic application are disclosed herein and depend on the route of administration. Suitable regimes for administration are also variable, but are typified by an initial administration followed by repeated doses at one or more intervals by a subsequent injection or other administration. Alternatively, continuous intravenous infusion sufficient to maintain concentrations in the blood in the ranges specified for in vivo therapies are contemplated.

### Efficacy of Treatment

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with, a disease or disorder. The term “treating” includes reducing or alleviating at least one adverse effect or symptom of a condition, disease or disorder associated with a chronic immune condition, such as, but not limited to a chronic infection or a cancer. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of at least slowing of progress or worsening of symptoms that would be expected in absence of treatment. Beneficial or desired clinical results include, but are not limited to alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. The term “treatment” of a disease also includes providing relief from the symptoms or side-effects of the disease (including palliative treatment).

For example, in some embodiments, the methods described herein comprise administering an effective amount of the agents described herein (e.g. ANG/or agonist thereof, population of hematopoietic cells derived upon ex vivo contact with or culturing in presence of ANG or agonist thereof or a population of hematopoietic cells in combination with ANG or agonist thereof) to a subject in order to alleviate a symptom of one or more disorders disclosed herein. As compared with an equivalent untreated control, such reduction or degree of prevention is at least 5%, 10%, 20%, 40%, 50%, 60%, 80%, 90%, 95%, or 100% as measured by any standard technique.

The term “effective amount” as used herein refers to the amount of an agent disclosed herein e.g., ANG or agonist thereof, needed to alleviate at least one or more symptom of the disease or disorder, and relates to a sufficient amount of pharmacological composition to provide a desired effect, e.g., increase in hematopoietic reconstitution in a subject having a blood cell deficiency. The term “therapeutically effective amount” therefore refers to an amount, that is sufficient to effect a particular effect when administered to a typical subject. An effective amount as used herein would also include an amount sufficient to delay the development of a symptom of the disease, alter the course of a symptom disease (for example but not limited to slow the progression of a symptom of the disease), or reverse a symptom of the disease. Thus, it is not possible to specify the exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of an agent disclosed herein, for example, ANG or agonist thereof), which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay. The dosage can be determined by a physician and adjusted, as necessary, to suit observed effects of the treatment.

### Kits

In another aspect, the technology disclosed herein provided kits containing a population of hematopoietic cells for expansion and effective amount of ANG or agonist thereof. In some embodiments, the kit can further comprise culture media and other necessary components for carrying out ex vivo culture and/or expansion methods described herein. Kits directed to use of the cell populations, expanded or unexpanded, for therapeutic applications are provided. The kits may further include, by way of example and not limitation, buffers, labels, reagents, and instructions for methods of using the kits. In an embodiment, a kit may comprise a starter population including LT-HSCs, myeloid restricted progenitor and a container. In another embodiment, a kit may further comprise growth factors and/or cytokines.

In another aspect, provided herein is an article of manufacture comprising packaging material and a pharmaceutical composition disclosed herein contained within the packaging material, wherein the pharmaceutical composition comprises compositions of populations of hematopoietic cells cultured in presence of ANG or agonist thereof, ANG or agonist thereof or a population of hematopoietic cells in contact with ANG or agonist thereof, or combinations thereof. The packaging material comprises a label or package insert which indicates that the compositions of cells can be used for blood transfusion, bone marrow transplantation, etc.

According to a further aspect of the technology herein there is provided a method of preserving stem cells. In one embodiment, the method is effected by handling the stem cell in at least one of the following steps: harvest, isolation and/or storage, in a presence of an effective amount of ANG or an agonist thereof.

According to still a further aspect of the technology described herein there is provided a cells collection/culturing bag. The cells collection/culturing bag of the present disclosure is supplemented with an effective amount of ANG or agonist thereof.

According to the technology described herein there is also provided a cells separation and/or washing buffer. The separation and/or washing buffer is supplemented with an effective amount ANG or agonist thereof. Thus, further according to the technology described herein there are provided stem cells collection bags and separation and washing buffers supplemented with an effective amount or concentration of ANG or agonist thereof, which increases quiescence of primitive hematopoietic stem cells and proliferation of myeloid progenitor cells. In some embodiments, the stem cells collection bags and separation and washing buffers can be further supplemented with nutrients and cytokines useful for growth and/or preservation of stem cells, non-limiting examples of which can include interleukins, granulocyte colony stimulating factor, granulacyte macrophage colony stimulating factor, erythropoietin.

It is understood that the foregoing detailed description and the following examples are illustrative only and are not to be taken as limitations upon the scope of the invention. Various changes and modifications to the disclosed embodiments, which will be apparent to those of skill in the art, may be made without departing from the spirit and scope of the invention. Further, all patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Embodiments of various aspects described herein can be defined in any of the following numbered paragraphs:
1. A method of increasing hematopoietic reconstitution in a human subject, the method comprising:
(i) contacting a population of hematopoietic cells ex vivo, with an effective amount of an Angiogenin (ANG) protein or an ANG agonist;
(ii) administering cells from step (i) to a subject, wherein the subject is in need of hematopoietic reconstitution.
2. The method of paragraph 1, wherein the population of hematopoietic cells are obtained from bone marrow, peripheral blood, cord blood, amniotic fluid, placental blood, embryonic stem cells (ESCs), or induced pluripotent stem cells (iPSCs).
3. The method of any one of paragraphs 1-2, wherein the population of hematopoietic cells are human.
4. The method of any one of paragraphs 1-3, wherein the population of hematopoietic cells comprises at least one or more of long-term hematopoietic stem cells (LT-HSCs), short-term hematopoietic stem cells (ST-HSCs), multipotent progenitors (MPPs), common myeloid progenitors (CMPs), common lymphoid progenitors (CLPs), granulocyte-macrophage progenitors (GMPs) and megakaryocyte-erythroid progenitors (MEPs).
5. The method of any one of paragraphs 1-4, wherein the population of hematopoietic cells are autologous or allogeneic to the subject.
6. The method of any one of paragraphs 1-5, further comprising culturing the population of hematopoietic cells in presence of ANG protein or ANG agonist prior to step (ii).
7. The method of paragraph 6, wherein the population of hematopoietic cells are cultured in presence of ANG protein or ANG agonist for at least 2 hrs.
8. The method of paragraph 6, wherein the population of hematopoietic cells are cultured in presence of ANG protein or ANG agonist for about 2 days or more.
9. The method of paragraph 6, wherein the population of hematopoietic cells are cultured in presence of ANG protein or ANG agonist for at least 7 days.
10. The method of paragraph 1, wherein the population of hematopoietic cells are cryopreserved prior to, or after, the contacting with ANG protein or ANG agonist.
11. The method of paragraph 1, wherein the population of hematopoietic cells are cryopreserved in the presence of ANG protein or ANG agonist.
12. The method of any one of paragraphs 1-11, wherein the subject is susceptible to, or has decreased levels of hematopoietic stem cells and hematopoietic progenitor cells as compared to a healthy subject.
13. The method of any one of paragraphs 1-12, wherein the subject has undergone, or will undergo a bone marrow or stem cell transplantation, or has undergone, or will undergo chemotherapy or radiation therapy.
14. The method of any one of paragraphs 1-13, wherein the subject has a disease or disorder selected from the group consisting of leukemia, lymphoma, myeloma, solid tumor, a blood disorder, myelodysplasia, immune disorders or anemia.
15. The method of paragraph 14, wherein the anemia is sickle cell anemia, thalassemia or aplastic anemia.
16. The method of any one of paragraphs 1-15, wherein the ANG protein is human ANG protein of at least 85% amino acid sequence identity to SEQ ID NO: 1 or a functional fragment thereof with a biological activity of at least 80% of human ANG protein to increase hematopoietic reconstitution in a human subject.
17. The method of paragraph 16 wherein the ANG protein is a human recombinant ANG polypeptide.
18. The method of any one of paragraphs 16-17, wherein the functional fragment comprises at least amino acids 1-147 of SEQ ID NO 1.
19. The method of any one of paragraphs 16-18, wherein the human ANG protein of at least 85% amino acid sequence identity to SEQ ID NO: 1 comprises a mutation K33A.
20. The method of any one of paragraphs 16-19, wherein the functional fragment comprises an amino acid sequence of at least 80% of human ANG of SEQ ID NO: 1.
21. The method of paragraph 20, wherein the functional fragment of human ANG protein comprises at least 80% sequence identity to amino acids 1-147 of SEQ ID NO 1.
22. The method of paragraph 20, wherein the functional fragment of human ANG protein comprises at least 90% sequence identity to amino acids 1-147 of SEQ ID NO 1.
23. The method of paragraph 20, wherein the functional fragment of human ANG protein comprises at least 95% sequence identity to amino acids 1-147 of SEQ ID NO 1.
24. The method of paragraph 20, wherein the functional fragment of human ANG comprises at least 98% sequence identity to amino acids 1-147 of SEQ ID NO 1.
25. The method of any one of paragraphs 1-24, wherein the hematopoietic reconstitution is multi-lineage hematopoietic reconstitution.
26. The method of any one of paragraphs 1-25, wherein the hematopoietic reconstitution is long-term multi-lineage hematopoietic reconstitution.
27. The method of any one of paragraphs 1-26, wherein the hematopoietic reconstitution comprises reconstitution of short-term hematopoietic stem cells (ST-HSC) and/or long-term (LT-HSC) hematopoietic stem cells.
28. A method for expanding a population of hematopoietic cells in a biological sample, the method comprising contacting the population of hematopoietic cells with an Angiogenin (ANG) protein or ANG agonist, wherein the population comprises primitive hematopoietic stem cells and myeloid restricted progenitors, and wherein the contacting is for a sufficient amount of time to allow for primitive hematopoietic stem cells quiescence and myeloid restricted progenitor proliferation.
29. The method of paragraph 28, wherein the primitive hematopoietic stem cells are selected from the group, LT-HSC, ST-HSC, MPP or a combination thereof.
30. The method of paragraph 28, wherein the myeloid restricted progenitor are selected from the group, CMP, GMP, MEP or a combination thereof.
31. The method of any one of paragraphs 28-30, wherein the biological sample is selected from the group consisting of; cord blood, bone marrow, peripheral blood, amniotic fluid, and placental blood.
32. The method of any one of paragraphs 28-31, further comprising collecting the population of expanded hematopoietic cells.
33. A population of primitive hematopoietic stem cells produced by the method of any one of paragraphs 28-32.
34. A population of myeloid restricted progenitors produced by the method of any one of paragraphs 28-32.
35. A cryopreserved population of hematopoietic cells comprising primitive hematopoietic stem cells and/or myeloid restricted progenitors in the presence of an Angiogenin protein or ANG agonist.
36. A blood bank comprising a population of hematopoietic cells according to paragraph 33 or paragraph 34.
37. A method of administering a population of hematopoietic cells to a subject, comprising administering an effective amount of the population of hematopoietic cells to the subject, wherein the population of hematopoietic cells have been contacted ex vivo or in vivo with an Angiogenin (ANG) protein or ANG agonist, wherein the population of hematopoietic cells comprises at least one or both of primitive hematopoietic stem cells and myeloid restricted progenitors, and wherein the Angiogenin protein or ANG agonist increases primitive hematopoietic stem cells quiescence and increases myeloid restricted progenitor proliferation.
38. A method of increasing reconstitution potential of transplanted hematopoietic stem cells and hematopoietic progenitor cells in a subject, the method comprising the step of administering an Angiogenin (ANG) protein or an ANG agonist to the subject, prior to, during or after transplantation of hematopoietic stem cells and hematopoietic progenitor cells, wherein the subject is a candidate for bone marrow or stem cell transplant.
39. Use of an Angiogenin (ANG) protein or ANG agonist to increase hematopoietic reconstitution potential of a population of hematopoietic cells in a human subject in need thereof.
40. The use of paragraph 39, wherein the population of hematopoietic cells are obtained from bone marrow, peripheral blood, cord blood, amniotic fluid, placental blood, embryonic stem cells (ESCs), or induced pluripotent stem cells (iPSCs).
41. The use of any one of paragraphs 39-40, wherein the population of hematopoietic cells are human.
42. The use of any one of paragraphs 39-41, wherein the population of hematopoietic cells comprises at least one or more of long-term hematopoietic stem cells (LT-HSCs), short-term hematopoietic stem cells (ST-HSCs), multipotent progenitors (MPPs), common myeloid progenitors (CMPs), common lymphoid progenitors (CLPs), granulocyte-macrophage progenitors (GMPs) and megakaryocyte-erythroid progenitors (MEPs).
43. The use of any one of paragraphs 39-42, wherein the population of hematopoietic cells are autologous or allogeneic to the subject.
44. The use of any one of paragraphs 39-43, wherein the population of hematopoietic cells is cultured in presence of the ANG protein or ANG agonist.
45. The use of paragraph 44, wherein the population of hematopoietic cells are cultured in presence of ANG protein or ANG agonist for at least 2 hrs.
46. The use of paragraph 44, wherein the population of hematopoietic cells are cultured in presence of ANG protein or ANG agonist for about 2 days or more.
47. The use of paragraph 44, wherein the population of hematopoietic cells are cultured in presence of ANG protein or ANG agonist for at least 7 days.
48. The use of any one of paragraphs 39-47, wherein the population of hematopoietic cells are cryopreserved prior to, or after, the contacting with ANG protein or ANG agonist.
49. The use of any one of paragraphs 39-47, wherein the population of hematopoietic cells are cryopreserved in the presence of ANG protein or ANG agonist.
50. The use of any one of paragraphs 39-49, wherein the subject is susceptible to, or has decreased levels of hematopoietic stem cells and hematopoietic progenitor cells as compared to a healthy subject.
51. The use of any one of paragraphs 39-50, wherein the subject has undergone, or will undergo a bone marrow or stem cell transplantation, or has undergone, or will undergo chemotherapy or radiation therapy.
52. The use of any one of paragraphs 39-51, wherein the subject has a disease or disorder selected from the group consisting of leukemia, lymphoma, myeloma, solid tumor, a blood disorder, myelodysplasia, immune disorders and anemia.
53. The use of paragraph 52, wherein the anemia is sickle cell anemia, thalassemia or aplastic anemia.
54. The use of any one of paragraphs 39-53, wherein the ANG protein is human ANG protein of at least 85% amino acid sequence identity to SEQ ID NO: 1 or a functional fragment thereof with a biological activity of at least 80% of human ANG protein to increase hematopoietic reconstitution in a human subject.
55. The use of paragraph 54 wherein the ANG protein is a human recombinant ANG polypeptide.
56. The use of any one of paragraphs 54-55, wherein the functional fragment comprises at least amino acids 1-147 of SEQ ID NO 1.
57. The use of any one of paragraphs 54-56, wherein the human ANG protein of at least 85% amino acid sequence identity to SEQ ID NO: 1 comprises a mutation K33A.
58. The use of any one of paragraphs 54-57, wherein the functional fragment comprises an amino acid sequence of at least 80% of human ANG of SEQ ID NO: 1.
59. The use of paragraph 58, wherein the functional fragment of human ANG protein comprises at least 80% sequence identity to amino acids 1-147 of SEQ ID NO 1.
60. The use of paragraph 58, wherein the functional fragment of human ANG protein comprises at least 90% sequence identity to amino acids 1-147 of SEQ ID NO 1.
61. The use of paragraph 58, wherein the functional fragment of human ANG protein comprises at least 95% sequence identity to amino acids 1-147 of SEQ ID NO 1.
62. The use of paragraph 58, wherein the functional fragment of human ANG comprises at least 98% sequence identity to amino acids 1-147 of SEQ ID NO 1.
63. The use of any one of paragraphs 39-62, wherein the hematopoietic reconstitution is multi-lineage hematopoietic reconstitution.
64. The use of any one of paragraphs 39-63, wherein the hematopoietic reconstitution is long-term multi-lineage hematopoietic reconstitution.
65. The use of any one of paragraphs 39-64, wherein the hematopoietic reconstitution comprises reconstitution of short-term hematopoietic stem cells (ST-HSC) and/or long-term (LT-HSC) hematopoietic stem cells.
66. A method of prevention or treatment of radiation injury by exposure to ionizing radiation in a subject, the method comprising administering an effective amount of an Angiogenin (ANG) protein or Angiogenin agonist to the subject.
67. The method of paragraph 66, wherein the subject has been exposed to, will be exposed to, or is at a risk of exposure to ionizing radiation.
68. The method of paragraph 66, wherein the subject is a mammal.
69. The method of paragraph 66, wherein the subject will undergo, or has undergone radiation therapy for the treatment of a disease or disorder.
70. The method of any of paragraphs 66-69, wherein the subject will undergo, or has undergone radiation therapy as part of an ablative regimen for hematopoietic stem and progenitor cell or bone marrow transplant or chemotherapy.
71. The method of any one of paragraphs 65-70, wherein the subject will undergo, or has undergone total body radiation.
72. The method of any of paragraphs 66-71, wherein the subject will undergo, or has been exposed to a radiation accident or chemotherapy.
73. The method paragraph of 70, wherein the hematopoietic stem and progenitor cells are selected from the group consisting of Long-term hematopoietic stem cells (LT-HSCs), Short-term hematopoietic stem cells (ST-HSCs), Multipotent progenitor cells (MPPs), Common myeloid progenitor (CMPs), CLPs, Granulocyte-macrophage progenitor (GMPs) and Megakaryocyte-erythroid progenitor (MEPs).
74. The method of any one of paragraphs 66-73, wherein the ANG protein or ANG agonist is administered to the subject prior to, during or after exposure, or a combination thereof, to an ionizing radiation.
75. The method of paragraph 74, wherein the ANG protein or ANG agonist is administered for between 12 hours and 3 days prior to exposure to ionizing radiation.
76. The method of paragraph 75, wherein the exposure to ionizing radiation occurs within about 24 hours after the last administration of the ANG protein or ANG agonist.
77. The method of paragraph 74, wherein the ANG protein or ANG agonist is administered immediately after the exposure to ionizing radiation.
78. The method of paragraph 74, wherein the ANG protein or ANG agonist is administered about 24 hours after exposure to ionizing radiation.
79. The method of paragraphs 77-78, wherein the ANG protein or ANG agonist is administered for at least 3 days or more.
80. The method of any one of paragraphs 66-79, wherein the administration of the effective amount of ANG protein or ANG agonist results in increased hematopoietic reconstitution after exposure to ionizing radiation as compared to in absence of administration.
81. The method of any one of paragraphs 66-80, wherein the administration of the effective amount of ANG protein or ANG agonist increases primitive hematopoietic stem cells quiescence and increases myeloid restricted progenitor proliferation as compared to in absence of administration.
82. The method of any one of paragraphs 66-81, wherein ANG protein is human ANG protein of at least 85% amino acid sequence identity to SEQ ID NO: 1 or a functional fragment thereof with a biological activity of at least 80% of human ANG protein to increase hematopoietic reconstitution in a human subject.
83. The method of paragraph 82, wherein the ANG protein is a human recombinant ANG polypeptide.
84. The method of any one of paragraphs 82-83, wherein the functional fragment comprises at least amino acids 1-147 of SEQ ID NO 1.
85. The method of any one of paragraphs 82-83, wherein the human ANG protein of at least 85% amino acid sequence identity to SEQ ID NO: 1 comprises a mutation K33A.
86. The method of any one of paragraphs 82-85, wherein the functional fragment comprises an amino acid sequence of at least 80% of human ANG of SEQ ID NO: 1.
87. The method of paragraph 86, wherein the functional fragment of human ANG protein comprises at least 80% sequence identity to amino acids 1-147 of SEQ ID NO 1.
88. The method of paragraph 86, wherein the functional fragment of human ANG protein comprises at least 90% sequence identity to amino acids 1-147 of SEQ ID NO 1.
89. The method of paragraph 86, wherein the functional fragment of human ANG protein comprises at least 95% sequence identity to amino acids 1-147 of SEQ ID NO 1.
90. The method of paragraph 86, wherein the functional fragment of human ANG comprises at least 98% sequence identity to amino acids 1-147 of SEQ ID NO 1.
91. A method of increasing the dose of an ionizing radiation treatment, comprising administering to the subject an effective amount of an Angiogenin (ANG) protein or Angiogenin agonist before, after or during the ionizing radiation, wherein the dose of the ionizing radiation treatment is higher as compared to the dose in absence of Angiogenin (ANG) protein or Angiogenin agonist administration.
92. A pharmaceutical composition comprising the population of hematopoietic cells of any one of paragraphs 33-35 and a pharmaceutically acceptable carrier.
93. A pharmaceutical composition comprising a population of hematopoietic cells and an effective amount of ANG protein or ANG agonist, wherein the population of hematopoietic cell comprises at least one or both of primitive hematopoietic stem cells and myeloid restricted progenitor cells and wherein the effective amount ANG protein or ANG agonist increases quiescence of primitive hematopoietic cells and proliferation of myeloid restricted cells.
94. The pharmaceutical composition of paragraph 93, wherein the primitive hematopoietic cells are selected from the group, long-term hematopoietic stem cells (LT-HSCs), short-term hematopoietic stem cells (ST-HSCs), multipotent progenitors (MPPs) or a combination thereof.
95. The pharmaceutical composition of paragraph 93, wherein the myeloid-restricted progenitor cells are selected from the group, common myeloid progenitors (CMPs), granulocyte-macrophage progenitors (GMPs), megakaryocyte-erythroid progenitors (MEPs) and combination thereof.
96. A pharmaceutical composition comprising an effective amount of ANG protein or ANG agonist for use in promoting hematopoietic reconstitution, wherein the effective amount is capable of increasing primitive hematopoietic cell quiescence and proliferation of myeloid restricted cells.
97. A pharmaceutical composition comprising an effective amount of ANG protein or ANG agonist for use in treatment of a disease or disorder characterized by decreased levels of hematopoietic stem cells and hematopoietic progenitor cells.
98. The pharmaceutical composition of paragraph 97, wherein the disease or disorder is selected from the group consisting of leukemia, lymphoma, myeloma, solid tumor, a blood disorder, myelodysplasia, immune disorders or anemia.
99. The pharmaceutical composition of paragraph 98, wherein the anemia is sickle cell anemia, thalassemia or aplastic anemia.
100. Stem cell collection bags, stem cell separation and stem cell washing buffers supplemented with an effective amount of ANG protein or ANG agonist, wherein the effective amount is capable of increasing primitive hematopoietic cell quiescence and proliferation of myeloid progenitor cells.
101. The stem cell collection bags of paragraph 100, further supplemented with nutrients and cytokines.
102. The stem cell collection bag of paragraph any one of paragraphs 100-101, wherein the cytokines are selected from the group consisting of granulocyte colony stimulating factor, granulocyte macrophage colony stimulating factor and erythropoietin.
103. A method of treating a subject suffering with a disease or disorder characterized by decreased in vivo levels of hematopoietic stem cells and progenitor cells or decreased in vivo hematopoietic reconstitution, the method comprising, administering an effective amount of ANG protein or ANG agonist to the subject, wherein the effective amount increases hematopoietic stem cell quiescence and proliferation of myeloid restricted progenitor cells, thereby increasing the in vivo levels of hematopoietic stem cells and progenitor cells or hematopoietic reconstitution.

### EXAMPLES

The following examples illustrate some embodiments and aspects of the invention. It will be apparent to those skilled in the relevant art that various modifications, additions, substitutions, and the like can be performed without altering the spirit or scope of the invention as defined in the claims which follow. The technology described herein is further illustrated by the following examples which is no way should be construed as being further limiting.

### Materials and Methods for Examples 1 to 3

Mice—

All animal experiments were approved by the Institutional Animal Care and Use Committee at Massachusetts General Hospital and Tufts University/Tufts Medical Center. Wild-type C57Bl/6, B6SJL, MTMG, IL18KO, IL18R1KO, NesCreERT2, NG2CreERT2, Col1a1CreERT2 mice were obtained from the Jackson laboratory. Col2.3GFP (Kalajzic et al., 2002) were previously described. Ang/conditional/KO mice were generated by the Hu laboratory. OsxCreERT2 mice were a kind gift of Dr H Kronenberg, Massachusetts General Hospital. For studies using ERT2 mice, tamoxifen (150 mg/kg, Sigma Aldrich) was injected intra-peritoneally daily for three days three times daily in both genotypes (Cre-positive +/+ or fl/fl, and BM was harvested 24 hours following the final injection. Age-matched (7-12 week old littermates were used)

Single OLC Harvesting and Single Cell RNA Seq

Newborn col2.3GFP animals were injected with DiI-labeled LKS CD34-Flk2-adult bone marrow cells, as described below, and sacrificed 48 hours after transplantation. Femurs were dissected, embedded in 10% low melting temperature agarose (Lonza) and sectioned at 100μ using a vibratome (Leica). Single OLC harvesting was performed using a physiology microscope BX51 (Olympus) equipped with filters to detect GFP and DiI fluorescence, DIC optics, micromanipulators (Eppendorff), real-time imaging camera, peristaltic pump, in-line heater, perfusion chamber (Harvard Apparatus and SAS Air Syringe (Research Instruments). Sections were pre-screened for the presence of rare GFP-labeled OLCs located next to single DiI-positive transplanted HSCPs, which were found in 1-2 out of 15 sections per animal. Once a target proximal OLC was identified, the section was rotated so that the target was directly opposite the aspiration pipette (Humagen). The section was secured against the bottom of the perfusion chamber using a horizontal portion of the holding pipette (Humagen). With the aspiration pipette just above the target, the section was perfused with warm (37° C. cell dissociation solution (Liberase™, Roche) for 8-10 minutes while the target cell was visually monitored. Then, applying positive pressure from the micropipette using Air Syringe, hematopoietic cells surrounding the target OLC were dislodged to create a 20-30μ clearing. Finally, the aspiration pipette was lowered onto the target OLC, the cell was gently detached from the endosteal surface and aspirated. The presence of GFP fluorescence in the aspirated cell inside the aspiration pipette was confirmed, the contents of the pipette was ejected into a PCR tube with the lysis buffer for the single cell RNA-Seq protocol, and frozen immediately at −80° C. Reverse transcription, cDNA amplification, library preparation and SOLiD RNA-Seq were performed as described (Tang et al., 2009).

FACS Analysis and Cell Sorting

Gating strategies, phenotypic studies, chimerism analyses, cell cycle assays, BrdU incorporation assays, Annexin V assays, and cell sorting were done as described below

BrdU Incorporation

BrdU was administered in drinking water at 0.35 mg/ml for 3 days. Cells were stained with cells surface markers as detailed above and BrdU antibody using BrdU FITC kit (BD) following fixation and permeabilization, as per manufacturer's instructions.

Bone Marrow Stem Cell Transplantation

Conditioning regimens and transplant procedures are described below.

5 Fluorouracil Treatment

8-week old age and gender matched WT or IL18KO mice were injected with 5-fluorouracil (APP at 150 mg/kg intra-peritoneally. Bone marrow was analyzed on day 8 by flow cytometry. For serial 5FU exposure, animals received weekly intra-peritoneal injections at the same dose.

Bioinformatics and Statistical Analysis

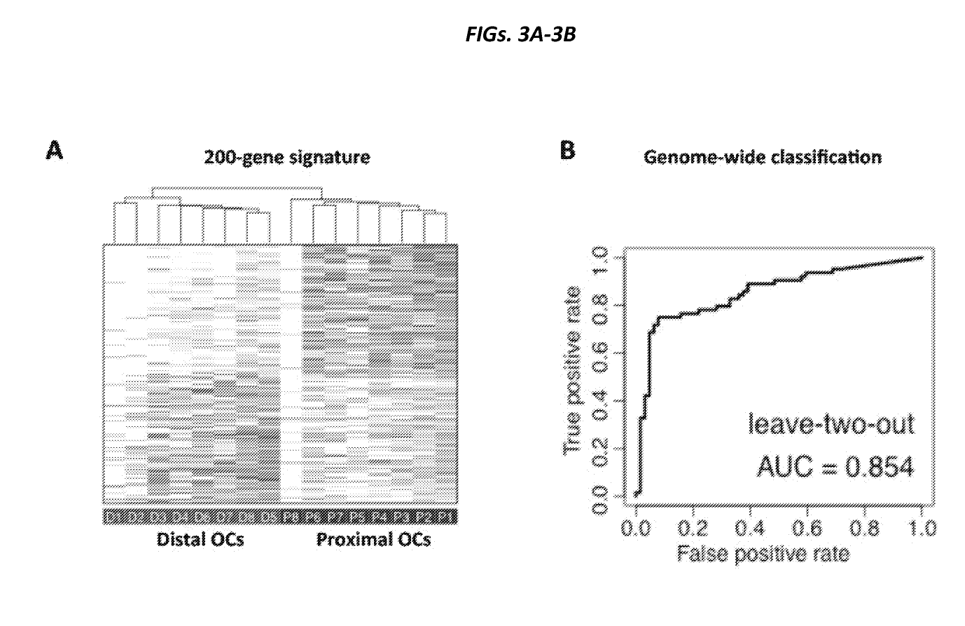

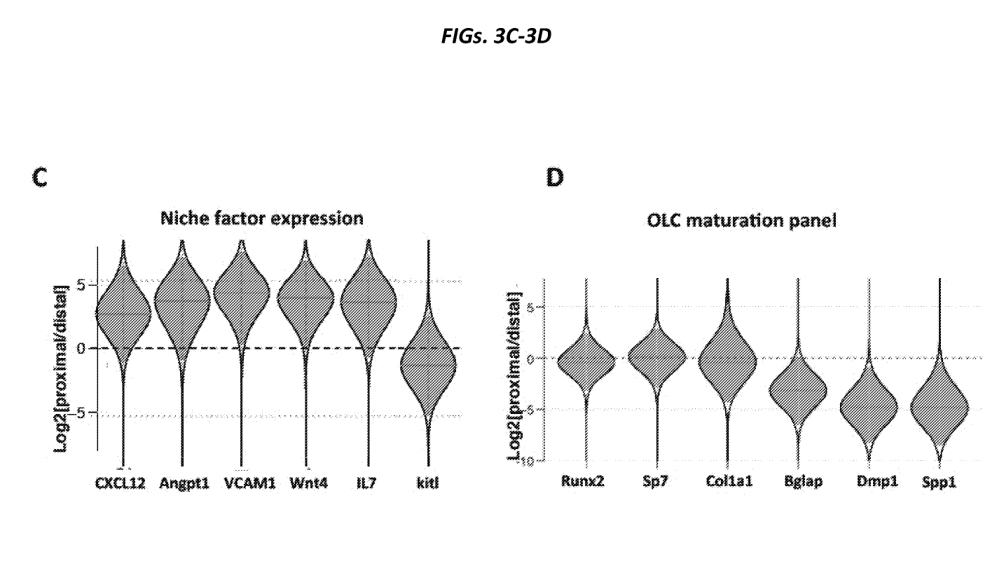

The differential expression estimates were obtained from single-cell RNA-seq data using the approach described (Kharchenko et al., 2014). The stability of differential expression signature/distinguishing OLC-proximal and distal cells was tested using support vector machine (SVM) classifier as follows: the SVM classifiers were constructed using all genes for which expression was detected in any of the examined cells; the ability to distinguish OLC-proximal and distal cells was/tested using leave-two-out validation: one OLC-proximal and one OLC-distal cell was excluded, and a v-classification SVM was constructed based on all remaining cells using e1071 R package. All possible pairs of OLC-proximal and distal cells were tested to evaluate the classification performance (FIG. 3B). Gene set enrichment analysis (GSEA) was performed using mouse GO annotations from Mouse Genome Database (2013.12.27 version, see http://www.informatics.jax.org/for gene listings). A total of 1590 GO categories (BP or CC) containing between 10 and 2000 genes were tested, taking into account the magnitude of the expression differences. In the analysis of the single-cell differential expression, the mode of the log-fold expression difference posterior distributions was used as a difference magnitude (with power factor p=0.5). The empirical P-values were determined based on 106 randomizations, with Q-values derived using Benjamini & Hochberg correction. RNA-Seq data from bulk-sorted samples was aligned to the NCBI mm9 annotation using TopHat. The expression fold-differences were estimated using HTSeq and DESeq. The GSEA was performed using signed expression difference Z-score (power factor p=2, 106 randomizations). To verify classification of the bulk samples based on the 200-gene signature (FIG. 3A), RPKM estimates were used, correcting for mouse batch effect using ComBat (Johnson et al., 2007). The classification was calculated using Ward method hierarchical clustering, with a Euclidean distance metric. The single cell and bulk analysis RNA-Seq data has been deposited in GEO under accession number GSE52359. The full differential expression analysis can be viewed via the following URL,

http://pklab.med.harvard.edu/sde/viewpost.html?dataset=olc.

Intravital Microscopy

WT C57Bl/6 mice or IL18KO mice were irradiated 950 cGy the night before and were intravenously injected with 50,000 LKS cells obtained from MTMG mice (for tdTomato labeling). Intravital imaging of calvarial bone marrow and data analysis were performed at 24 hours post-transplant, as previously described (Lo Celso et al., 2009).

Anti Embigin Mobilization

10 week old C57Bl/6 mice were injected intravenously via tail vein with 2 mg/kg/day of functional grade anti-Embigin antibody (clone G7.43.1; E-bioscience) or IgG2b control antibody for 3 days. Twenty four hours after the last injection peripheral blood was collected via cardiac puncture and phenotypic progenitors determined by flow cytometry and functional progenitors determined by colony assays in methylcellulose as we have previously described (Hoggatt et al.)

Cell Sorting and Flow Cytometry

Whole bone marrow mononuclear cells (BMMNC) were collected by crushing tibias, femurs and hips and stained with the following monoclonal antibodies: c Kit APC, CD34 FITC (e Bioscience), Sca1 BV421, Flk2 PE, IL18Rα/CD218a (E Bioscience), CD48 APCCy7 (BD), lineage cocktail biotin (B220, Mac1, Ter119, CD3, CD4, CD8 at 1:1:1:1:1:1 followed by streptavidin Pacific Orange (Invitrogen). LT HSCs, ST HSCs and MPP were gated as described. For the lineage analysis, red cell depleted BMMNC or peripheral blood samples were stained with CD3 APC (e Bioscience), Mac1FITC, Gr1 PeCy7 and B220 PE (BD). For CLP enumeration, BMMNC were stained with FITC conjugated antibodies against Mac1, Gr1, CD19, Ter119, CD3 Pacific Blue, Flk2 PE, B220 PE Cy7 and biotin conjugated IL7R/CD127, followed by streptavidin PerCP Cy5.5 (all from BD. For CLP cell cycle analysis, BMMNC were stained with lineage cocktail biotin (B220, Mac1, Ter119, CD3, CD4, CD8 at 1:1:1:1:1:1 followed by PE Texas Red conjugate (Invitrogen), B220 PE Cy5, CD127PE, Flk2APC and DAPI. For post-transplant chimerism analysis, CD45.1 AF700 and CD45.2 Pacific Blue (BD) were added. 7 AAD (BD or DAPI (Invitrogen) were used as viability dyes. At least 2×10⁶ events per sample were acquired for progenitor analysis and 104 events for lineage analysis using a BD LSRII flow cytometer. For cell cycle analysis, BMMNC were stained with monoclonal antibodies for HSPC markers, as described above. The cells were permeabilized using Cytofix/Cytoperm Fixation/Permeabilization Kit (BD) according to the manufacturer's instructions and stained with Ki 67 FITC (BD), Hoechst 33342 or DAPI (Invitrogen). For FACS analysis/sorting of osteolineage cells, bone fragments were obtained by gently crushing tibiae, femora, humeri and pelvic bones of 4 6 weeks old col2.3GFP mice. After rinsing away the bone marrow cells, the fragments were incubated with 0.25% Collagenase (Stem Cell Technologies) at 37° C. with gentle agitation for 1 hour. The samples were vortexed several times during the incubation, then filtered through 0.45 micron mesh and stained with CD45 APC Cy7, Ter 119 APC Cy7 (BD), Embigin PE (E Bioscence) and CD106 APC (R D Systems). The samples were analysed using LSRII (BD) or FACS sorted using Aria (BD). Compensation and data analysis were performed using Flowjo 7.6 software. For the RNA Seq analysis of GFP+Embbright VCAM 1+ cells, lethally irradiated col2.3GFP mice were injected with 10,000 LKS CD34 Flk2 LT HSCs, lin kit+Sca progenitors or PBS and sacrificed 48 hours later. GFP+Embbright VCAM 1+ cells and remaining GFP+cells were sorted directly into the lysis buffer for the single cell RNA Seq protocol, and frozen immediately at 80° C. Reverse transcription, cDNA amplification, library preparation, SOLiD RNA Seq were performed as described for the single cell RNA Seq samples, except for the reduction in the initial PCR amplification cycle number from 20 to 18. Three biological replicates for each sample group were sequenced. For FACS analysis of IL18 receptor expression in human primitive hematopoietic cells, CD34 enriched bone marrow or cord blood cells were stained with the following antibodies: CD34 APC Cy7, CD38 FITC, CD45RA APC, CD10 BV510, CD49f BV650, CD90 BV421 (all from BD and CD218a/IL18R1 PE (E Bioscience), as described (Notta et al., 2011).

Bone Marrow/Stem Cell Transplantation

Adult recipients (CD45.2) were irradiated 950 cGy the evening before and transplanted with 500K total bone marrow cells (CD45.1) via retro orbital injection. For LKS cell transplantation, lethally irradiated animals were intravenously injected with 8,000 CD45.1 LKS cells and CD45.2 support cells for IL18KO experiments, 8000 CD45.2 LKS cells and CD45.2 support cells from for IL18 receptor KO experiments. For the transplants which involved Ang conditional knock out strains, 500K bone marrow cells from Ang deleted animals (45.2) were co transplanted with 500K bone marrow cells from CD45.1 animals into lethally irradiated CD45.1 recipients. For non-competitive transplants, 106 CD45.1 bone marrow cells were used. Recipients' peripheral blood chimerism was assessed at 4 weekly intervals after transplantation. For neonatal transplantation, col2.3GFP P2 pups were irradiated 450 cGy the evening before. Adult bone marrow LKS 34 Flk2 cells were isolated as described and labeled with DiI according to manufacturer's instructions. 5000-7000 cells per animal were injected in a 50 μl volume via anterior facial vein.

### Methods for Examples 4 to 8

Experimental Procedures

Animal Studies

Ang−/− mice were generated in-house. B6.SJL and NSG mice were purchased from The Jackson Laboratory. For aged animal experiments, 22-month old WT (NIH/NIA) and Ang−/− mice were used. For all other studies, age-matched 7-12 week old mice were used. Littermates and gender-matched animals were used whenever possible. All procedures were performed in accordance with protocols approved by Institutional Animal Care and Use Committee of Tufts University/Tufts Medical Center.

Statistical Analyses

All bar graphs represent mean±SEM and all heatmaps represent mean. All data are derived from 2-4 independent experiments. For comparisons of two experimental groups, an unpaired two-tailed Student's t-test was used (Excel). Kaplan-Meier survival curves were analyzed using log rank tests (Prism 6). Heatmaps were generated using RStudio. LDA was assessed by ELDA (http://bioinf.wehi.edu.au/software/elda/). For all analyses, *p<0.05, **p<0.01, ***p<0.001, and ns=not significant.

Bone Marrow Cellularity

Femurs were dissected and flushed with 5 ml phosphate buffered saline (PBS) supplemented with 2% fetal bovine serum (FBS, Mediatech). Cells were resuspended by pipetting and vortexing. White blood cell counts were obtained by VetScan HM5 instrumentation (Abaxis Veterinary Diagnostics).

Generation of ANG

Mouse and human recombinant ANG protein were generated by a pET E. coli expression system and purified to homogeneity by HPLC in-house (Shapiro et al., 1988). Angiogenic and ribonucleolytic activity of each batch of ANG preparation was confirmed (data not shown). ANG variants (R33A, K40Q, and R70A) were generated through site-directed mutagenesis followed by expression in pET system and purification by HPLC.

In Vivo and In Vivo ANG Treatment

Unless otherwise indicated (in dose response experiments), 300 ng/ml ANG was used for in vivo treatments. For all in vivo ANG treatments, 1.25 mg/kg was injected intraperitoneally at the indicated time points.

5-Fluorouracil (5-FU) Treatment

For 5-FU rebound experiments, 5-FU (150 mg/kg) was injected intraperitoneally once and BM harvested for analysis on Day 7. For serial 5-FU treatments, 5-FU (150 mg/kg) was injected intraperitoneally every 7 days until 100% animal mortality was achieved.

Histology

Femurs were dissected from animals and fixed overnight in 10% neutral buffered formalin.

Bones were prepared, decalcified, and stained with Hematoxylin and Eosin (H&E) by the Tufts

Animal Histology Core.

Genotyping

Genotyping was performed by PCR with Hot Start Green PCR Master Mix (Thermo Scientific), using standard PCR conditions on an iCycler PCR machine (Biorad). The Ang primers for Ang−/− mice were as follows: Forward, 5′-AGCGAATGGAAGCCCTTACA-3′ (SEQ ID NO: 2); reverse, 5′-CTCATCGAAGTGGACAGGCA-3′ (SEQ ID NO: 3). The primers for the LoxP site (F12/B6) were as follows: Forward, 5′-AGGGTGGAACTTCAGGATTCAAG-3′ (SEQ ID NO: 4); reverse, 5′-GAAGTTATCCGCGGGAAGTTC-3′ (SEQ ID NO: 5).

Complete Blood Counts

Peripheral blood was harvested from mice by retro-orbital bleeding using heparinized micro-hematocrit capillary tubes (Fisherbrand). Blood was collected directly into EDTA-coated Microtainer tubes (BD) and automated complete blood counts were assessed by VetScan HM5 instrumentation.

Flow Cytometry and Cell Sorting

Whole bone marrow mononuclear cells (BMMNC) were obtained by crushing tibias and femurs in PBS/2% FBS and straining cellular suspension through 0.45 μm mesh. Red blood cells were depleted using ACK Lysis Buffer (Lonza). Briefly, 2 ml buffer was added to cell pellet and incubated on ice for 5 minutes with periodic vortexing Cells were washed once and resuspended in 200 μl PBS/2% FBS for staining using 1:200 dilutions of primary antibodies unless otherwise indicated. Gating was established by the following phenotypic cell surface markers, based on standard gating approaches:

Methods Table 1. Surface markers for
gating of various cell populations.

| Cell Type | Cell Surface Markers |
|---|---|
| LKS | Lin−c-Kit+Scal+ |
| Myeloid-restricted progenitor | Lin−c-Kit+Scal− |
| LT-HSC | Flk2−CD34− LKS |
| ST-HSC | Flk2−CD34+ LKS |
| MPP | Flk2+CD34+ LKS |
| HSC | CD150+CD48−CD135−CD34− LKS |
| MPP1 | CD150+CD48−CD135−CD34+ LKS |
| MPP2 | CD150+CD48+CD135−CD34+ LKS |
| MPP3 | CD150−CD48+CD135−CD34+ LKS |
| MPP4 | CD150+CD48+CD135+CD34+ LKS |
| CLP | Lin− IL7R+ Flk2+ B220− |
| Pre-pro B | Lin− IL7R+ Flk2+ B220+ |
| CMP | Lin−c-Kit+Scal−CD34+CD16/32− |
| GMP | Lin−c-Kit+Scal−CD34+CD16/32+ |
| MEP | Lin−c-Kit+Scal−CD34−CD16/32− |

For stem and progenitor staining, red cell-depleted BMMNCs were stained with antibodies against cKit BV711 (BD), Sca1 PE-Cy5 (eBioscience), Flk2 PE (BD), CD34 e660 (eBioscience), IL7R APC-Cy7 (eBioscience), B220 BV785 (Biolegend), CD16/32 AF700 (eBioscience) and a biotinylated lineage cocktail (B220, CD3, CD4, CD8, Mac1, and Ter119 at 1:1:1:1:1:1). Cells were stained for 90 minutes on ice, followed by streptavidin PE-Cy7 (Biolegend) for 15 minutes on ice. Cells were analyzed using a FACSAria flow cytometer (BD).

For lineage analysis, red cell-depleted BMMNCs were stained for 30 minutes on ice with antibodies against CD11b PE-Cy7 (Biolegend), Gr1 PE (eBioscience, 1:400), CD45R/B220 FITC (BD), CD3ε APC-Cy7 (Biolegend), and Ter119 APC (eBioscience). Cells were analyzed using a LSRII flow cytometer (BD).

For chimerism studies, peripheral blood was obtained by retro-orbital bleeding and depleted of red blood cells. Samples were stained for 30 minutes on ice with antibodies against CD45.1 APC (eBioscience), CD45.2 Pacific Blue (Biolegend), CD11b PE-Cy7, Gr1 PE, CD45R/B220 FITC, and CD3ε APC-Cy7. Cells were analyzed using a LSRII flow cytometer.

For sorting LKS cells or myeloid-restricted progenitors, red cell-depleted BMMNCs were stained with antibodies against cKit APC (eBioscience), Sca1 PE (eBioscience), and a FITC lineage cocktail for 30 minutes on ice. Cells were sorted using FACSAria or MoFlow Astrios (Beckman Coulter) flow cytometers. For sorting LT-HSCs, red cell-depleted BMMNCs were stained with antibodies against cKit APC-eF780 (eBioscience), Sca1 PE-Cy5, Flk2 PE, CD34 e660, and a biotinylated lineage cocktail. Cells were stained for 90 minutes on ice, followed by streptavidin PE-Cy7 (Biolegend) for 15 minutes on ice. Cells were sorted using a FACSAria flow cytometer.

For all analyses, 4′,6-diamidino-2-phenylindole (DAPI, Molecular Probes) or 7-aminoactinomycin d (7-AAD, BD) were used as viability dyes, per manufacturer's instructions. At least 2×10⁶ events per sample were acquired for bone marrow stem and progenitor analysis and 3×10⁴ events for lineage analysis. Data were analyzed using FlowJo X (Tree Star).

Cell Cycle Analysis

For cell cycle, 1×107 red cell-depleted BMMNCs were stained with cell surface markers as described above and fixed and permeabilized using Cytofix/Cytoperm Fixation/Permeabilization Kit (BD) per manufacturer's instructions. Cells were then stained with Ki67 FITC (BD, 1:10 in BD Perm/Wash buffer) and DAPI (2 μg/ml for 10 minutes prior to analysis), and analyzed using a FACSAria flow cytometer, acquiring 2×10⁶ events per sample.

BrdU Incorporation

BrdU was administered in drinking water (0.35 mg/ml) for 3 days. Volume of drinking water was assessed to confirm equal water intake among cages. Mice were sacrificed and red cell-depleted BMMNCs were stained with antibodies against cell surface markers (1:200) as follows:

For HSPCs, cells were stained with c-Kit APC-eF780, Sca1 PE-Cy5, Flk2 PE, CD34 e660 and a biotinylated lineage cocktail. Cells were stained for 90 minutes on ice, followed by streptavidin Pacific Orange (Invitrogen) for 15 minutes on ice.

For lymphoid-restricted progenitors, cells were stained with c-Kit APC-eF780, Sca1 PE-Cy7 (Biolegend), IL7R PE (eBioscience), B220 PE-Cy5 (eBioscience), and a biotinylated lineage cocktail. Cells were stained for 90 minutes on ice, followed by streptavidin Pacific Orange for 15 minutes on ice.

For myeloid-restricted progenitors, cells were stained with c-Kit APC-eF780, Sca1 PE-Cy5, CD16/32 BV605 (BD), CD34 e660 and a biotinylated lineage cocktail. Cells were stained for 90 minutes on ice, followed by streptavidin Pacific Orange for 15 minutes on ice.

Following cell surface stain, cells were fixed and permeabilized, and stained with BrdU FITC (BD), per manufacturer's instructions. For all stains, cells were analyzed using a FACSAria flow cytometer, acquiring 2×10⁶ events per sample. BrdU gating was established by cells isolated from mice not administered BrdU and BrdU fluorescence-minus-one controls.

Annexin V Analysis

To assess apoptotic activity, red cell-depleted BMMNCs were stained for cell surface markers as above, and stained with Annexin V FITC (BD), per manufacturer's instructions. Briefly, cells were resuspended in 1× Binding buffer (BD) at 1×10⁶ cells/ml and stained for 15 min at room temperature (RT) in the dark. Four hundred μ1 of 1× Binding buffer was added to each tube analyzed on a LSRII or FACSAria flow cytometer within 1 hour. Annexin V-positive gates were established by Annexin V fluorescence-minus-one controls.

Mouse and Human Methylcellulose Colony Assays

For myeloid progenitor quantification, 2×10⁴ whole BMMNCs were plated in MethoCult M3434 methylcellulose (Stem Cell Technologies), per manufacturer's instructions. Colonies were scored by visualization on Day 12.

For serial re-plating assays, 2×10⁴ whole BMMNCs were plated in MethoCult M3434 methylcellulose and colonies were scored at Day 7. Colonies were the harvested, per manufacturer's instructions, 2×10⁴ whole BMMNCs were again plated in methylcellulose. Colonies were subsequently scored on Day 14.

For pre-pro B progenitor quantification, 5×10⁴ whole BMMNCs were plated in MethoCult M3630 methylcellulose (Stem Cell Technologies), per manufacturer's instructions. Colonies were scored by visualization on Day 7.

For human progenitor quantification, 2×10⁴ human CD34+ cord blood cells (Stem Cell Technologies, mixed donors) were plated in MethoCult H4034 methylcellulose in the presence or absence of 300 ng/ml human ANG. Colonies were scored by visualization on Day 15.

All assays were cultured in untreated 35-mm culture dishes (Stem Cell Technologies) and maintained for the duration of the experiment at 37° C./5% CO2, per manufacturer's instructions. For all experiments, data were presented as frequency of total number of plated cells.

Quantitative RT-PCR Analyses

Total RNA was extracted from sorted or treated hematopoietic cell populations using RNeasy Plus Micro Kit (Qiagen), and was reverse transcribed into cDNA with Quantitech Reverse Transcription Kit (Qiagen), per manufacturer's instructions. For qRT-PCR analysis of rRNA species, random primers (IDT) were used during reverse transcription. For all other analyses, Oligo(d)T primers (IDT) were used. qRT-PCR analysis was performed on a LightCycler 480 II (Roche) using SYBR Green PCR mix (Roche). Relative expression was determined by the 2-ΔΔCt method, using β-actin as an internal control. Primer sequences were adapted from the following sources: mouse p21, p27, and p57 (Chakkalakal et al., 2014); mouse GATA3, Bmi1, and vWF (Kent et al., 2009); mouse a1, Bcl2, Bcl-xl, Mcl1, Bak, Bax, Bid, Bim, Noxa, Puma, and β-Actin (Mohrin et al., 2010); human p21 (Zhu et al., 2011); human p27 (Bryant et al., 2006); human p57 (Giovannini et al., 2012); human GATA3 (Wang et al., 2014); human vWF (Poon et al., 2012); human Bmi1 (Abdouh et al., 2009); human cyclin D1 (Ding et al., 2009); and human β-Actin (Sheng et al., 2014). Tables 2 and 3, below, for primer information.

Mouse LT-HSC Culture

For 2 hour treatments in PBS, LT-HSCs were sorted directly into PBS and cultured in the presence or absence of 300 ng/ml ANG. For other cell proliferation and qRT-PCR analyses, LT-HSCs were sorted into 96-well plates and cultured in S-clone SF-O3 (Sanko Junyaku), supplemented with 0.5% bovine serum albumin (Gibco Life Technologies), 50 ng/ml thrombopoietin (Peprotech), 50 ng/ml stem cell factor (Peprotech) and 50 μM 2-mercaptoethanol (Gibco Life Technologies), in the presence or absence of 300 ng/ml ANG. For 2- or 7 day treatments, 1× Penicillin/Streptomycin (Corning) was included in culture medium. Cells were cultured at 37° C./5% CO2.

For proliferation studies, cell number was determined by hemocytometer. For qRT-PCR studies, cells were harvested and analyzed as described under “Quantitative RT-PCR Analyses”. For BM transplantation, cells were harvested, washed with PBS, and counted. Equal cell numbers were transplanted as described under “Mouse Bone Marrow Transplantation.”

Human CD34+ Cord Blood Cell Culture Human

CD34+ cord blood cells (Stem Cell Technologies) were thawed per manufacturer's instructions. For 2 hour treatments, cells were cultured in PBS in the presence or absence of 300 ng/ml hANG. For 7 day culture, cells were cultured in StemSpan SFEM (Stem Cell Technologies), supplemented with stem cell factor, Flt3 ligand, IL6, and thrombopoietin (100 ng/ml, R&D), in the presence or absence of 300 ng/ml hANG. Cells were cultured at 37° C./5% CO2. Commercial human ANG (R&D) was also tested at 300 ng/ml and shown to neither have as strong induction of candidate self-renewal transcripts nor as strong reduction in proliferation, consistent with our previous findings that the biological activity of commercial ANG is about 10% of our in house ANG preps (data not shown). Human ANG variants (K40Q, R70A, R33A) were used at the same concentration of 300 ng/ml. For proliferation studies, cell number was determined by hemocytometer. For qRT-PCR studies, cells were harvested and analyzed as described under “Quantitative RT-PCR Analyses”.

Mouse Bone Marrow Transplantation

For all mouse transplant studies, recipient mice were lethally-irradiated 16 hours prior to transplantation with 12 Gy total body irradiation (TBI, split dose 3 hours apart). All mice were irradiated in a pie cage (Braintree Scientific) with rotation (JL Shepherd irradiator). For each experiment, mice from different experimental groups were simultaneously irradiated to ensure equal irradiation among groups.

For serial transplantation of LT-HSCs into ANG-deficient hosts, 400 sorted LT-HSCs from CD45.1 donor mice were co-injected with 1×10⁶ CD45.2 whole BM support cells into lethally-irradiated WT or Ang−/− (CD45.2) recipient mice. After 24 months, BM was harvested, 400 LT-HSCs were re-sorted and transplanted again into WT or Ang−/− (CD45.2) secondary recipients with 1×10⁶ CD45.2 whole BM support cells.

For serial transplantation of WBM into ANG-deficient hosts, 1×106 whole BM cells were transplanted into lethally-irradiated WT or Ang−/− (CD45.2) recipient mice. After 24 months, BM was harvested and 1×10⁶ whole BM cells (CD45.1) were transplanted again into WT or Ang−/− (CD45.2) secondary recipients.

For direct 1:1 competitive transplantation studies using 22 month old WT or Ang−/− mice, 5×10⁵ whole BMMNCs (CD45.2) were intravenously co-injected with 5×105 B6.SJL (CD45.1) support cells into lethally-irradiated B6.SJL (CD45.1) recipient mice.

For ex vivo reconstitution assays, WT and Ang−/− LT-HSCs (CD45.2), either freshly sorted or cultured with or without 300 ng/ml ANG for 2 hours or 7 days, were washed in PBS, and 400 donor cells were intravenously co-injected with 1×10⁶ B6.SJL (CD45.1) support cells into lethally-irradiated B6.SJL (CD45.1) recipient mice. For secondary transplantation in ex vivo reconstitution assays, C57BL/6 LT-HSCs (CD45.2) were sorted from primary recipients that were transplanted with fresh LT-HSCs or LT-HSCs treated with or without ANG for 2 hours. Four hundred LT-HSCs from primary recipients were then intravenously co-injected with 1×10⁶ B6.SJL (CD45.1) support cells into lethally-irradiated B6.SJL (CD45.1) recipient mice.

For transplantation of tiRNA-transfected LKS cells, 3,000 sorted C57BL/6 LKS (CD45.2) were transfected as described under “tiRNA Transfection”, and intravenously co-injected with 1×106 B6.SJL (CD45.1) support cells into lethally-irradiated B6.SJL (CD45.1) recipient mice.

For transplantation of irradiated BM (pre-treatment group), C57BL/6 (CD45.2) mice were pretreated daily for three successive days with ANG and irradiated (4 Gy TBI) 24 hours following the final ANG treatment. BM was harvested at Day 7, donor BMMNCs were pooled and intravenously co-injected with B6.SJL (CD45.1) support cells (1:1) into lethally-irradiated B6.SJL (CD45.1) recipient mice. For the delayed treatment group, C57BL/6 (CD45.2) mice were irradiated (4 Gy) and treated with ANG daily for three successive days, beginning 24 hours post-irradiation. BMMNCs were harvested and transplanted as in the pre-treatment group.

For all transplants, except for irradiation reconstitution assays, peripheral blood was taken by retro-orbital bleeding at 4-week time intervals, up through 16 or 24 weeks, as indicated. For irradiation assays, peripheral blood was taken by retro-orbital bleeding at 16 weeks post-transplant. Reconstitution units (RU) per femur, corresponding to the HSC content per 1×10⁵ BM cells, was calculated as previously described (Purton and Scadden, 2007; Winkler et al., 2012).

Human CD34+ Cord Blood Cell Transplantation

NSG mice were purchased from The Jackson Laboratory and maintained in sterile housing. Recipient NSG mice were sublethally irradiated (2.5 Gy TBI) 16 hours prior to transplantation. Human CD34+ cord blood cells from mixed donors were treated with or without 300 ng/ml human ANG for 2 hours in PBS at 37° C./5% CO2. Cells were washed once in PBS and intravenously injected in three doses: 100, 1,000, and 10,000 cells. Both male and female mice were used as recipients for all treatments and doses. No significant differences were observed among experimental groups between male and female mice, different from a previous report (McDermott et al., 2010). At 16 weeks post-transplant, red cell-depleted BMMNCs were surface stained with the following antibodies for 30 minutes on ice (1:200 dilution): human CD45 Pacific Blue (Biolegend), Mouse CD45 APC-e780 (eBioscience), Human CD19 PE-Cy7 (BD), Human CD33 PE (BD). Samples were analyzed using a FACSAria flow cytometer. Engraftment was assessed by the frequency of human CD45 cells. All samples demonstrating greater than or equal to 0.1% hCD45 expression were considered to be positively-engrafted, in keeping with prior studies (Boitano et al., 2010).

Homing Assay

Homing assays were performed as described previously (Hoggatt et al., 2009). For homing assays using WT or Ang−/− mice as recipients, 2×10⁶ CD45.1 Lin− cells were labeled with CFSE (Molecular Probes) per manufacturer's instructions, and transplanted into lethally-irradiated WT or Ang−/− (CD45.2) recipient mice. Cells were harvested 16 hours post-transplant, stained with antibodies against cell-surface markers as described above, and analyzed on a FACSAria flow cytometer. Percent CFSE-positive LKS cells and myeloid-restricted progenitors was determined. For homing assays using ANG-treated cells, 2×10⁶ CD45.2 Lin− cells were treated with 300 ng/ml ANG in PBS for 2 hours at 37° C./5% CO2. Cells were labeled with CFSE, as above, and transplanted into lethally-irradiated B6.SJL (CD45.1) recipient mice. Cells were harvested 16 hours post-transplant, stained with antibodies against cell-surface markers as described above, and analyzed on a FACSAria flow cytometer. Percent CFSE-positive LKS cells and myeloid-restricted progenitors was determined.

Protein Synthesis Analyses

Determination of protein synthesis rates in BM cells was done using OP-Puro as described in reference (Signer et al., 2014). For in vivo analyses, LKS cells or myeloid-restricted progenitors were sorted as described above, and plated in DMEM (Sigma) in the presence or absence of 300 ng/ml ANG. Cells were cultured for 2 hours at 37° C./5% CO2. Cells were washed once with Ca2+- and Mg2+-free PBS and cultured for 1 hour with OP-Puro (50 Medchem Source). Cells were fixed in 0.5 ml of 1% paraformaldehyde (Affymetrix) in PBS for 15 minutes on ice, washed once with PBS, and then permeabilized with 200 μl PBS supplemented with 3% FBS and 0.1% saponin (Sigma) for 5 minutes at room temperature (RT). Click-iT Cell Reaction Buffer Kit (Life Technologies) was used for azide-alkyne cycloaddition of AF488-conjugated azide (5 μM, Life Technologies), per manufacturer's instructions. Cells were washed twice in PBS/3% FBS/0.1% saponin and analyzed using a FACSAria flow cytometer.

For in vivo analyses, OP-Puro was injected intraperitoneally (50 mg/kg in PBS). One hour post-injection, BM was collected from sacrificed mice and red cell-depleted BMMNCs were stained as follows. Unless otherwise indicated, primary antibodies were used at 1:200 dilution. For stem and progenitor staining, 5×106 cells were stained with cKit BV711, Sca1 APC-Cy7 (Biolegend, 1:80), Flk2 APC (Biolegend, 1:50), CD34 e450 (eBioscience, 1:50), and a biotinylated lineage cocktail. Cells were stained for 90 minutes on ice, followed by streptavidin Pacific Orange for 15 minutes on ice. For lymphoid-restricted progenitor staining, 5×10⁶ cells were stained with cKit BV711, Sca1APC-Cy7, Flk2 APC, IL7R PerCP-Cy5.5 (eBioscience, 1:80), B220 BV650 (Biolegend, 1:80) and a biotinylated lineage cocktail. Cells were stained for 90 minutes on ice, followed by streptavidin Pacific Orange for 15 minutes on ice. For myeloid-restricted progenitor staining, 5×10⁶ cells were stained with cKit BV711, Sca1 APC-Cy7, CD16/32 BV605 (BD. 1:80), CD34 e450 and a biotinylated lineage cocktail. Cells were stained for 90 minutes on ice, followed by streptavidin Pacific Orange for 15 minutes on ice.

For lineage staining, 5×10⁵ cells were stained with Mac1 APC (eBioscience), Gr1 PE (1:400), CD3ε Pacific Blue (Biolegend, 1:100), and Ter119 APC-Cy7 (Biolegend, 1:100) for 30 minutes on ice. Following surface staining, cells were washed twice with Ca2+- and Mg2+-free PBS and resuspended in 1 ml PBS. One μl UV-fixable eFluor 455 viability dye was added (eBioscience), cells were incubated for 30 minutes at 4° C. in the dark, and washed once with PBS, per manufacturer's instructions. Following staining, cells were fixed and permeabilized and cycloaddition of AF488-conjugated azide (Life Technologies) was performed as described above. Cells were analyzed using a FACSAria flow cytometer, acquiring 2×10⁶ events per sample for BM stem and progenitor analysis and at least 3×10⁴ events for lineage analysis. Treated samples were compared to mice or cells not administered OP-Puro and/or OP-Puro fluorescence-minus-one controls. Relative rate of protein synthesis was determined as described previously (Signer et al., 2014). Briefly, background fluorescence was subtracted from OP-Puromycin AF488 geometric means and normalized relative to whole BM or WT controls for in vivo and in vivo experiments, respectively.

tiRNA Gel Electrophoresis

For all RNA work, equipment was sterilized according to standard laboratory protocol and diethylpyrocarbonate-treated water was used for all procedures. Total RNA was isolated and pooled from sorted LKS cells, myeloid-restricted progenitors, or lineage-positive cells for each experimental parameter. Total RNA was diluted in 2× Novex TBE-Urea sample buffer (Invitrogen), heated to 65° C. for 5 minutes and cooled briefly to RT prior to loading. A 15% TBE-Urea Gel (Invitrogen) was pre-run at 74 V for 60 minutes and samples were electrophoresed to the bottom of the gel at 100 V in 0.5×TBE running buffer. A low molecular weight marker (10-100 nt, Affymetrix) was simultaneously run to compare RNA band sizes.

Following electrophoresis, the gel was equilibrated in 0.5×TBE for 5 minutes and stained with SYBR Gold solution (Invitrogen) diluted in 20 ml of 0.5×TBE buffer for 60 minutes with agitation, per manufacturer's instructions. Gels were imaged on a Kodak Electrophoresis Documentation and Analysis System 120 using UV illumination. Images were quantified by Image J software (NIH) and multiple independent experiments were normalized and averaged. For oxidative stress experiments, cells were treated with 500 μM sodium arsenite (Sigma Aldrich) for 2 hours in the presence or absence of 300 ng/ml ANG. For irradiation experiments, WT C57BL/6 mice were irradiated with 4.0 Gy TBI. Twenty four hours post-TBI, LKS cells or myeloid-restricted progenitors were sorted and treated in vivo with 300 ng/ml ANG for 2 hours in PBS at 37° C./5% CO2. For culture experiments, sorted LKS cells were either immediately stimulated with ANG or cultured for 7 days in the presence or absence of ANG in S-clone media, as indicated above. On Day 7, cells cultured in the presence or absence of ANG were harvested, washed once in PBS, and again stimulated with or without 300 ng/ml ANG for 2 hours in PBS at 37° C./5% CO2.

Northern Blotting

Total RNA was isolated from ANG-treated LKS cells or myeloid-restricted progenitors and subjected to electrophoresis, as described above. RNA was transferred to a Pall Biodyne nylon membrane (Promega) using wet transfer. Briefly, a transfer cassette was assembled with the following pre-wet components: sponge, 3 pieces Whatman chromatography paper, gel, membrane, 3 pieces Whatman chromatography paper, and sponge. The apparatus was then transferred in pre-chilled 0.5×TBE at 80 V for 60 minutes at 4° C. Following transfer, the apparatus was disassembled and the membrane rinsed in 1×TBE. Transfer efficiency was confirmed by post-transfer staining of the gel with SYBR Gold, as described above. RNA was fixed to the blot by baking at 80° C. for 2 hours. The membrane was rinsed in pre-warmed digoxigenin (DIG) Easy Hyb buffer (Roche) for 30 minutes at 50° C. with rotation and then hybridized in DIG Easy Hyb buffer containing DIG-labeled DNA Probe (IDT) at 25 ng/ml. For 5′-Gly-GCC the HPLC-purified DIG-labeled probe with the sequence of 5′-GGCGAGAATTCTACCACTGAACCACCAA-3′ (SEQ ID NO: 6) was used. The probe was heat-denatured for 5 minutes prior to hybridization. Following overnight hybridization, membranes were rinsed once in 2×SSC/0.1% SDS for 10 minutes at 60° C., twice in 0.5×SSC/0.1% SDS for 20 minutes at 60° C. and once for 5 minutes in Washing Buffer (Roche) at RT, all with agitation. Following stringency washes, the membranes were blocked for 30 minutes, rocking at RT in blocking solution (Roche), probed with alkaline phosphatase-labeled anti-DIG antibody (Roche) for 30 minutes at RT, washed twice for 20 minutes per wash with washing buffer (Roche), equilibrated for 5 minutes in detection buffer (Roche), and visualized with CSPD (Roche), per manufacturer's instruction.

tiRNA Transfection

Active 5′-P-tiRNA-Gly-GCC (5′-P-AUUGGUGGUUCAGUGGUAGAAUUCUCGCCUGCC-3′ (SEQ ID NO: 7)) was commercially synthesized (IDT). Inactive, 5′-dephosphorylated (d)5′-P-tiRNA was generated by treating active 5′-P-tiRNA with acid phosphatase (Sigma). Sorted LKS cells or myeloid-restricted progenitors were transfected with 1 μM of 5′-P-tiRNA-Gly-GCC or (d)5′-P-tiRNA-Gly-GCC using Lipofectamine 2000 (Invitrogen), as previously described (Yamasaki et al., 2009; Ivanov et al., 2011).

Immunofluorescence and Confocal Microscopy

LKS cells or myeloid-restricted progenitors were sorted directly onto poly-L-lysine coated slides (Thermo Scientific). Cells were allowed to settle onto the slide for 20 minutes, fixed in methanol at RT for 10 minutes, washed once with PBS, and blocked with 30 mg/ml BSA/PBS at 37° C. for 1 hour. Cells were stained with primary antibody in a humidified chamber at 4° C. overnight. For ANG/PABP localization, cells were stained with R163 rabbit polyclonal antibody (pAb) of ANG (10 μg/ml) and F-20 goat pAb of PABP (Santa Cruz #sc-18611, 1:50 dilution), followed with AF488-conjugated goat anti-rabbit (Thermo Scientific A11070, 1:600 dilution) and AF555-conjugated donkey anti-goat (Thermo Scientific A21432, 1:600 dilution). For RNH1/PABP localization, cells were stained with R127 rabbit pAb of RNH1 (5 μg/ml) and F-20 goat pAb of PABP followed with AF488-conjugated goat anti-rabbit AF488 and AF555-conjugated donkey anti-goat. For ANG/RNH1 localization, cells were stained with an in-house made mouse ANG-specific C527 monoclonal antibody (10 μg/ml) and R127 rabbit pAb of RNH1 (5 μg/ml), followed with AF488-conjugated rabbit anti-mouse (Thermo Scientific A11059, 1:600 dilution) and AF555-conjugated goat anti-rabbit (Thermo Scientific A21428, 1:600 dilution). Appropriate isotype controls were used at the same concentration. Images were acquired using Nikon A1R confocal microscopy.

Fluorescence Resonance Energy Transfer (FRET)

FRET was performed using the acceptor photo-bleaching method, as previously described (Pizzo et al., 2013). Briefly, AF488 was used as the donor and AF555 as the acceptors. Signals were photobleached to less than 10% of the initial fluorescent measurement. ROI measurements from LKS cells and myeloid-restricted progenitors were taken from 10 individual cells. FRET efficiency was calculated using the formula E=(IDA−ID)/ID, where ID and IDA are fluorescence intensities before and after photobleaching, respectively. FRET was performed using Leica SP2 confocal microscopy.

METHODS TABLE 2
Mouse qRT-PCR Primer Sequences (Table 2 discloses Forward Primers as SEQ ID
NOS 8-29 and Reverse Primers as SEQ ID NOS 30-51, respectively, in order of appearance)

| Gene | Forward Primer (5′ to 3′) | Reverse Primer (5′ to 3′) |
|---|---|---|
| p21 | TGGAGTCAGGCGCAGATCCAC (SEQ ID NO: 8) | CGCCATGAGCGCATCGCAATC (SEQ ID NO: 30) |
| p27 | AGGCAAACTCTGAGGACCGGCA (SEQ ID NO: 9) | TGCTCCACAGTGCCAGCGTTC (SEQ ID NO: 31) |
| p57 | CGAGGAGCAGGACGAGAATC (SEQ ID NO: 10) | GAAGAAGTCGTTCGCATTGGC (SEQ ID NO: 32) |
| GATA3 | GGTATCCTCCGACCCACCAC (SEQ ID NO: 11) | CCAGCCAGGGCAGAGATCC (SEQ ID NO: 33) |
| vWF | GGCGAGGATGGAGTTCGACA (SEQ ID NO: 12) | TGACAGGGCTGATGGTCTGG (SEQ ID NO: 34) |
| Bmi1 | AAACCAGACCACTCCTGAACA (SEQ ID NO: 13) | TCTTCTTCTCTTCATCTCATTTTTGA (SEQ ID NO: 35) |
| Cyclin D1 | GCGTACCCTGACACCAATCTCCTC (SEQ ID NO: 14) | ACCTCCTCTTCGCACTTCTGCTCC (SEQ ID NO: 36) |
| 47S | TCCCGACTACTTCACTCCTG (SEQ ID NO: 15) | CAAGAGAACACAACGAGCGAC (SEQ ID NO: 37) |
| 28S | CGCGACCTCAGATCAGACGT (SEQ ID NO: 16) | GCTCTTCCCTGTTCACTCGC (SEQ ID NO: 38) |
| A1 | GCTTGTTTCTCCCGATTGCG (SEQ ID NO: 17) | ACACATCCACAAGGACCACG (SEQ ID NO: 39) |
| A1-CT | GCGCACTTTTCTCAAGTGGT (SEQ ID NO: 18) | TGAAACACGTGAGGGCACAA (SEQ ID NO: 40) |
| a1 | CCCTGGCTGAGCACTACCTT (SEQ ID NO: 19) | CTGCATGCTTGGCTTGGA (SEQ ID NO: 41) |
| BcL2 | TGGGATGCCTTTGTGGAACT (SEQ ID NO: 20) | ACAGCCAGGAGAAATCAAACAG (SEQ ID NO: 42) |
| BcL-x1 | GGCTGGGACACTTTTGTGGAT (SEQ ID NO: 21) | GCGCTCCTGGCCTTTCC (SEQ ID NO: 43) |
| Mcl1 | CCCTCCCCCATCCTAATCAG (SEQ ID NO: 22) | AGTAACAATGGAAAGCATGCCAAT (SEQ ID NO: 44) |
| Bak | AATGGCATCTGGACAAGGAC (SEQ ID NO: 23) | GTTCCTGCTGGTGGAGGTAA (SEQ ID NO: 45) |
| Bax | TGGAGCTGCAGAGGATGATTG (SEQ ID NO: 24) | AGCTGCCACCCGGAAGA (SEQ ID NO: 46) |
| Bid | GAAGACGAGCTGCAGACAGATG (SEQ ID NO: 25) | AATCTGGCTCTATTCTTCCTTGGTT (SEQ ID NO: 47) |
| Bim | TTGGAGCTCTGCGGTCCTT (SEQ ID NO: 26) | CAGCGGAGGTGGTGTGAAT (SEQ ID NO: 48) |
| Noxa | GGAGTGCACCGGACATAACT (SEQ ID NO: 27) | TTGAGCACACTCGTCCTTCA (SEQ ID NO: 49) |
| Puma | GCGGCGGAGACAAGAAGA (SEQ ID NO: 28) | AGTCCCATGAAGAGATTGTACATGAC (SEQ ID NO: 50) |
| β-Actin | GACGGCCAGGTCATCACTATTG (SEQ ID NO: 29) | AGGAAGGCTGGAAAAGAGCC (SEQ ID NO: 51) |

METHODS TABLE 3
Human qRT-PCR Primer Sequences (Table 3 discloses Forward Primers as SEQ ID
NOS 52-59 and Reverse Primers as SEQ ID NOS 60-67, respectively, in order of appearance)

| Gene | Forward Primer (5′ to 3′) | Reverse Primer (5′ to 3′) |
|---|---|---|
| p21 | GTCACTGTCTTGTACCCTTGTG (SEQ ID NO: 52) | CGGCGTTTGGAGTGGTAGAAA (SEQ ID NO: 60) |
| p27 | TGCAACCGACGATTCTTCTACTCAA (SEQ ID NO; 53) | CAAGCAGTGATGTATCTGATAAACAAGGA (SEQ ID NO: 61 |
| p57 | AGAGATCAGCGCCTGAGAAG (SEQ ID NO: 54) | GGGCTCTTTGGGCTCTAAAC (SEQ ID NO: 62) |
| GATA3 | ACCACAACCACACTCTGGAGGA (SEQ ID NO: 55) | TCGGTTTCTGGTCTGGATGCCT (SEQ ID NO: 63) |
| vWF | CGGCTTGCACCATTCAGCTA (SEQ ID NO: 56) | TGCAGAAGTGAGTATCACAGCCATC (SEQ ID NO: 64) |
| Bmi1 | AATCCCCACCTGATGTGTGT (SEQ ID NO: 57) | GCTGGTCTCCAGGTAACGAA (SEQ ID NO: 65) |
| Cyclin | AGCTCCTGTGCTGCGAAGTGGAAAC (SEQ ID NO: 30) | AGTGTTCAATGAAATCGTGCGGGGT (SEQ ID NO: 66) |
| β-Actin | AGCGAGCATCCCCCAAAGTT (SEQ ID NO: 59) | GGGCACGAAGGCTCATCATT (SEQ ID NO: 67) |

### Example 1

Experimental Platform for Proximity Based Study of HSPC Niche.

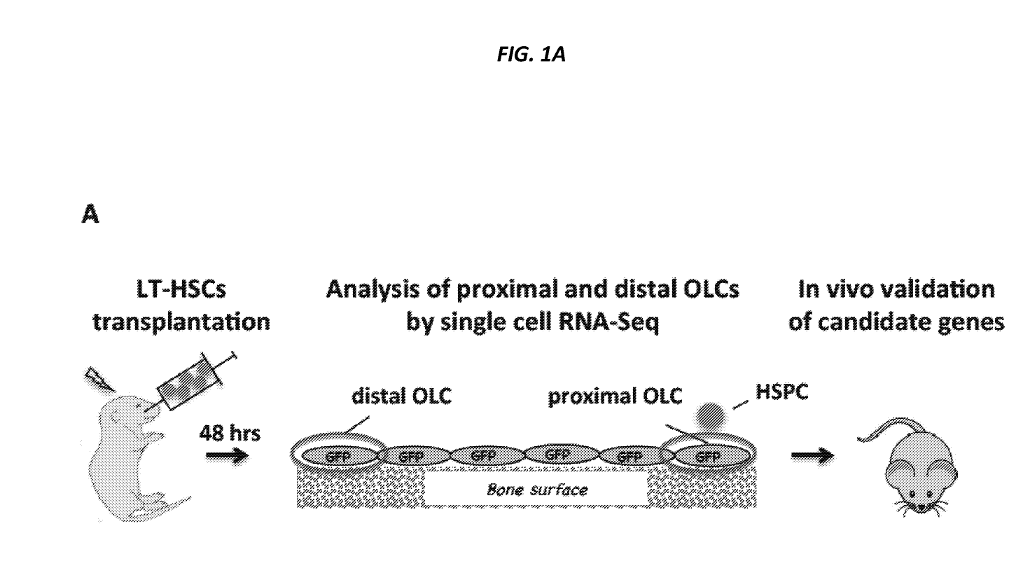

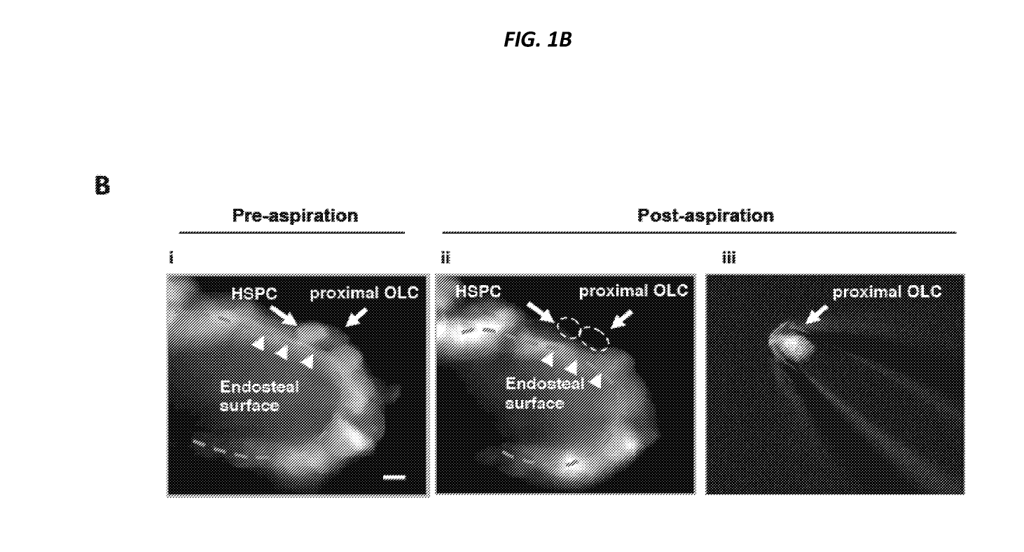

To test our hypothesis, we adapted the experimental platform used in the above-mentioned in vivo imaging experiments (Lo Celso et al., 2009 by intravenously injecting adult bone marrow LT-HSCs (lineage-negative (lin− kit+ Sca1+ [LKS] CD34−Flk2− fluorescently labeled with a lipophilic membrane-bound dye, DiI, into irradiated col2.3GFP mice (Kalajzik et al, 2002) (FIG. 1, top panel. However, experiments were performed in neonatal col2.3GFP recipients, which offered a technical advantage of being able to isolate OLCs without bone decalcification, which would have made the samples unsuitable for the transcriptome analysis. Forty-eight hours after LT-HSC injection, the animals were sacrificed; femoral bones were dissected and immediately sectioned on a vibratome. Upon examination of multiple sections, rare instances were identified where single DiI-positive transplanted HSPCs were seen immediately adjacent to individual OLCs at the endosteal surface. Contrary to other transplanted cells, these cells had not formed clusters forty-eight hours after transplantation; we therefore assumed that they remained quiescent throughout this time and would therefore serve as precise spatial “pointers” towards putative quiescence-regulating OLCs.

In order to retrieve OLCs directly from a section of neonatal trabecular bone, we modified the standard patch clump microscopy platform by introducing additional steps for tissue immobilization and in/situ enzymatic digestion under direct visual control [see Methods]. The tip diameter and micropipette geometry were optimized to enable aspiration of intact OLCs without cell membrane damage (as verified by the presence of cytoplasmic GFP signal to prevent mRNA leakage. Individual proximal and distal OLCs were harvested as shown (FIG. 1, bottom panel, and performed comparative transcriptome analysis by single cell RNA-Seq (Tang et al., 2009).

Proximal OLCs have a Distinct Transcriptional Signature

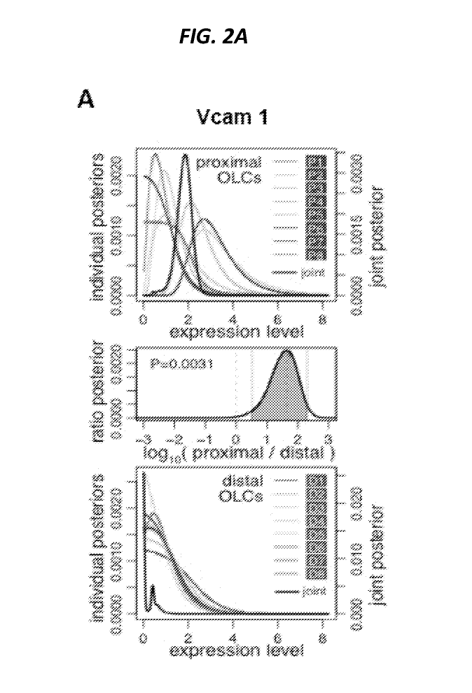

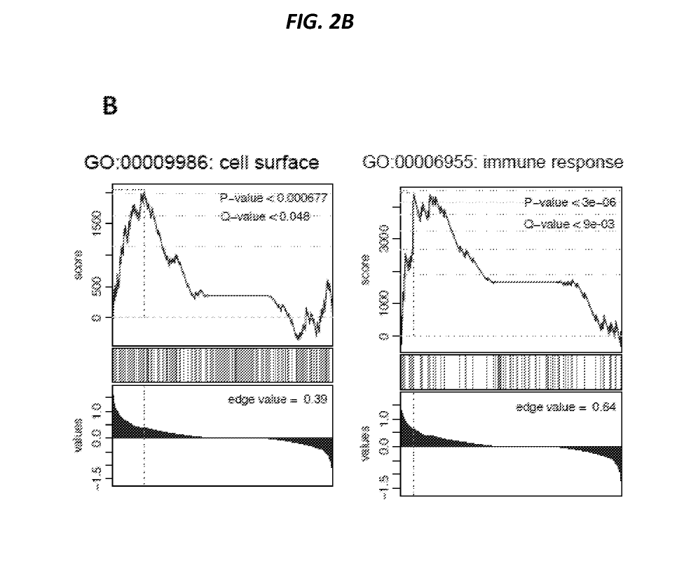

Given the rarity of proximal OLCs in tissue sections, a maximum of two proximal OLCs and distal OLCs controls were harvested per each transplanted animal. In total, sixteen proximal OLCs and sixteen distal OLCs were retrieved. Following cDNA amplification and quality control [see Methods], eight cells from each group were selected for single cell RNA-Seq analysis. In order to accommodate for biological and technical noise commonly observed in single cell RNA-Seq experiments, a probabilistic method was developed, which uses Bayesian approach to estimate the likelihood of expression magnitude based on the observed reads for a gene in question and the overall error characteristics within the transcriptome of a particular single cell sample—Single Cell Differential Expression (SCDE (Kharchenko et al., 2014). By comparing combined probabilistic estimates from single cell transcriptomes across the samples in each group, the method estimated the likelihood that the level of expression of a given gene differed between proximal and distal OLCs (Vcam-1 gene shown as a representative example, FIG. 2A. Using the top 200 differentially expressed genes, we found that profiles of proximal OLCs are clustered separately from the profiles of distal OLCs (FIG. 3A). To test whether proximal and distal OLCs could be distinguished in an unbiased manner based on a genome-wide transcriptional signature, we performed cross-validation tests using the “leave-two-out” strategy. Specifically, transcriptional signatures of one proximal and one distal OLC were “left out” from the 16-cell dataset, a machine-learning classifier was trained on the remaining cells, and the ability of the classifier to correctly assign the transcriptomes of the “left-out” cells to either proximal or distal group was evaluated (Rizzo, 2007). The process was repeated for all proximal-distal cell pairs (64 possible combinations in total). Despite a small sample size, the majority of “left-out” samples were correctly classified (FIG. 3B), area under the curve [AUC]=0.854, p<10-5 indicating that the proximal and distal OLCs displayed stable genome-wide transcriptional differences. In particular, gene set enrichment analysis showed that proximal OLCs displayed a significant up-regulation of genes encoding cell surface proteins (p-value 6.8×10⁻⁴, Q-value 0.048; top genes: Vcam1, Adam9, Amot and those involved in immune response (p-value 3.1×10-6, Q-value 0.0090; top genes: Map3k14, Cxcl12, Il18, supporting their role in intercellular communications (FIG. 2B). At the level of individual genes, we found that with the exception of c-kit, proximal OLCs had significantly higher expression levels of niche-associated molecules (most notably Cxcl12 and Vcam-1 as compared to distal OLCs. Further, in accordance with prior studies of a regulatory OLC phenotype, proximal OLCs were lineage-committed (Runx2⁺, Sp7/osterix⁺, col1a1⁺ but less mature (Spp1/osteopontin^{low}, Bglap/osteocalcin^{low}, Dmp1low than distal OLCs (FIG. 3C,D).

Taken together, these data demonstrate that a proximity-based approach enabled identification of the OLC fraction which is transcriptionally distinct from the remaining OLCs and whose signature is consistent with HSPC regulatory function. Our ability to detect consistent transcriptional features of proximal OLCs despite a limited sample number and inter-sample variability indicates that cellular proximity acts as a powerful and reliable discriminator between molecularly distinct subset within an apparently homogeneous, lineage-restricted cell population.

Based on these findings, we set out to test whether the proximal OLC signature could be used as a resource for identification of novel non cell-autonomous HSPC regulators in vivo. Among membrane-bound and secreted factors that were preferentially expressed in proximal OLCs, we chose three molecules from distinct functional groups for further validation. These included secreted RNase angiogenin (ANG), pro-inflammatory cytokine interleukin 18 (IL18, and cell adhesion molecule Embigin. ANG derived from committed osteoprogenitors, mesenchymal progenitors and peri arteriolar sheath cells, but not mature osteoblasts, regulates LT HSC quiescence. ANG is a secreted ribonuclease with established roles in promoting tumor angiogenesis and cellular proliferation (Kishimoto et al., 2005). It also acts as a neuronal pro-survival factor in the context of amyotrophic lateral sclerosis (ALS (Greenway et al., 2006).

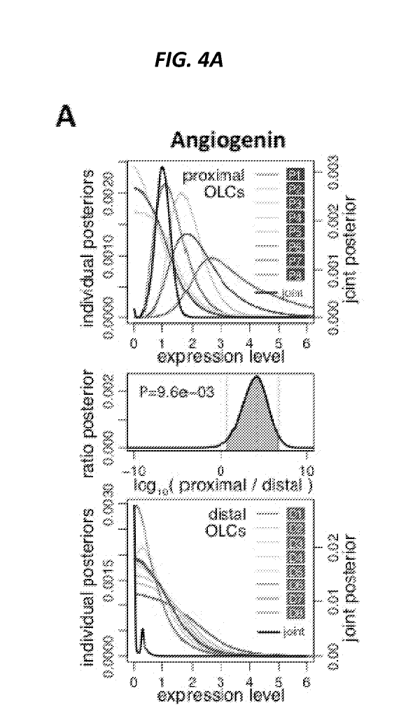

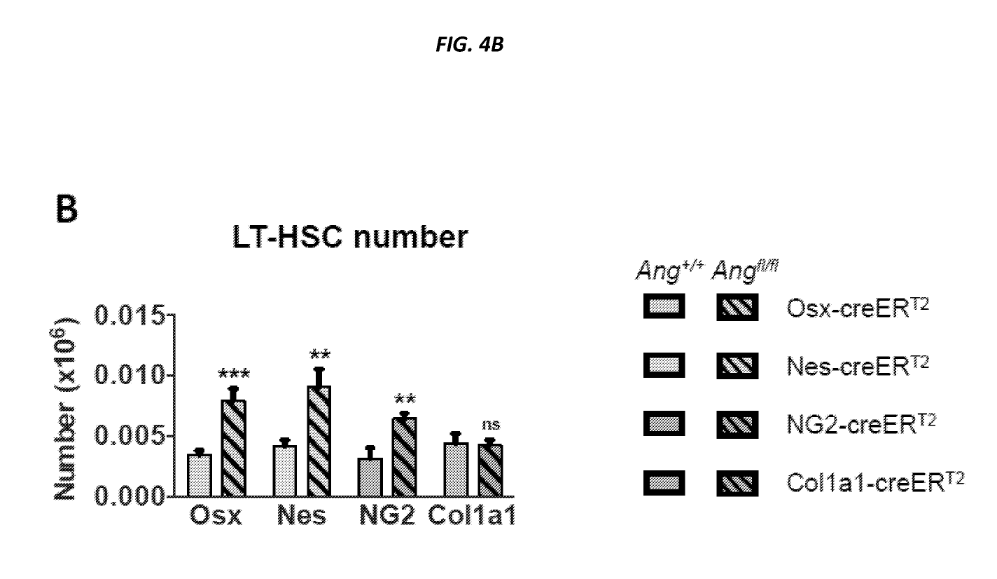

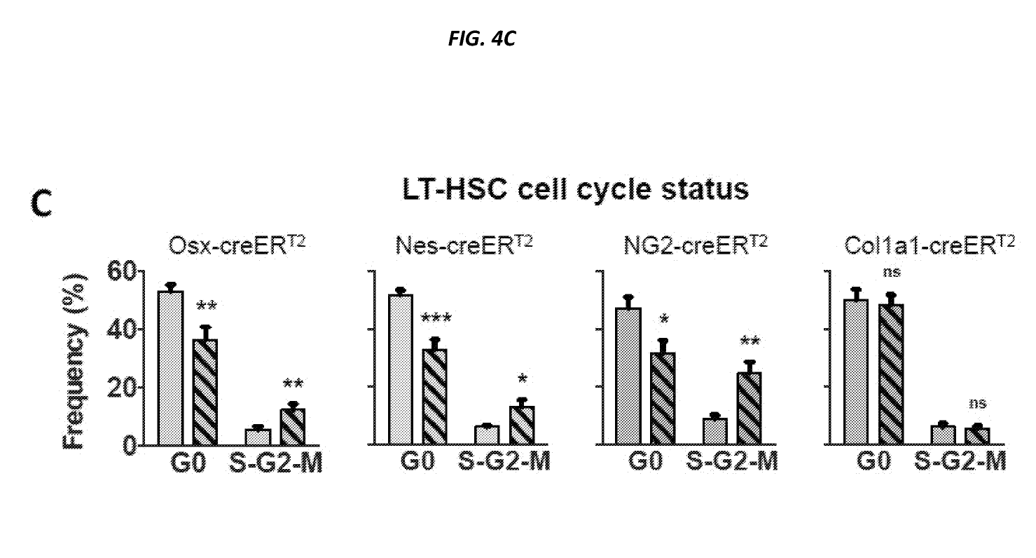

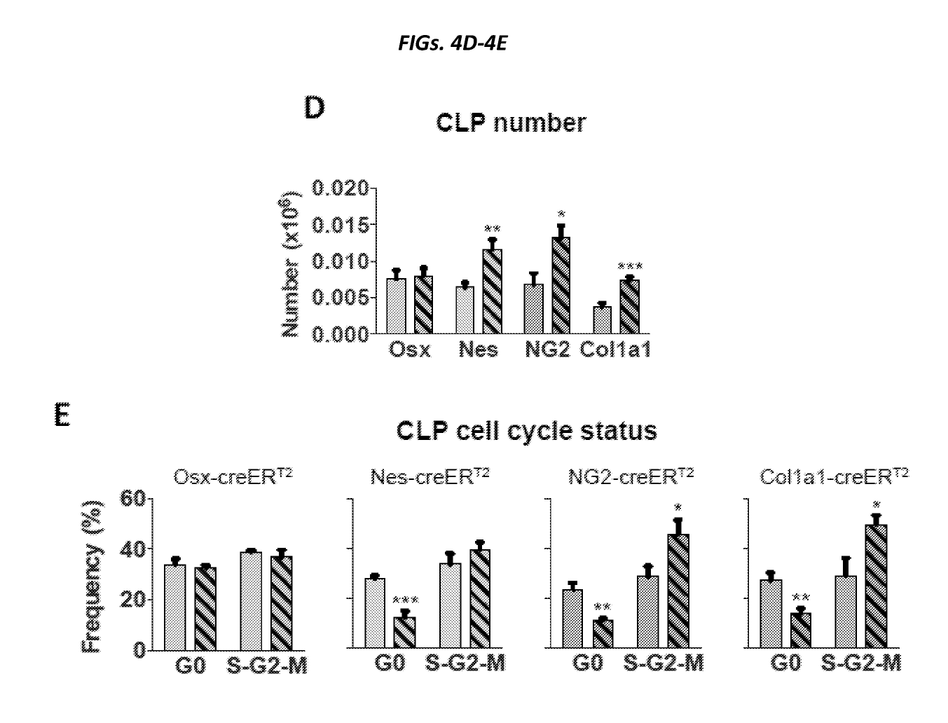

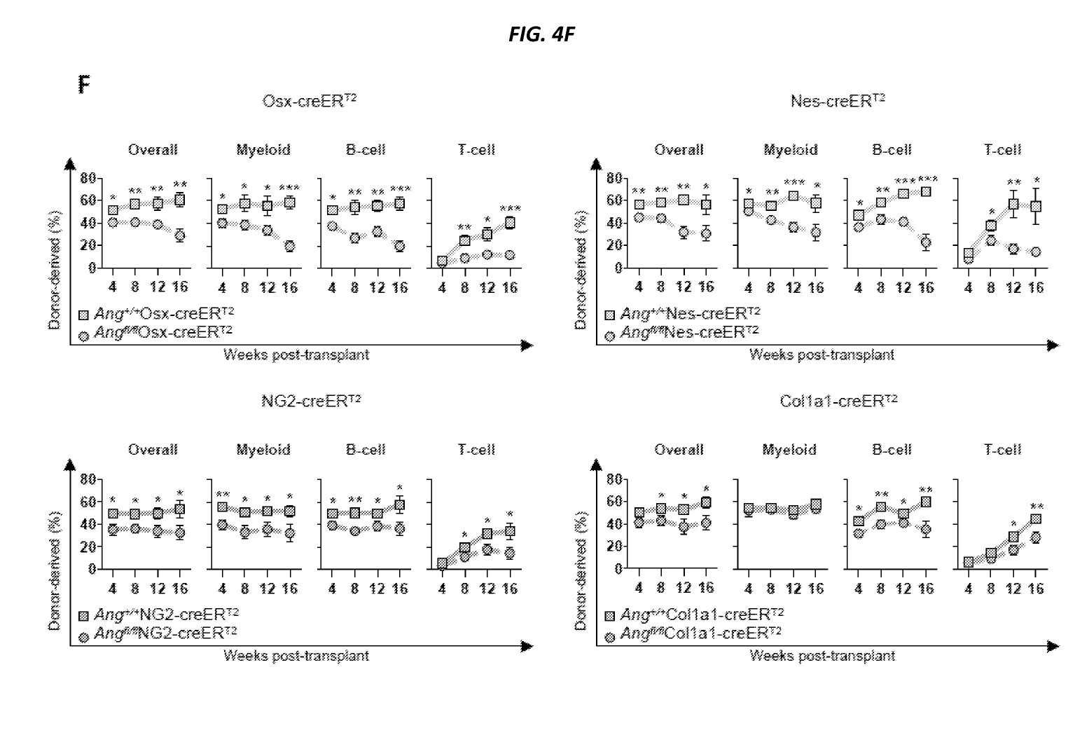

We found that Ang was expressed at a higher level in proximal OLCs (FIG. 4A) and undertook a functional evaluation of its role in the bone marrow niche using AngKO mice (as described in the accompanying manuscript by Goncalves et al or mice in which Ang/was conditionally deleted from distinct niche cell subsets. We crossed Ang “floxed” mice with animals in which tamoxifen-inducible Cre-recombinase was driven by the promoters targeting specific mesenchymal cells—committed osteoprogenitors (Osx) (Mizoguchi et al., 2014), mesenchymal progenitors (nestin) (Mendez-Ferrer et al., 2010), periarteriolar sheath cells (NG2) (Zhu et al., 2011) and mature osteoblasts (Col1a1 (Kim et al., 2004). Ang transcripts were detectable in Osx+ cells by Q-PCR (data not shown); Ang expression in other niche cell subsets mentioned above has been previously documented (Kunisaki et al., 2013) (Paic et al., 2009).

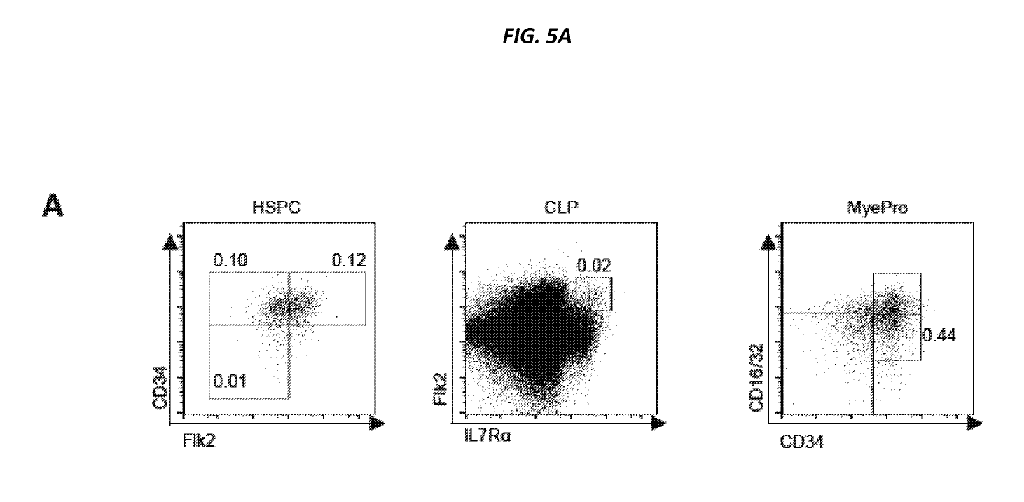

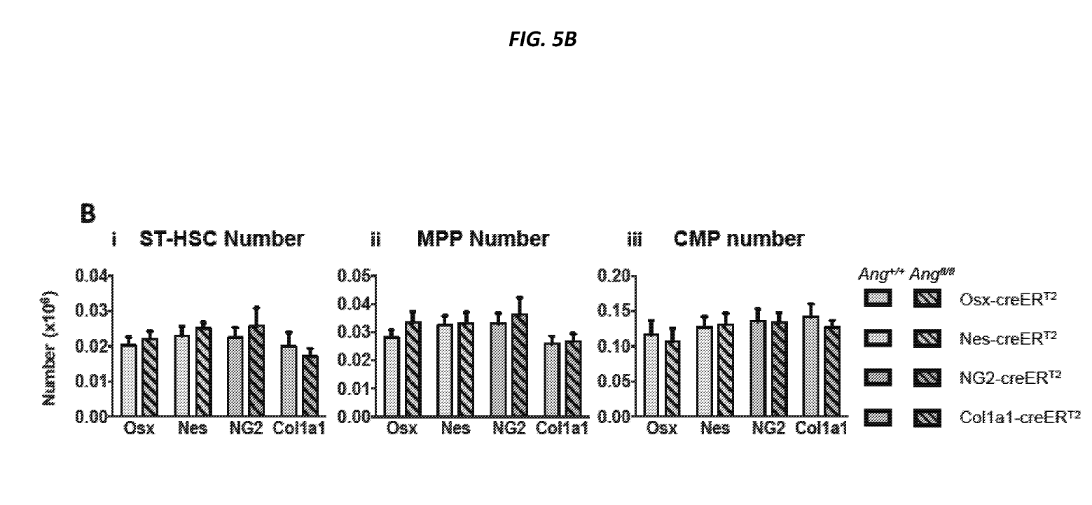

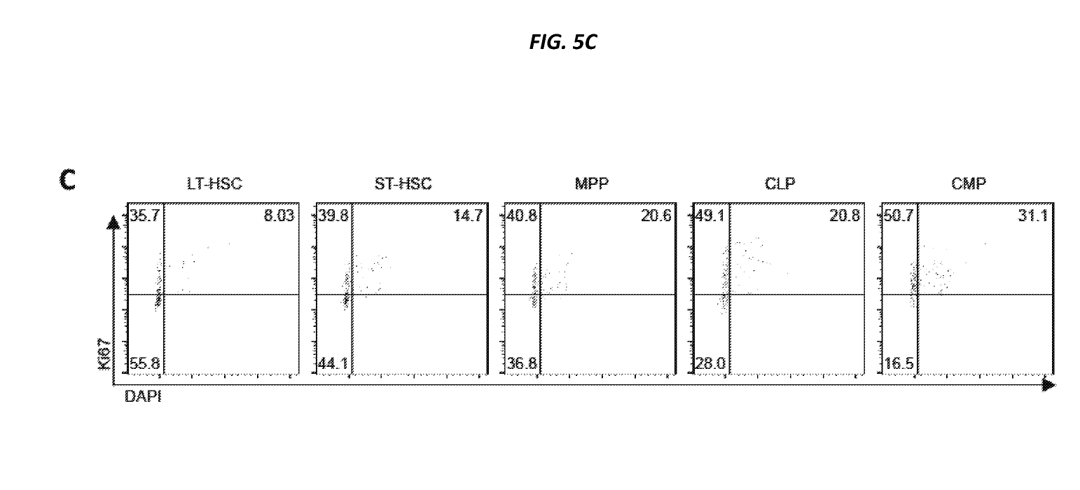

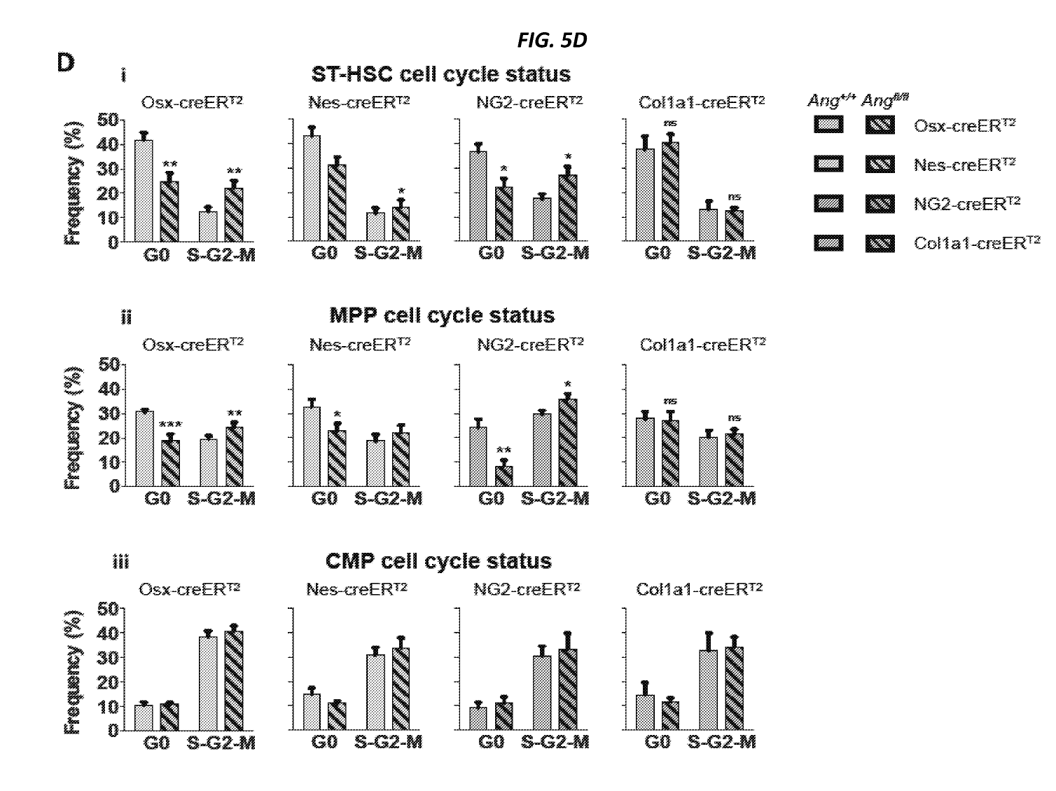

All conditional knock-outs demonstrated no significant changes in peripheral blood or bone marrow changes, apart from mild lymphocytosis (Table 1). However, immunophenotypic analysis of primitive hematopoietic cells (FIG. 5A) revealed that deletion of Ang from Osx+, Nes+ and NG2+ cells resulted in an increase of the number of LT-HSC and more active cycling of LT-HSC, short-term HSC (ST-HSC) and multi-potent progenitors (MPP) (FIG. 4B, 5C and FIG. 5Bi,ii, 5C, 5Di,ii). In contrast, Ang deletion with col1a1Cre had no effect on these cell populations, but was associated with an increase in number and more active cycling of common lymphoid progenitors (CLP), as was also seen upon Ang/deletion from Nes+ and NG2+ cells (FIG. 4D,E). The number and cell cycle status of the myeloid progenitors in any of the above strains were unaffected by the Ang deletion (FIG. 5B, 5D).

TABLE 4
Baseline bone marrow and peripheral blood profiles of conditional Ang-deleted mouse strains.

| | | | Osx-creER^{T2} | | Nestin-creER^{T2} | |
|---|---|---|---|---|---|---|
| Organ | Parameter | Unit | Ang^{+/+} | Ang^{fl/fl} | Ang^{+/+} | Ang^{fl/fl} |
| Blood | WBC | 10³/μl | 11.2 ± 1.13 | 13.3 ± 0.70 | 11.4 ± 0.88 | 13.6 ± 0.65* |
| | LYM | 10³/μl | 9.81 ± 1.05 | 11.4 ± 0.73 | 8.90 ± 0.8 | 10.9 ± 0.38* |
| | MON | 10³/μl | 0.20 ± 0.02 | 0.20 ± 0.07 | 0.18 ± 0.02 | 0.15 ± 0.03 |
| | NEU | 10³/μl | 1.22 ± 0.08 | 1.67 ± 0.31 | 2.32 ± 0.29 | 2.56 ± 0.36 |
| | RBC | 10⁶/μl | 9.92 ± 0.97 | 9.76 ± 0.85 | 9.67 ± 0.54 | 10.6 ± 1.20 |
| | HGB | g/dl | 13.6 ± 1.15 | 13.1 ± 1.17 | 11.6 ± 1.00 | 12.1 ± 0.76 |
| | HCT | % | 44.0 ± 1.92 | 45.1 ± 2.43 | 38.7 ± 1.04 | 36.7 ± 4.28 |
| | MCV | fL | 44.2 ± 0.40 | 43.6 ± 0.71 | 42.6 ± 1.02 | 42.7 ± 1.69 |
| | MCH | pg | 14.3 ± 0.17 | 14.1 ± 0.52 | 15.3 ± 0.38 | 14.9 ± 0.41 |
| | MCHC | g/dl | 33.2 ± 0.72 | 32.1 ± 0.56 | 33.4 ± 0.60 | 32.7 ± 0.90 |
| | RDWc | % | 20.9 ± 1.85 | 20.4 ± 0.37 | 19.0 ± 0.34 | 19.5 ± 0.44 |
| | PLT | 10³/μl | 663 ± 60 | 606 ± 76 | 721 ± 65.4 | 647 ± 76.6 |
| | Mac1⁺Gr1⁺ | 10³/μl | 1.34 ± 0.20 | 1.15 ± 0.12 | 1.33 ± 0.19 | 1.63 ± 0.10 |
| | B220⁺ | 10³/μl | 6.69 ± 0.75 | 8.83 ± 0.56* | 6.36 ± 0.44 | 8.43 ± 0.50** |
| | CD3e⁺ | 10³/μl | 2.19 ± 0.32 | 2.42 ± 0.27 | 2.20 ± 0.26 | 2.90 ± 0.16* |
| Bone | Cellularity | 10⁶/femur | 25.8 ± 1.40 | 26.8 ± 1.12 | 25.5 ± 1.09 | 26.0 ± 1.30 |
| Marrow | Mac1⁺Gr1⁺ | 10⁶/femur | 13.6 ± 0.86 | 14.0 ± 0.82 | 12.1 ± 0.70 | 11.2 ± 0.99 |
| | Ter119⁺ | 10⁶/femur | 2.92 ± 0.28 | 2.70 ± 0.32 | 3.03 ± 0.24 | 3.54 ± 0.52 |
| | B220⁺ | 10⁶/femur | 5.30 ± 0.47 | 5.84 ± 0.76 | 5.67 ± 0.77 | 6.65 ± 0.62 |
| | CD3e⁺ | 10⁶/femur | 0.55 ± 0.07 | 0.66 ± 0.10 | 0.49 ± 0.07 | 0.52 ± 0.06 |

| | | NG2-creER^{T2} | | Col1a1-creER^{T2} | |
|---|---|---|---|---|---|
| Organ | Parameter | Ang^{+/+} | Ang^{fl/fl} | Ang^{+/+} | Ang^{fl/fl} |
| Blood | WBC | 9.99 ± 1.27 | 13.8 ± 1.10* | 11.0 ± 0.96 | 13.8 ± 0.70* |
| | LYM | 9.11 ± 1.16 | 12.7 ± 1.07* | 9.22 ± 1.32 | 12.1 ± 0.62* |
| | MON | 0.34 ± 0.09 | 0.42 ± 0.07 | 0.23 ± 0.01 | 0.30 ± 0.07 |
| | NEU | 0.54 ± 0.14 | 0.63 ± 0.08 | 1.58 ± 0.50 | 1.34 ± 0.15 |
| | RBC | 9.51 ± 1.34 | 9.21 ± 1.37 | 8.82 ± 0.74 | 9.37 ± 0.22 |
| | HGB | 12.5 ± 0.44 | 11.6 ± 0.55 | 13.2 ± 0.55 | 13.0 ± 0.22 |
| | HCT | 42.3 ± 0.73 | 41.6 ± 2.28 | 41.8 ± 0.15 | 42.3 ± 0.78 |
| | MCV | 43.5 ± 1.23 | 44.3 ± 0.56 | 43.5 ± 0.87 | 42.6 ± 0.96 |
| | MCH | 13.4 ± 0.51 | 12.3 ± 0.74 | 13.8 ± 0.37 | 13.5 ± 0.50 |
| | MCHC | 32.1 ± 0.51 | 29.9 ± 2.68 | 33.0 ± 0.90 | 32.6 ± 0.73 |
| | RDWc | 19.5 ± 0.80 | 21.3 ± 0.89 | 19.8 ± 0.48 | 21.0 ± 0.70 |
| | PLT | 579 ± 100 | 617 ± 81.1 | 694 ± 154 | 774 ± 72.5 |
| | Mac1⁺Gr1⁺ | 1.05 ± 0.21 | 1.61 ± 0.33 | 1.14 ± 0.03 | 1.47 ± 0.28 |
| | B220⁺ | 4.34 ± 0.86 | 6.65 ± 0.58* | 6.16 ± 0.83 | 8.63 ± 0.37** |
| | CD3e⁺ | 1.12 ± 0.25 | 2.04 ± 0.46 | 1.55 ± 0.19 | 2.33 ± 0.34 |
| Bone | Cellularity | 25.2 ± 1.21 | 26.9 ± 1.20 | 25.6 ± 0.92 | 25.6 ± 1.52 |
| Marrow | Mac1⁺Gr1⁺ | 11.4 ± 0.56 | 11.7 ± 1.08 | 12.3 ± 0.65 | 12.0 ± 1.21 |
| | Ter119⁺ | 3.28 ± 0.95 | 3.20 ± 1.21 | 1.39 ± 0.39 | 1.55 ± 0.31 |
| | B220⁺ | 4.55 ± 0.56 | 6.77 ± 0.71* | 4.87 ± 0.23 | 6.66 ± 0.52* |
| | CD3e⁺ | 0.87 ± 0.30 | 0.88 ± 0.36 | 0.63 ± 0.06 | 0.75 ± 0.06 |

Data represent mean ± SEM.
Statistical significance was assessed by two-tailed Student's t-test.
*p < 0.05,
**p < 0.01
Osx-creERT2 n = 8-9
Nes-creERT2 n = 9-10
NG2-creERT2 n = 6
Cola1a-creERT2 n = 4-8

To assess the effect of the above-noted changes on long-term hematopoietic reconstitution, we competitively transplanted the bone marrow from Angfl/^{fl}OsxCre, Ang^{fl}/^{fl}NesCre, Ang^{fl}/^{fl}NG2Cre, and Ang^{fl}/^{fl}Col1a1Cre mice and corresponding controls into congenic WT recipients (FIG. 4F). We observed significantly reduced long-term multi-lineage reconstitution in the recipients of the bone marrow from Ang^{fl}/^{fl}OsxCre, Ang^{fl}/^{fl}NesCre, Ang^{fl}/^{fl}NG2Cre mice while the animals which were transplanted with Ang^{fl}/^{fl}Col1a1Cre bone marrow displayed only a lymphoid reconstitution defect.

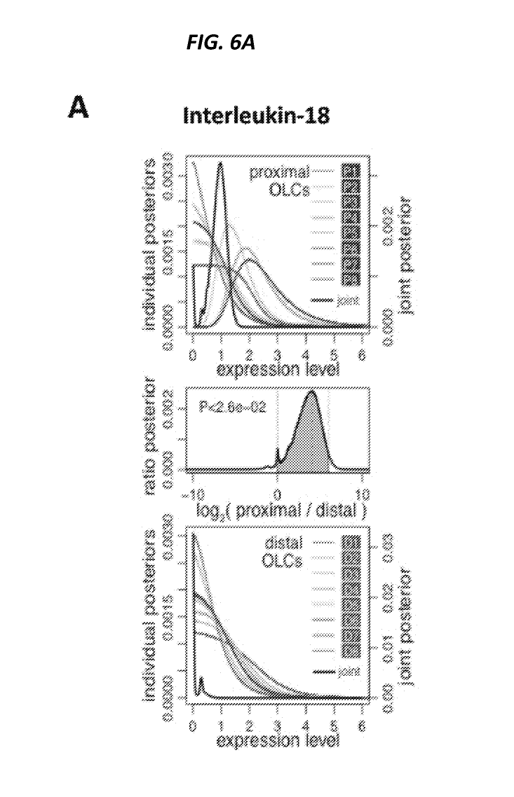

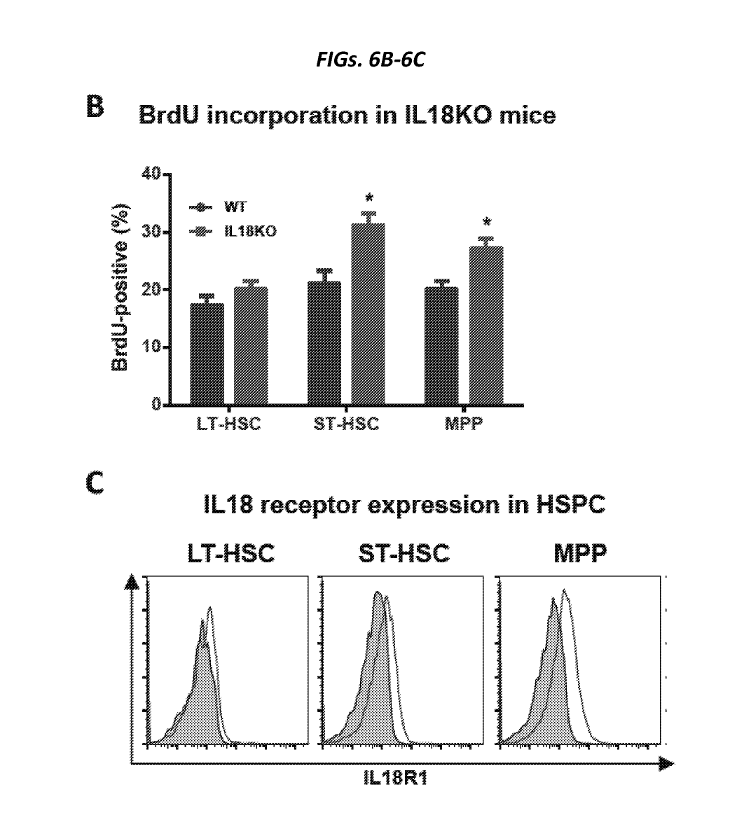

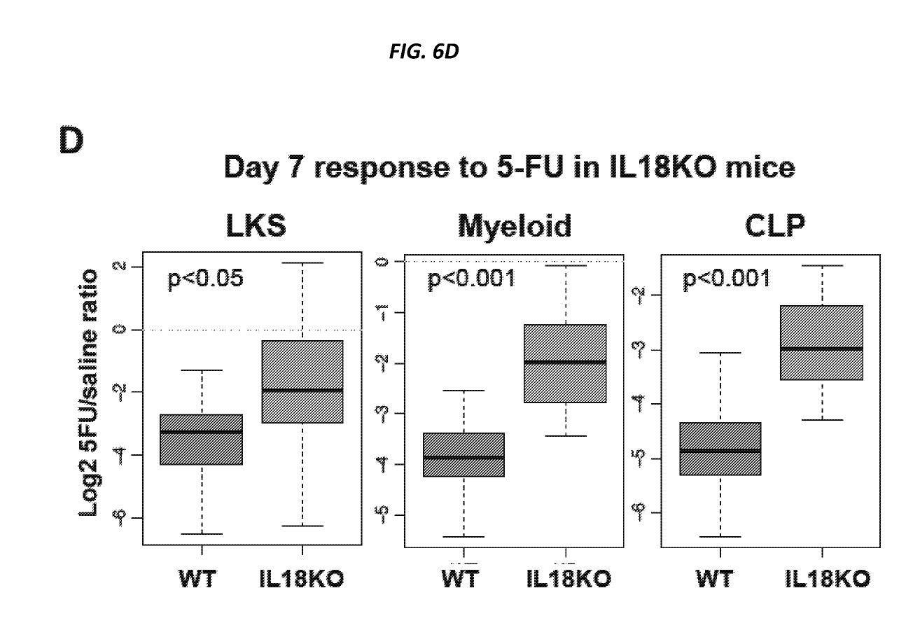

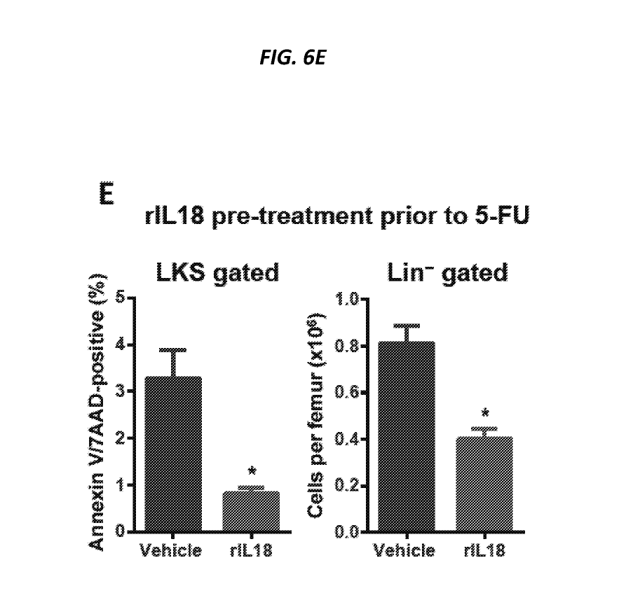

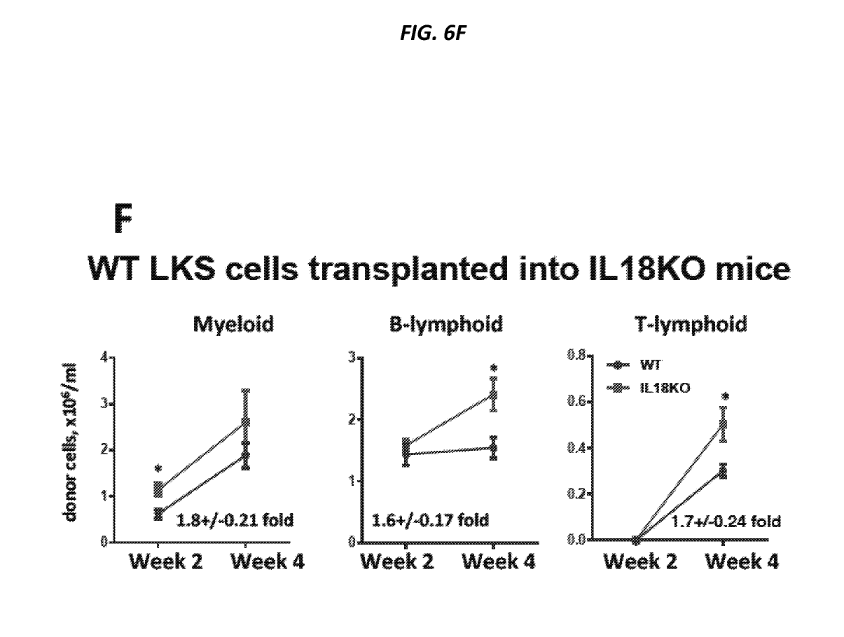

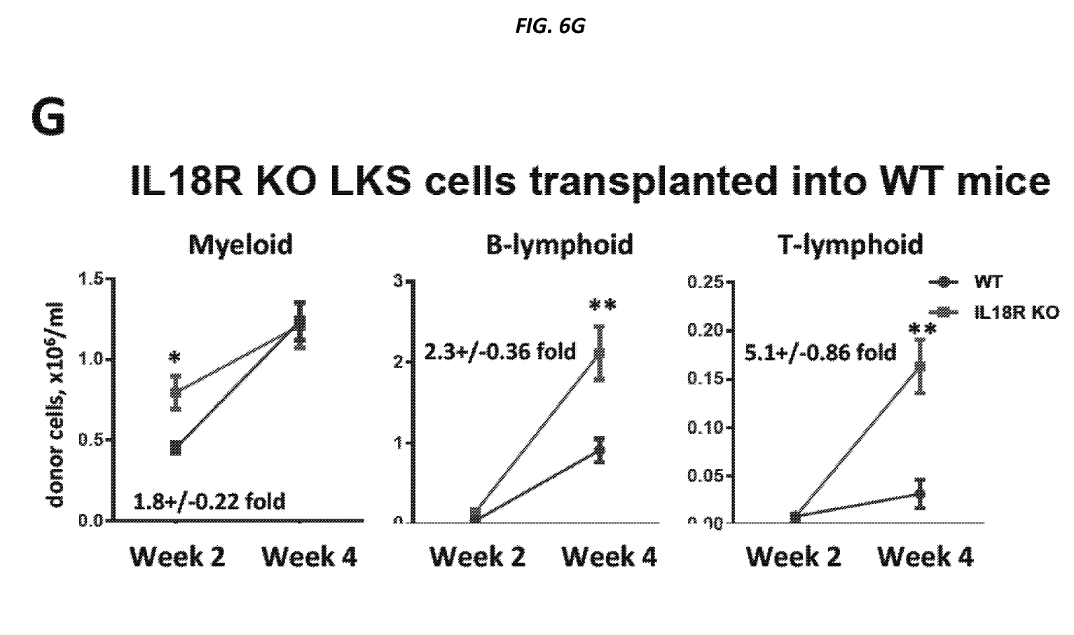

Taken together, our observations reveal the role of ANG as a niche-derived quiescence regulator of LT-HSC, ST-HSC, MPP and CLP and highlight differences in the target cell populations depending on a cellular source: ANG produced by mesenchymal progenitors, committed osteoprogenitors and peri-arteriolar sheath cells regulates quiescence and repopulating ability of LT-HSC, while ANG derived from mature osteoblasts regulates lymphoid progenitors. IL-18 regulates quiescence of short-term, hematopoietic, progenitors; IL 18 is a pro-inflammatory cytokine, which acts as a regulator of T-cell function through induction of interferon-gamma production (Okamura et al., 1995). It also serves as a regulator of stress response by the immune system. IL18 is expressed in multiple cell types within and outside the bone marrow (Novick et al., 2013; Sugama and Conti, 2008). Proximity-based analysis revealed IL18 expression in proximal OLCs, while none of the distal OLCs had detectable IL18 transcripts (FIG. 6A).

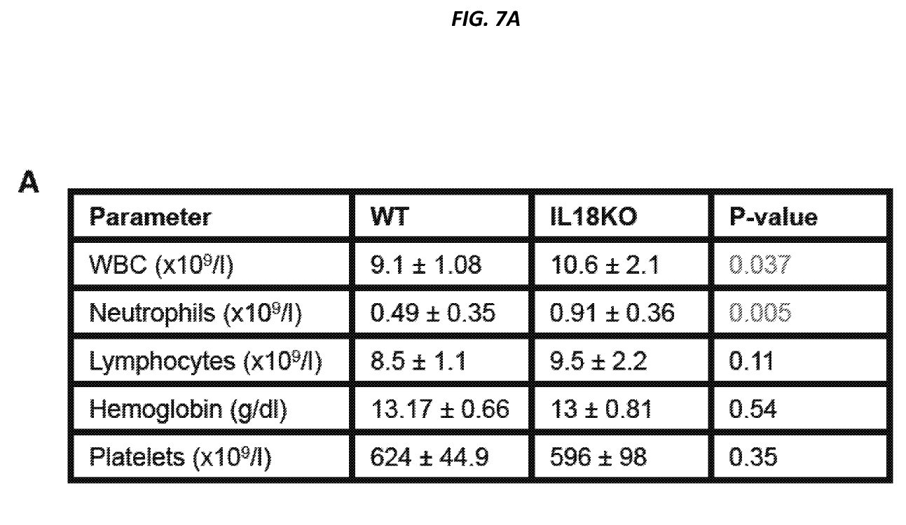

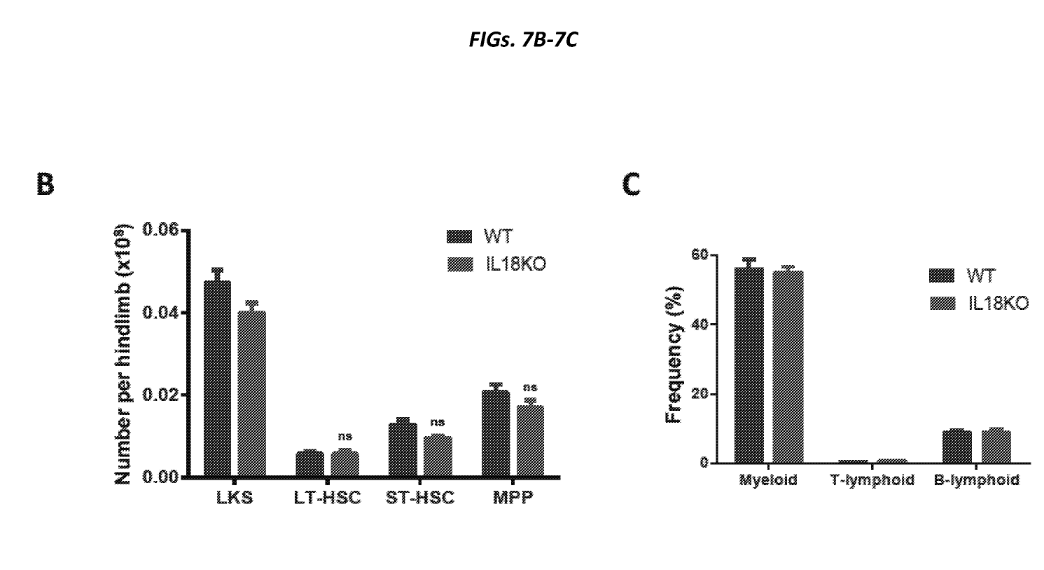

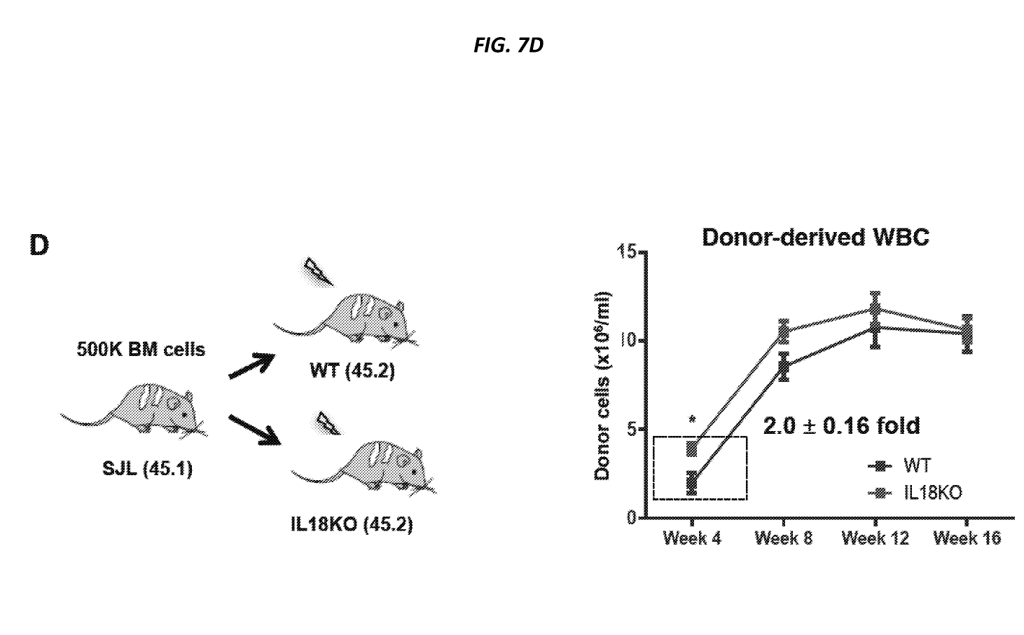

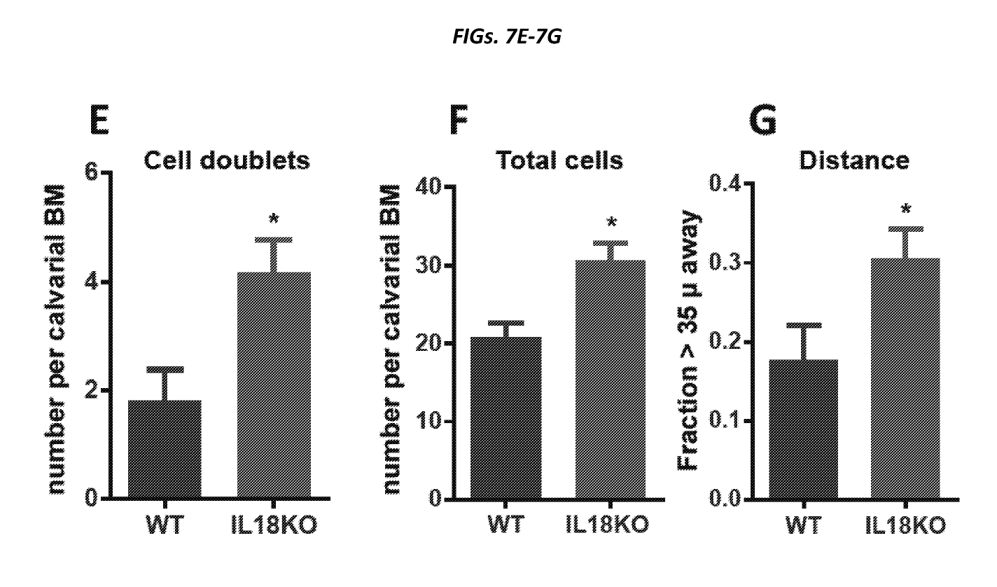

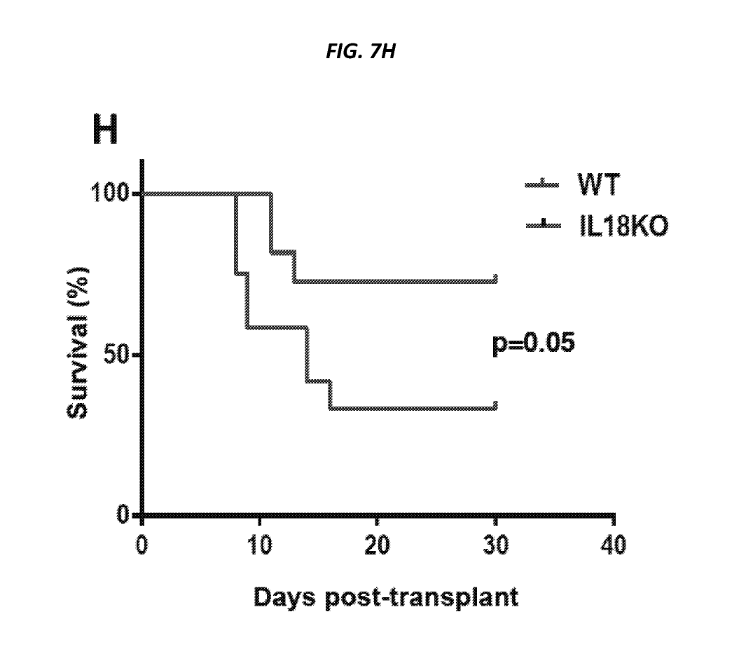

We used IL18 knock-out (IL18KO mice) to investigate a functional role of IL18 in hematopoiesis. These animals displayed no apparent abnormalities in the bone marrow and peripheral blood, apart from modest neutrophilia (FIG. 7A-7C). However, BrdU incorporation studies showed an increased uptake in short-term hematopoietic progenitors—ST-HSC and MPP—but not in LT-HSC (FIG. 6B). These changes mirrored the pattern of the IL18 receptor (IL18R1) expression, which was undetectable in LT-HSCs but present in short-term progenitors (FIG. 6C). These observations indicated that IL18 regulates quiescence of short-term progenitors.

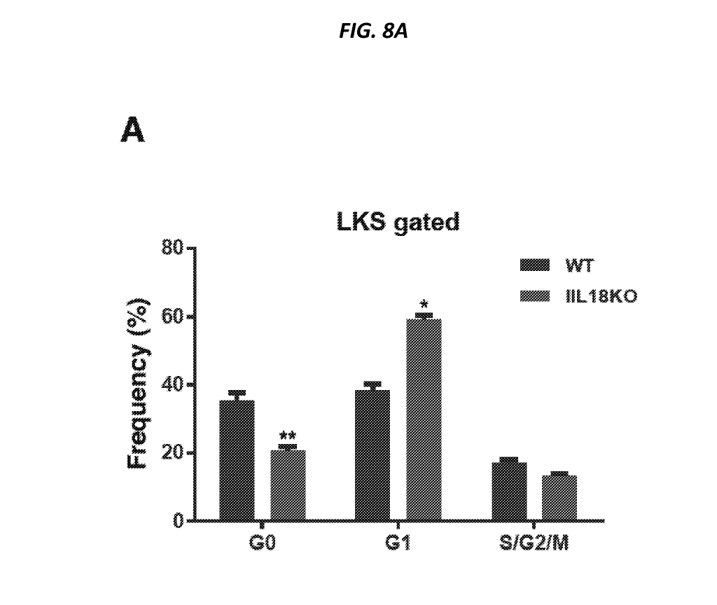

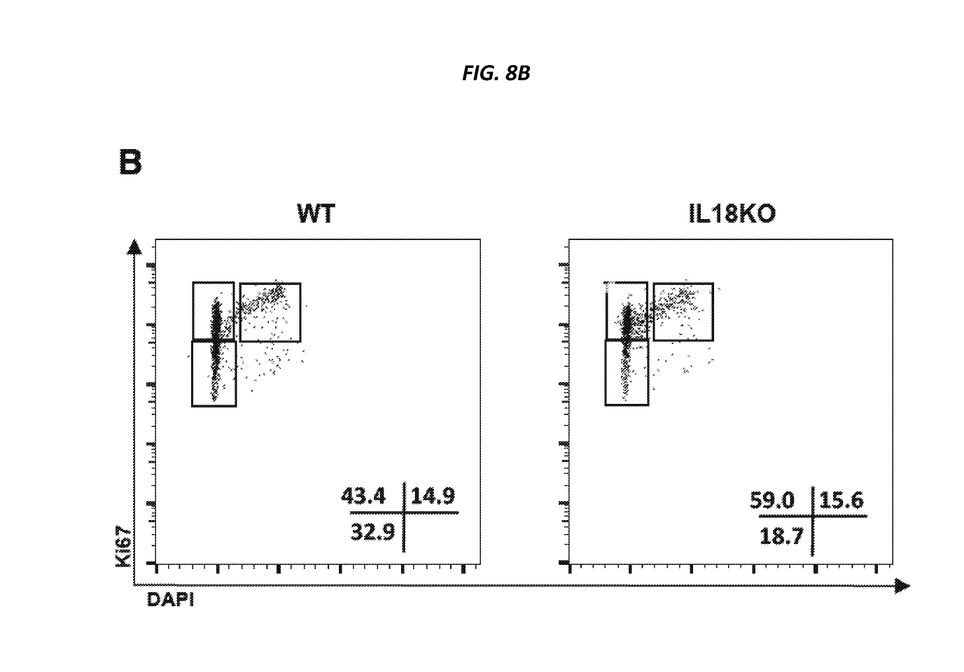

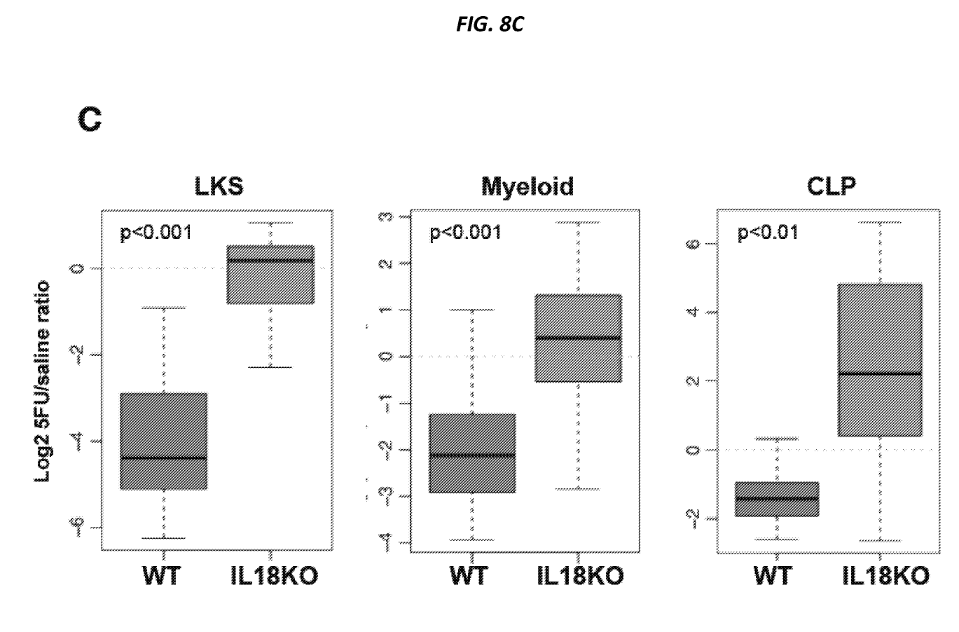

Functionally, these cells are critical for replenishing blood cells following bone marrow injury. Quantification of progenitor cell subsets on 7 days post-exposure to 5-FU (Broxmeyer et al., 2012 showed a significantly increased frequency of LKS cells, lin−kit+Sca1− myeloid progenitors and CLPs in IL18KO mice, as compared to 5-FU-treated WT controls (FIG. 6D). In newborn IL18KO animals, loss of HSPC quiescence at baseline and exaggerated response to genotoxic injury (busulphan exposure in utero (Bruscia et al., 2006) were also observed (FIG. 8A-C). Taken together, these data demonstrate that IL18 normally constrains progenitor proliferation. Consistent with this, exogenous administration of recombinant IL18 protected LKS cells from 5-FU-induced apoptosis, but also resulted in decreased frequency of lineage-negative cells in rIL18-treated animals (FIG. 6E), indicating the IL18 can suppress progenitor response to injury and restrain hematopoietic recovery.

To test if the quiescence-inducing effect of IL18 on short-term progenitors is exerted in a non-cell-autonomous fashion, WT (CD45.1) bone marrow cells were transplanted into lethally irradiated IL18KO or WT recipients (CD45.2). We found that IL18-deficient microenvironment in the recipient animals conferred a significantly faster short-term hematopoietic recovery without affecting long-term reconstitution (FIG. 7D). In keeping with this, transplantation of progenitor-enriched WT bone marrow fraction (LKS cells into IL18KO hosts was accompanied by approximately 2-fold increase in both myeloid (week 2) and lymphoid (week 4) cells in peripheral blood of the recipient animals, which was no longer detectable at week 16 (FIG. 6F). The finding of enhanced early post-transplant reconstitution in the absence of IL18 signaling was recapitulated in a reciprocal experiment, when sorted LKS cells from IL18 receptor knock-out animals were transplanted into WT hosts (FIG. 6G), indicating that the effect of IL18 on short-term progenitors is likely to be direct. Interestingly, faster proliferation of transplanted LKS cells in IL18KO recipients was already evident at 24 hours, as shown by intra-vital imaging studies, and was associated with homing further away from the endosteal surface indicating that IL18 also regulates progenitor localization in the niche (FIG. 7E-7G).

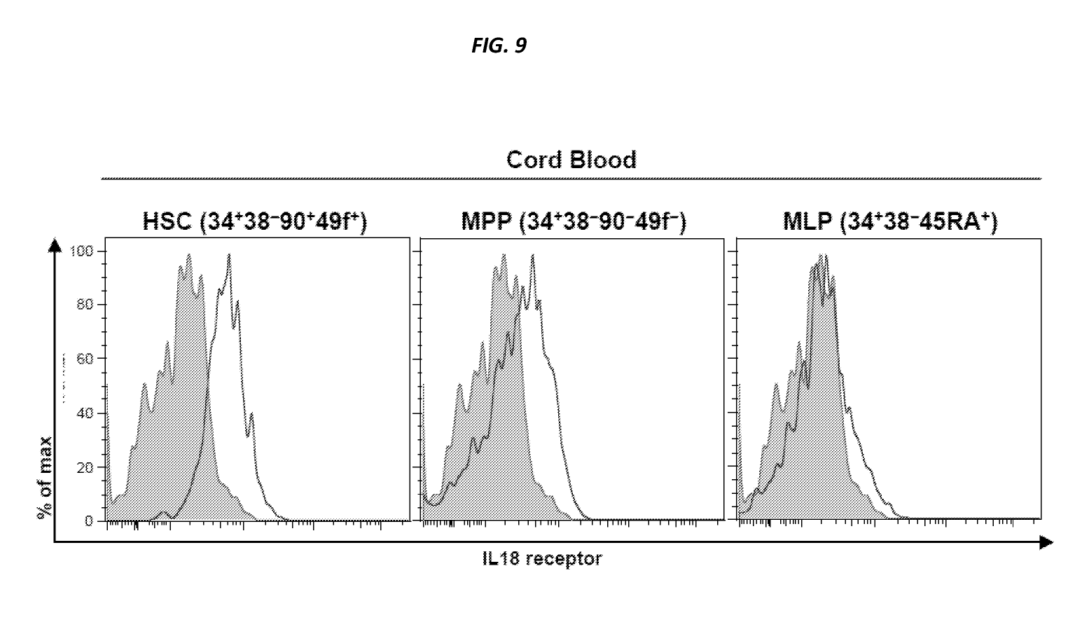

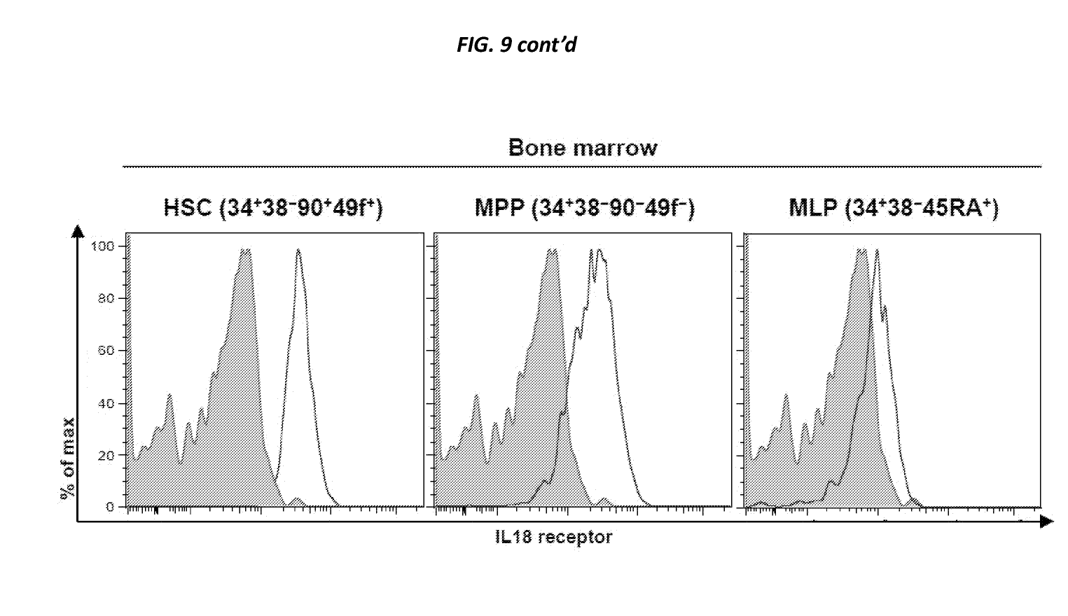

To test if the effect of IL18 on post-transplant progenitor expansion can be explored therapeutically, we transplanted lethally irradiated IL18KO and WT recipients with a limiting dose of WT bone marrow and found improved survival in the IL18KO group (FIG. 7H). This raises a possibility that IL18 neutralization might be a means of reducing post-transplant cytopenias—a major cause of morbidity and mortality in patients. Given that in humans, the highest level of IL18R expression is observed in the most primitive HSPC (FIG. 9), IL18 blockade may have an additional effect on post-transplant long-term HSC expansion.

Embigin Regulates Localization and Quiescence of Long-Term HSC and Short-Term Progenitors

Embigin is a cell adhesion molecule of immunoglobulin superfamily (Huang et al., 1990, 1993). Embigin is thought to enhance integrin-dependent cell substrate adhesion and was also shown to promote neuromuscular synapse formation (Lain et al., 2009). Embigin is widely expressed within the hematopoietic system, including primitive hematopoietic cells (Pridans et al., 2008), but its function remains obscure.

### Example 2

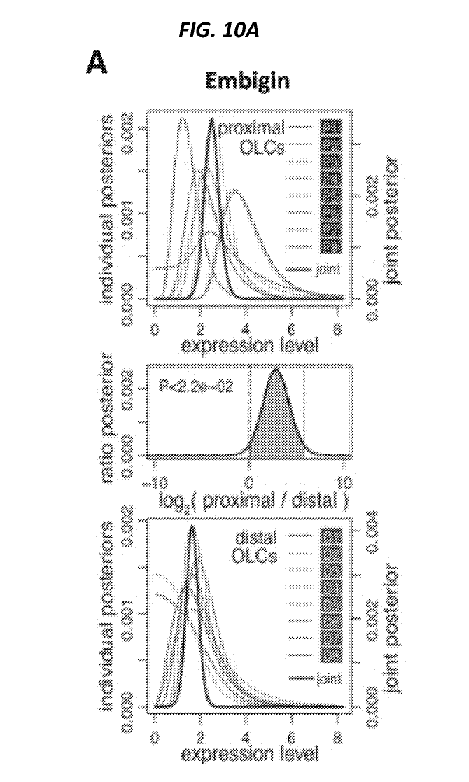

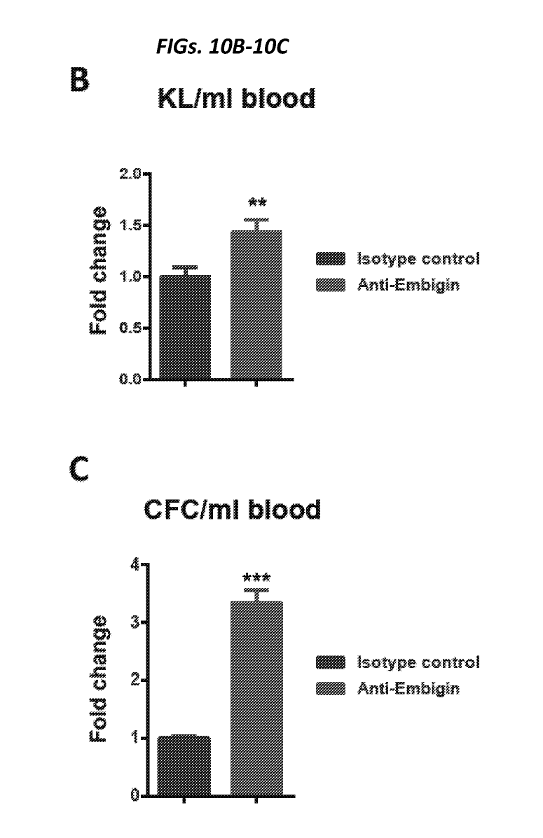

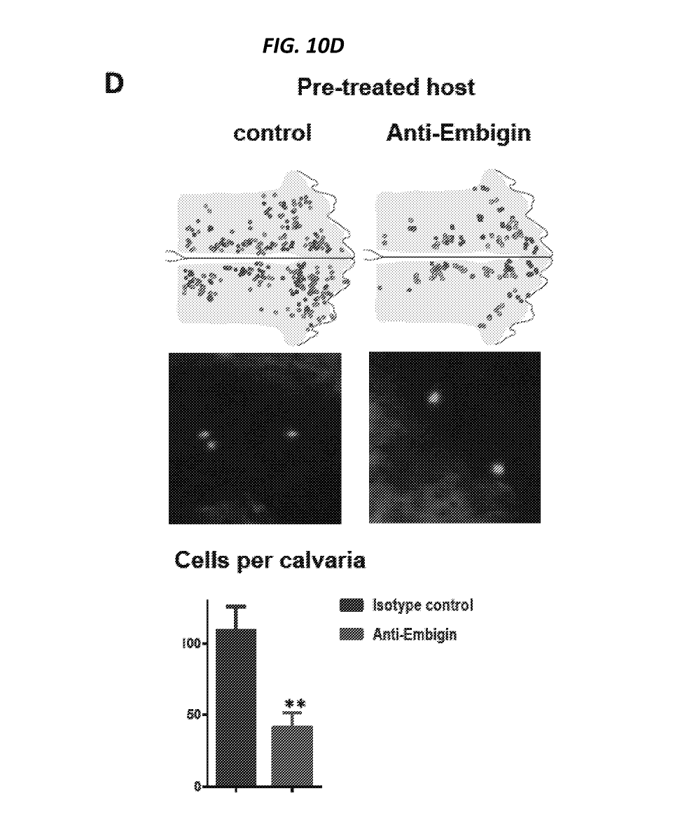

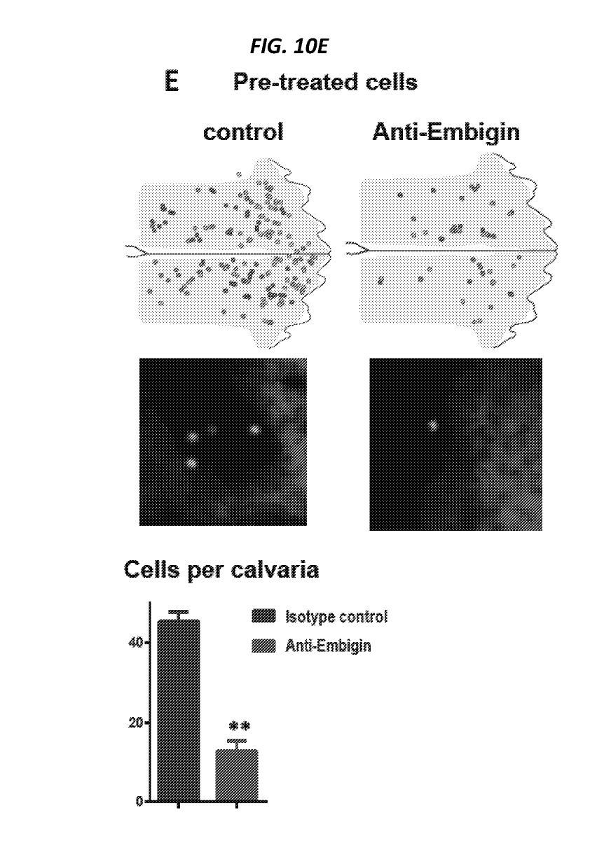

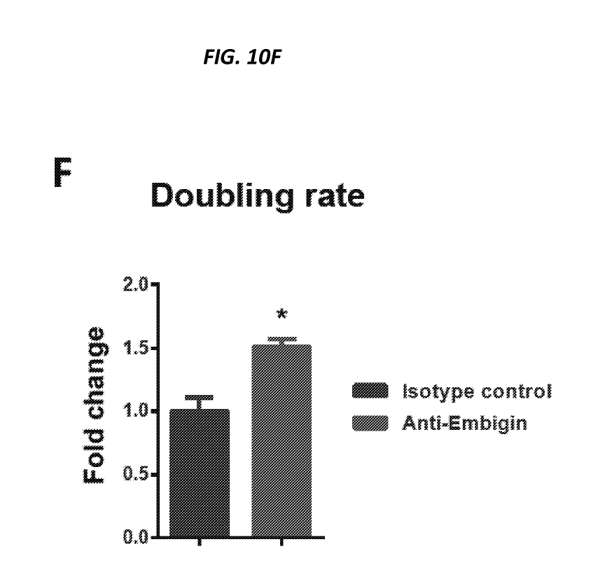

Our proximity-based analysis showed that proximal OLCs had a significantly higher level of Embigin expression compared to distal OLCs (FIG. 10A), and we undertook in vivo functional studies to evaluate its role as a hematopoietic regulator. In the absence of an established genetic model, we used a neutralizing antibody against Embigin for these experiments (Pridans et al., 2008).

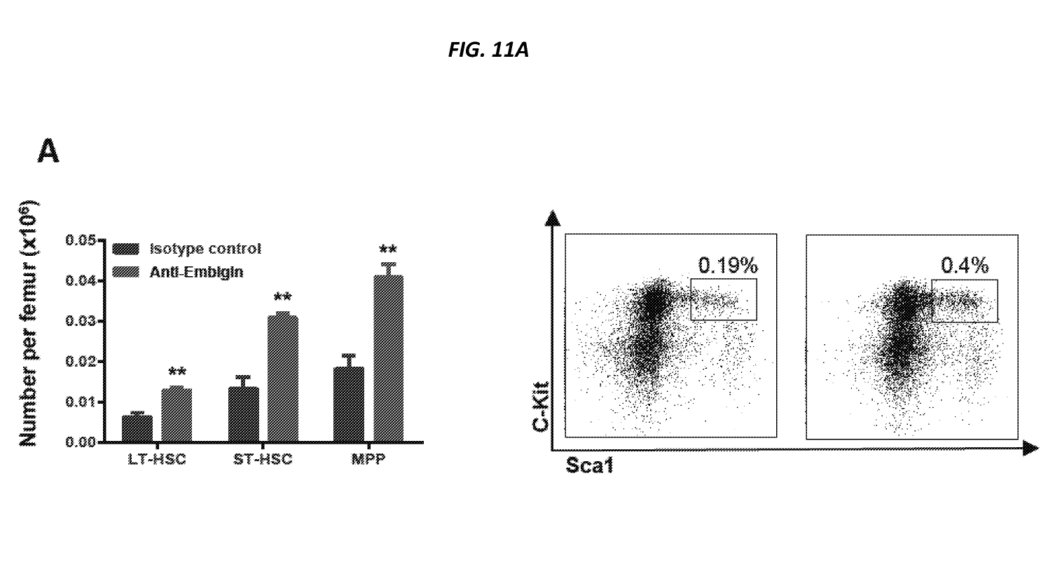

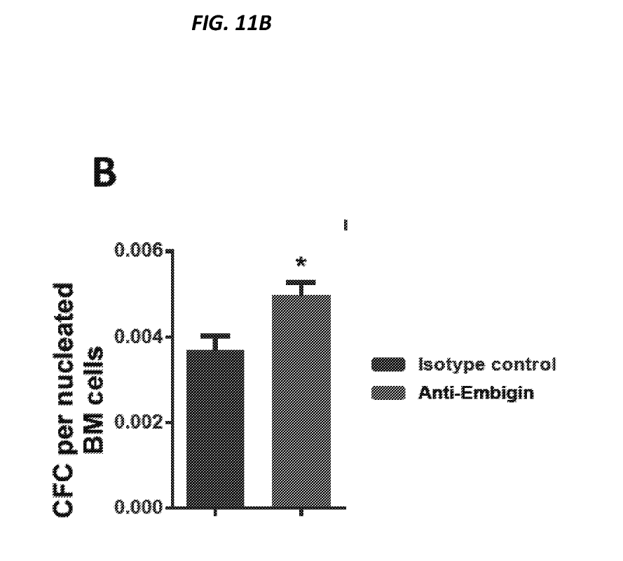

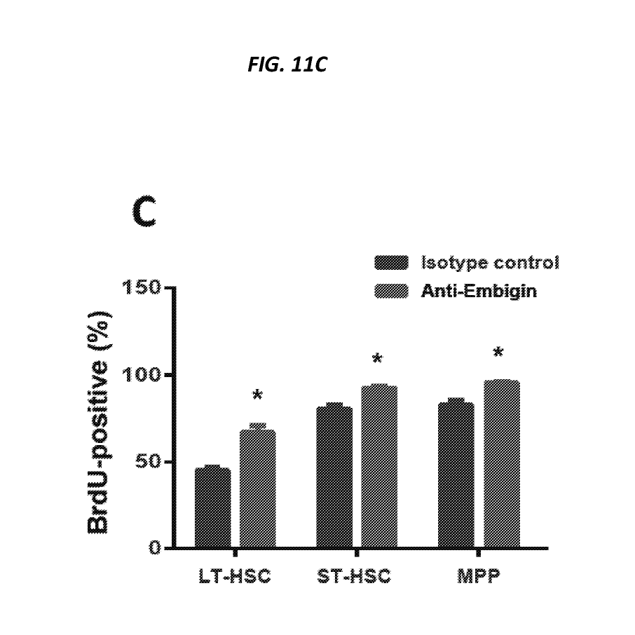

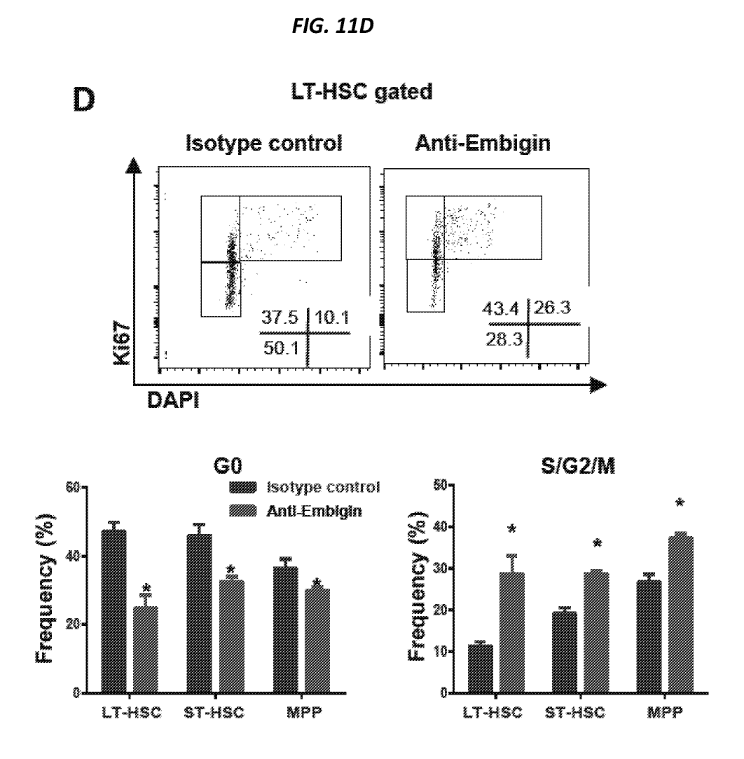

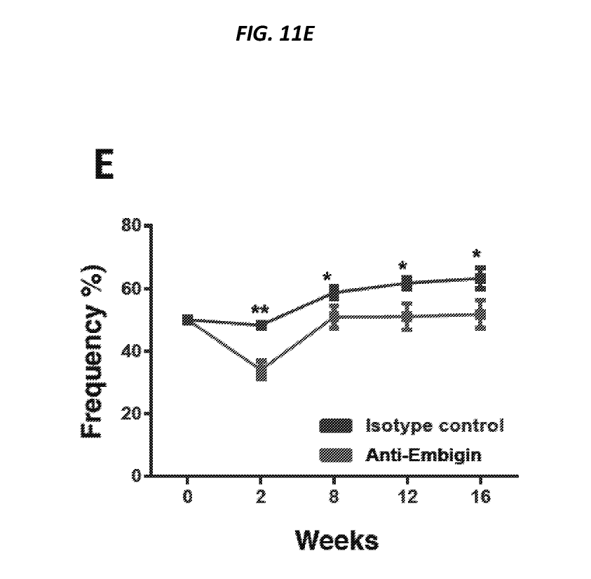

Given that Embigin is a cell adhesion molecule, the inventors assessed the effect of Embigin on HSPC localization. We found that injection of anti-Embigin resulted in mobilization of myeloid progenitors and colony-forming cells (CFC into the blood (FIG. 10B,10C). On the other hand, intra-vital microscopy studies revealed that pre-transplant Embigin blockade—either by in vivo incubation of LKS cells [known to express Embigin] (Forsberg et al., 2010 with anti-Embigin or by injecting anti-Embigin into lethally irradiated hosts—resulted in a significantly lower number of transplanted LKS cells reaching calvarial bone marrow as compared to an isotype control (FIG. 10D,10E), thus identifying Embigin as a homing molecule. We also observed that WT LKS cells transplanted into anti-Embigin pre-treated recipients displayed a higher proliferation rate (FIG. 10F), indicating that Embigin may also regulate HSPC quiescence. To examine this further, we performed cell cycle and BrdU incorporation studies following injection of WT animals with anti-Embigin or isotype-control antibody and found an approximately 2-fold increase in the frequency of LT-HSCs, ST-HSCs, MPP and colony-forming cells in anti-Embigin treated animals (FIG. 11A, 11B). This was associated with increased BrdU incorporation by primitive hematopoietic cells (FIG. 11C), a reduction in the proportion of cells in G0 phase of the cell cycle (FIG. 11D) with a corresponding increase in S/G2/M phase. Consistent with the above findings, we found that bone marrow from anti-Embigin treated animals reconstituted poorly when competitively transplanted into irradiated recipients as compared to isotype-control treated marrow, likely due to the impaired HSPC homing and increased cell cycling (FIG. 11E). Taken together, these results identify Embigin as a regulator of HSPC homing and quiescence and create the rationale for future mechanistic studies to examine the role of Embigin in HSPC regeneration.

### Example 3

Our approach illustrates several important methodological and biological principles. First, it applies single cell approach to the study of the bone marrow niche and by doing so, identifies a subset of osteolineage cells (proximal OLCs which are highly enriched for membrane-bound and secreted molecules, including known HSPC regulatory molecules and those characterized by us as niche factors in the current manuscript. Thus, we show that by using single cell transcriptome comparison between individual cells which belong to the same lineage but differ only by their proximity to HSPC, a previously unrecognized heterogeneity within a cell lineage can be revealed, and a molecularly relevant and highly specialized cell subset can be defined. More fundamentally, we demonstrate that positional relationship to a heterologous cell type serves as a powerful predictor of cellular heterogeneity in vivo.

Secondly, our approach to niche factor identification was unbiased. Of the factors that were identified herein as niche regulators, none has been previously implicated in extrinsic regulation of hematopoiesis. By comparing the effect of these factors on HSPC in/vivo, we find that despite marked functional distinctions between them (cytokine, cell adhesion molecule, secreted RNase) they converge on the same role in the niche as regulators of HSPC quiescence. Notably, Embigin and ANG regulate quiescence of all primitive hematopoietic cells while IL18 acts predominantly on short-term progenitors, yet all of them are derived from the same proximal OLC signature. This demonstrates that bone marrow niches may not be restricted to a specific cell type, but rather control a distinct cellular state, such as quiescence. Moreover, this control is achieved through multiple, previously unappreciated molecular pathways, some of which have been uncovered by our unbiased proximity-based approach. From a purely technical angle, we demonstrate that combining micropipette-assisted single cell extraction from a defined location in a tissue section with single cell RNA-Seq is feasible and enables generation of single cell cDNA libraries whose complexity closely matches that of freshly dissociated or sorted single cells (Patel et al., 2014 (Shalek et al., 2014). Further, it has several advantages over laser capture microscopy (LCM, an established method for transcriptional analysis of spatially-defined cells (Espina et al., 2006). Firstly, it enables preservation of fluorescent labeling, which/would have been lost during ethanol fixation and subsequent drying of the section in preparation for LCM procedure. Secondly, the tissue architecture and micro-anatomical relationship between the cells are more accurately represented since our method uses thicker tissue sections as compared to LCM. Finally, the ability to harvest the whole intact cell, as opposed to the cell which would have been transected during tissue preparation for LCM, reduces cross-contamination from the neighboring cells and RNA loss, which have been noted as major technical drawbacks of the LCM procedure (Shapiro et al., 2013).

The inventors focused on bone marrow transplantation herein because of its clinical relevance and the importance of finding new ways to enhance post-transplant bone marrow recovery, for example IL18-blockade. We found that all three factors which we characterized act as regulators of HSPC quiescence in the transplant context. Surprisingly, the inventors also discovered that they have a measurable effect on HSPC quiescence under homeostatic conditions, indicating that despite marked differences in unconditioned and post-irradiation bone marrow niche, our platform is suitable for identification of niche factors which are active not only under conditions of stress but also in steady-state hematopoiesis.

As disclosed herein, the inventors have identified HSPC regulators based on the analysis of OLCs. IL18/Embigin/and/Ang transcripts are detectable in several other niche cell types found in close apposition to HSPC, such as perivascular cells (Kunisaki et al., 2013), which likely act as non-redundant sources of these factors, as has been previously demonstrated for CXCL12 (Ding and Morrison, 2013; Greenbaum et al., 2013), stem cell factor (Ding et al., 2012) and now shown for ANG in the current manuscript. Whether proximal OLCs also serve as a source of unique, OLC-specific niche factors remains an open question, which will be addressed by functional validation of multiple other candidate molecules which are present in the proximal OLC signature.

In summary, the inventors demonstrate that single cell proximity-based analysis serves as unbiased strategy for identification of niche-derived regulators, offers new insights into the molecular regulation of HSPC quiescence and opens unexplored avenues for translational approaches to enhance HSPC regeneration. Recent advances in in/situ transcriptome analysis methodology offered by TEVA (Lovatt et al., 2014), Fisseq (Lee et al., 2014) or MERFISH (Chen et al., 2015), will facilitate application of the proximity-based analysis which was designed and validated by the current study, to define the molecules and cell subsets intimately involved in inter-cellular communications in healthy and diseased tissue.

### Example 4

ANG is a Non-Cell Autonomous Regulator of LT-HSC Quiescence and Self-Renewal

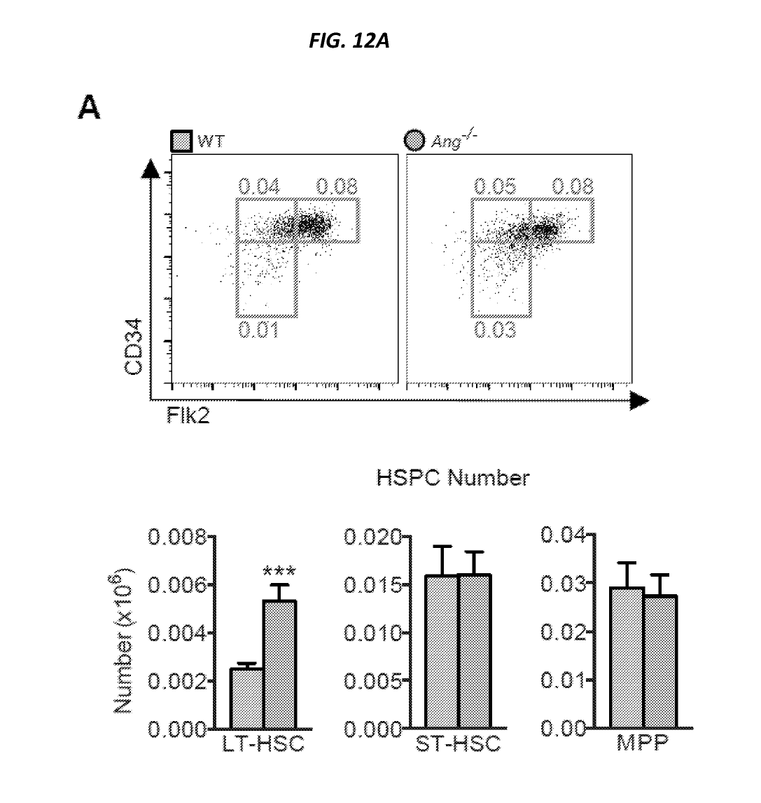

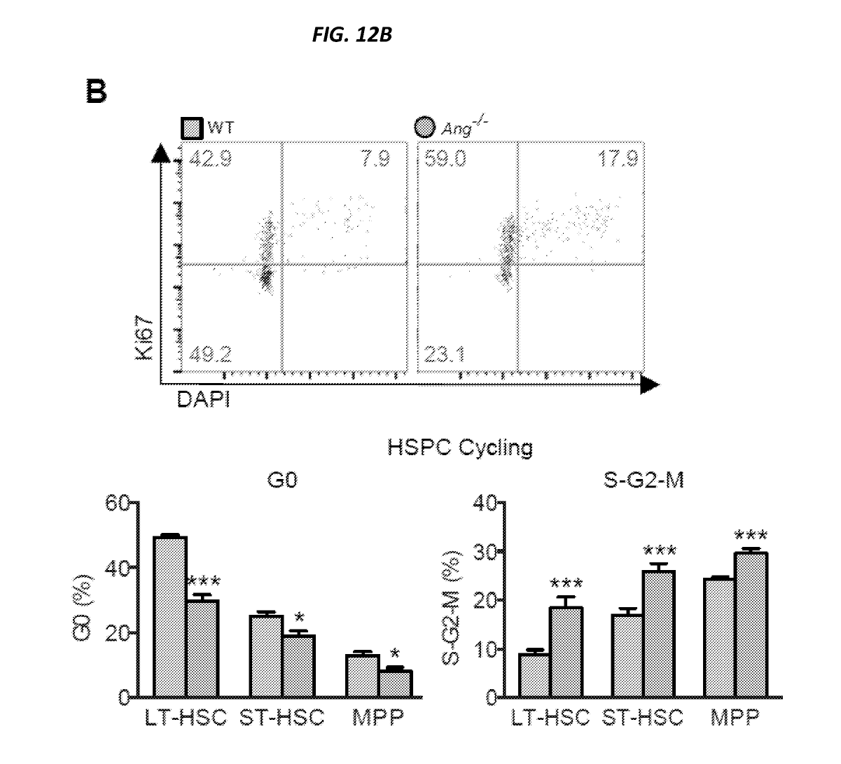

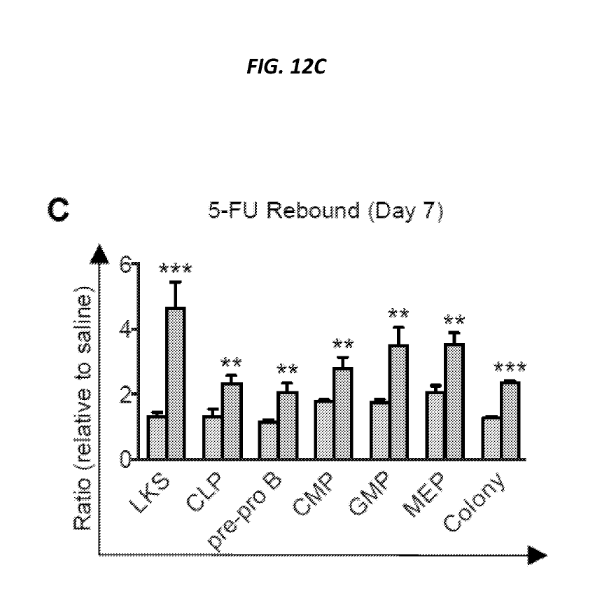

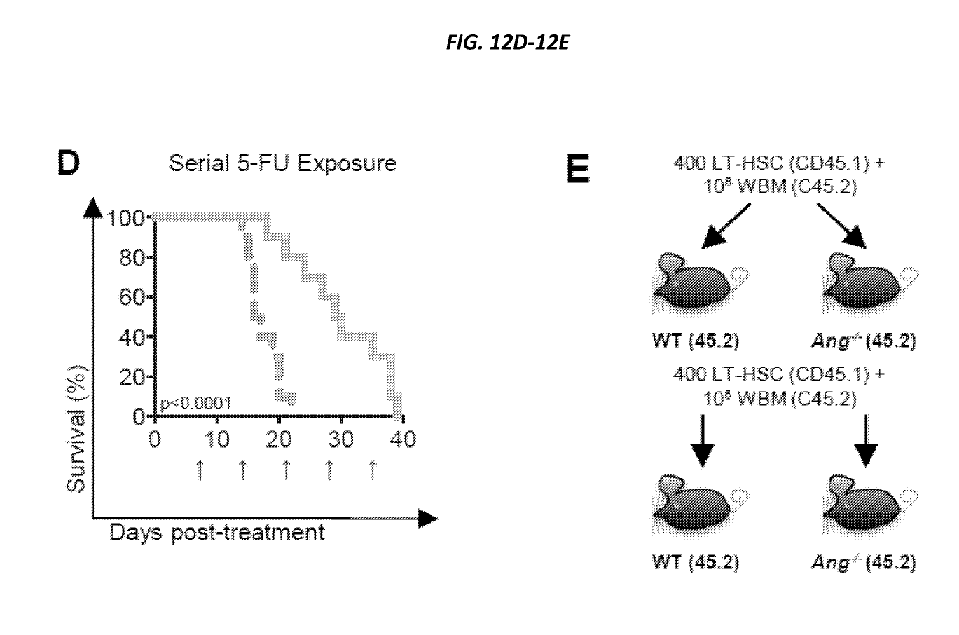

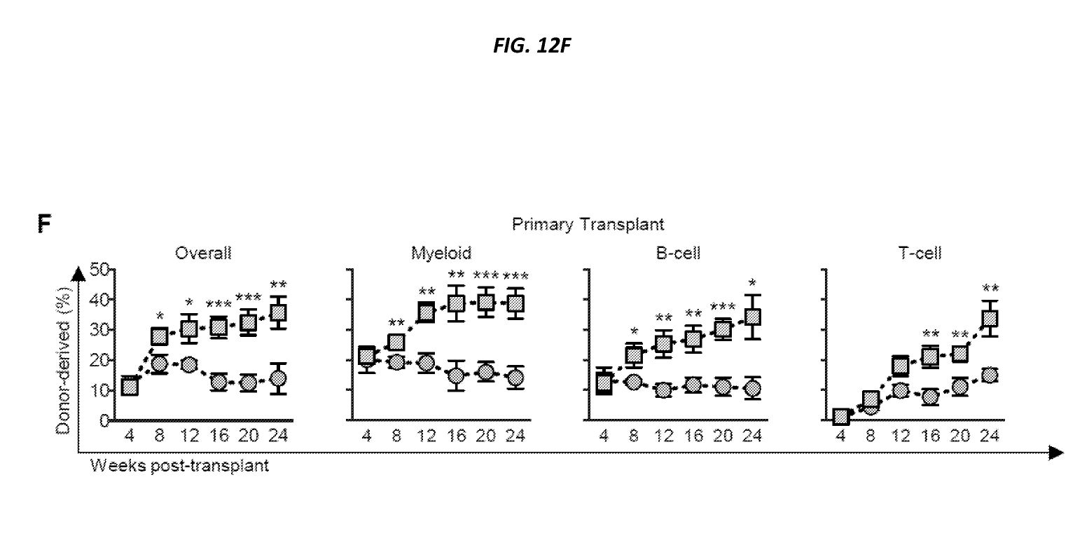

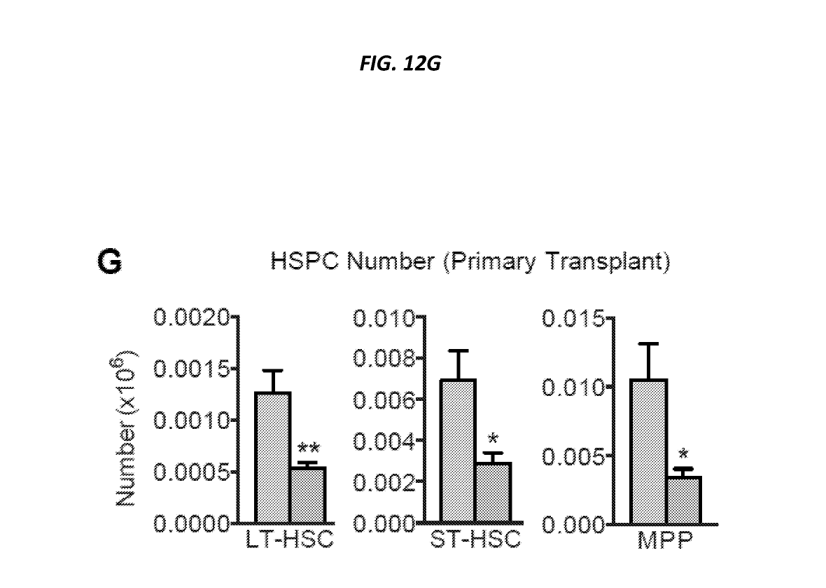

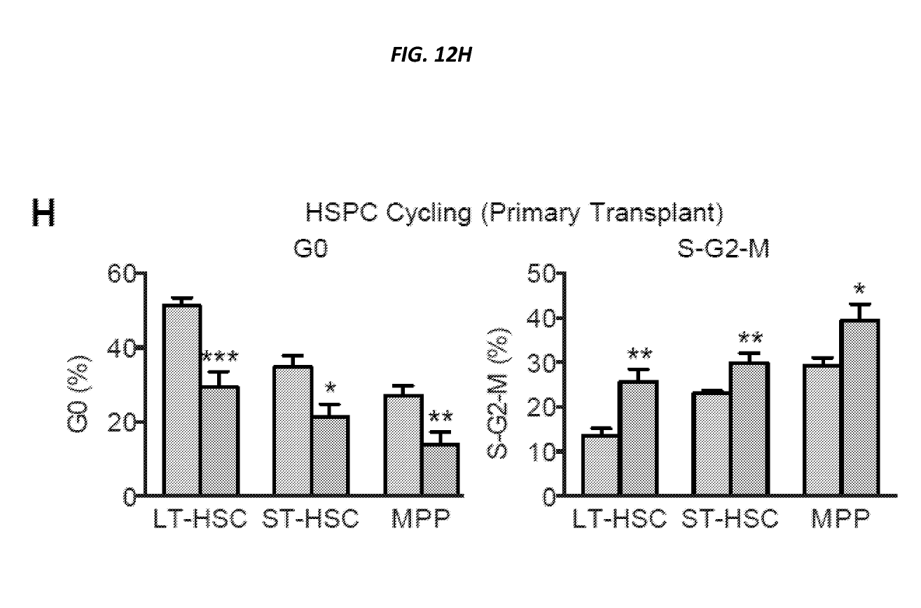

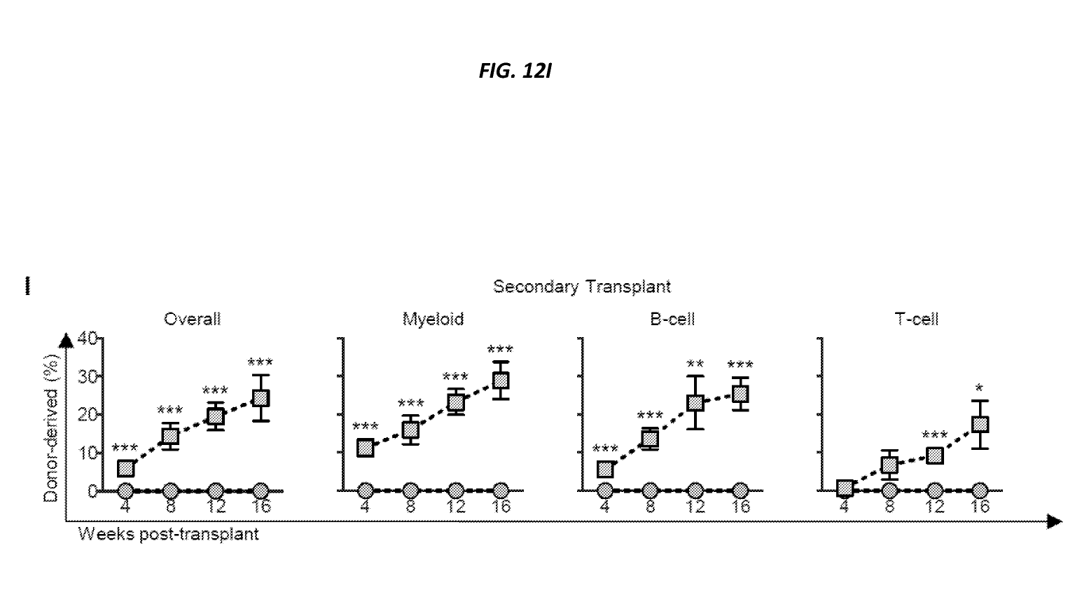

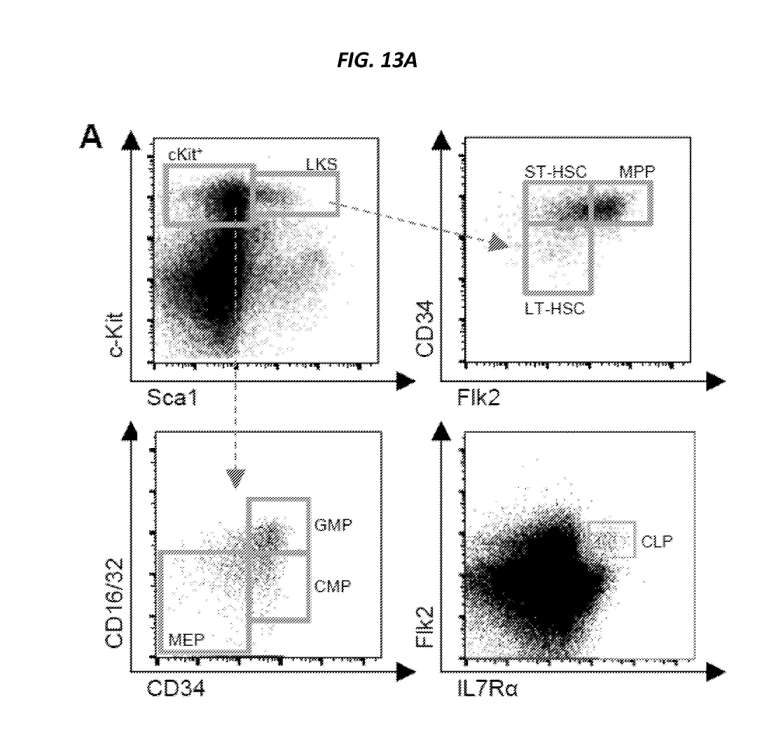

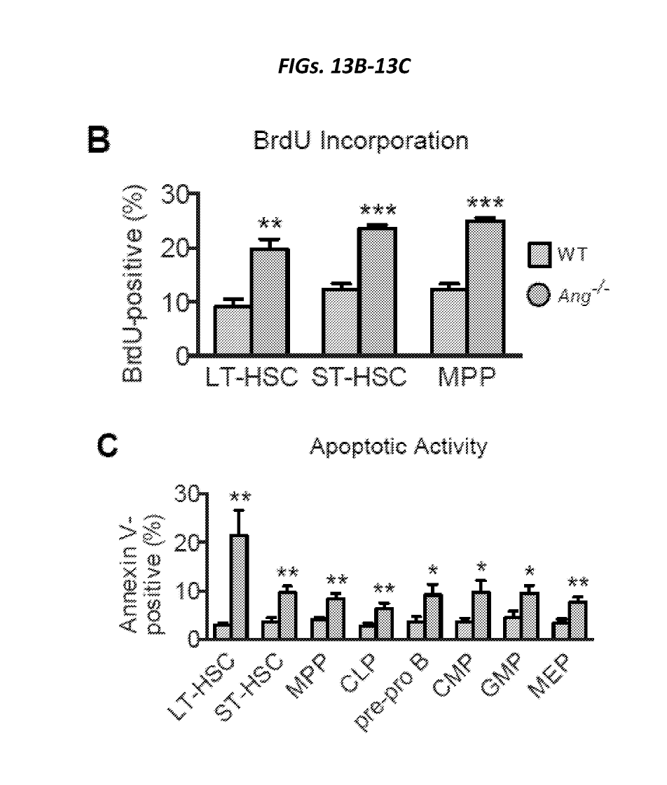

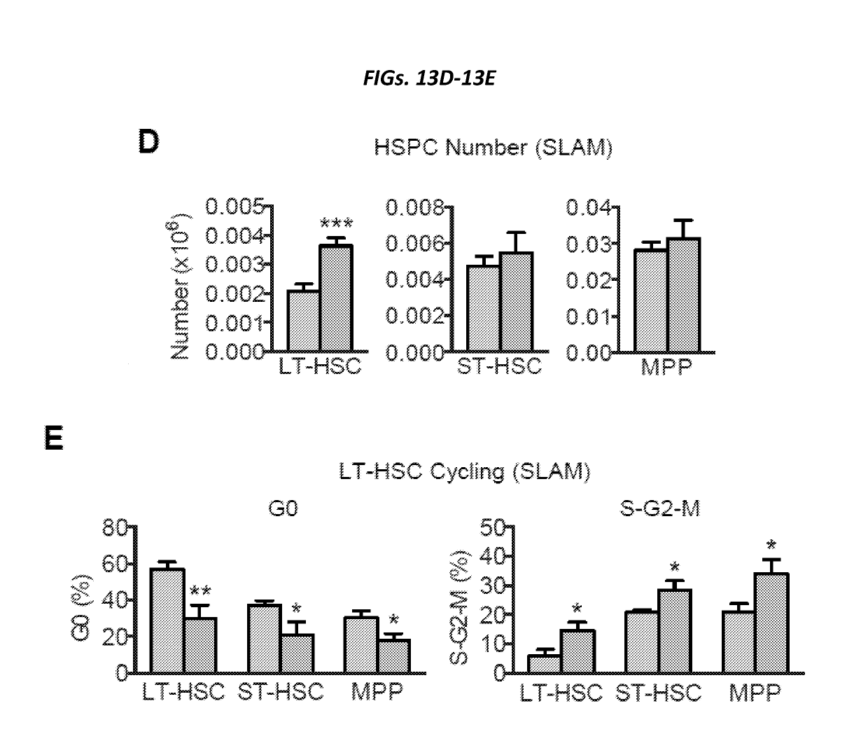

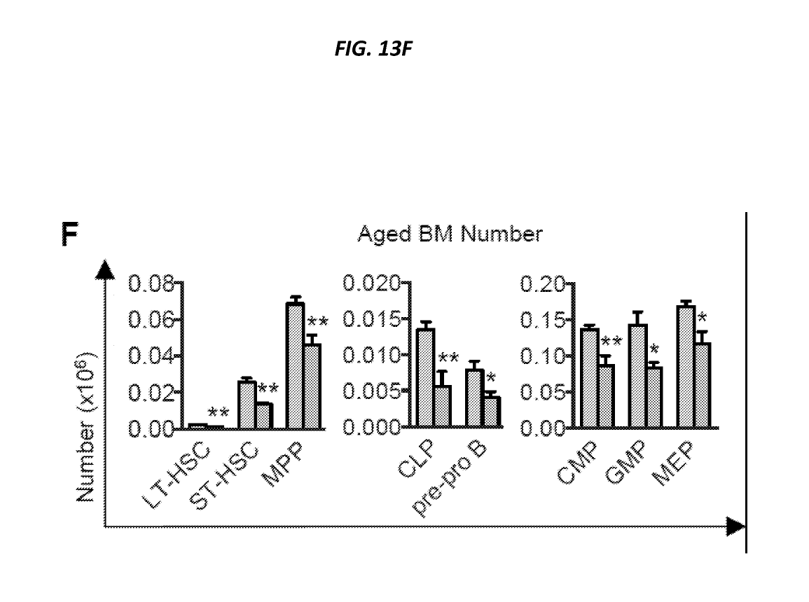

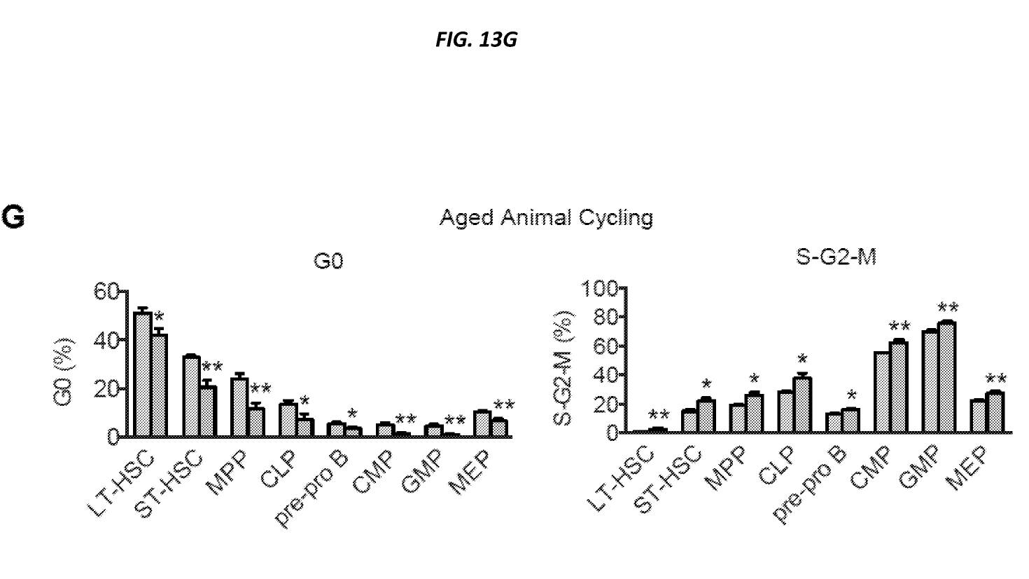

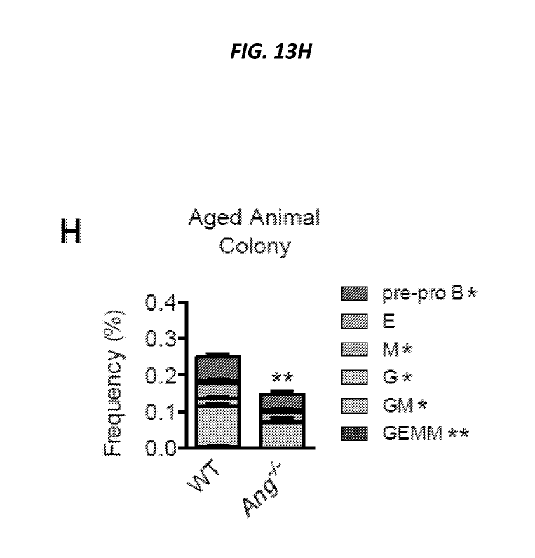

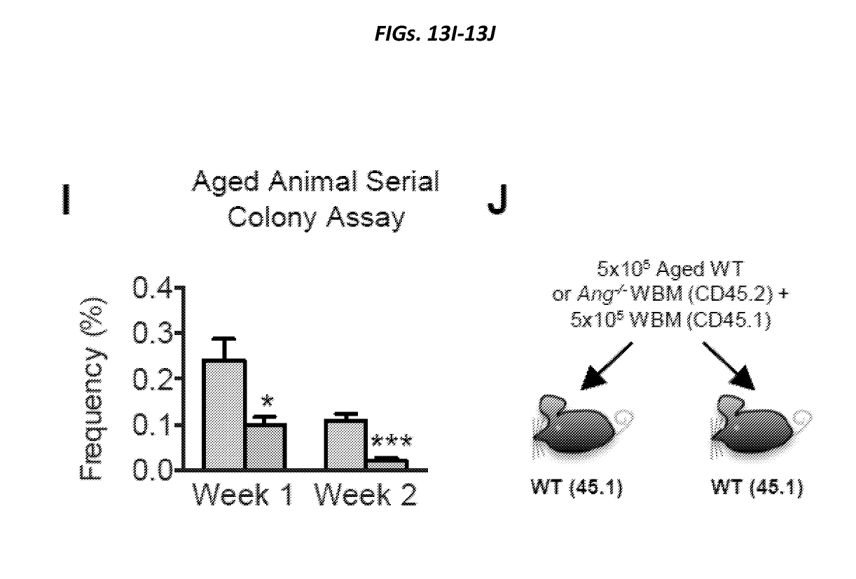

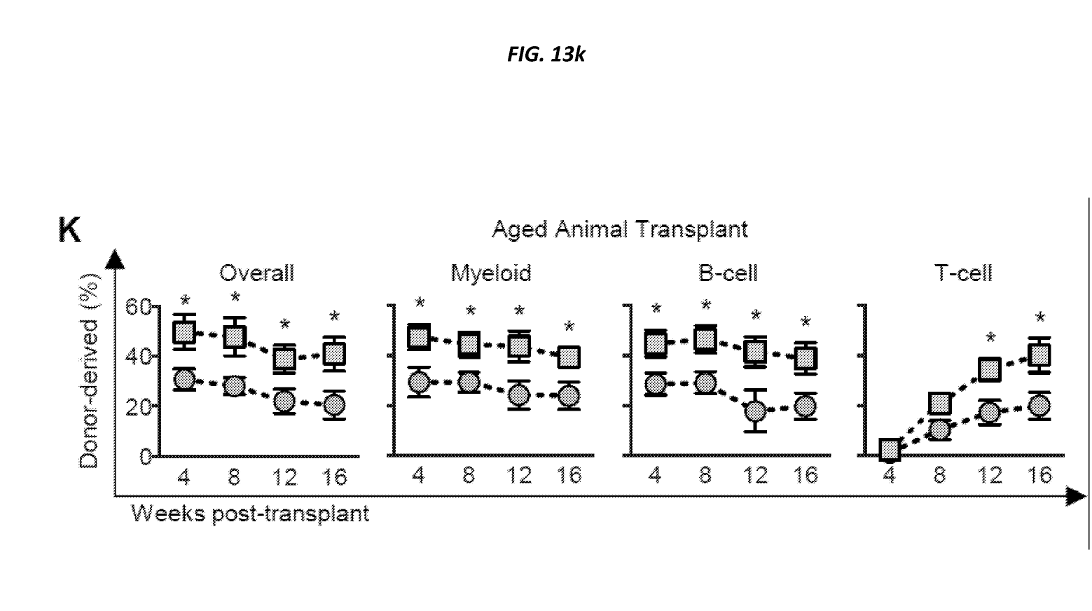

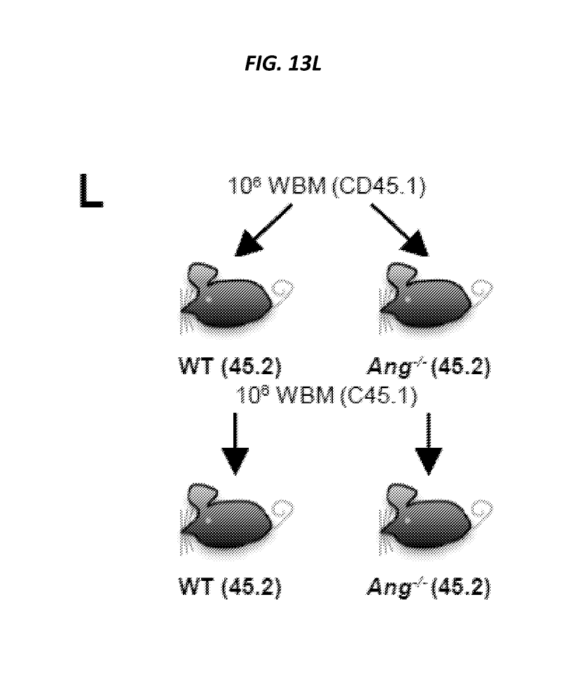

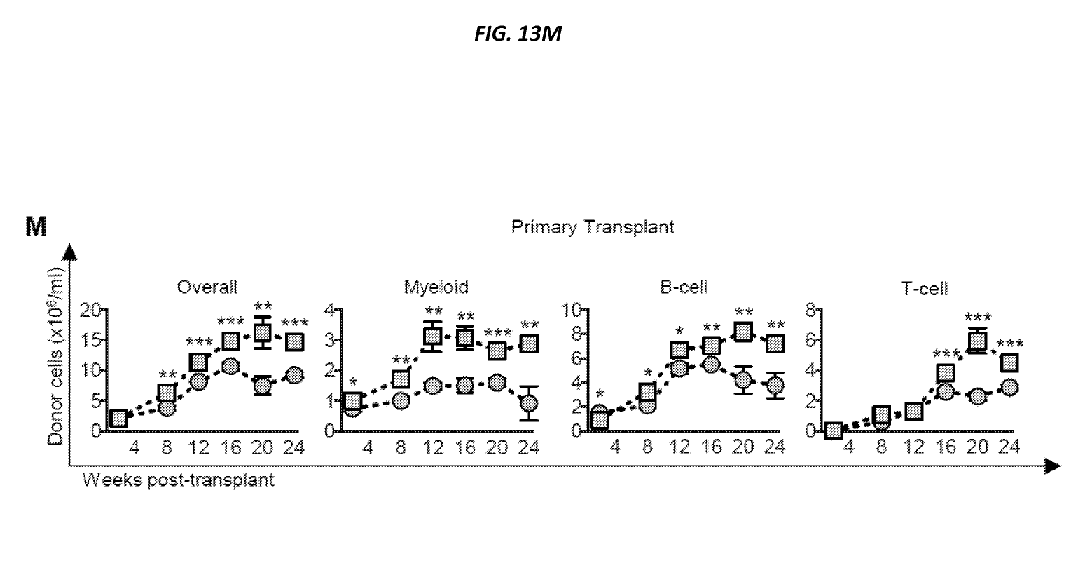

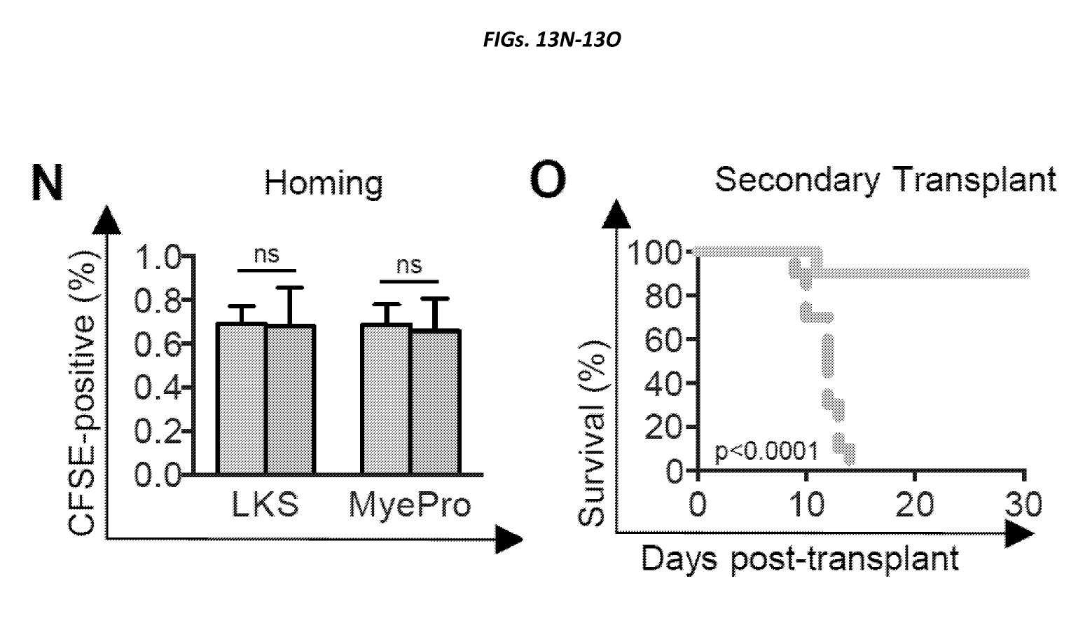

To functionally and mechanistically characterize the role of ANG in hematopoiesis, we first profiled HSPC in the BM of Ang knockout (Ang−/−) mice and found a 2-fold increase in the number of LT-HSCs (Flk2−CD34− Lin−c-Kit+Sca1+ [LKS]), but not short-term (ST)-HSCs (Flk2−CD34+ LKS) or multi-potent progenitors (MPP; Flk2+CD34+ LKS) in Ang−/− BM (FIG. 12A; detailed gating scheme in FIG. 13A). Consistently, a reduction in G0 phase and a corresponding increase in S/G2/M phases of the cell cycle (FIG. 12B), as well as enhanced BrdU incorporation (FIG. 13B) was observed in Ang−/− LT-HSCs. Ang−/− ST-HSCs and MPPs also displayed increased cycling (FIG. 12B, 13B) but curiously no difference in cell number (FIG. 12A), which could be attributed, at least in part, to elevated apoptosis across hematopoietic lineages in Ang−/− mice (FIG. 13C). This observation is consistent with the anti-apoptotic function of ANG in other cell types (Kieran et al., 2008; Li et al., 2010). These patterns were also observable by other commonly used cell surface markers (FIGS. 13D-13E), confirming that LT-HSCs in Ang−/− BM cycle more actively than in WT BM. Despite the dramatic increase in LT-HSC number in Ang−/− BM (FIGS. 12A, 13D), only mild lymphocytosis was apparent at baseline in 8-12 week old mice (Table 5). However, under conditions of stress, progenitor response to the genotoxic agent, 5-fluorouracil (5-FU), was markedly exaggerated in Ang−/− mice (FIG. 12C). Further, exposure of these animals to serial proliferative stress, such as weekly injections of 5-FU, resulted in excess animal morality (FIG. 12D). Consistent with the phenotype of stress-induced exhaustion (Orford and Scadden, 2008), aged 22 month old Ang−/− mice developed leukopenia (Table 6) and showed a marked reduction in the number of primitive hematopoietic cells in the BM (FIG. 13F), accompanied by more active HSPC cycling (FIG. 13G). Aged Ang−/− mice also displayed reduced functional capabilities by in vivo methylcellulose assays (FIGS. 13H-13I) and in vivo competitive transplantation (FIGS. 13J-13K). To further characterize the functional significance of ANG-deficiency-induced loss of HSPC quiescence, transplant experiments were performed by injecting either total BM (FIG. 13L) or purified LT-HSCs (FIG. 12E) into lethally-irradiated WT or Ang−/− hosts. In both experiments, impaired long-term multi-lineage reconstitution was observed in Ang−/− hosts (FIG. 12F, 13M) with particularly pronounced impairment at later time points. Notably, WT HSPC in the ANG-deficient microenvironment displayed dramatically reduced HSPC number, accompanied by more active cycling (FIG. 12G-12H). To rule out a homing defect as a cause of impaired reconstitution in Ang−/− hosts, CD45.1 lineage-negative cells were injected into irradiated WT or Ang−/− recipients, and no difference in the percentage of LKS cells or Lin− c-Kit+ Sca1− myeloid-restricted progenitors in the BM of these animals was observed 16 hours after transplantation (FIG. 13N). In order to evaluate the effect of niche-derived ANG on HSC self-renewal, we carried out serial transplantation experiments. When performed non-competitively, injection of an equal number of whole BM cells from primary Ang−/− recipients strikingly resulted in death of all secondary Ang−/− recipients (FIG. 13O), while competitive transplantation demonstrated no detectable hematopoietic contribution by LT-HSCs that had been passaged through ANG-deficient primary recipients (FIG. 12I). The marked inability to reconstitute in both transplant settings indicates severe loss of HSC self-renewal capacity in ANG-deficient hosts. Taken together, these data demonstrate that ANG acts as a non-cell autonomous regulator of quiescence and self-renewal of primitive hematopoietic cells, particularly LT-HSC.

TABLE 5
Cell counts for 8-12 week old Ang^{-/-} mice

| Organ | Parameter | Unit | WT | Ang^{-/-} |
|---|---|---|---|---|
| Blood | WBC | ×10³/μl | 8.60 ± 1.07 | 12.0 ± 1.18* |
| | LYM | ×10³/μl | 5.57 ± 0.89 | 8.90 ± 1.21* |
| | MON | ×10³/μl | 0.93 ± 0.30 | 0.80 ± 0.24 |
| | NEU | ×10³/μl | 2.10 ± 0.38 | 2.29 ± 0.39 |
| | PLT | ×10³/μl | 568 ± 60.2 | 665 ± 103 |
| | Mac1⁺Gr1⁺ | ×10³/μl | 0.77 ± 0.08 | 0.53 ± 0.06* |
| | B220⁺ | ×10³/μl | 4.21 ± 0.54 | 6.36 ± 1.88* |
| | CD3e⁺ | ×10³/μl | 2.08 ± 0.34 | 2.74 ± 0.35 |
| Bone Marrow | Cellularity | ×10⁸/femur | 21.0 ± 0.65 | 20.2 ± 1.59 |
| | Mac1⁺Gr1⁺ | ×10⁸/femur | 12.3 ± 0.65 | 9.78 ± 0.96* |
| | Ter119⁺ | ×10⁸/femur | 2.15 ± 0.35 | 2.03 ± 0.26 |
| | B220⁺ | ×10⁸/femur | 4.69 ± 0.31 | 6.23 ± 0.45* |
| | CD3e⁺ | ×10⁸/femur | 0.57 ± 0.04 | 0.58 ± 0.06 |

Data represent mean ± SEM.
*p < 0.05
n = 9

TABLE 6
Cell counts for 22 month old Ang^{-/-} mice

| Organ | Parameter | Unit | WT | Ang^{-/-} |
|---|---|---|---|---|
| Blood | WBC | ×10³/μl | 9.03 ± 1.72 | 4.67 ± 0.56* |
| | LYM | ×10³/μl | 6.89 ± 1.28 | 3.27 ± 0.64* |
| | MON | ×10³/μl | 0.22 ± 0.08 | 0.14 ± 0.02 |
| | NEU | ×10³/μl | 1.92 ± 0.55 | 1.26 ± 0.12 |
| | PLT | ×10³/μl | 960 ± 71.9 | 1038 ± 89.1 |
| | Mac1⁺Gr1⁺ | ×10³/μl | 0.38 ± 0.07 | 0.17 ± 0.02* |
| | B220⁺ | ×10³/μl | 6.18 ± 1.34 | 2.63 ± 0.37* |
| | CD3e⁺ | ×10³/μl | 0.69 ± 0.13 | 0.60 ± 0.11 |
| Bone | Cellularity | ×10⁸/femur | 31.0 ± 1.17 | 27.3 ± 1.01* |
| Marrow | Mac1⁺Gr1⁺ | ×10⁸/femur | 16.3 ± 0.77 | 12.6 ± 0.40** |
| | Ter119⁺ | ×10⁸/femur | 4.05 ± 0.44 | 2.73 ± 0.28* |
| | B220⁺ | ×10⁸/femur | 5.01 ± 0.35 | 3.03 ± 0.34** |
| | CD3e⁺ | ×10⁸/femur | 1.05 ± 0.14 | 0.49 ± 0.08** |

Data represent mean ± SEM.
*p < 0.05,
**p < 0.01
n = 5

### Example 5

ANG Enhances Myeloid-Restricted Progenitor Cell Proliferation while Keeping HSPC Quiescent

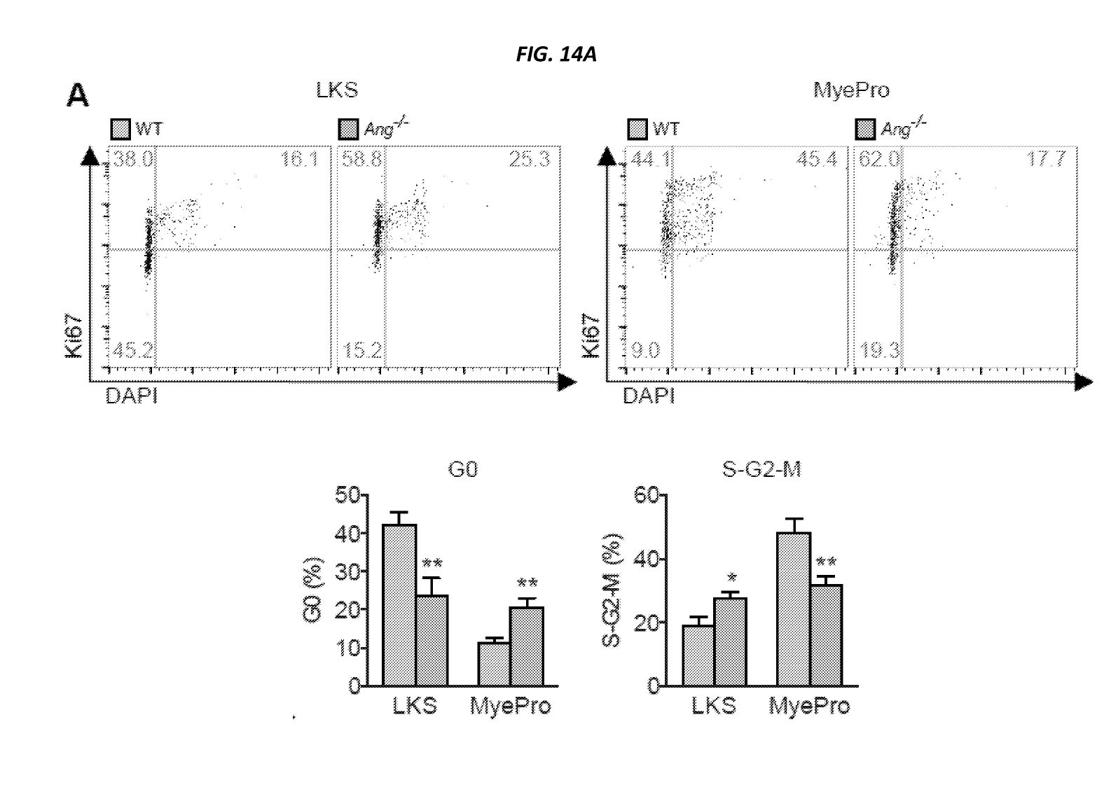

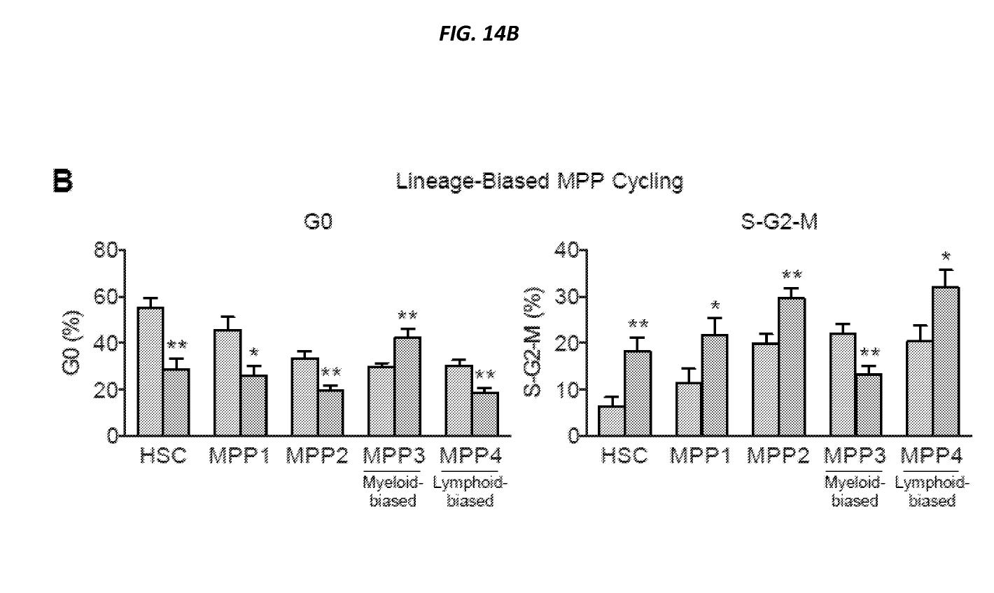

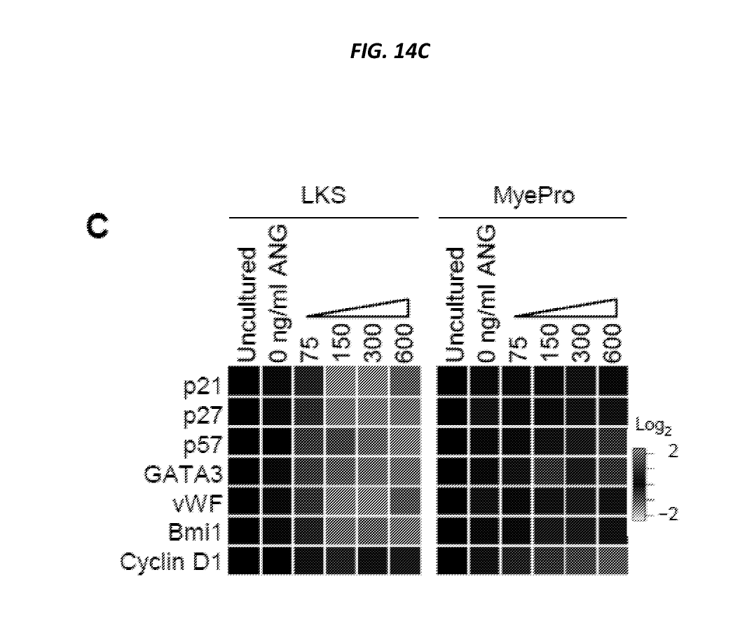

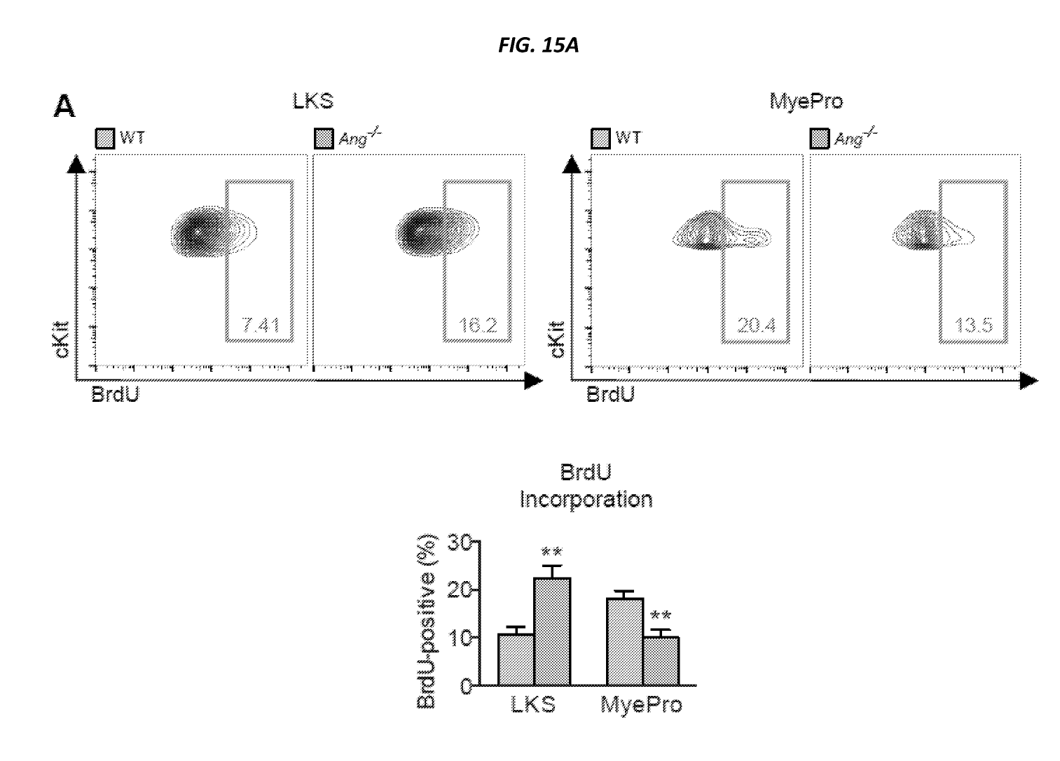

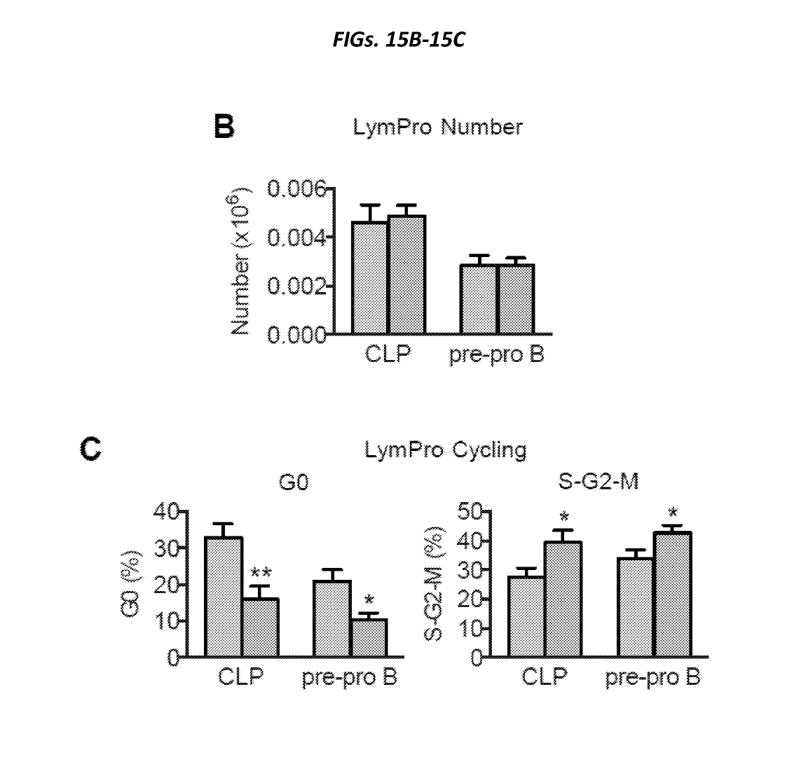

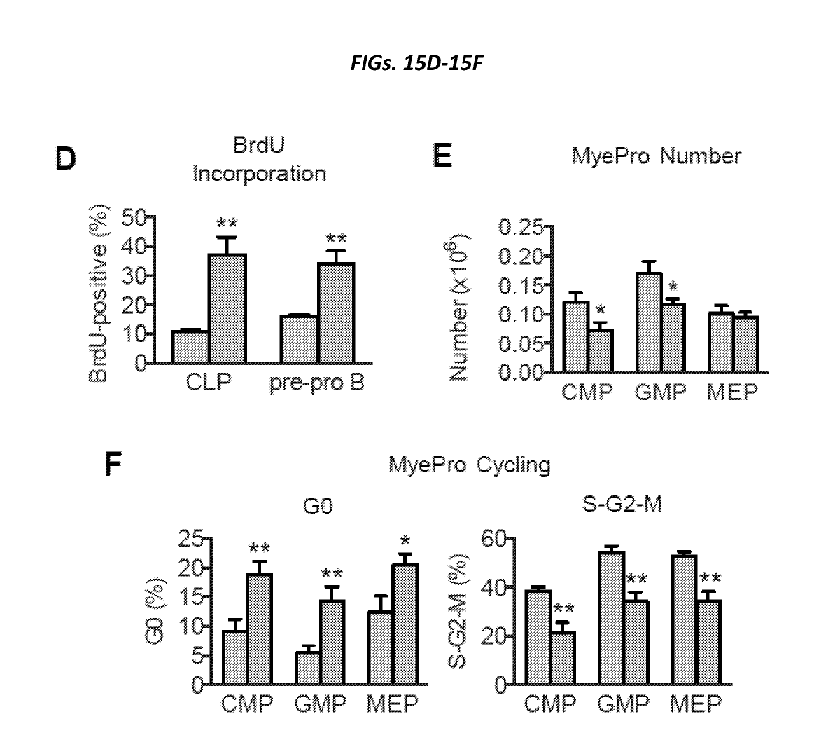

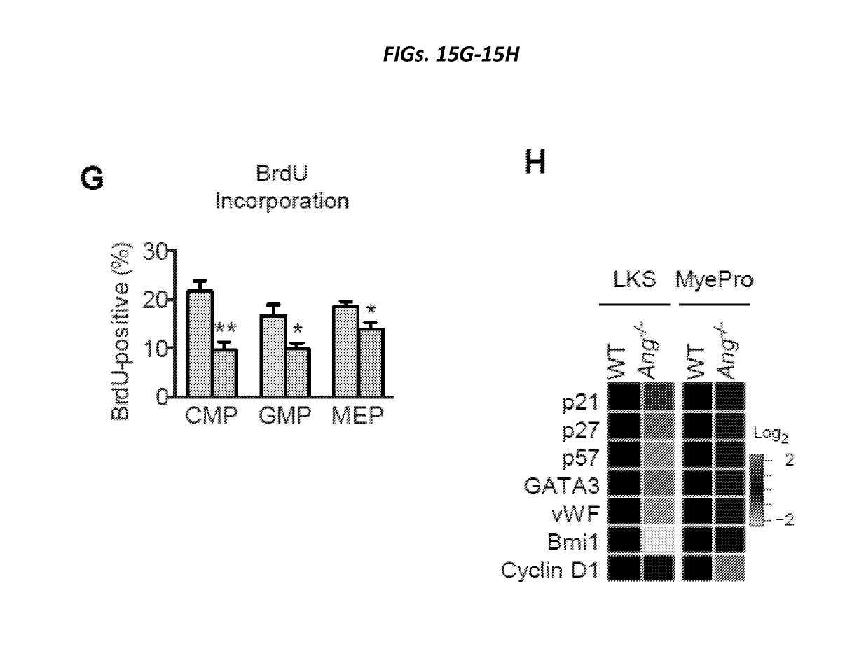

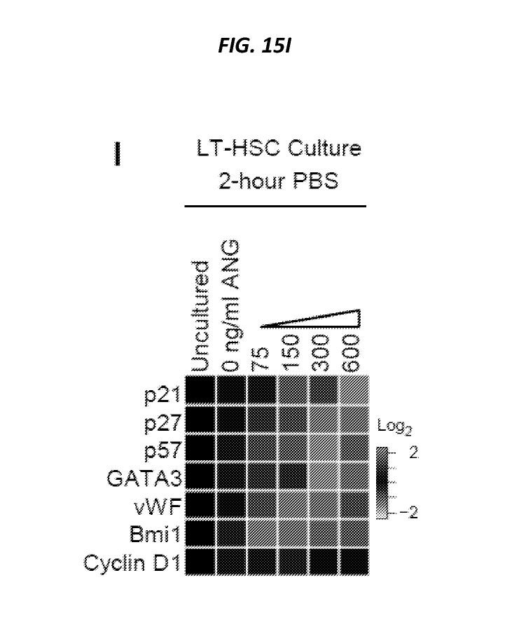

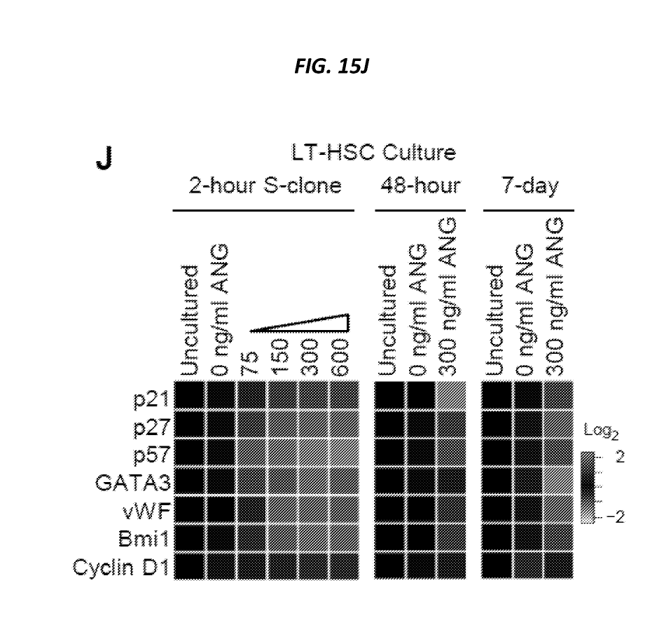

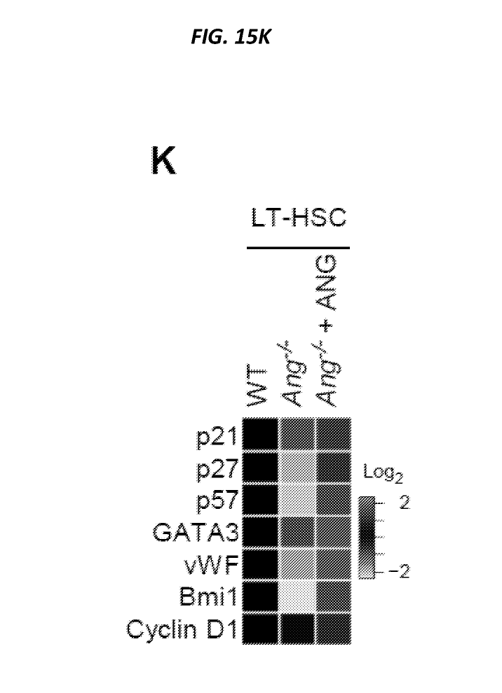

The finding that ANG restricts cell cycling of HSPC is the first evidence for a suppressive activity of ANG on cell proliferation, as all previous studies showed that ANG promotes cell proliferation (Li and Hu, 2010). We therefore examined cell-type specific effects of ANG in various cells of the hematopoietic lineage. We observed that while Ang−/− LKS cells cycle more actively, Ang−/− myeloid-restricted progenitors showed restricted, rather than enhanced, cycling (FIG. 14A). Consistently, we observed an increase of in vivo BrdU incorporation in LKS cells but a marked decrease in myeloid-restricted progenitors in Ang−/− mice, relative to WT controls (FIG. 15A). The cell-context specificity of ANG was further illustrated by analyzing lymphoid-restricted and myeloid-restricted progenitors including common lymphoid progenitors (CLP; Lin−IL7R+Flk2+B220−), pre-pro B cells (Lin−IL7R+Flk2+B220+), common myeloid progenitors (CMP; Lin−c-Kit+Sca1−CD34+CD16/32−), granulocyte-macrophage progenitors (GMP; Lin−c-Kit+Sca1−CD34+CD16/32+), and megakaryocyte-erythroid progenitors (MEP; Lin−c-Kit+Sca1−CD34−CD16/32−). The inventors discovered that Ang−/− CLPs and pre-pro B cells (FIG. 15B) resemble HSPC by displaying more active cycling (FIG. 15C) and incorporating more BrdU (FIG. 15D), demonstrating that ANG restricts lymphoid progenitor proliferation. In contrast, myeloid-restricted progenitors, including CMP, GMP, and MEP, all displayed less active cycling (FIG. 15F) and reduced BrdU incorporation (FIG. 15G), accompanied by a reduction of CMP and GMP number (FIG. 15E) in Ang−/− mice. Importantly, restricted proliferation of myeloid-biased MPP3s (CD150−CD48+CD135−CD34+LKS) was detected and more active cycling of lymphoid-biased MPP4s (CD150+CD48+CD135+CD34+LKS; FIG. 14B) (Cabezas-Wallscheid et al., 2014) in Ang−/− mice was observed. Together, these data indicate that the function of ANG is cell context-specific: while ANG restricts cell proliferation in primitive HSCs and lymphoid-restricted progenitors, it promotes proliferation of myeloid-restricted progenitors. This transition occurs within the earliest phenotypically-defined lineage-biased progenitor cell types between MMP3 and MPP4.

Cell context-specific regulation of ANG was confirmed by the fact that Ang deletion resulted in decreased expression of cycle checkpoint or self-renewal genes including p21, p27, p57, GATA3, vWF, Bmi1 (Cheng et al., 2000; Frelin et al., 2013; Kent et al., 2009; Matsumoto et al., 2011; Park et al., 2003) in LKS cells but not in myeloid-restricted progenitors (FIG. 15H). In contrast, the cell cycle-related gene, cyclin D1, was decreased in myeloid-restricted progenitors but not in LKS cells upon Ang deletion (FIG. 15H). Testing whether they might be clinically relevant to these findings, the inventors assessed the effect of recombinant ANG protein on cultured stem and progenitor cells. Remarkably, culture with ANG for 2 hours in PBS led to a dose-dependent increase in the expression of pro-self-renewal genes in LKS cells (FIG. 14C). No such change was noted in myeloid-restricted progenitors. In contrast, cyclin D1 was enhanced by ANG in myeloid-restricted progenitors but not in LKS cells (FIG. 14C). A similar pattern was observed in LT-HSCs cultured with ANG for 2 hours in PBS (FIG. 15I) or under longer culture conditions in S-clone media (FIG. 15J). Notably, addition of exogenous ANG rescued the reduced pro-self-renewal transcripts observed in Ang−/− LKS cells (FIG. 15K). Together, these data demonstrate that ANG differentially regulates gene expression in HSC and progenitors, including genes relevant for proliferation and self-renewal.

ANG Dichotomously Regulates Protein Synthesis in LKS and Myeloid-Restricted Progenitor Cells

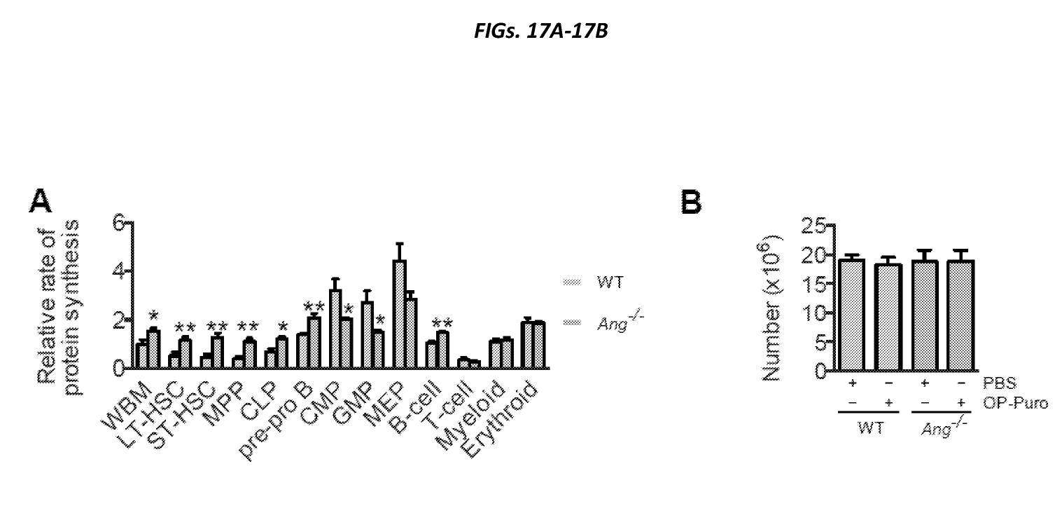

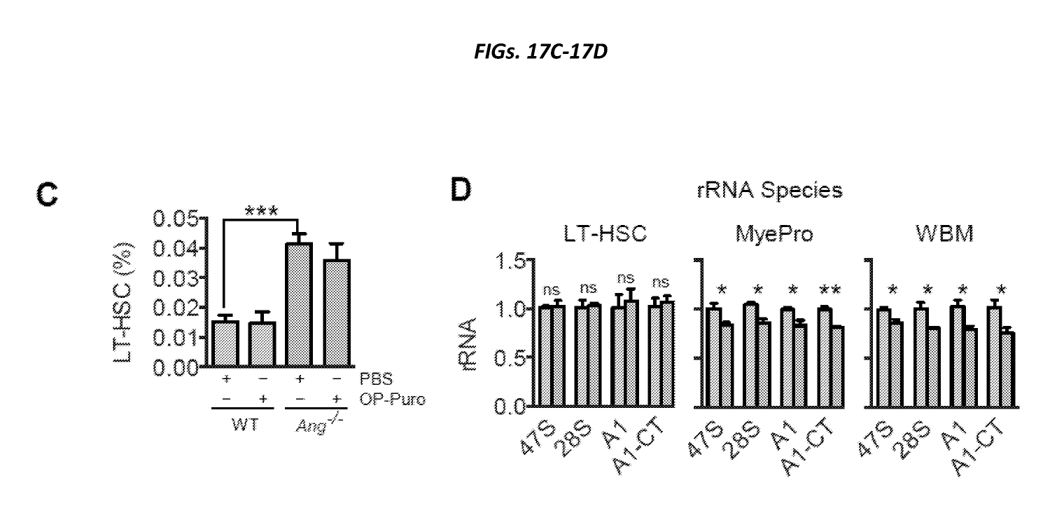

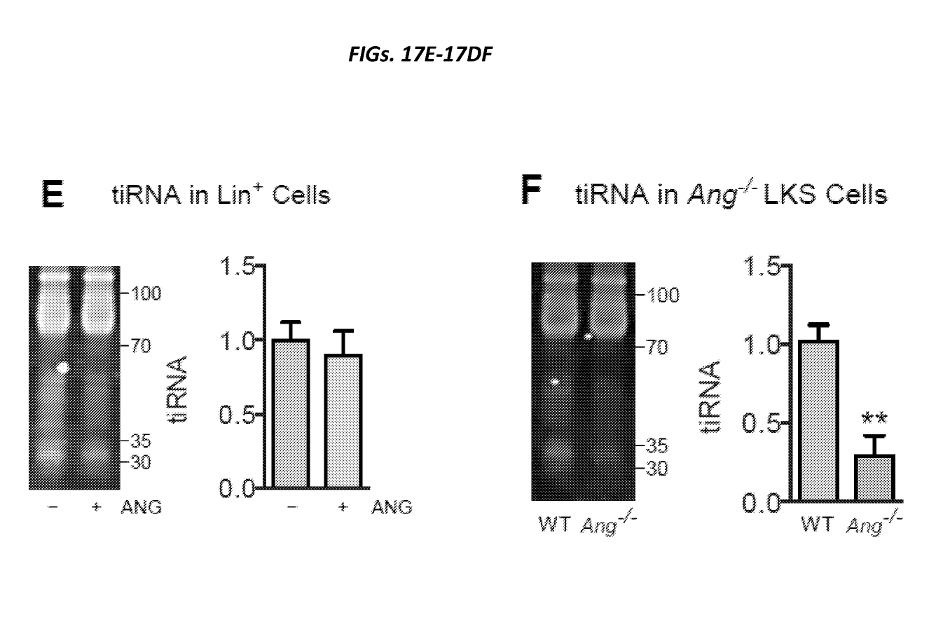

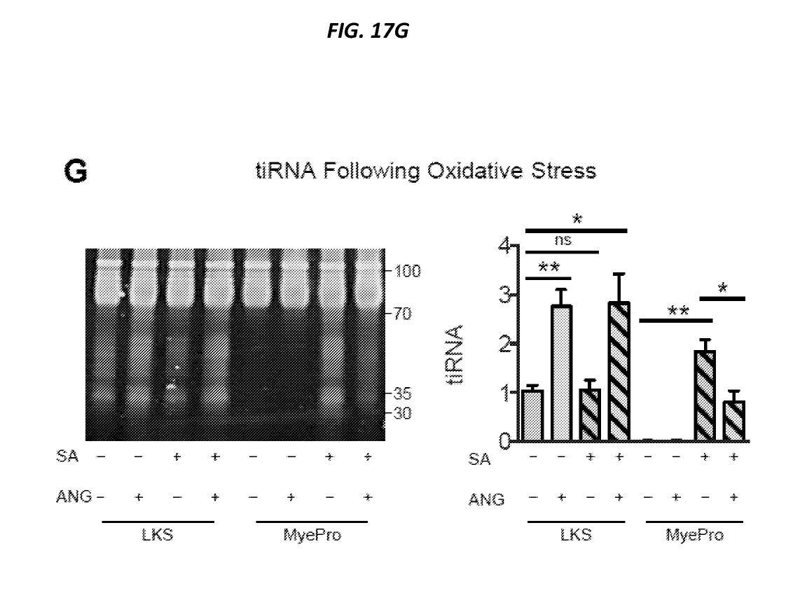

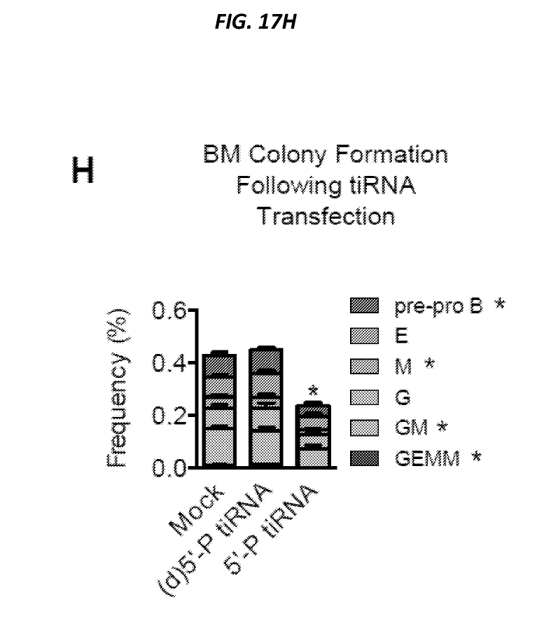

ANG has been shown in other cell types to regulate global protein synthesis, a housekeeping function recently shown to be tightly regulated in primitive HSCs (Signer et al., 2014). To determine whether ANG regulates protein synthesis in HSPC, we assessed in vivo protein synthesis in Ang−/− mice by a fluorogenic assay using 0-propargyl-puromycin (OP-Puro) (Signer et. al., 2014). Consistent with increased cell cycling, Ang−/− LKS cells showed a higher rate of protein synthesis while Ang−/− myeloid-restricted progenitors demonstrated reduced protein synthesis (FIG. 16A). This cell context specificity was also evident when BM was analyzed with more specific markers for HSPC, lineage-restricted progenitors, and mature hematopoietic cells (FIG. 17A). In vivo administration of OP-Puro did not alter BM cellularity or LT-HSC frequency (FIGS. 17B-17C). Significantly, in vivo culture of LKS cells with ANG led to reduced protein synthesis, while ANG addition to myeloid-restricted progenitors enhanced protein synthesis (FIG. 16B). Together, these data demonstrate that the effect of ANG on protein synthesis is cell-context specific.

### Example 6

The Restrictive Function of ANG in HSPC is Mediated by tiRNA

To reveal the biochemical mechanism for this dichotomous effect of ANG on protein synthesis, we first assessed rRNA transcription, which is stimulated by ANG in other cell types (Ibaragi et al., 2009; Kishimoto et al., 2005; Tsuji et al., 2005). Addition of ANG led to enhanced rRNA transcription in myeloid-restricted progenitors and whole BM cells, but not in LKS cells (FIG. 16C). Further, Ang deletion resulted in a reduction in rRNA transcription in myeloid-restricted progenitors and whole BM but not in LKS cells (FIG. 17D). These findings are consistent with the elevated protein synthesis rate and pro-proliferative status of myeloid-restricted progenitors following ANG treatment.

ANG has been shown to reprogram protein synthesis as a stress response to promote survival under adverse conditions. This function of ANG is mediated by tiRNA, a noncoding small RNA that specifically permits translation of anti-apoptosis genes while global protein translation is suppressed so that stressed cells have adequate time and energy to repair damage, collectively promoting cell survival (Emara et al., 2010; Fu et al., 2009; Ivanov et al., 2011; Yamasaki et al., 2009). To assess whether ANG-mediated regulation of protein synthesis is tiRNA-dependent, we assessed bulk small RNA production by electrophoresis. LKS cells exhibited dramatically higher small RNA production over myeloid-restricted progenitors at baseline (FIG. 18A). tiRNA was undetectable in differentiated cell types under these conditions and was visible only when 15 μg total RNA was loaded (FIG. 17E). Importantly, addition of ANG led to markedly elevated tiRNA levels in LKS cells (FIG. 18A). Equal loading was affirmed by tRNA levels (indicated by arrows, FIG. 18A). Addition of ANG to lineage-positive cells did not result in an increase in tiRNA levels, in contrast to significantly elevated tiRNA levels following ANG treatment of HSPC (FIG. 17E, compared to FIG. 18A). Consistently, Ang−/− LKS cells exhibited reduced levels of tiRNA relative to WT LKS cells (FIG. 17F).

Further, an increase in tiRNA production in myeloid-restricted progenitors, but not in LKS cells, was observed following oxidative stress induced by sodium arsenite (FIG. 17G). Interestingly, ANG enhanced tiRNA in LKS cells under oxidative stress, but rather suppressed oxidative stress-induced tiRNA in myeloid-restricted progenitors. These results demonstrate that ANG differentially regulates tiRNA in LKS and myeloid-restricted progenitors under both homeostatic and stress conditions.

To ensure that the bulk small RNA reflect tiRNA, we analyzed the levels of a representative tiRNA, tiRNA-Gly-CCC, by Northern blotting in ANG-treated LKS cells and myeloid-restricted progenitors. tiRNA-Gly-GCC was previously shown to be expressed in hematopoietic tissues, including BM and spleen, but was neither examined in primitive hematopoietic cells nor functionally-validated (Dhahbi et al., 2013). FIG. 18B shows that tiRNA-Gly-GCC was significantly elevated in LKS cells, relative to myeloid-restricted progenitors, and was further enhanced by exogenous ANG. Together, these data identify tiRNA as a distinct RNA species that is abundantly expressed in HSPC and that is regulated by ANG. To determine whether tiRNA is responsible for restricted protein synthesis in HSPC, we transfected synthetic tiRNA-Gly-GCC in LKS and myeloid-restricted progenitors, and assessed protein synthesis in vivo using OP-Puro. As tiRNA requires its 5′-phosphate to suppress protein synthesis (Ivanov et al., 2011), we used an inactive, dephosphorylated synthetic tiRNA-Gly-GCC, termed (d)5′-P-tiRNA, as a negative control. Expectedly, transfection of active 5′-P tiRNA, but not of inactive (d)5′-P-tiRNA, led to a significant reduction in the rate of protein synthesis in both LKS cells and myeloid-restricted progenitors (FIG. 18C). Thus, tiRNA transfection phenocopies exogenous ANG on restriction of protein synthesis in LKS cells, as has been shown in FIG. 3B. We also found that myeloid and lymphoid progenitor colony formation was restricted upon transfection of whole BM with active 5′-P tiRNA (FIG. 17H). Moreover, transfection of active tiRNA led to upregulation of self-renewal and pro-survival genes, and downregulation of pro-apoptotic genes, in both LKS cells and myeloid-restricted progenitors (FIG. 18D).

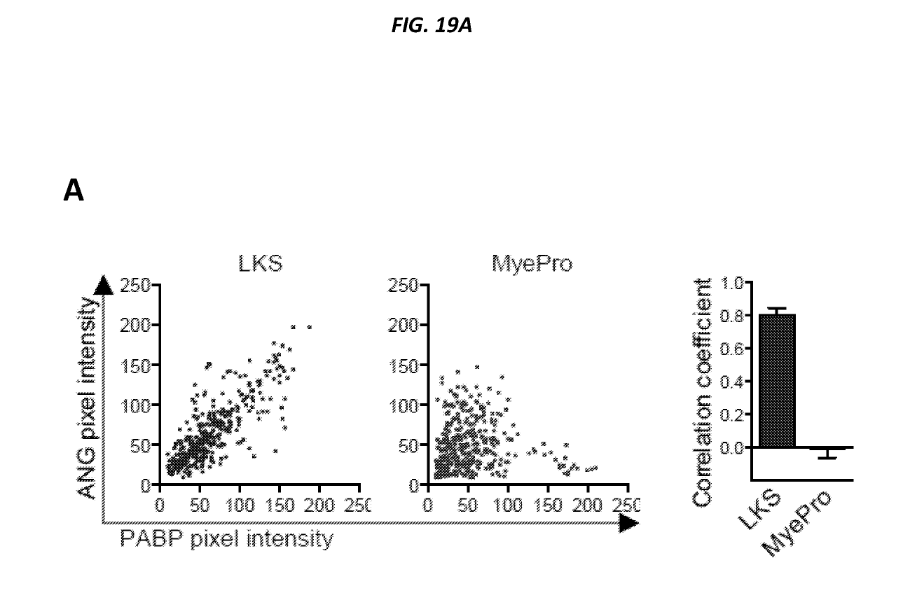

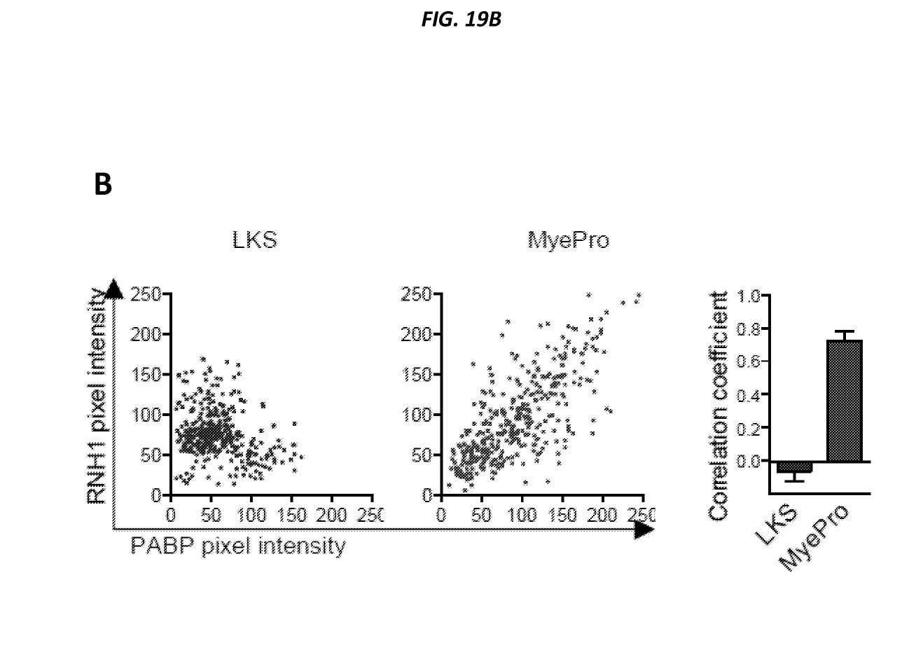

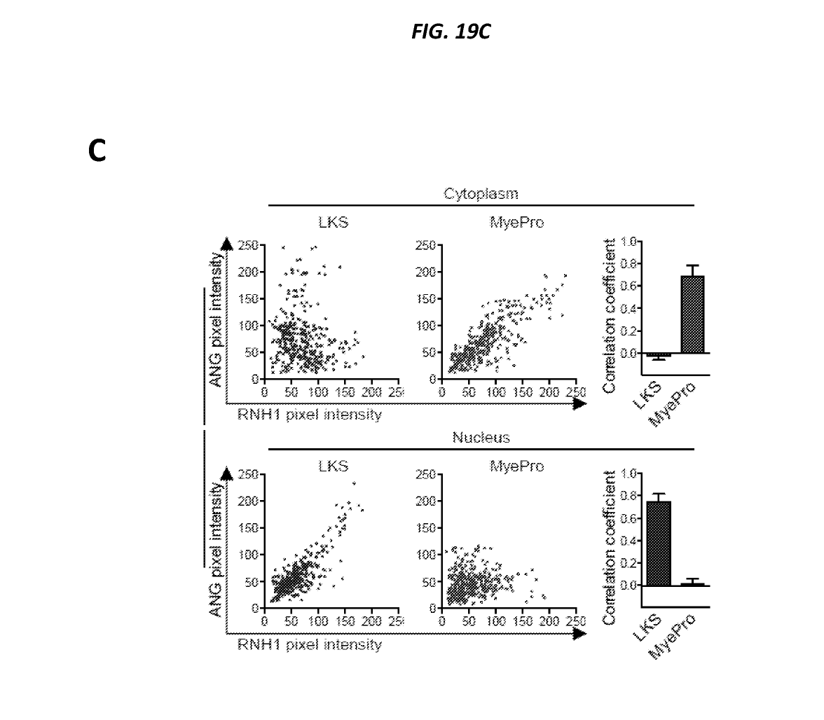

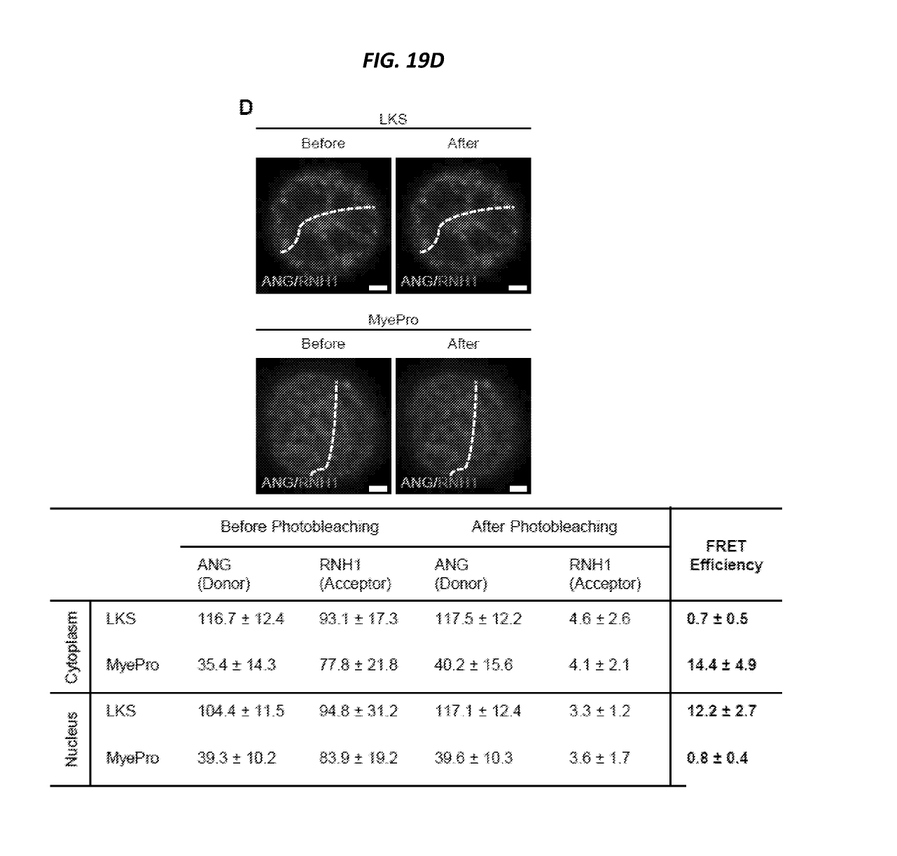

The exact subcellular compartment where tiRNA is produced by ANG is currently unknown, but it has been shown that tiRNA production is correlated to SG localization of ANG in stressed cells (Pizzo et al., 2013). The finding that ANG produces tiRNA and restricts protein synthesis only in LKS cells prompted us to examine differential localization of ANG in SGs between LKS and myeloid-restricted progenitors. It was found that ANG was colocalized with PABP, a SG marker, in LKS cells, but not in myeloid-restricted progenitors (FIG. 19A). Further, we found that RNase/ANG inhibitor 1 (RNH1), an endogenous ANG inhibitor that has been shown to regulate subcellular localization of ANG and tiRNA production (Pizzo et al., 2013), is localized in SGs in myeloid-restricted progenitors, but not in LKS cells (FIG. 19B). This opposing localization pattern of RNH1 and ANG was further examined by double immunofluorescence (FIG. 19C) and fluorescence resonance energy transfer (FRET, FIG. 19D), which showed that ANG and RNH1 colocalize and interact in the nucleus, but not cytoplasm of LKS cells, and in the cytoplasm but not nucleus of myeloid-restricted progenitors.

Thus, RNH1, which is known to stoichiometrically inhibit ANG with a femto-molar Kd (Lee et al., 1989), likely inhibits nuclear ANG but not cytoplasmic ANG in LKS cells, permitting tiRNA production, whereas it inhibits cytoplasmic ANG but not nuclear ANG in myeloid-restricted progenitors to allow rRNA transcription. It is conceivable that RNH1 is an integral player in the dichotomous regulation of ANG in HSPC versus myeloid-restricted progenitor cells. To assess whether tiRNA-mediated regulation of protein synthesis affects HSPC function, we transfected LKS cells with synthetic tiRNA and competitively transplanted those cells into WT hosts. Significantly, the inventors discovered enhanced long-term multi-lineage post-transplant reconstitution of cells transfected with synthetic tiRNA, relative to untreated LKS cells or cells transfected with inactive tiRNA (FIG. 18E). As ANG stimulates tiRNA production in LKS cells, these data strongly demonstrate that ANG may enhance the regenerative potential of HSPC by tiRNA-mediated alterations of protein synthesis.

ANG is a Pro-Regenerative Factor after Radio-Damage

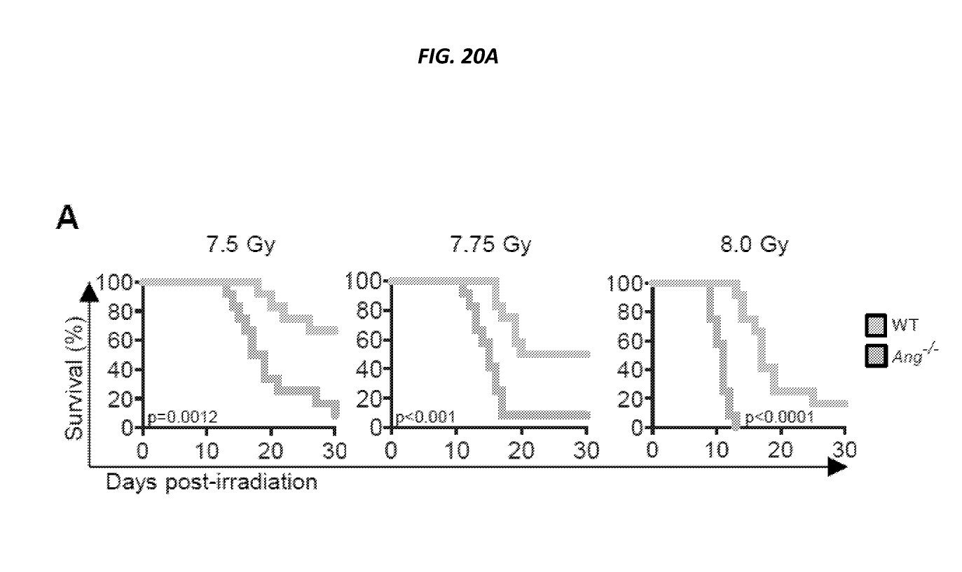

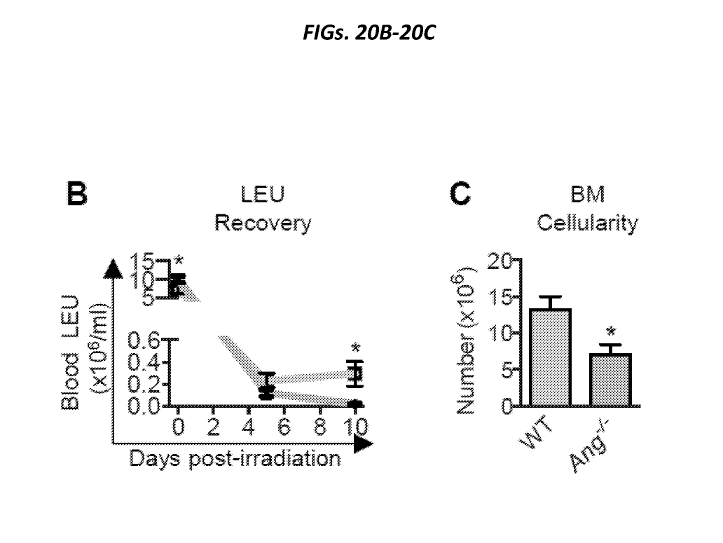

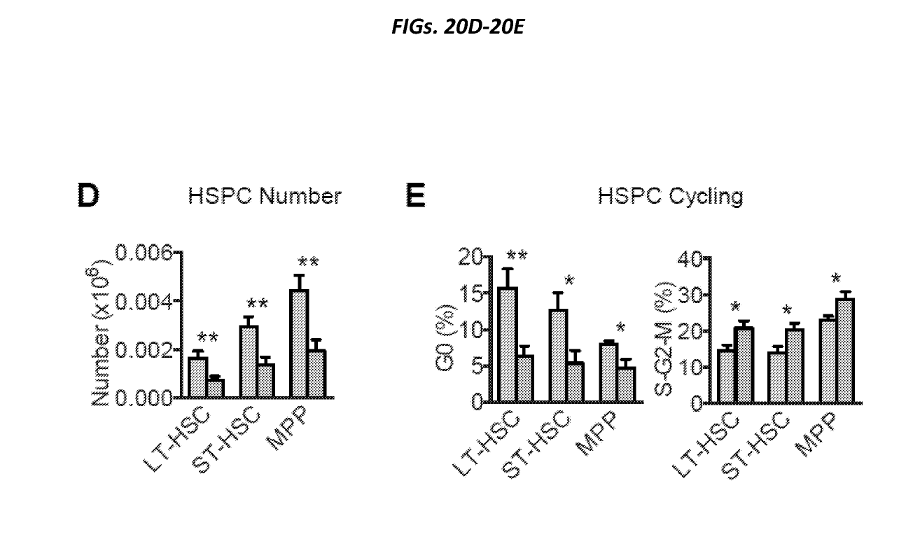

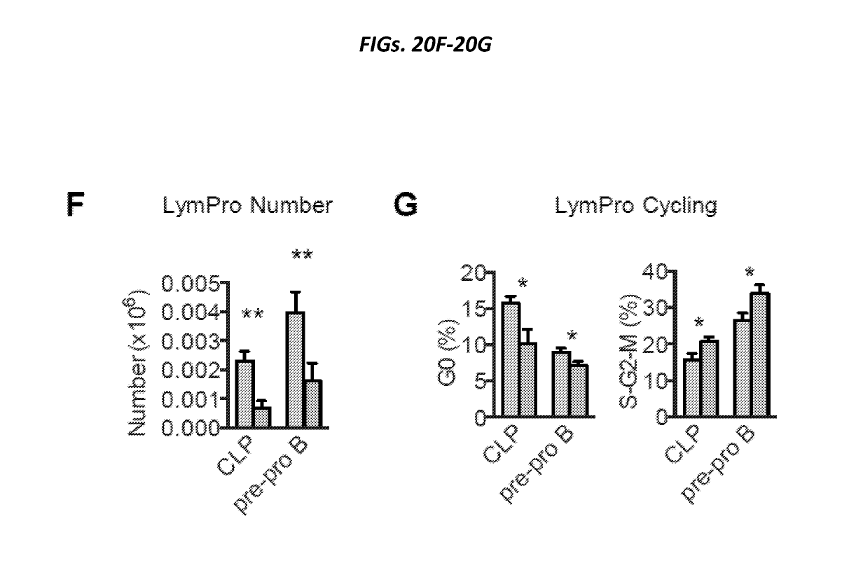

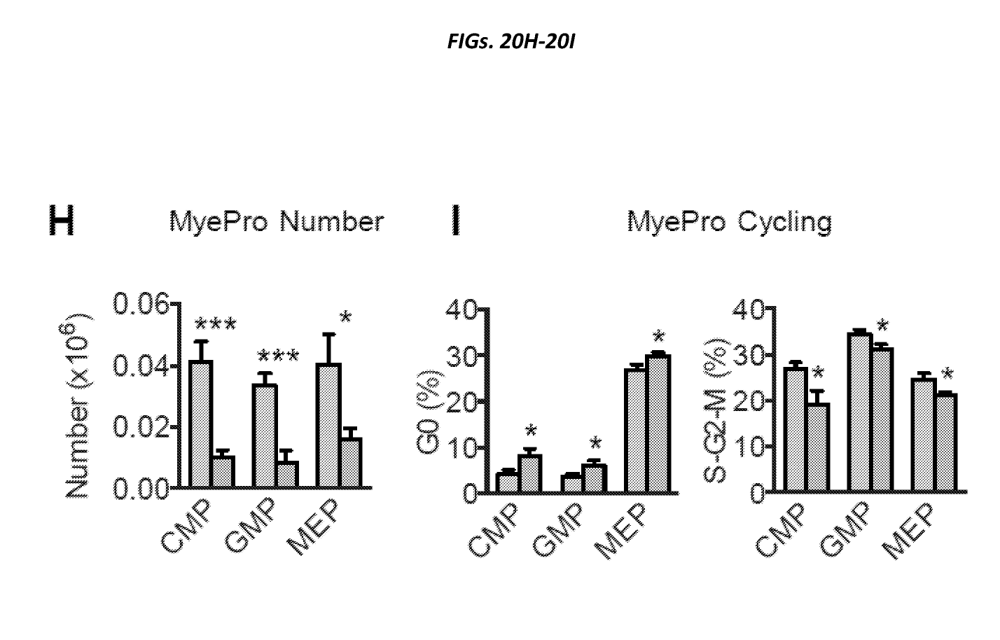

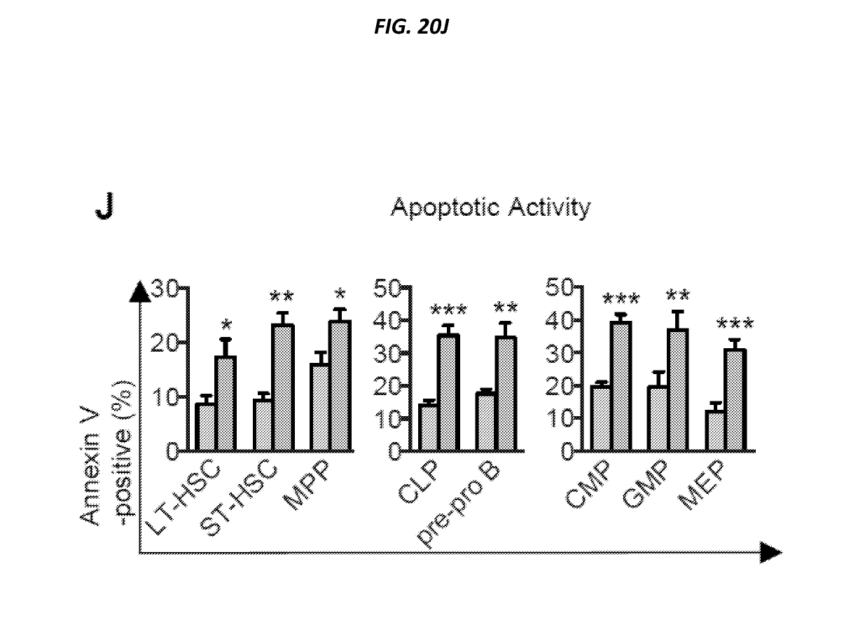

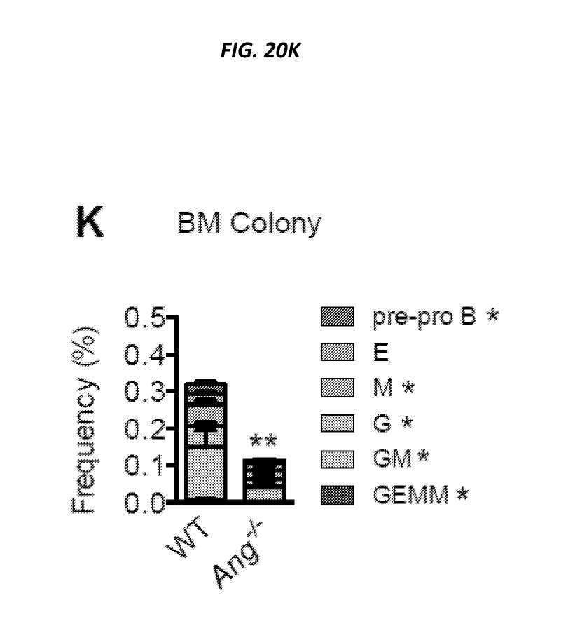

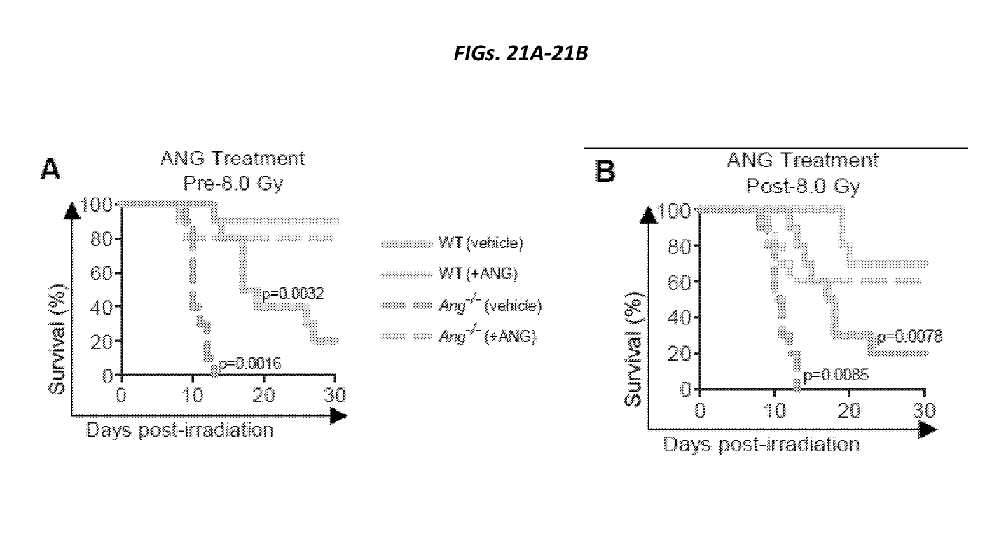

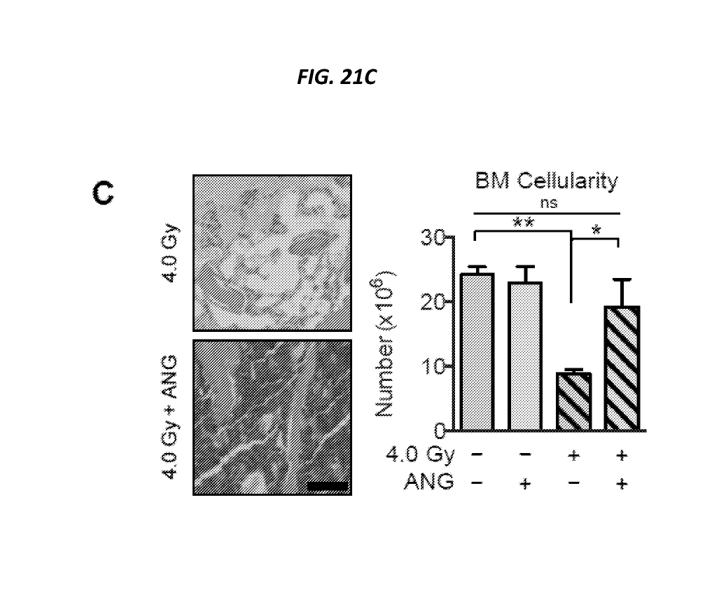

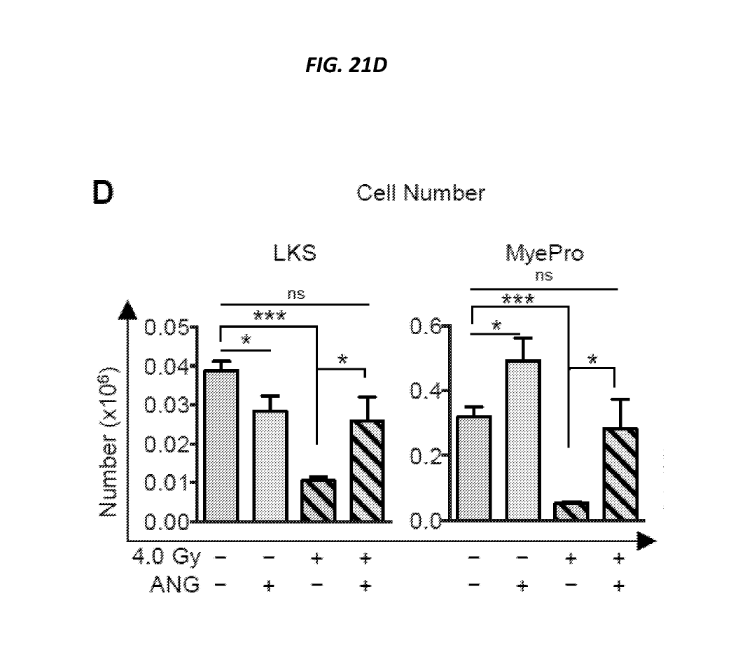

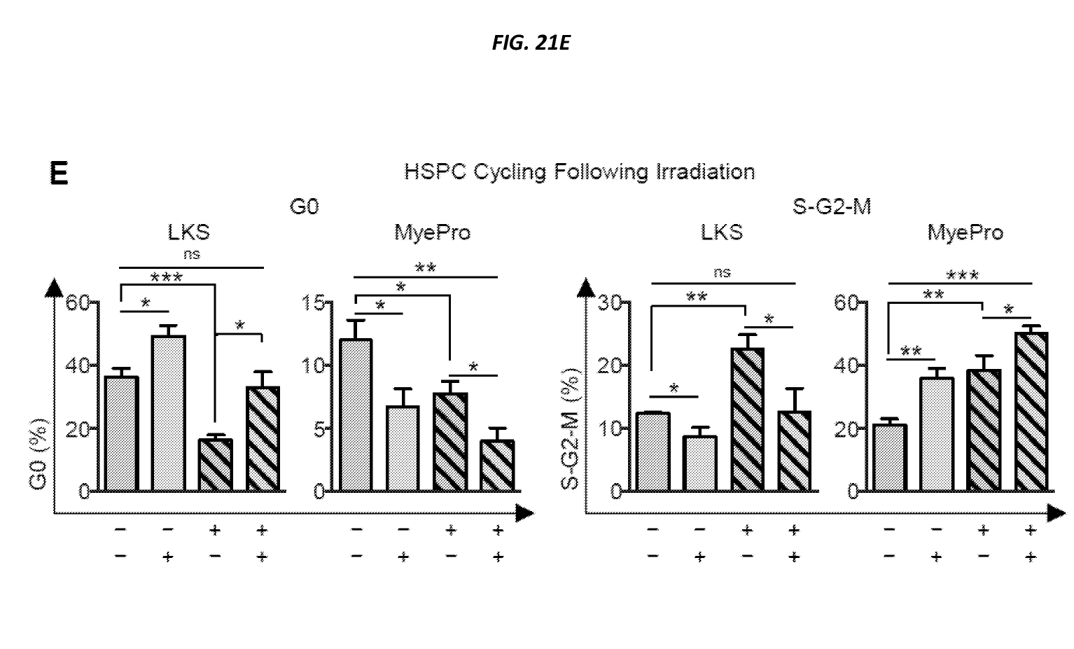

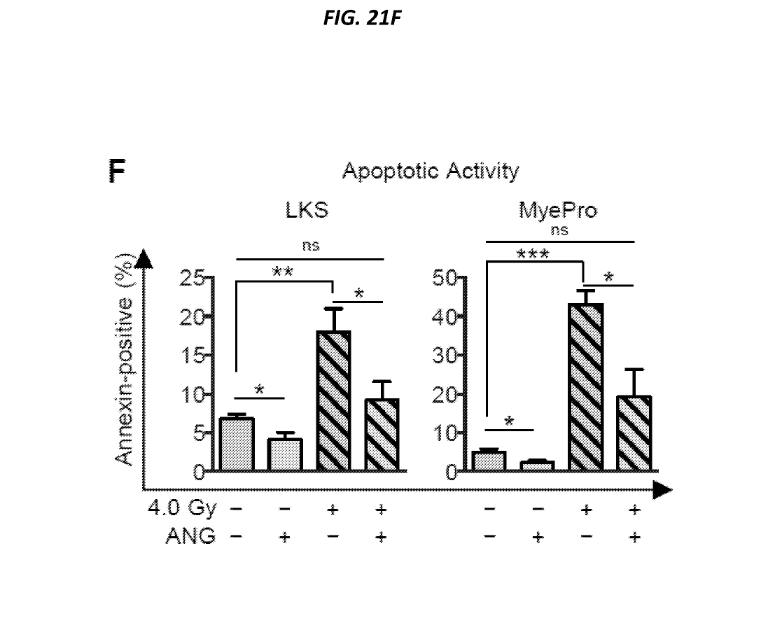

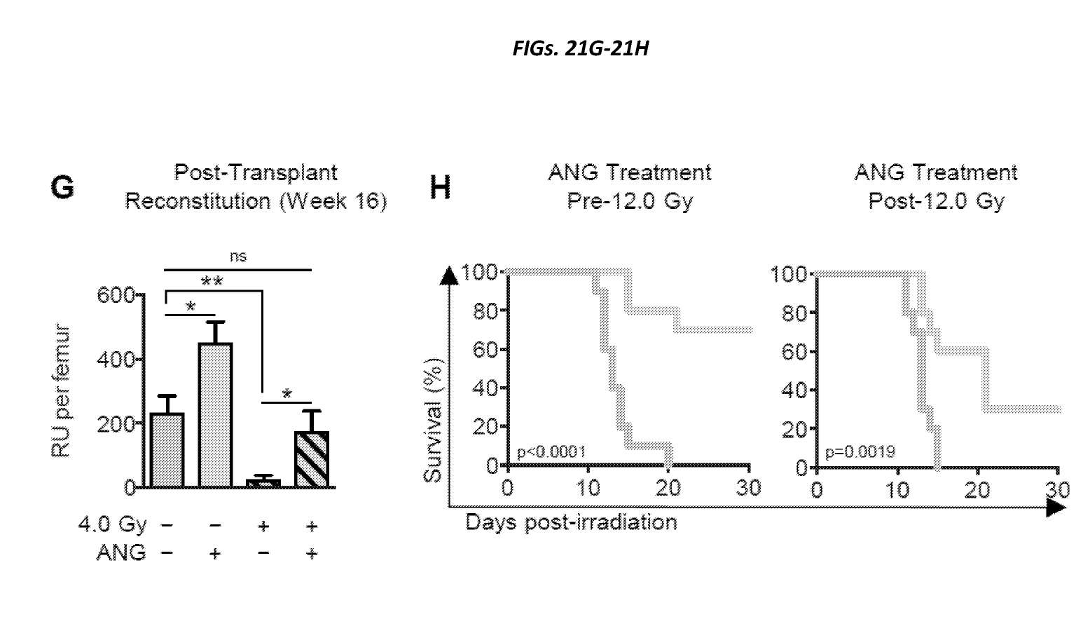

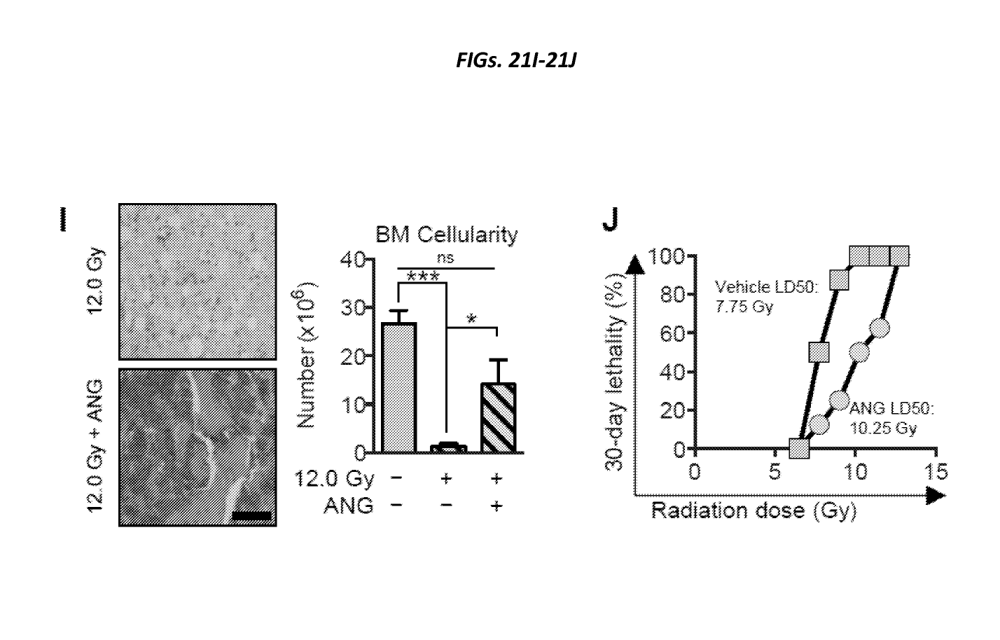

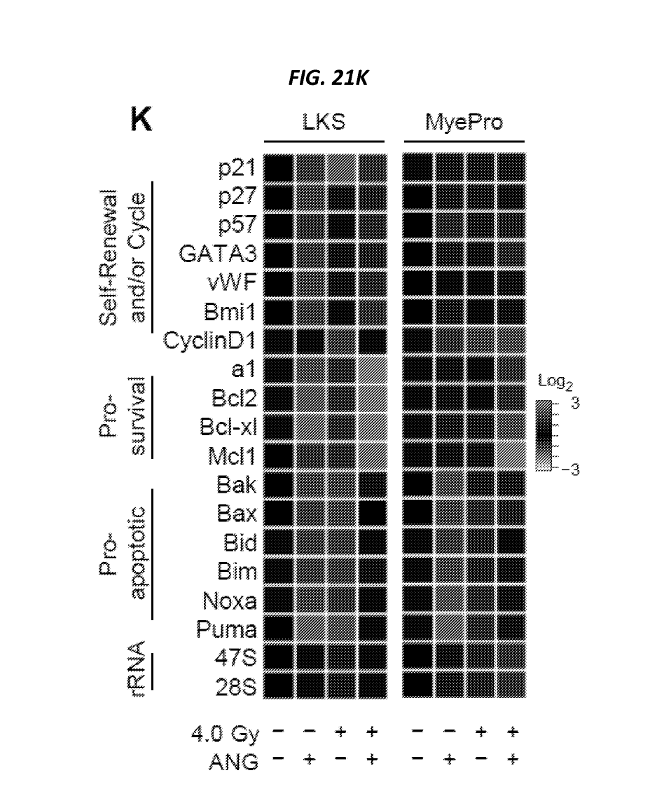

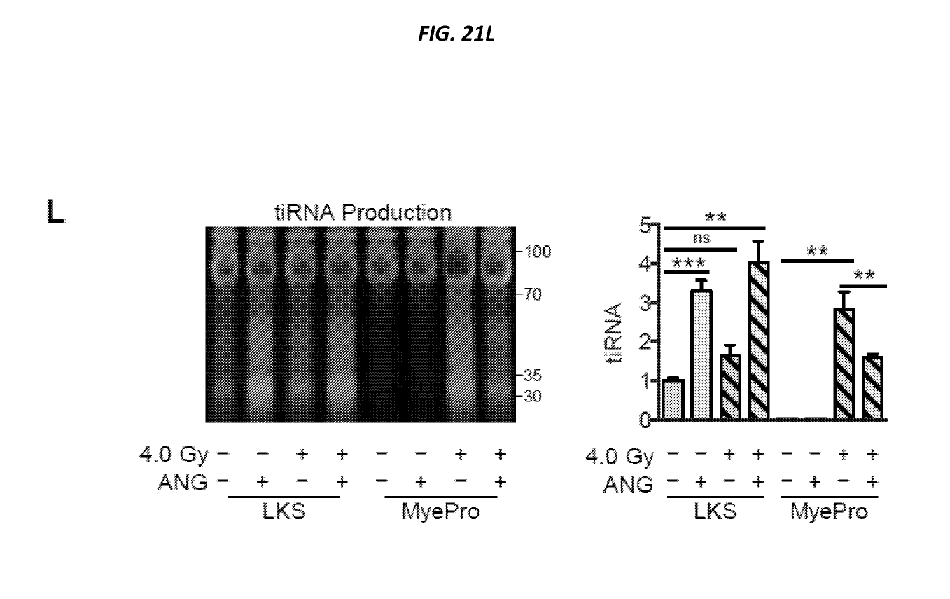

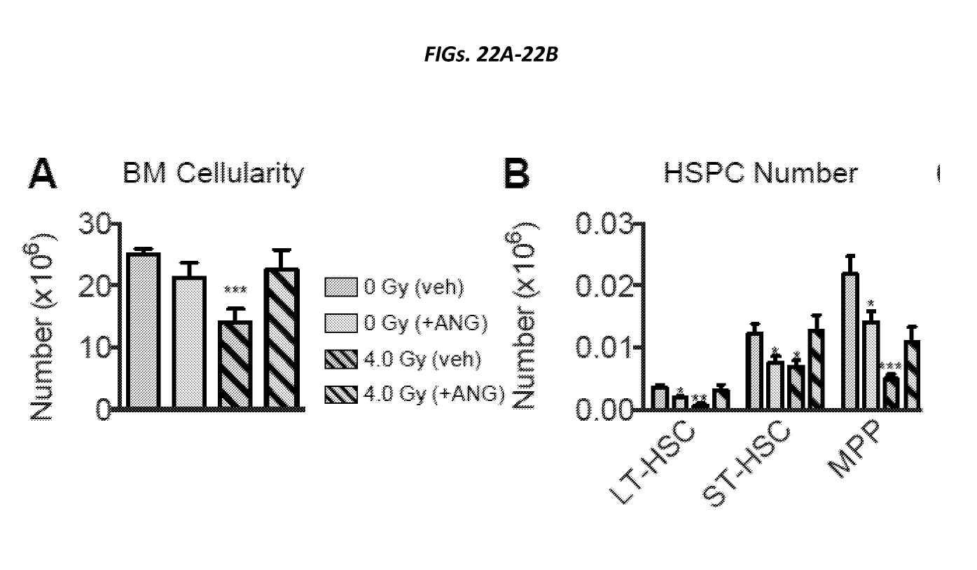

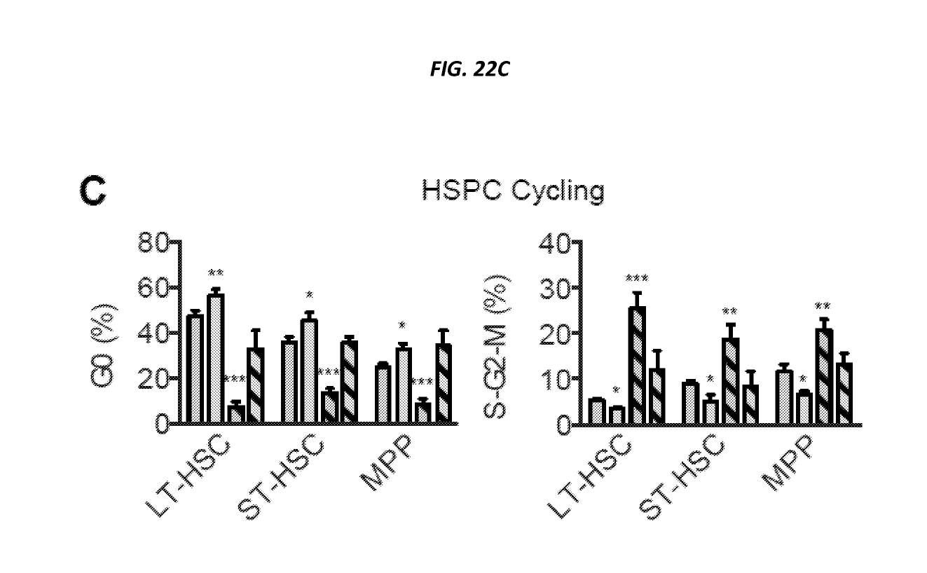

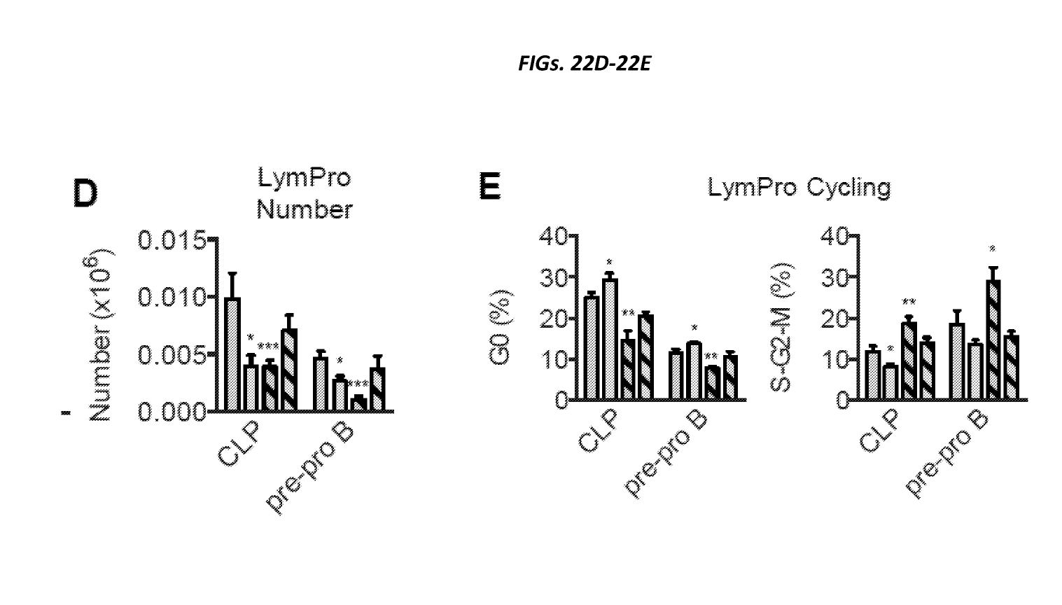

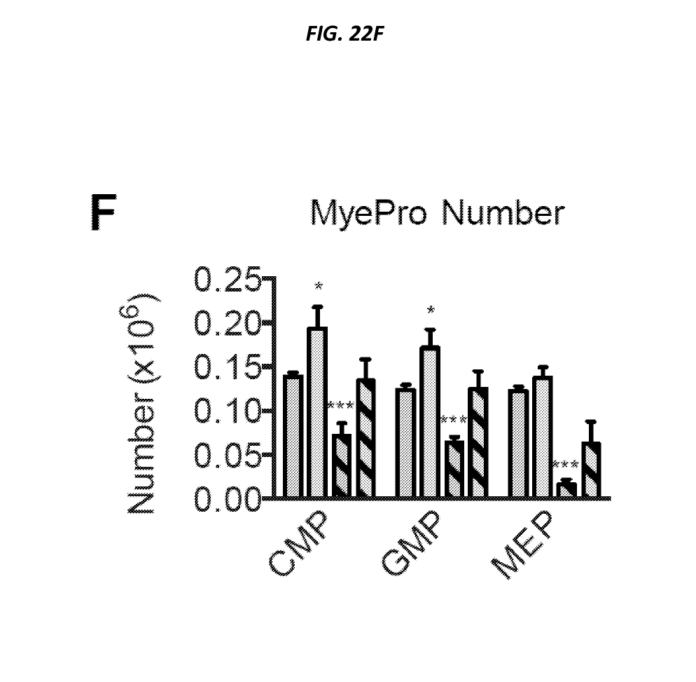

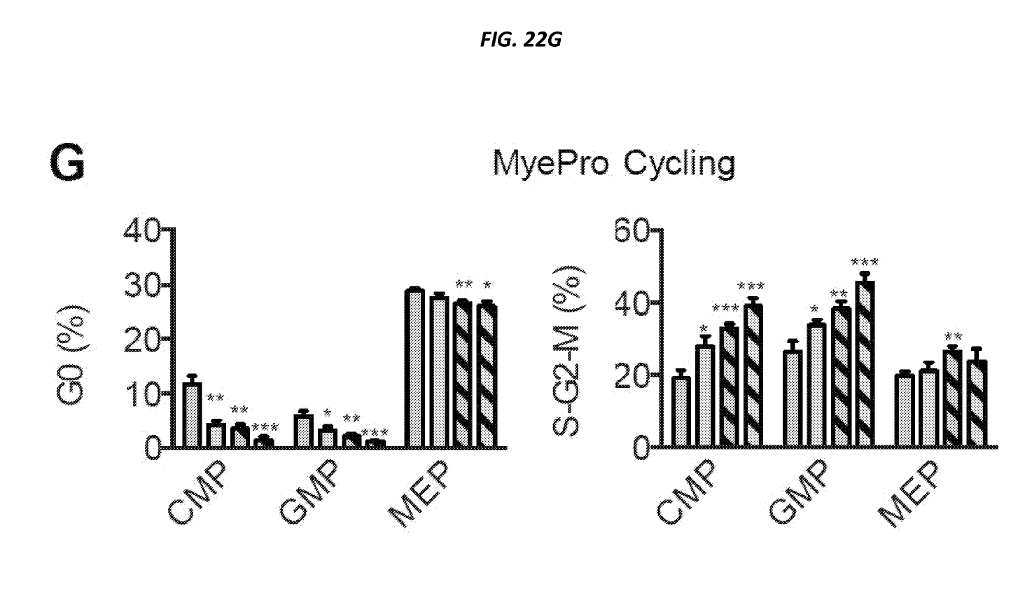

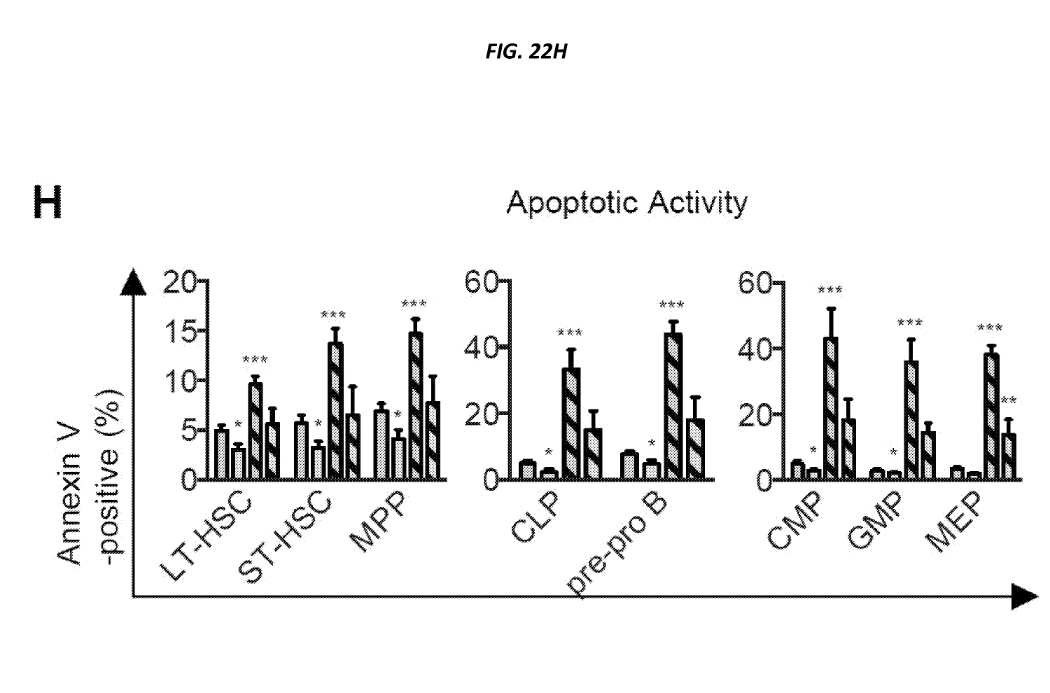

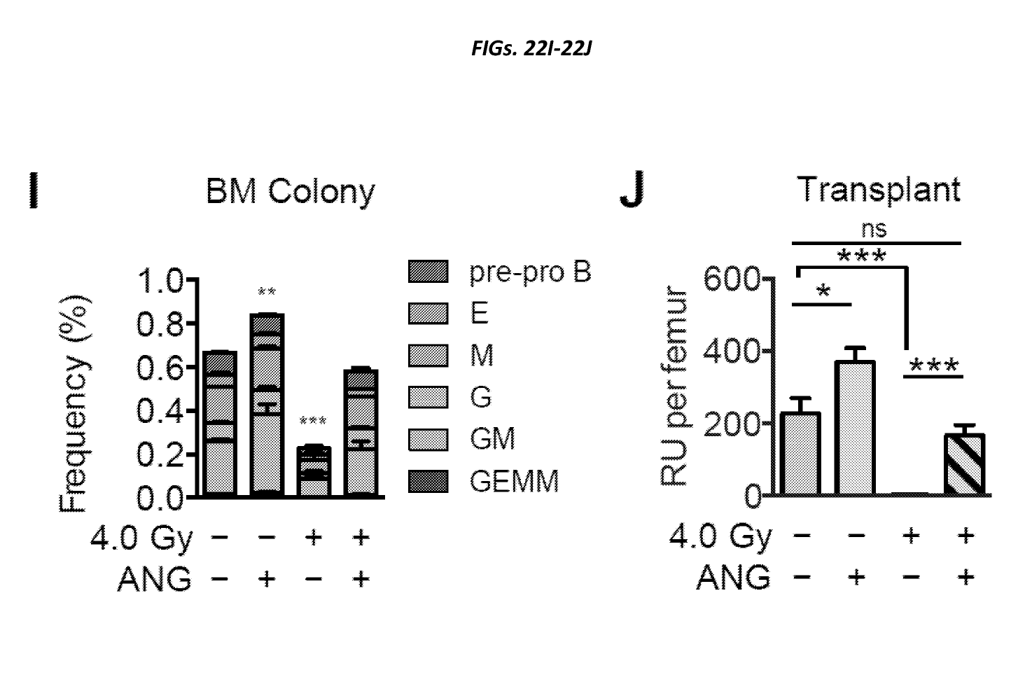

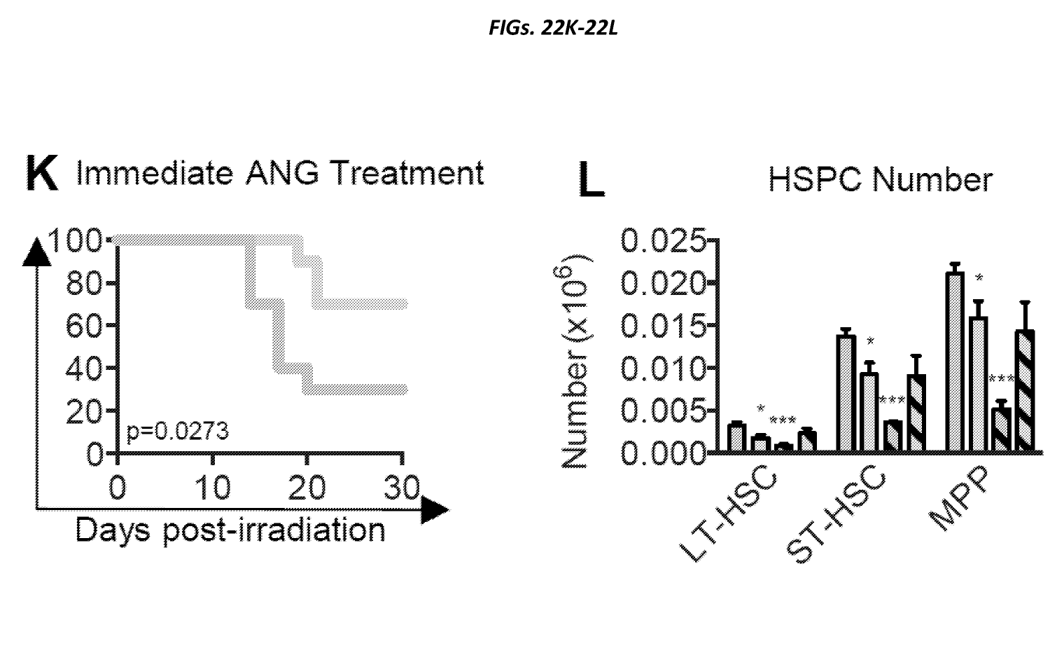

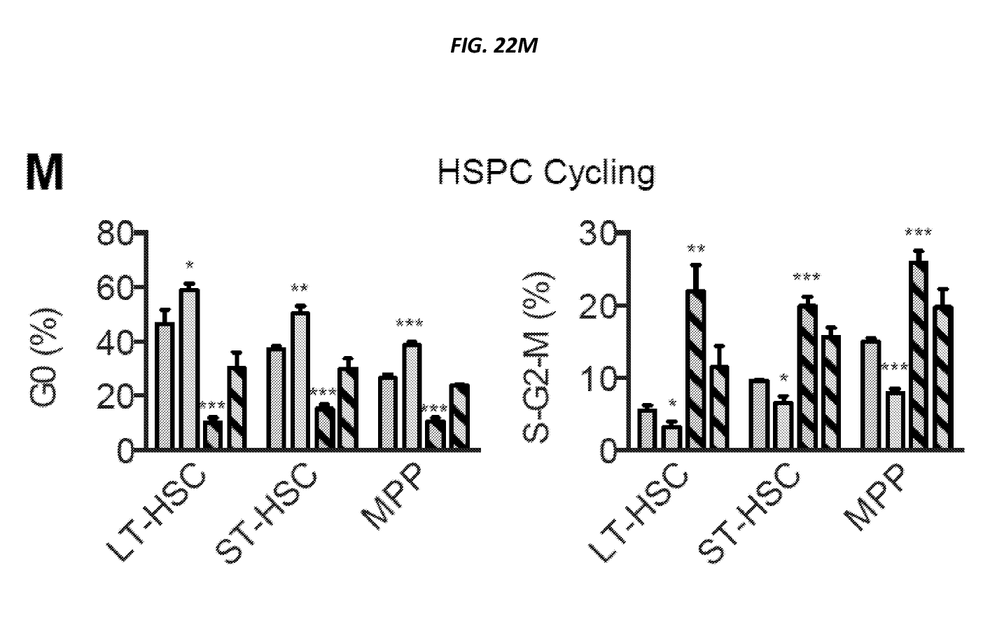

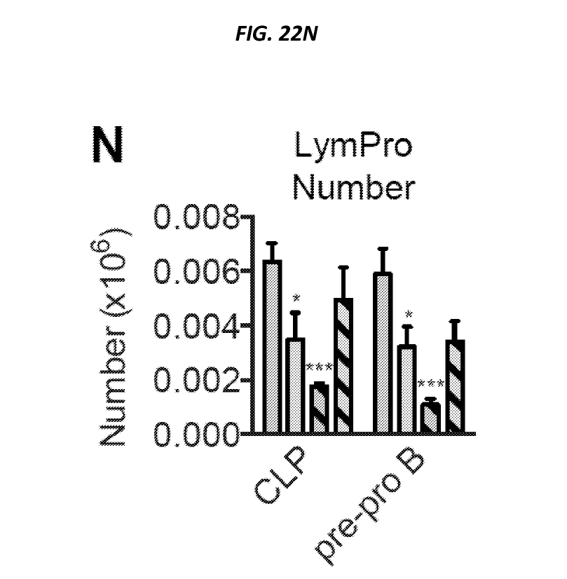

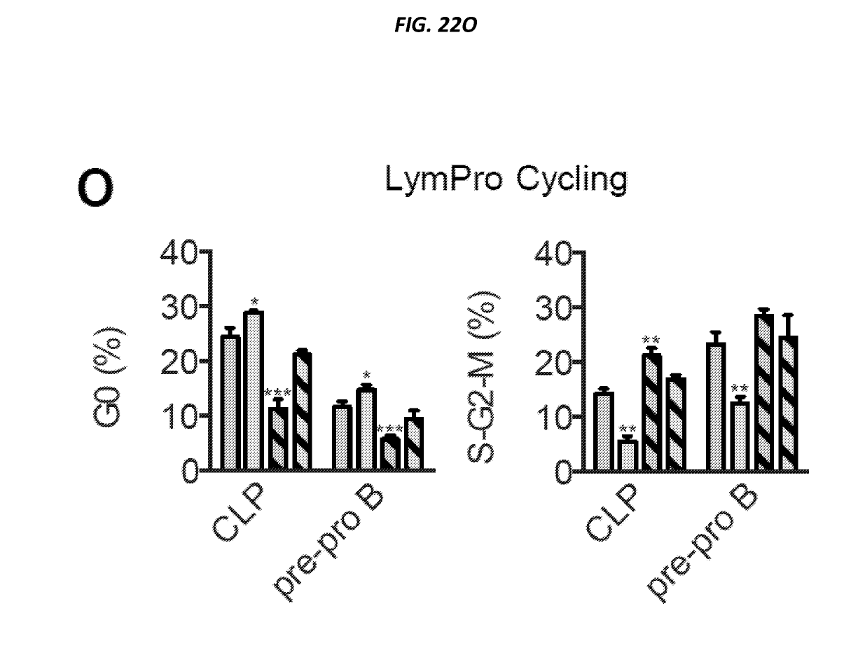

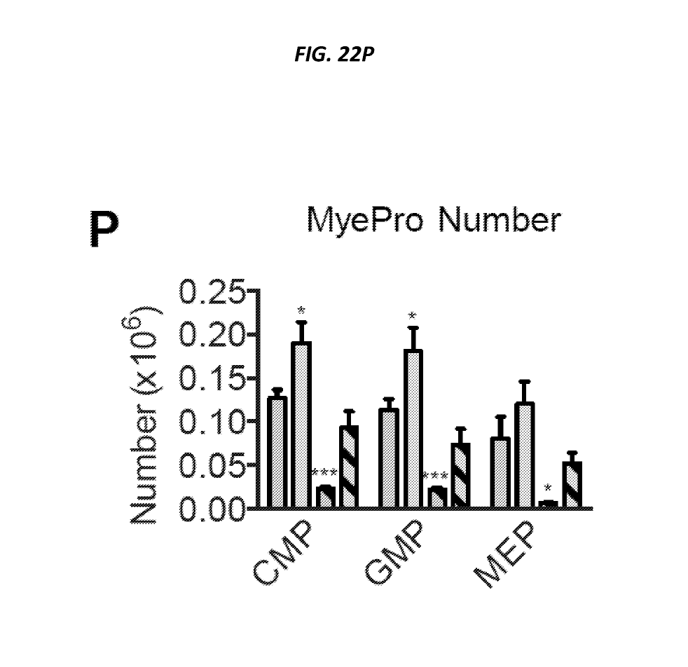

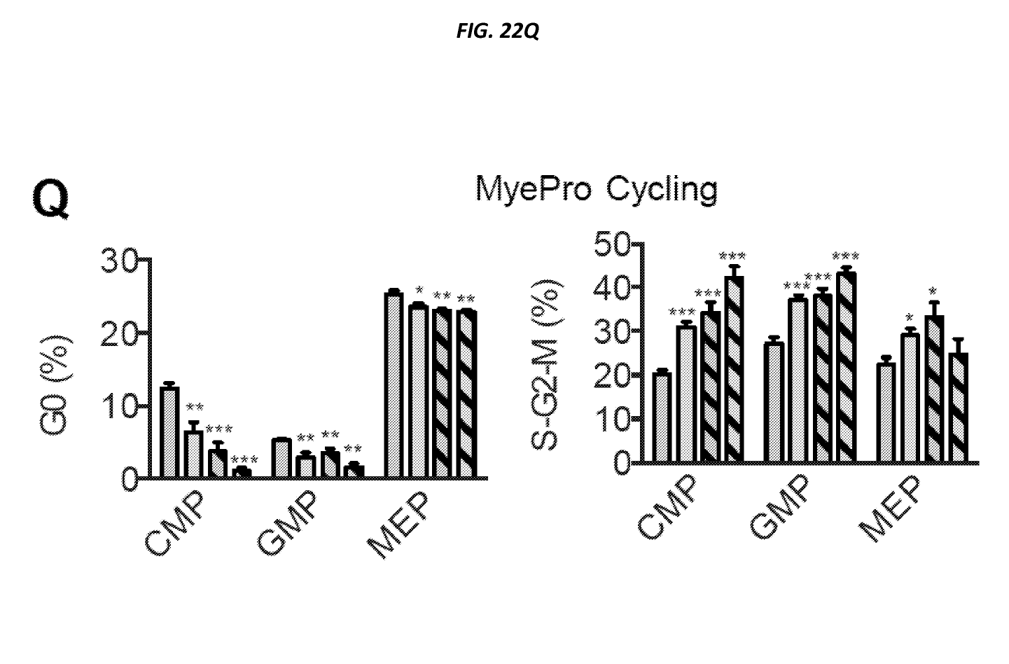

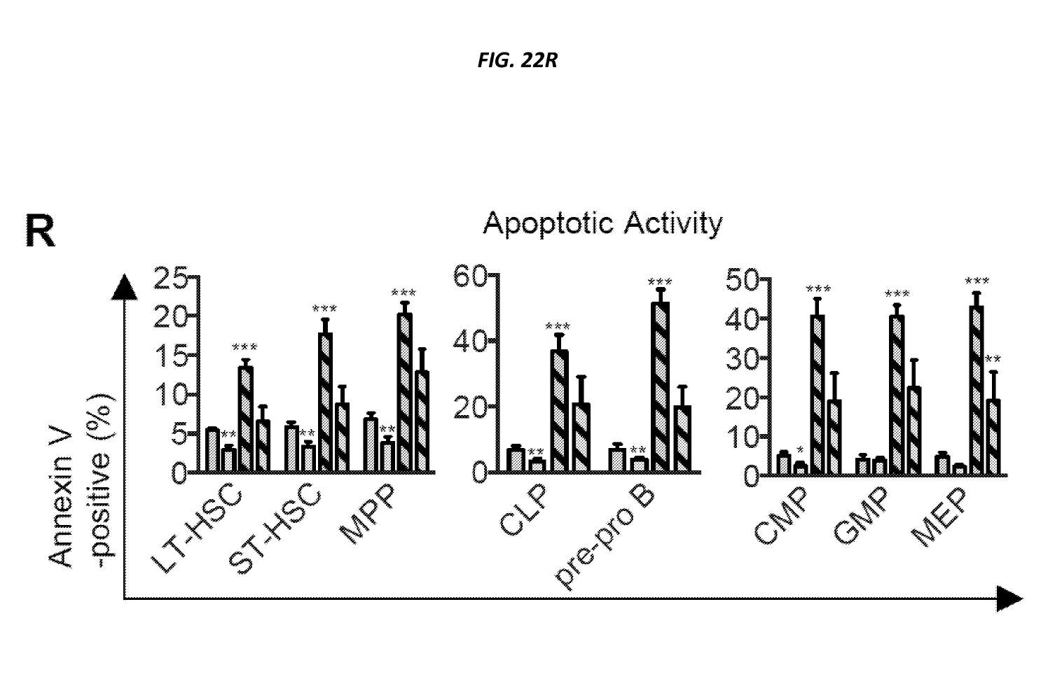

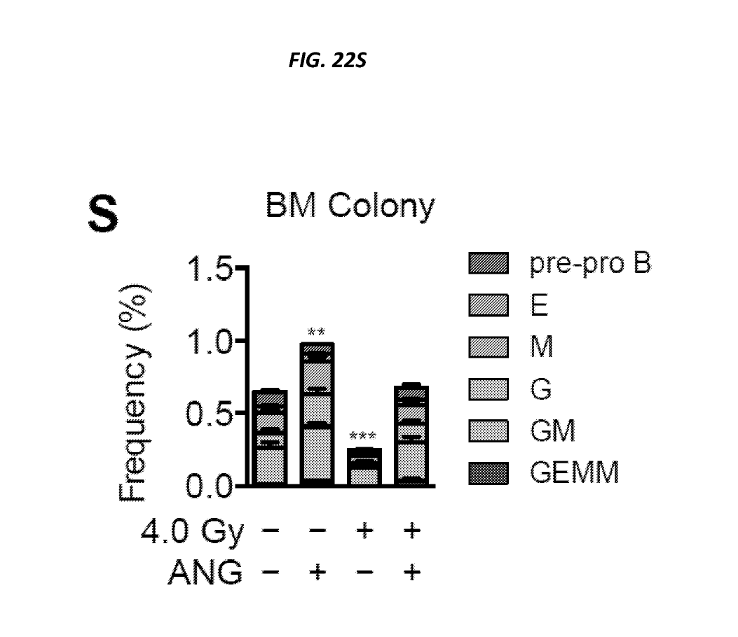

To begin to assess the pro-regenerative role of ANG, we first examined the function of ANG in the context of radiation-induced cell damage. Ang−/− mice displayed reduced survival following exposure to various doses of γ-radiation (FIG. 20A), accompanied by decreased blood leukocyte recovery, reduced total BM cellularity, reduced HSPC and lymphoid-restricted progenitor number, and more active cycling (FIGS. 20B-20G, Table 7). These data are consistent with the quiescence-inducing effect of ANG on HSPC, as discussed previously. In contrast, myeloid-restricted progenitors in Ang−/− mice showed reduced cell number, but restricted proliferation following total body irradiation (TBI) (FIG. 20H-20I) indicating that, normally, ANG would promote myeloid reconstitution. Ang−/− mice also demonstrated increased apoptosis in all cell types, as well as reduced lymphoid and myeloid colony formation in response to γ-radiation (FIG. 20J-20K). Together, these data demonstrate that ANG deficiency leads to reduced animal survival, accompanied by diminished cell number, perturbed cell cycling, and elevated apoptotic activity in hematopoietic cells. To determine whether treatment with ANG enhances survival, WT or Ang−/− mice were pretreated with ANG daily for three successive days and irradiated mice with 8.0 Gy 24 hours following the final ANG treatment. Significantly, the 30-day survival rate increased from 20% to 90% after ANG treatment, indicating that ANG is radioprotective (FIG. 21A). Importantly, 80% of Ang−/− mice also survived following ANG pretreatment whereas 100% of untreated Ang−/− mice died. Pre-treatment with ANG protected against TBI (4 Gy)-induced loss of cell number and increase in cycling of HSPC and lymphoid-restricted progenitors (FIGS. 22A-22E, Table 8). In contrast, ANG pre-treatment not only prevented the loss of myeloid-restricted progenitors but also promoted their proliferation (FIGS. 22F-22G), again demonstrating a dichotomous effect of ANG in regulating HSPC and myeloid-restricted progenitors under stress conditions. Moreover, ANG protected against TBI-induced apoptosis in all cell types, and led to enhanced colony formation and post-transplant reconstitution (FIGS. 22H-22J). Together, these data demonstrate the protective function of ANG against radiation-induced BM damage, likely through induction of HSPC quiescence and promotion of myeloid-restricted progenitor proliferation.

To assess a potential therapeutic use of ANG as a radio-mitigating agent, we irradiated mice with 8.0 Gy and began ANG treatment 24 hours later. Significantly, the majority of ANG-treated mice survived, including ANG-treated Ang−/− mice, suggesting that ANG has radio-mitigating capabilities (FIG. 21B). A similar enhancement of survival was observed when ANG treatment was begun immediately following irradiation (FIG. 22K). Importantly, treatment with ANG 24 hours post-irradiation prevented TBI-induced reduction of overall BM cellularity, as well as LKS cells and myeloid-restricted progenitors (FIGS. 21C-21D, Table 8). Consistent with its dichotomous role in cell cycle kinetics, ANG restricted proliferation of LKS cells, and simultaneously enhanced proliferation of myeloid-restricted progenitors (FIG. 21E). Further, ANG prevented TBI-induced apoptosis in both LKS cells and myeloid-restricted progenitors (FIG. 21F). These effects on cell number, cycling, and apoptosis were also apparent using more specific cell-surface markers for stem and progenitor cell populations (FIGS. 22L-22R). Significantly, defects in colony formation and post-transplant reconstitution can be rescued by in vivo ANG treatment (FIGS. 21G, 22S). We also assessed the protective and mitigative effect of ANG in lethally-irradiated animals and found that ANG treatment either before or after lethal irradiation improved survival, and enhanced BM cellularity, as well as peripheral blood content (FIGS. 21H-21I, Table 9). Moreover, ANG significantly increased the LD50 when treatment was begun 24 hours post-TBI (FIG. 21J). Further, treatment with ANG upregulated pro-self-renewal genes in LKS cells and led to enhanced pro-survival transcript levels and reduced pro-apoptotic transcripts in both LKS cells and myeloid-restricted progenitors (FIG. 21K). Importantly, ANG treatment enhanced rRNA transcription only in myeloid-restricted progenitors (FIG. 21K) and tiRNA production only in LKS cells (FIG. 21L) following TBI, consistent with its dichotomous role in promoting and restricting cell proliferation in these two cell types. Together, these results establish a model by which ANG simultaneously stimulates proliferation of rapidly-responding myeloid-restricted progenitors and preserves HPSC stemness, in association with enhanced hematopoietic regeneration and improved survival.

TABLE 7
Cell counts for irradiated Ang^{-/-} mice

| Cohort | Organ | Parameter | Unit | WT | Ang^{-/-} |
|---|---|---|---|---|---|
| WT vs | Bone | Mac1⁺Gr1⁺ | 10⁸/femur | 2.79 ± 0.54 | 0.98 ± 0.19* |
| Ang^{-/-} | Marrow | Ter119⁺ | 10⁸/femur | 1.22 ± 0.17 | 0.65 ± 0.10* |
| | | B220⁺ | 10⁸/femur | 2.15 ± 0.29 | 1.24 ± 0.28* |
| | | CD3e⁺ | 10⁸/femur | 0.22 ± 0.03 | 0.11 ± 0.02* |

p-value relative to WT group
n = 6

TABLE 8
Cell counts for irradiated mice

| Cohort | Organ | Parameter | Unit | Untreated | +ANG | 4 Gy | 4 Gy + ANG |
|---|---|---|---|---|---|---|---|
| ANG | Bone | Mac1⁺Gr1⁺ | 10⁶/femur | 11.3 ± 0.65 | 11.2 ± 1.17 | 6.39 ± 1.13 ** | 10.7 ± 1.54 |
| Treatment | Marrow | Ter119⁺ | 10⁶/femur | 3.80 ± 0.17 | 3.49 ± 0.43 | 1.64 ± 0.38 *** | 2.27 ± 0.55 * |
| Pre- | | B220⁺ | 10⁶/femur | 6.04 ± 0.33 | 5.10 ± 0.72 | 3.26 ± 0.45 ** | 4.57 ± 0.65 |
| Irradiation | | CD3e⁺ | 10⁶/femur | 0.58 ± 0.02 | 0.58 ± 0.17 | 0.36 ± 0.06 *** | 0.62 ± 0.18 |
| ANG | Bone | Mac1⁺Gr1⁺ | 10⁶/femur | 11.9 ± 0.44 | 11.4 ± 1.38 | 3.19 ± 0.23 *** | 7.98 ± 1.92 |
| Treatment | Marrow | Ter119⁺ | 10⁶/femur | 2.99 ± 0.57 | 2.69 ± 0.41 | 0.78 ± 0.07 ** | 1.55 ± 0.41 |
| Post- | | B220⁺ | 10⁶/femur | 5.68 ± 0.33 | 4.54 ± 0.58 | 2.17 ± 0.20 ** | 4.50 ± 1.14 |
| Irradiation | | CD3e⁺ | 10⁶/femur | 0.54 ± 0.05 | 0.51 ± 0.15 | 0.28 ± 0.03 *** | 0.41 ± 0.10 |

p-value relative to untreated group
n = 6

TABLE 9
Cell counts for lethally-irradiated mice with ANG pre-treatment

| | | | Day 0 | | Day 5 | | Day 10 | |
|---|---|---|---|---|---|---|---|---|
| Organ | Parameter | Unit | Vehicle | +ANG | Vehicle | +ANG | Vehicle | +ANG |
| Blood | WBC | 10³/μl | 6.93 ± 0.069 | 6.76 ± 0.69 | 0.99 ± 0.36 | 3.50 ± 0.82** | 0.99 ± 0.59 | 4.23 ± 1.09** |
| | LYM | 10³/μl | 4.99 ± 0.67 | 4.92 ± 0.46 | 0.54 ± 0.20 | 1.18 ± 0.28 | 0.57 ± 0.32 | 1.40 ± 0.36 |
| | MON | 10³/μl | 0.47 ± 0.11 | 0.42 ± 0.08 | 0.03 ± 0.01 | 0.27 ± 0.06** | 0.09 ± 0.05 | 0.30 ± 0.08* |
| | NEU | 10³/μl | 1.47 ± 0.29 | 1.44 ± 0.21 | 0.41 ± 0.15 | 2.05 ± 0.48** | 0.32 ± 0.21 | 2.53 ± 0.65** |
| | PLT | 10³/μl | 826 ± 55 | 845 ± 69 | 243 ± 53 | 780 ± 97*** | 176 ± 57 | 555 ± 122* |
| | Mac1⁺Gr1⁺ | 10³/μl | 0.75 ± 0.09 | 0.75 ± 0.08 | 0.06 ± 0.02 | 0.48 ± 0.11 ** | 0.004 ± 0.001 | 0.83 ± 0.22 ** |
| | B220⁺ | 10³/μl | 3.61 ± 0.448 | 3.55 ± 0.34 | 0.04 ± 0.01 | 0.72 ± 0.25 * | 0.050 ± 0.039 | 0.88 ± 0.26 ** |
| | CD3e⁺ | 10³/μl | 1.94 ± 0.25 | 1.88 ± 0.18 | 0.02 ± 0.01 | 1.49 ± 0.49 ** | 0.005 ± 0.004 | 1.73 ± 0.58 ** |

n = 10
Dose: 12.0 Gy
ANG Treatment: 125 mg/kg, three times daily pre-irradiation

### Example 8

Ex Vivo Treatment of LT-HSCs with Recombinant ANG Enhances Post-Transplant Reconstitution

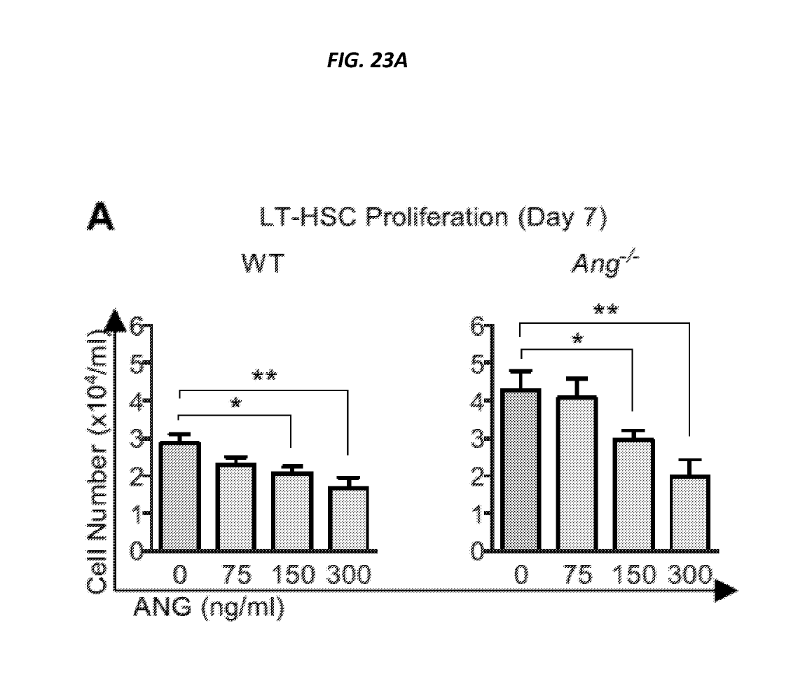

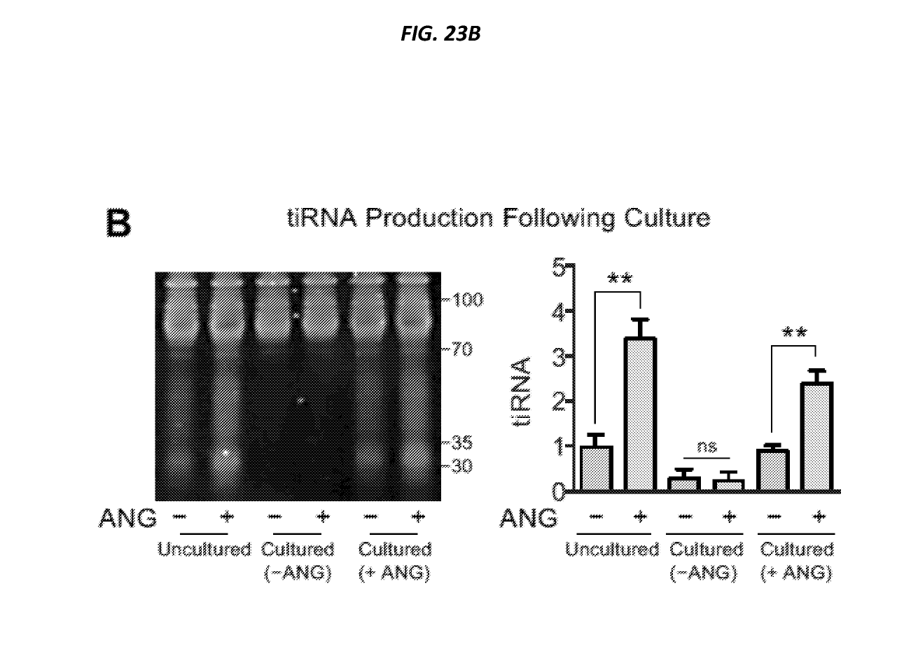

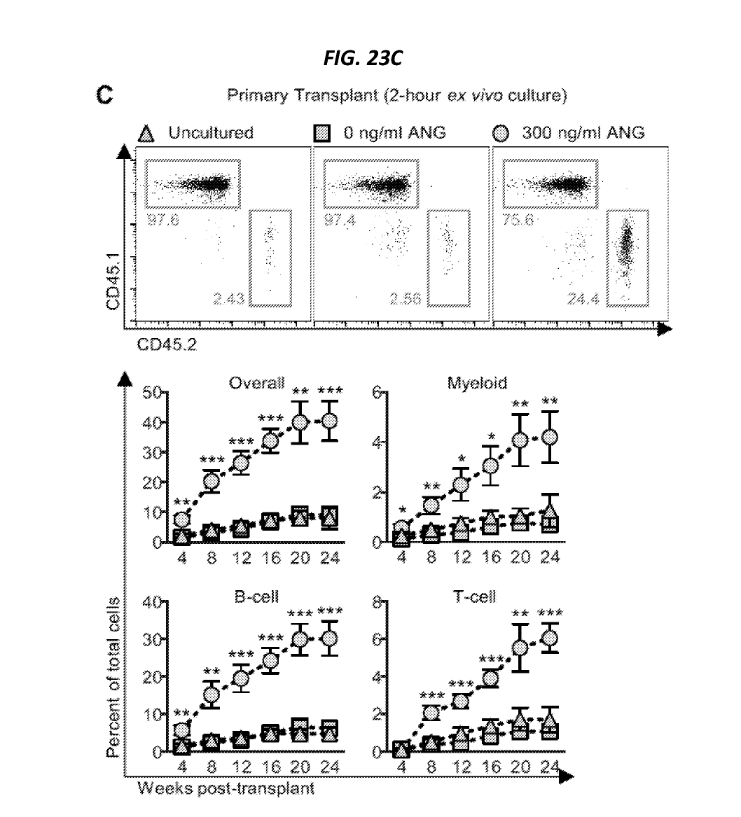

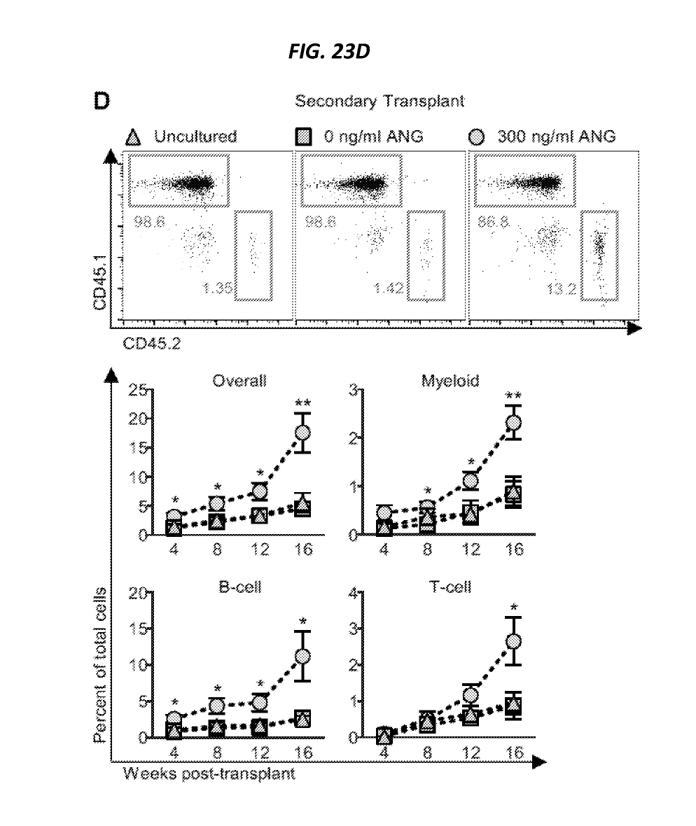

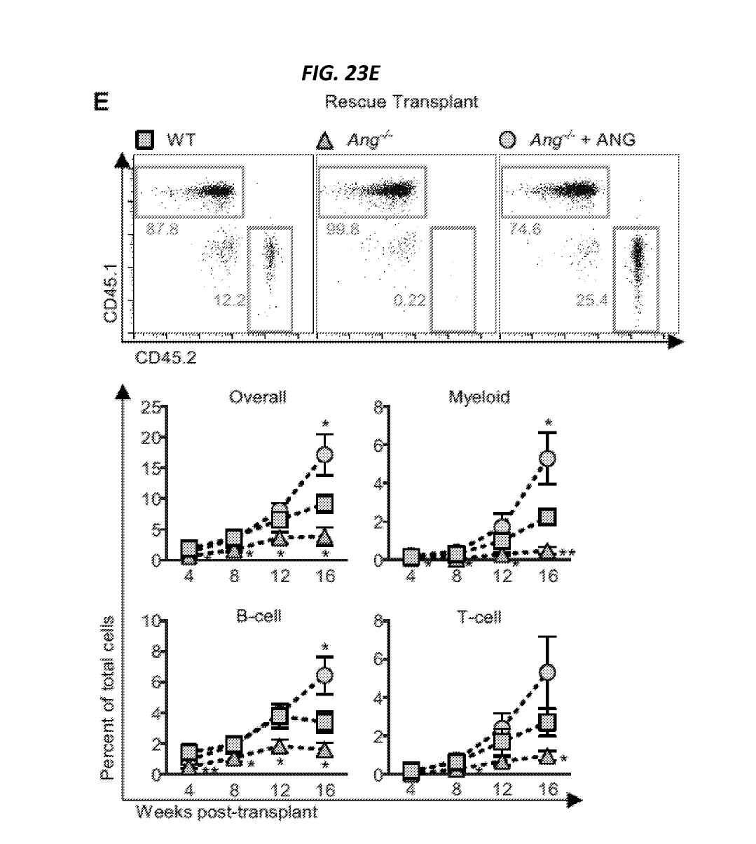

The in vivo (FIGS. 14C, 15H-K) and in vivo (FIGS. 20, 21, 22) activity of ANG in preserving HSPC stemness and in enhancing regeneration prompted us to assess its capacity in improving SCT and its potential for clinical development. Treatment of LT-HSCs with ANG in culture for 7 days led to a dose-dependent decrease of cell proliferation in WT and Ang−/− cells (FIG. 23A), consistent with its ability to restrict HSC proliferation. Significantly, LKS cells cultured in the absence of ANG resulted in a reduction of tiRNA expression relative to uncultured cells (FIG. 23B). In contrast, cells cultured in the presence of ANG not only maintained baseline tiRNA levels, but also their responsiveness to further ANG treatment.

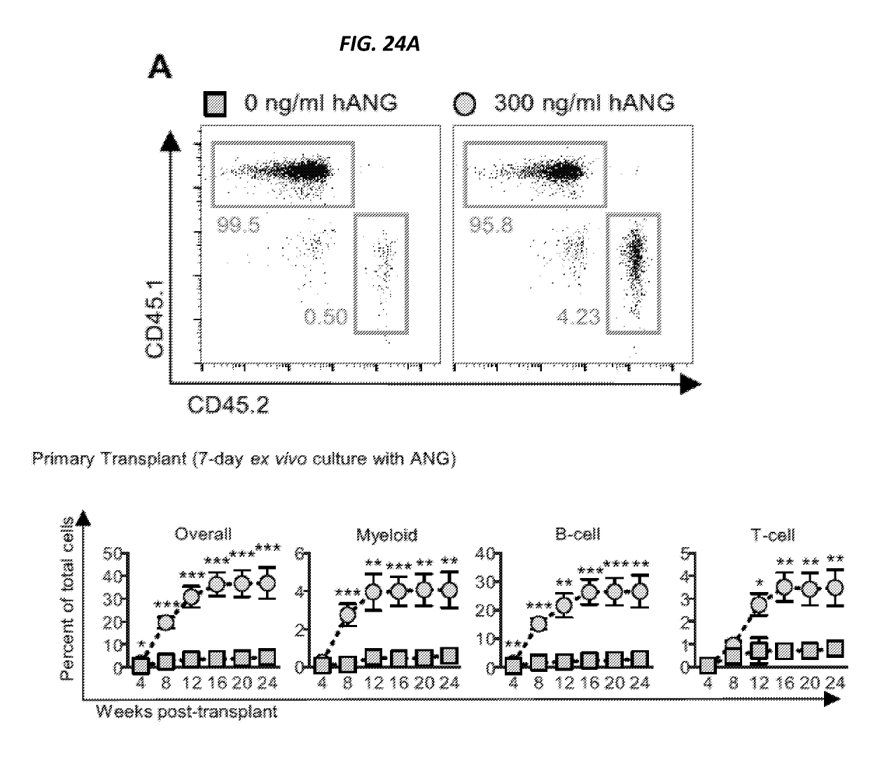

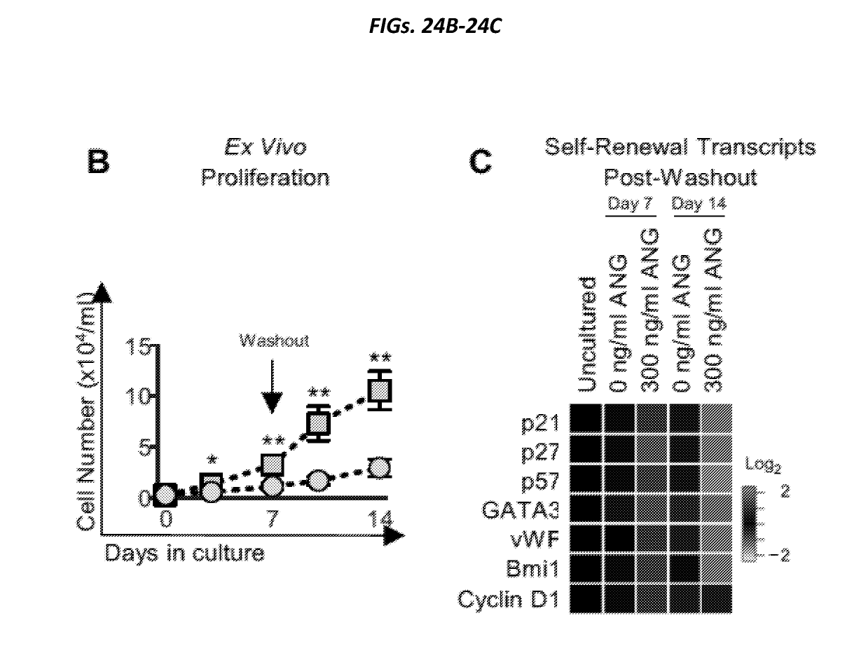

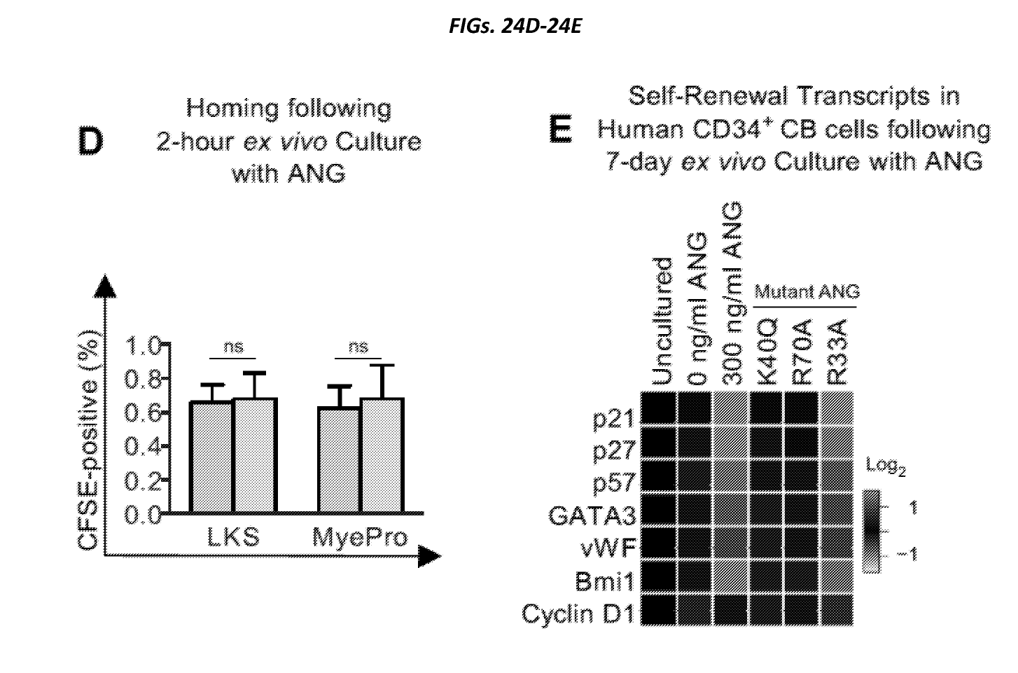

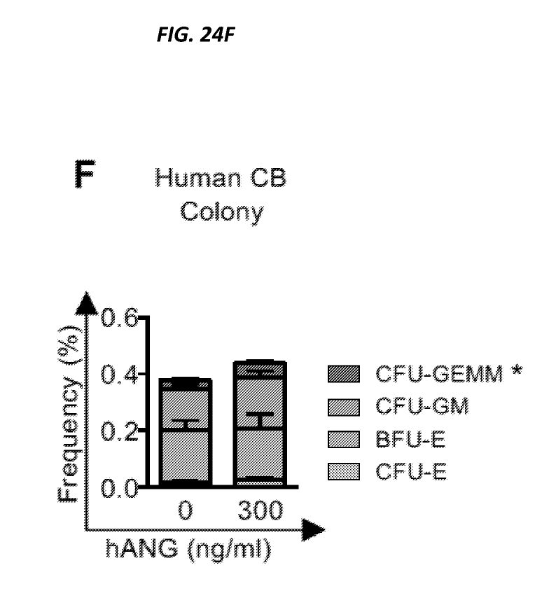

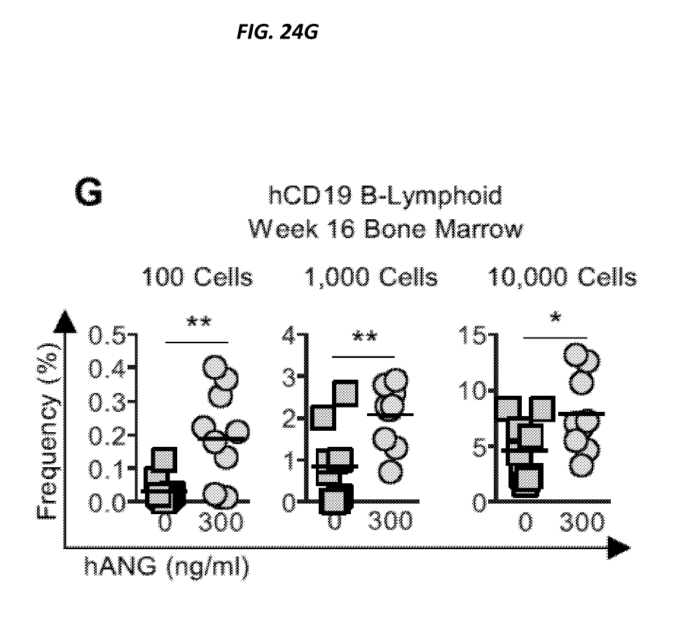

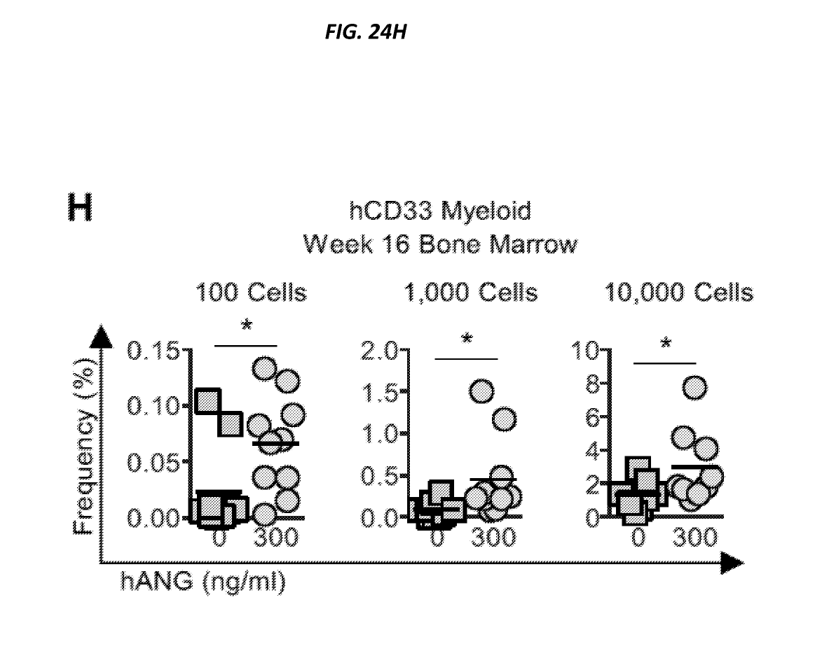

To test whether restriction of proliferation would enhance transplantation efficiency, we competitively transplanted LT-HSCs that were either freshly isolated or had been cultured with or without 300 ng/ml ANG for 2 hours. Significantly, treatment with ANG led to a dramatic increase in multi-lineage post-transplant reconstitution over 24 weeks (FIG. 23C). A similar enhancement in transplant efficiency was observed with LT-HSCs cultured with ANG for 7 days (FIG. 24A) Enhanced regeneration was observed over 16 weeks upon secondary transplant without further ANG treatment (FIG. 23D). Significantly, removal of ANG from the media after 7 days in culture did not induce proliferation (FIG. 24B) and enhanced levels of pro-self-renewal transcripts were retained (FIG. 24C). To confirm that improved reconstitution is not due to enhanced homing of ANG-treated cells, we transplanted ANG-treated, CFSE-labeled CD45.2 Lin− cells into irradiated CD45.1 recipients, and found no difference in homing capability, as indicated by a similar number of CFSE-positive LKS cells and myeloid-restricted progenitors in the BM 16 hours post-transplant (FIG. 24D). Importantly, treatment of Ang−/−

LT-HSCs with exogenous ANG ameliorated post-transplant reconstitution defect of Ang−/− cells, and led to enhanced reconstitution over WT cells by week 16 (FIG. 23E). Together, these data demonstrate that treatment of LT-HSCs with exogenous ANG significantly enhances their regenerative capabilities upon relatively short exposure, and this effect is long-lasting.

ANG Improves Regeneration of Human Cells

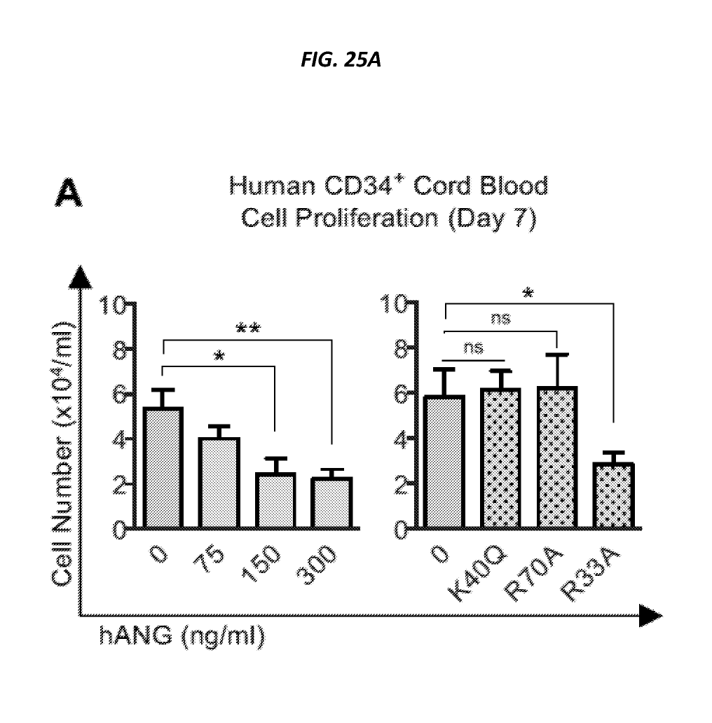

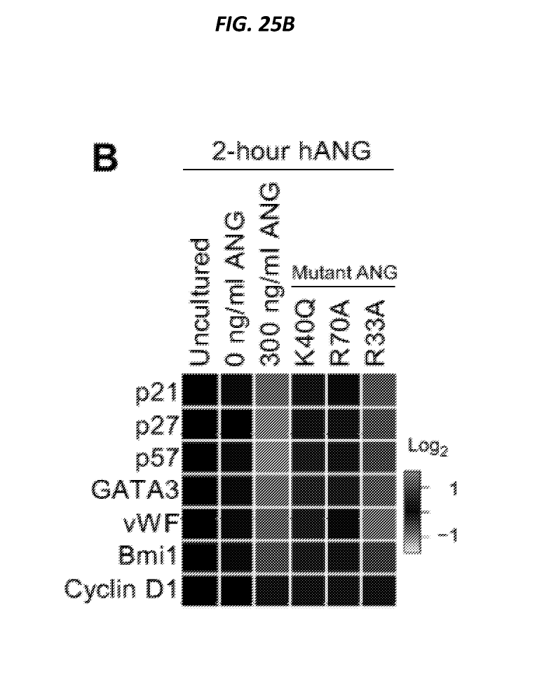

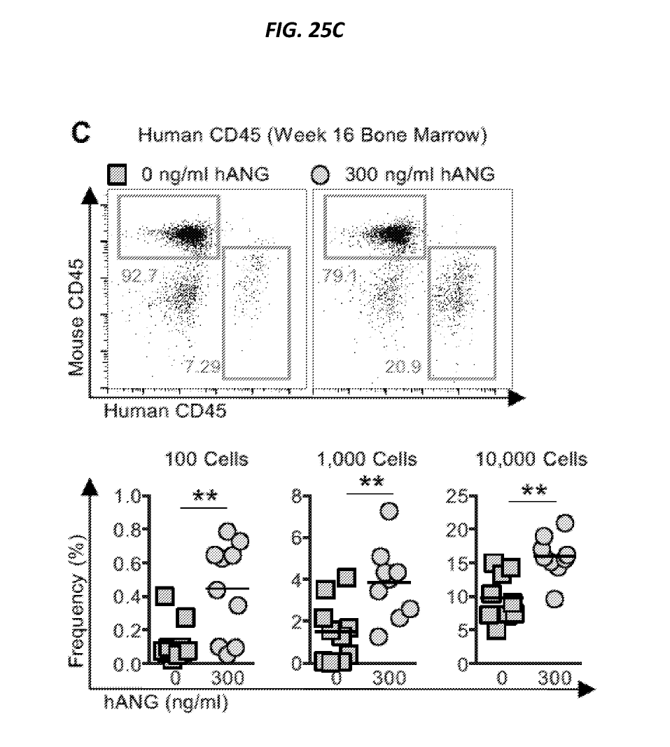

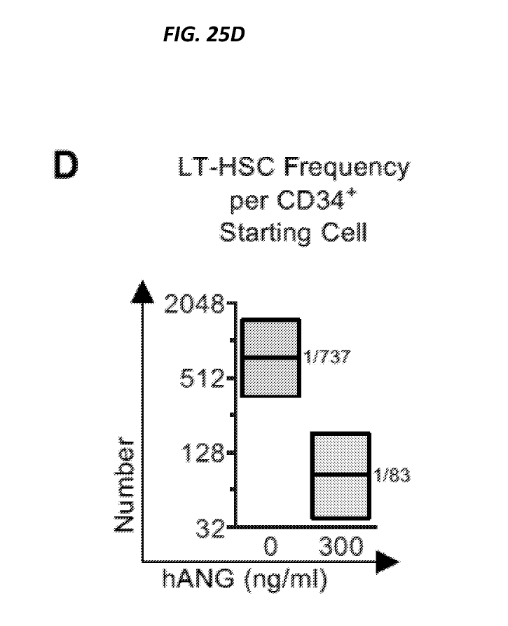

Given that ANG significantly improved transplantation efficiency of mouse LT-HSCs, we next examined whether human ANG has similar pro-regenerative capabilities in human cells. Consistent with the anti-proliferative effect of ANG on mouse LT-HSCs, treatment with human ANG led to a dose-dependent reduction of human CD34+ CB cell proliferation over 7 days (FIG. 25A) and elevated level of pro-self-renewal transcripts (FIG. 25E), whereas ANG variants that are defective in its ribonucleolytic activity (K40Q) or in receptor binding (R70A) were inactive (FIGS. 25A, 24E). Interestingly, R33A ANG, despite having a defective nuclear localization sequence, recapitulated the effect of WT ANG in restricting proliferation and enhancing self-renewal signature (FIGS. 25A, 24E). It is significant to note that a 2 hour exposure to human ANG is adequate for CD34+ human CB cells to up-regulate pro-self-renewal genes (FIG. 25B), which greatly enhances the translational capability of ANG in improving SCT. The fact that R33A ANG variant is as active as WT ANG points to the dispensable role of nuclear ANG in HSPC, reinforcing the finding that cytoplasmic localization of ANG is important in preservation of HSPC stemness. Further, ANG treatment of CB cells led to slightly elevated numbers of primitive colonies (FIG. 24F). Together, these data importantly indicate that in vivo properties of mouse ANG faithfully translate in a human setting, and suggest that the cellular mechanisms underlying mouse HSC regeneration may also translate into human cells.

To assess whether ANG improves transplantation efficiency of human cells, we transplanted CD34+ CB cells that had been cultured for 2 hours in the presence or absence of ANG into NSG mice at limit dilution and found that treatment with ANG led to elevated frequencies of human CD45+ cells across all doses examined in BM 16 weeks post-transplant (FIG. 25C). Importantly, enhanced regeneration was multi-lineage, as confirmed by the presence of both CD19 B-lymphoid cells and CD33 myeloid cells in BM (FIG. 24G-24H). Remarkably, calculated LT-HSC frequency was 8.9-fold higher in ANG-treated human CD34+ CB cells relative to untreated cells (FIG. 25D). Together, these data highlight the translational capacity of ANG in preservation and expansion of clinically-relevant human cells for transplantation.

### Example 9

The inventors have made several important discoveries. First, ANG has a cell context-specific role in regulating proliferation of HSPC versus myeloid-restricted progenitor cells: while promoting quiescence in the former, ANG stimulates proliferation in the latter. Second, recombinant ANG recapitulates the growth suppressive properties in vivo, and can remarkably improve post-transplant reconstitution of mouse LT-HSCs and human CD34+ CB cells in vivo. Previous studies have identified numerous factors that expand stem cell number in vivo by promoting cell proliferation (Boitano et al., 2010; Delaney et al., 2010; Fares et al., 2014; Frisch et al., 2009; Himburg et al., 2010; Hoggatt et al., 2009; North et al., 2007). However, it has been noted that cycling HSPC engraft less well upon transplantation and undergo faster exhaustion (Nakamura-Ishizu et al., 2014; Passegue et al., 2005), likely as a consequence of more active cycling, differentiation, and loss of stemness. Herein, the inventors demonstrate an improvement in regeneration by dichotomously restricting cell proliferation of more primitive HSPC while enabling increased proliferation of more mature myeloid-restricted progenitor cells. The success of SCT depends upon rapid reconstitution of mature blood cell pools to avoid infections and bleeding complications and long-term generation of mature cells from a durable cell source (Doulatov et al., 2012; Smith and Wagner, 2009). These two functions are provided by progenitor and stem cell populations, respectively.

Third, the ability of ANG to serve as a radio-mitigant is also of considerable interest, particularly given its in a model of IR injury to prevent IR injury and ability to rescue animals when administered 24 hours post-irradiation injury. Translation of this ability to humans to reduce mortality following radiation exposure is of considerable significance. Currently, there are no FDA-approved drugs to treat severely irradiated individuals (Singh et al., 2015). A number of hematopoietic growth factors have been shown in various animal models to mitigate hematopoietic syndrome of acute radiation syndrome, however only pleiotrophin has been demonstrated to improve survival when administered 24 hours post-irradiation (Himburg et al., 2014), an efficacy requirement mandated by The Radiation and Nuclear Countermeasures Program at the National Institute of Allergy and Infectious Diseases. Moreover, current standard-of-care approaches, including granulocyte colony-stimulating factor (G-CSF) and its derivatives, target a limited progenitor cell pool and requires repeated doses to combat radiation-induced neutropenia (Singh et al., 2015). In this regard, the invention herein discovered that ANG can be used as a medical countermeasure for radiation exposure, as in a mouse model, only three ANG treatments are needed for improved animal survival, even if started 24 hours after a lethal radiation (12.0 Gy) dose.

A fourth important finding is that the technology herein identified a novel RNA-based mechanism by which hematopoiesis is regulated. Importantly, ANG promotes tiRNA production in LKS cells, in association with enhanced stemness in vivo and in vivo. Further, the invention here demonstrated that increased tiRNA production results in reduced levels of global protein synthesis in HSPC. In contrast, ANG stimulates rRNA transcription in myeloid-restricted progenitors, but not in HSPC, leading to increased protein synthesis and proliferation.

The discoveries herein are of particular importance given recent reports demonstrating tight regulation of protein synthesis in hematopoiesis, with HSCs demonstrating a reduced rate of protein synthesis relative to more lineage-restricted cell types (Signer et al., 2014). Further, a number of mutations or defects in ribosome function or protein synthesis have been shown to either promote or resist malignant hematopoiesis (Cai et al., 2015; Narla and Ebert, 2010).

Modulating tiRNA to alter protein synthesis and cell fate is unique among prior reports of regulatory mechanisms and is of particular interest because of its ability to be affected by a cell exogenous source. The notion that tiRNA can be cell state-specific in regulating hematopoiesis offers the possibility that similar distinct mechanisms may apply to other tissue types. This is of considerable biologic and, potentially, therapeutic interest.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated can be further modified to incorporate features shown in any of the other embodiments disclosed herein.

### REFERENCES

1. Broxmeyer, H. E., Hoggatt, J., O'Leary, H. A., Mantel, C., Chitteti, B. R., Cooper, S., Messina-Graham, S., Hangoc, G., Farag, S., Rohrabaugh, S. L., et al. (2012). Dipeptidylpeptidase 4 negatively regulates colony-stimulating factor activity and stress hematopoiesis. Nature medicine 18, 1786-1796.
2. Bruscia, E. M., Ziegler, E. C., Price, J. E., Weiner, S., Egan, M. E., and Krause, D. S. (2006). Engraftment of donor-derived epithelial cells in multiple organs following bone marrow transplantation into newborn mice. Stem cells 24, 2299-2308.
3. Byrd, D. T., and Kimble, J. (2009). Scratching the niche that controls Caenorhabditis elegans germline stem cells. Seminars in cell & developmental biology 20, 1107-1113.
4. Calvi, L. M., Adams, G. B., Weibrecht, K. W., Weber, J. M., Olson, D. P., Knight, M. C., Martin, R. P., Schipani, E., Divieti, P., Bringhurst, F. R., et al. (2003). Osteoblastic cells regulate the hematopoietic stem cell niche. Nature 425, 841-846.
5. Chakkalakal, J. V., Jones, K. M., Basson, M. A., and Brack, A. S. (2012). The aged niche disrupts muscle stem cell quiescence. Nature 490, 355-360.
6. Chen, K. H., Boettiger, A. N., Moffitt, J. R., Wang, S., and Zhuang, X. (2015). RNA imaging. Spatially resolved, highly multiplexed RNA profiling in single cells. Science 348, aaa6090.
7. Ding, L., and Morrison, S. J. (2013). Haematopoietic stem cells and early lymphoid progenitors occupy distinct bone marrow niches. Nature 495, 231-235.
8. Ding, L., Saunders, T. L., Enikolopov, G., and Morrison, S. J. (2012). Endothelial and perivascular cells maintain hematopoietic stem cells. Nature 481, 457-462.
9. Espina, V., Wulfkuhle, J. D., Calvert, V. S., VanMeter, A., Zhou, W., Coukos, G., Geho, D. H., Petricoin, E. F., 3rd, and Liotta, L. A. (2006). Laser-capture microdissection. Nature protocols 1, 586-603.
10. Forsberg, E. C., Passegue, E., Prohaska, S. S., Wagers, A. J., Koeva, M., Stuart, J. M., and Weissman, I. L. (2010). Molecular signatures of quiescent, mobilized and leukemia-initiating hematopoietic stem cells. PloS one 5, e8785.
11. Fuentealba, L. C., Obernier, K., and Alvarez-Buylla, A. (2012). Adult neural stem cells bridge their niche. Cell Stem Cell 10, 698-708.
12. Greenbaum, A., Hsu, Y. M., Day, R. B., Schuettpelz, L. G., Christopher, M. J., Borgerding, J. N., Nagasawa, T., and Link, D. C. (2013). CXCL12 in early mesenchymal progenitors is required for hematopoietic stem-cell maintenance. Nature 495, 227-230.
13. Greenway, M. J., Andersen, P. M., Russ, C., Ennis, S., Cashman, S., Donaghy, C., Patterson, V., Swingler, R., Kieran, D., Prehn, J., et al. (2006). ANG mutations segregate with familial and ‘sporadic’ amyotrophic lateral sclerosis. Nature genetics 38, 411-413.
14. Hoggatt, J., Mohammad, K. S., Singh, P., Hoggatt, A. F., Chitteti, B. R., Speth, J. M., Hu, P., Poteat, B. A., Stilger, K. N., Ferraro, F., et al. (2013). Differential stem- and progenitor-cell trafficking by prostaglandin E2. Nature 495, 365-369.
15. Hsu, Y. C., Li, L., and Fuchs, E. (2014). Emerging interactions between skin stem cells and their niches. Nature medicine 20, 847-856.
16. Huang, R. P., Ozawa, M., Kadomatsu, K., and Muramatsu, T. (1990). Developmentally regulated expression of embigin, a member of the immunoglobulin superfamily found in embryonal carcinoma cells. Differentiation 45, 76-83.
17. Huang, R. P., Ozawa, M., Kadomatsu, K., and Muramatsu, T. (1993). Embigin, a member of the immunoglobulin superfamily expressed in embryonic cells, enhances cell-substratum adhesion. Dev Biol 155, 307-314.
18. Johnson, W. E., Li, C., and Rabinovic, A. (2007). Adjusting batch effects in microarray expression data using empirical Bayes methods. Biostatistics 8, 118-127. 26
19. Kalajzic, Z., Liu, P., Kalajzic, I., Du, Z., Braut, A., Mina, M., Canalis, E., and Rowe, D. W. (2002). Directing the expression of a green fluorescent protein transgene in differentiated osteoblasts: comparison between rat type I collagen and rat osteocalcin promoters. Bone 31, 654-660.
20. Kharchenko, P., Silberstein, L., and Scadden, D. T. (2014). Bayseian approach to single cell differential expression analysis. Nature Methods.
21. Kim, J. E., Nakashima, K., and de Crombrugghe, B. (2004). Transgenic mice expressing a ligand inducible cre recombinase in osteoblasts and odontoblasts: a new tool to examine physiology and disease of postnatal bone and tooth. The American journal of pathology 165, 1875-1882.
22. Kishimoto, K., Liu, S., Tsuji, T., Olson, K. A., and Hu, G. F. (2005). Endogenous angiogenin in endothelial cells is a general requirement for cell proliferation and angiogenesis. Oncogene 24, 445-456.
23. Kode, A., Manavalan, J. S., Mosialou, I., Bhagat, G., Rathinam, C. V., Luo, N., Khiabanian, H., Lee, A., Murry, V. V., Friedman, R., et al. (2014). Leukaemogenesis induced by an activating beta-catenin mutation in osteoblasts. Nature 506, 240-244.
24. Krause, D. S., Fulzele, K., Catic, A., Sun, C. C., Dombkowski, D., Hurley, M. P., Lezeau, S., Attar, E., Wu, J. Y., Lin, H. Y., et al. (2013). Differential regulation of myeloid leukemias by the bone marrow microenvironment. Nature medicine 19, 1513-1517.
25. Kunisaki, Y., Bruns, I., Scheiermann, C., Ahmed, J., Pinho, S., Zhang, D., Mizoguchi, T., Wei, Q., Lucas, D., Ito, K., et al. (2013). Arteriolar niches maintain hematopoietic stem cell quiescence. Nature 502, 637-643.
26. Lain, E., Carnejac, S., Escher, P., Wilson, M. C., Lomo, T., Gajendran, N., and Brenner, H. R. (2009). A novel role for embigin to promote sprouting of motor nerve terminals at the neuromuscular junction. The Journal of biological chemistry 284, 8930-8939.
27. Lee, J. H., Daugharthy, E. R., Scheiman, J., Kalhor, R., Yang, J. L., Ferrante, T. C., Terry, R., Jeanty, S. S., Li, C., Amamoto, R., et al. (2014). Highly multiplexed subcellular RNA sequencing in situ. Science 343, 1360-1363.
28. Li, L., and Clevers, H. (2010). Coexistence of quiescent and active adult stem cells in mammals. Science 327, 542-545.
29. Lo Celso, C., Fleming, H. E., Wu, J. W., Zhao, C. X., Miake-Lye, S., Fujisaki, J., Cote, D., Rowe, D. W.,
30. Lin, C. P., and Scadden, D. T. (2009). Live-animal tracking of individual hematopoietic stem/progenitor cells in their niche. Nature 457, 92-96.
31. Lovatt, D., Ruble, B. K., Lee, J., Dueck, H., Kim, T. K., Fisher, S., Francis, C., Spaethling, J. M., Wolf, J. A., Grady, M. S., et al. (2014). Transcriptome in vivo analysis (TIVA) of spatially defined single cells in live tissue. Nat Methods 11, 190-196.
32. Mendelson, A., and Frenette, P. S. (2014). Hematopoietic stem cell niche maintenance during homeostasis and regeneration. Nature medicine 20, 833-846.
33. Mendez-Ferrer, S., Michurina, T. V., Ferraro, F., Mazloom, A. R., Macarthur, B. D., Lira, S. A., Scadden, D. T., Ma'ayan, A., Enikolopov, G. N., and Frenette, P. S. (2010). Mesenchymal and hematopoietic stem cells form a unique bone marrow niche. Nature 466, 829-834.
34. Mizoguchi, T., Pinho, S., Ahmed, J., Kunisaki, Y., Hanoun, M., Mendelson, A., Ono, N., Kronenberg, H. M., and Frenette, P. S. (2014). Osterix marks distinct waves of primitive and definitive stromal progenitors during bone marrow development. Developmental cell 29, 340-349.
35. Moore, K. A., and Lemischka, I. R. (2006). Stem cells and their niches. Science 311, 1880-1885.
36. Morrison, S. J., and Scadden, D. T. (2014). The bone marrow niche for hematopoietic stem cells. Nature 505, 327-334.
37. Novick, D., Kim, S., Kaplanski, G., and Dinarello, C. A. (2013). Interleukin-18, more than a Th1 cytokine. Seminars in immunology 25, 439-448.
38. Okamura, H., Tsutsi, H., Komatsu, T., Yutsudo, M., Hakura, A., Tanimoto, T., Torigoe, K., Okura, T., Nukada, Y., Hattori, K., et al. (1995). Cloning of a new cytokine that induces IFN-gamma production by T cells. Nature 378, 88-91.27
39. Paic, F., Igwe, J. C., Nori, R., Kronenberg, M. S., Franceschetti, T., Harrington, P., Kuo, L., Shin, D. G., Rowe, D. W., Harris, S. E., et al. (2009). Identification of differentially expressed genes between osteoblasts and osteocytes. Bone 45, 682-692.
40. Patel, A. P., Tirosh, I., Trombetta, J. J., Shalek, A. K., Gillespie, S. M., Wakimoto, H., Cahill, D. P., Nahed, B. V., Curry, W. T., Martuza, R. L., et al. (2014). Single-cell RNA-seq highlights intratumoral heterogeneity in primary glioblastoma. Science 344, 1396-1401.
41. Pridans, C., Holmes, M. L., Polli, M., Wettenhall, J. M., Dakic, A., Corcoran, L. M., Smyth, G. K., and Nutt, S. L. (2008). Identification of Pax5 target genes in early B cell differentiation. Journal of immunology 180, 1719-1728. Raaijmakers, M. H., Mukherjee, S., Guo, S., Zhang, S., Kobayashi, T., Schoonmaker, J. A., Ebert, B. L. Al-Shahrour, F., Hasserjian, R. P., Scadden, E. O., et al. (2010). Bone progenitor dysfunction induces myelodysplasia and secondary leukaemia. Nature 464, 852-857.
42. Scadden, D. T. (2014). Nice neighborhood: emerging concepts of the stem cell niche. Cell 157, 41-50.
43. Shalek, A. K., Satija, R., Shuga, J., Trombetta, J. J., Gennert, D., Lu, D., Chen, P., Gertner, R. S., Gaublomme, J. T., Yosef, N., et al. (2014). Single-cell RNA-seq reveals dynamic paracrine control of cellular variation. Nature 510, 363-369.
44. Shapiro, E., Biezuner, T., and Linnarsson, S. (2013). Single-cell sequencing-based technologies will revolutionize whole-organism science. Nature reviews Genetics 14, 618-630.
45. Sugama, S., and Conti, B. (2008). Interleukin-18 and stress. Brain research reviews 58, 85-95.
46. Sugimura, R., He, X. C., Venkatraman, A., Arai, F., Box, A., Semerad, C., Haug, J. S., Peng, L., Zhong, X. B., Suda, T., et al. (2012). Noncanonical Wnt signaling maintains hematopoietic stem cells in the niche. Cell 150, 351-365.
47. Tang, F., Barbacioru, C., Wang, Y., Nordman, E., Lee, C., Xu, N., Wang, X., Bodeau, J., Tuch, B. B., Siddiqui, A., et al. (2009). mRNA-Seq whole-transcriptome analysis of a single cell. Nat Methods 6, 377-382.
48. Yu, V. W., Saez, B., Cook, C., Lotinun, S., Pardo-Saganta, A., Wang, Y. H., Lymperi, S., Ferraro, F., Raaijmakers, M. H., Wu, J. Y., et al. (2015). Specific bone cells produce DLL4 to generate thymusseeding progenitors from bone marrow. The Journal of experimental medicine 212, 759-774.
49. Zhang, J., Niu, C., Ye, L., Huang, H., He, X., Tong, W. G., Ross, J., Haug, J., Johnson, T., Feng, J. Q., et al. (2003). Identification of the hematopoietic stem cell niche and control of the niche size. Nature 425, 836-841.
50. Zhu, X., Hill, R. A., Dietrich, D., Komitova, M., Suzuki, R., and Nishiyama, A. (2011). Age-dependent fate and lineage restriction of single NG2 cells. Development 138, 745-753.
51. Boitano, A. E., Wang, J., Romeo, R., Bouchez, L. C., Parker, A. E., Sutton, S. E., Walker, J. R., Flaveny, C. A., Perdew, G. H., Denison, M. S., et al. (2010). Aryl hydrocarbon receptor antagonists promote the expansion of human hematopoietic stem cells. Science 329, 1345-1348.
52. Cabezas-Wallscheid, N., Klimmeck, D., Hansson, J., Lipka, D. B., Reyes, A., Wang, Q., Weichenhan, D., Lier, A., von Paleske, L., Renders, S., et al. (2014). Identification of regulatory networks in HSCs and their immediate progeny via integrated proteome, transcriptome, and DNA methylome analysis. Cell Stem Cell 15, 507-522.
53. Cai, X., Gao, L., Teng, L., Ge, J., Oo, Z. M., Kumar, A. R., Gilliland, D. G., Mason, P. J., Tan, K., and Speck, N. A. (2015). Runx1 Deficiency Decreases Ribosome Biogenesis and Confers Stress Resistance to Hematopoietic Stem and Progenitor Cells. Cell Stem Cell 17, 165-177.
54. Cheng, T., Rodrigues, N., Shen, H., Yang, Y., Dombkowski, D., Sykes, M., and Scadden, D. T. (2000). Hematopoietic stem cell quiescence maintained by p21cip1/waf1. Science 287, 1804-1808.
55. Delaney, C., Heimfeld, S., Brashem-Stein, C., Voorhies, H., Manger, R. L., and Bernstein, I.D. (2010). Notch-mediated expansion of human cord blood progenitor cells capable of rapid myeloid reconstitution. Nat. Med. 16, 232-236.
56. Dhahbi, J. M., Spindler, S. R., Atamna, H., Yamakawa, A., Boffelli, D., Mote, P., and Martin, D. I. (2013). 5′ tRNA halves are present as abundant complexes in serum, concentrated in blood cells, and modulated by aging and calorie restriction. BMC Genomics 14, 298-312.
57. Doulatov, S., Notta, F., Laurenti, E., and Dick, J. E. (2012). Hematopoiesis: a human perspective. Cell Stem Cell 10, 120-136.
58. Emara, M. M., Ivanov, P., Hickman, T., Dawra, N., Tisdale, S., Kedersha, N., Hu, G. F., and Anderson, P. (2010). Angiogenin-induced tRNA-derived stress-induced RNAs promote stress-induced stress granule assembly. J. Biol. Chem. 285, 10959-10968.
59. Fares, I., Chagraoui, J., Gareau, Y., Gingras, S., Ruel, R., Mayotte, N., Csaszar, E., Knapp, D. J., Miller, P., Ngom, M., et al. (2014). Cord blood expansion. Pyrimidoindole derivatives are agonists of human hematopoietic stem cell self-renewal. Science 345, 1509-1512.
60. Fett, J. W., Strydom, D. J., Lobb, R. R., Alderman, E. M., Bethune, J. L., Riordan, J. F., and Vallee, B. L. (1985). Isolation and characterization of angiogenin, an angiogenic protein from human carcinoma cells. Biochemstry 24, 5480-5486.
61. Frelin, C., Herrington, R., Janmohamed, S., Barbara, M., Tran, G., Paige, C. J., Benveniste, P., Zuniga-Pflucker, J. C., Souabni, A., Busslinger, M., et al. (2013). GATA-3 regulates the self-renewal of long-term hematopoietic stem cells. Nat. Immunol. 14, 1037-1044.
62. Frisch, B. J., Porter, R. L., Gigliotti, B. J., Olm-Shipman, A. J., Weber, J. M., O'Keefe, R. J., Jordan, C. T., and Calvi, L. M. (2009). In vivo prostaglandin E2 treatment alters the bone marrow microenvironment and preferentially expands short-term hematopoietic stem cells. Blood 114, 4054-4063.
63. Fu, H., Feng, J., Liu, Q., Sun, F., Tie, Y., Zhu, J., Xing, R., Sun, Z., and Zheng, X. (2009). Stress induces tRNA cleavage by angiogenin in mammalian cells. FEBS Lett. 583, 437-442.
64. Himburg, H. A., Muramoto, G. G., Daher, P., Meadows, S. K., Russell, J. L., Doan, P., Chi, J. T., Salter, A. B., Lento, W. E., Reya, T., et al. (2010). Pleiotrophin regulates the expansion and regeneration of hematopoietic stem cells. Nat. Med. 16, 475-482.
65. Himburg, H. A., Yan, X., Doan, P. L., Quarmyne, M., Micewicz, E., McBride, W., Chao, N.J., Slamon, D. J., and Chute, J. P. (2014). Pleiotrophin mediates hematopoietic regeneration via activation of RAS. J. Clin. Invest. 124, 4753-4758.
66. Hoggatt, J., Singh, P., Sampath, J., and Pelus, L. M. (2009). Prostaglandin E2 enhances hematopoietic stem cell homing, survival, and proliferation. Blood 113, 5444-5455.
67. Hooper, L. V., Stappenbeck, T. S., Hong, C. V., and Gordon, J. I. (2003). Angiogenins: a new class of microbicidal proteins involved in innate immunity. Nat. Immunol. 4, 269-273.
68. Ibaragi, S., Yoshioka, N., Kishikawa, H., Hu, J. K., Sadow, P. M., Li, M., and Hu, G.-f. (2009). Angiogenin-stimulated Ribosomal RNA Transcription Is Essential for Initiation and Survival of AKT-induced Prostate Intraepithelial Neoplasia. Mol. Cancer Res. 7, 415-424.
69. Ito, K., and Suda, T. (2014). Metabolic requirements for the maintenance of self-renewing stem cells. Nat. Rev. Mol. Cell. Biol. 15, 243-256.
70. Ivanov, P., Emara, M. M., Villen, J., Gygi, S. P., and Anderson, P. (2011). Angiogenin-Induced tRNA Fragments Inhibit Translation Initiation. Mol. Cell 43, 613-623.
71. Kent, D. G., Copley, M. R., Benz, C., Wohrer, S., Dykstra, B. J., Ma, E., Cheyne, J., Zhao, Y., Bowie, M. B., Zhao, Y., et al. (2009). Prospective isolation and molecular characterization of hematopoietic stem cells with durable self-renewal potential. Blood 113, 6342-6350.
72. Kieran, D., Sebastia, J., Greenway, M. J., King, M. A., Connaughton, D., Concannon, C. G., Fenner, B., Hardiman, O., and Prehn, J. H. (2008). Control of motoneuron survival by angiogenin. J. Neurosci. 28, 14056-14061.
73. Kishimoto, K., Liu, S., Tsuji, T., Olson, K. A., and Hu, G. F. (2005). Endogenous angiogenin in endothelial cells is a general requirement for cell proliferation and angiogenesis. Oncogene 24, 45-456.
74. Lee, F., Shapiro, R., and Vallee, B. (1989). Tight-Binding Inhibition of Angiogenin and Ribonuclease A by Placental Ribonuclease Inhibitor. Biochemistry 1989, 225-230.
75. Li, P., Lahvic, J. L., Binder, V., Pugach, E. K., Riley, E. B., Tamplin, O. J., Panigrahy, D., Bowman, T. V., Barrett, F. G., Heffner, G. C., et al. (2015). Epoxyeicosatrienoic acids enhance embryonic haematopoiesis and adult marrow engraftment. Nature 523, 468-471.
76. Li, S., and Hu, G. F. (2010). Angiogenin-mediated rRNA transcription in cancer and neurodegeneration. Int. J. Biochem. Mol. Biol. 1, 26-35.
77. Li, S., Yu, W., Kishikawa, H., and Hu, G. F. (2010). Angiogenin prevents serum withdrawal-induced apoptosis of P19 embryonal carcinoma cells. FEBS J. 277, 3575-3587.
78. Mantel, C. R., O'Leary, H. A., Chitteti, B. R., Huang, X., Cooper, S., Hangoc, G., Brustovetsky, N., Srour, E. F., Lee, M. R., Messina-Graham, S., et al. (2015) Enhancing Hematopoietic Stem Cell Transplantation Efficacy by Mitigating Oxygen Shock. Cell 161, 1553-1565.
79. Matsumoto, A., Takeishi, S., Kanie, T., Susaki, E., Onoyama, I., Tateishi, Y., Nakayama, K., and Nakayama, K. I. (2011). p57 is required for quiescence and maintenance of adult hematopoietic stem cells. Cell Stem Cell 9, 262-271.
80. Mendelson, A., and Frenette, P. S. (2014). Hematopoietic stem cell niche maintenance during homeostasis and regeneration. Nat. Med. 20, 833-846.
81. Morrison, S. J., and Scadden, D. T. (2014). The bone marrow niche for hematopoietic stem cells. Nature 505, 327-334.
82. Nakamura-Ishizu, A., Takizawa, H., and Suda, T. (2014). The analysis, roles and regulation of quiescence in hematopoietic stem cells. Development 141, 4656-4666.
83. Narla, A., and Ebert, B. L. (2010). Ribosomopathies: human disorders of ribosome dysfunction. Blood 115, 3196-3205.
84. North, T. E., Goessling, W., Walkley, C. R., Lengerke, C., Kopani, K. R., Lord, A. M., Weber, G. J., Bowman, T. V., Jang, I. H., Grosser, T., et al. (2007). Prostaglandin E2 regulates vertebrate hematopoietic stem cell homeostasis. Nature 447, 1007-1011.
85. Orford, K. W., and Scadden, D. T. (2008). Deconstructing stem cell self-renewal: genetic insights into cell-cycle regulation. Nature reviews Genetics 9, 115-128.
86. Park, I., Qian, D., Kiel, M., Becker, M. W., Pihalja, M., Weissman, I. L., Morrison, S. J., and Clarke, M. F. (2003). Bmi-1 is required for maintenance of adult self-renewing hematopoietic stem cells. Nature 423, 302-305.
87. Passegue, E., Wagers, A. J., Giuriato, S., Anderson, W. C., and Weissman, I. L. (2005). Global analysis of proliferation and cell cycle gene expression in the regulation of hematopoietic stem and progenitor cell fates. J. Exp. Med. 202, 1599-1611.
88. Pizzo, E., Sarcinelli, C., Sheng, J., Fusco, S., Formiggini, F., Netti, P., Yu, W., D'Alessio, G., and Hu, G. F. (2013). Ribonuclease/angiogenin inhibitor 1 regulates stress-induced subcellular localization of angiogenin to control growth and survival. J. Cell Sci. 126, 4308-4319.
89. Signer, R. A., Magee, J. A., Salic, A., and Morrison, S. J. (2014). Haematopoietic stem cells require a highly regulated protein synthesis rate. Nature 509, 49-54.
90. Singh, V. K., Newman, V. L., and Seed, T. M. (2015). Colony-stimulating factors for the treatment of the hematopoietic component of the acute radiation syndrome (H-ARS): a review. Cytokine 71, 22-37.
91. Smith, A. R., and Wagner, J. E. (2009). Alternative hematopoietic stem cell sources for transplantation: place of umbilical cord blood. Br. J. Haematol. 147, 246-261.
92. Subramanian, V., Crabtree, B., and Acharya, K. R. (2008). Human angiogenin is a neuroprotective factor and amyotrophic lateral sclerosis associated angiogenin variants affect neurite extension/pathfinding and survival of motor neurons. Hum. Mol. Genet. 17, 130-149.
93. Subramanian, V., and Feng, Y. (2007). A new role for angiogenin in neurite growth and pathfinding: implications for amyotrophic lateral sclerosis. Hum. Mol. Genet. 16, 1445-1453.
94. Tsuji, T., Sun, Y., Kishimoto, K., Olson, K. A., Liu, S., Hirukawa, S., and Hu, G. F. (2005). Angiogenin is translocated to the nucleus of HeLa cells and is involved in ribosomal RNA transcription and cell proliferation. Cancer Res. 65, 1352-1360.
95. Xu, Z. P., Tsuji, T., Riordan, J. F., and Hu, G. F. (2003). Identification and characterization of an angiogenin-binding DNA sequence that stimulates luciferase reporter gene expression. Biochemistry 42, 121-128.
96. Yamasaki, S., Ivanov, P., Hu, G. F., and Anderson, P. (2009). Angiogenin cleaves tRNA and promotes stress-induced translational repression. J. Cell Biol. 185, 35-42.
97. Yoshioka, N., Wang, L., Kishimoto, K., Tsuji, T., and Hu, G. F. (2006). A therapeutic target for prostate cancer based on angiogenin-stimulated angiogenesis and cancer cell proliferation. Proc. Natl. Acad. Sci. USA 103, 14519-14524.
98. Abdouh, M., Facchino, S., Chatoo, W., Balasingam, V., Ferreira, J., and Bernier, G. (2009). BMI1 sustains human glioblastoma multiforme stem cell renewal. J Neurosci 29, 8884-8896.
99. Bryant, P., Zheng, Q., and Pumiglia, K. (2006). Focal adhesion kinase controls cellular levels of p27/Kip1 and p21/Cip1 through Skp2-dependent and -independent mechanisms. Mol Cell Biol 26, 4201-4213.
100. Chakkalakal, J. V., Christensen, J., Xiang, W., Tierney, M. T., Boscolo, F. S., Sacco, A., and Brack, A. S. (2014). Early forming label-retaining muscle stem cells require p27kip1 for maintenance of the primitive state. Development 141, 1649-1659.
101. Ding, J., Ning, B., Gong, W., Wen, W., Wu, K., Liang, J., He, G., Huang, S., Sun, W., Han, T., et al. (2009). Cyclin D1 induction by benzo[a]pyrene-7,8-diol-9,10-epoxide via the phosphatidylinositol 3-kinase/Akt/MAPK- and p70s6k-dependent pathway promotes cell transformation and tumorigenesis. J Biol Chem 284, 33311-33319.
102. Giovannini, C., Gramantieri, L., Minguzzi, M., Fornari, F., Chieco, P., Grazi, G. L., and Bolondi, L. (2012). CDKN1C/P57 is regulated by the Notch target gene Hes' and induces senescence in human hepatocellular carcinoma. Am J Pathol 181, 413-422.
103. McDermott, S. P., Eppert, K., Lechman, E. R., Doedens, M., and Dick, J. E. (2010). Comparison of human cord blood engraftment between immunocompromised mouse strains. Blood 116, 193-200.
104. Mohrin, M., Bourke, E., Alexander, D., Warr, M. R., Barry-Holson, K., Le Beau, M. M., Morrison, C. G., and Passegue, E. (2010). Hematopoietic stem cell quiescence promotes error-prone DNA repair and mutagenesis. Cell Stem Cell 7, 174-185.
105. Purton, L. E., and Scadden, D. T. (2007). Limiting factors in murine hematopoietic stem cell assays. Cell Stem Cell 1, 263-270. Shapiro, R., Harper, J., Fox, E., Jansen, H., Hein, F., and Uhlmann, E. (1988). Expression of Met-(−1) Angiogenin in Escherichia coli: Conversion to the Authentic <Glu-1 Protein. Analytical Biochemistry 175, 450-461.
106. Sheng, J., Yu, W., Gao, X., Xu, Z., and Hu, G. F. (2014). Angiogenin stimulates ribosomal RNA
107. transcription by epigenetic activation of the ribosomal DNA promoter. J Cell Physiol 229, 521-529.
108. Wang, X., Fu, A. Q., McNerney, M. E., and White, K. P. (2014). Widespread genetic epistasis among cancer genes. Nat Commun 5, 4828

1.-27. (canceled)

28. A method for expanding a population of hematopoietic cells in a biological sample, the method comprising contacting the population of hematopoietic cells with an Angiogenin (ANG) protein or ANG agonist, wherein the population comprises primitive hematopoietic stem cells and myeloid restricted progenitors, and wherein the contacting is for a sufficient amount of time to allow for primitive hematopoietic stem cells quiescence and myeloid restricted progenitor proliferation.

29. The method of claim 28, wherein the primitive hematopoietic stem cells are selected from the group of: long-term hematopoietic stem cells (LT-HSCs), short-term hematopoietic stem cells (ST-HSCs), multipotent progenitors (MPPs) or a combination thereof.

30. The method of claim 28, wherein the myeloid restricted progenitor are selected from the group of: common myeloid progenitors (CMPs), common lymphoid progenitors (CLPs), granulocyte-macrophage progenitors (GMPs) and megakaryocyte-erythroid progenitors (MEPs) or a combination thereof.

31. The method of claim 28, wherein the biological sample is selected from the group consisting of cord blood, bone marrow, peripheral blood, amniotic fluid, and placental blood.

32. The method of claim 28, further comprising collecting the population of expanded hematopoietic cells.

33. (canceled)

34. (canceled)

35. A population of hematopoietic cells comprising primitive hematopoietic stem cells and/or myeloid restricted progenitors, or both, in the presence of an exogenous Angiogenin (ANG) protein or exogenous ANG agonist.

36. (canceled)

37. A method of administering a population of hematopoietic cells to a subject, comprising administering an effective amount of the population of hematopoietic cells to the subject, wherein the population of hematopoietic cells have been contacted ex vivo or in vivo with an Angiogenin (ANG) protein or ANG agonist, wherein the population of hematopoietic cells comprises at least one or both of primitive hematopoietic stem cells and myeloid restricted progenitors, and wherein the Angiogenin protein or ANG agonist increases primitive hematopoietic stem cells quiescence and increases myeloid restricted progenitor proliferation.

38. (canceled)

39. (canceled)

40. The method of claim 28, wherein the population of hematopoietic cells are obtained from bone marrow, peripheral blood, cord blood, amniotic fluid, placental blood, embryonic stem cells (ESCs), or induced pluripotent stem cells (iPSCs).

41. The method of claim 28, wherein the population of hematopoietic cells are human.

42. (canceled)

43. The method of claim 37, wherein the population of hematopoietic cells are autologous or allogeneic to the subject.

44. (canceled)

45. The method of claim 28, wherein the population of hematopoietic cells are cultured in presence of the ANG protein or the ANG agonist for any of:
a. at least 2 hrs;
b. about 2 days or more;
c. at least 7 days.

46. (canceled)

47. (canceled)

48. The method of claim 28, wherein the population of hematopoietic cells are cryopreserved prior to, or after, the contacting with ANG protein or ANG agonist.

49. The population of hematopoietic cells of claim 35, wherein the population of hematopoietic cells are cryopreserved in the presence of ANG protein or ANG agonist.

50. The method of claim 37, wherein the subject is selected as being
a. susceptible to, or has decreased levels of hematopoietic stem cells and hematopoietic progenitor cells as compared to a healthy subject;
b. has undergone, or will undergo a bone marrow or stem cell transplantation, or has undergone, or will undergo chemotherapy or radiation therapy;
c. has a disease or disorder selected from the group consisting of: leukemia, lymphoma, myeloma, solid tumor, a blood disorder, myelodysplasia or an immune disorder; or
d. has anemia, sickle cell anemia, thalassemia or aplastic anemia.

51. (canceled)

52. (canceled)

53. (canceled)

54. The method of claim 28, wherein the ANG protein is human ANG protein, or a functional fragment thereof, and is selected from any of:
a. a polypeptide having at least 85% amino acid sequence identity to SEQ ID NO: 1 or a functional fragment thereof with a biological activity of at least 80% of human ANG protein to increase hematopoietic reconstitution in a human subject;
b. a human recombinant ANG polypeptide;
c. a polypeptide comprising at least amino acids 1-147 of SEQ ID NO 1;
d. a polypeptide having at least 85% amino acid sequence identity to SEQ ID NO: 1 and comprises the mutation K33A;
e. a polypeptide comprising an amino acid sequence of at least 80% of human ANG protein of SEQ ID NO: 1;
f. a polypeptide comprising at least 80%, or at least 90%, or at least 95%, or at least 98% sequence identity to amino acids 1-147 of SEQ ID NO 1.

55.-65. (canceled)

66. A method comprising administering an effective amount of an Angiogenin (ANG) protein or Angiogenin agonist to the subject, wherein the subject is selected from any of:
a. a subject that has been exposed to ionizing radiation, or has a radiation injury;
b. a subject at risk of being exposed to ionizing radiation, or at risk of having a radiation injury;
c. a subject that has undergone, or will undergo, or is undergoing a transplantation of hematopoietic stem cells or hematopoietic progenitor cells, or both;
d. a subject with a disease or disorder characterized by decreased in vivo levels of hematopoietic stem cells and progenitor cells, or decreased in vivo hematopoietic reconstitution;
e. a subject in need of increased hematopoietic reconstitution, or has decreased levels of hematopoietic cells and hematopoietic cells as compared to a healthy subject.

67. (canceled)

68. (canceled)

69. The method of claim 66, wherein the subject of any of (a) to (e) will undergo or has undergone any of the following:
a. radiation therapy for the treatment of a disease or disorder;
b. radiation therapy as part of an ablative regimen for hematopoietic stem and progenitor cell or bone marrow transplant or chemotherapy;
c. total body radiation; or
d. exposure to a radiation accident or chemotherapy.

70. (canceled)

71. (canceled)

72. (canceled)

73. The method claim of 66, wherein the hematopoietic stem and progenitor cells are selected from the group consisting of Long-term hematopoietic stem cells (LT-HSCs), Short-term hematopoietic stem cells (ST-HSCs), Multipotent progenitor cells (MPPs), Common myeloid progenitor (CMPs), CLPs, Granulocyte-macrophage progenitor (GMPs) and Megakaryocyte-erythroid progenitor (MEPs).

74. (canceled)

75. (canceled)

76. (canceled)

77. The method of claim 66, wherein the ANG protein or ANG agonist is administered to the subject at any of the following times:
a. prior to, during or after exposure, or a combination thereof, to an ionizing radiation;
b. between 12 hours and 3 days prior to the subject being exposed to an ionizing radiation;
c. immediately after the exposure to ionizing radiation;
d. about 24 hrs before exposure to ionizing radiation;
e. about 24 hrs after exposure to ionizing radiation; or
f. for at least 3 days or more.

78. (canceled)

79. (canceled)

80. (canceled)

81. The method of claim 66, wherein the administration of the effective amount of ANG protein or ANG agonist results in any one or more of:
a. an increase in primitive hematopoietic stem cell quiescence as compared to in absence of administration;
b. an increase in myeloid restricted progenitor proliferation as compared to in absence of administration; or
c. an increase in hematopoietic reconstitution as compared to in absence of administration.

82. The method of claim 66, wherein ANG protein is a human ANG protein or a functional fragment thereof, and is selected from any of:
a. a polypeptide having at least 85% amino acid sequence identity to SEQ ID NO: 1 or a functional fragment thereof with a biological activity of at least 80% of human ANG protein to increase hematopoietic reconstitution in a human subject;
b. a human recombinant ANG polypeptide;
c. a polypeptide comprising at least amino acids 1-147 of SEQ ID NO 1;
d. a polypeptide having at least 85% amino acid sequence identity to SEQ ID NO: 1 and comprises the mutation K33A;
e. a polypeptide comprising an amino acid sequence of at least 80% of human ANG of SEQ ID NO: 1;
f. a polypeptide comprising at least 80%, or at least 90%, or at least 95%, or at least 98% sequence identity to amino acids 1-147 of SEQ ID NO 1.

83.-92. (canceled)

93. The population of hematopoietic cells of claim 35, wherein the ANG protein or ANG agonist are present in an effective amount to increase quiescence of the primitive hematopoietic cells or increase the proliferation of myeloid restricted cells, or both.

94. The population of hematopoietic cells of claim 35, wherein
the primitive hematopoietic cells are selected from the group, long-term hematopoietic stem cells (LT-HSCs), short-term hematopoietic stem cells (ST-HSCs), multipotent progenitors (MPPs) or a combination thereof, and
the myeloid-restricted progenitor cells are selected from the group, common myeloid progenitors (CMPs), granulocyte-macrophage progenitors (GMPs), megakaryocyte-erythroid progenitors (MEPs) and combination thereof.

95.-103. (canceled)

104. The method of claim 66, wherein the hematopoietic reconstitution is any of: multi-lineage hematopoietic reconstitution, long-term multi-lineage hematopoietic reconstitution, reconstitution of short-term hematopoietic stem cells (ST-HSC) or long-term (LT-HSC) hematopoietic stem cells, or both.
